SEQUENCE LISTING (67 sequences)

SEQ ID NO: 1 - DNA, 10924 bp, Homo sapiens
ccggcccagcggctctcctggcctcgcgctgcacattctctcctggcggcggcgccacctgcagtagcgttcgcccgaacatggcgacacggagcagcaggagggagtcgcgactcccgttcctattcaccctggtcgcactgctgccgcccggagctctctgcgaagtctggacgcagaggctgcacggcggcagcgcgcccttgccccaggaccggggcttcctcgtggtgcagggcgacccgcgcgagctgcggctgtgggcgcgcggggatgccaggggggcgagccgcgcggacgagaagccgctccggaggaaacggagcgctgccctgcagcccgagcccatcaaggtgtacggacaggttagtctgaatgattcccacaatcagatggtggtgcactgggctggagagaaaagcaacgtgatcgtggccttggcccgagatagcctggcattggcgaggcccaagagcagtgatgtgtacgtgtcttacgactatggaaaatcattcaagaaaatttcagacaagttaaactttggcttgggaaataggagtgaagctgttatcgcccagttctaccacagccctgcggacaacaagcggtacatctttgcagacgcttatgcccagtacctctggatcacgtttgacttctgcaacactcttcaaggcttttccatcccatttcgggcagctgatctcctcctacacagtaaggcctccaaccttctcttgggctttgacaggtcccaccccaacaagcagctgtggaagtcagatgactttggccagacctggatcatgattcaggaacatgtcaagtccttttcttggggaattgatccctatgacaaaccaaataccatctacattgaacgacatgaaccctctggctactccactgtcttccgaagtacagatttcttccagtcccgggaaaaccaggaagtgatccttgaggaagtgagagattttcagcttcgggacaagtacatgtttgctacaaaggtggtgcatctcttgggcagtgaacagcagtcttctgtccagctctgggtctcctttggccggaagcccatgagagcagcccagtttgtcacaagacatcctattaatgaatattacatcgcagatgcctccgaggaccaggtgtttgtgtgtgtcagccacagtaacaaccgcaccaatttatacatctcagaggcagaggggctgaagttctccctgtccttggagaacgtgctctattacagcccaggaggggccggcagtgacaccttggtgaggtattttgcaaatgaaccatttgctgacttccaccgagtggaaggattgcaaggagtctacattgctactctgattaatggttctatgaatgaggagaacatgagatcggtcatcacctttgacaaagggggaacctgggagtttcttcaggctccagccttcacgggatatggagagaaaatcaattgtgagctttcccagggctgttcccttcatctggctcagcgcctcagtcagctcctcaacctccagctccggagaatgcccatcctgtccaaggagtcggctccaggcctcatcatcgccactggctcagtgggaaagaacttggctagcaagacaaacgtgtacatctctagcagtgctggagccaggtggcgagaggcacttcctggacctcactactacacatggggagaccacggcggaatcatcacggccattgcccagggcatggaaaccaacgagctaaaatacagtaccaatgaaggggagacctggaaaacattcatcttctctgagaagccagtgtttgtgtatggcctcctcacagaacctggggagaagagcactgtcttcaccatctttggctcgaacaaagagaatgtccacagctggctgatcctccaggtcaatgccacggatgccttgggagttccctgcacagagaatgactacaagctgtggtcaccatctgatgagcgggggaatgagtgtttgctgggacacaagactgttttcaaacggcggaccccccatgccacatgcttcaatggagaggactttgacaggccggtggtcgtgtccaactgctcctgcacccgggaggactatgagtgtgacttcggtttcaagatgagtgaagatttgtcattagaggtttgtgttccagatccggaattttctggaaagtcatactcccctcctgtgccttgccctgtgggttctacttacaggagaacgagaggctaccggaagatttctggggacacttgtagcggaggagatgttgaagcgcgactggaaggagagctggtcccctgtcccctggcagaagagaacgagttcattctgtatgctgtgaggaaatccatctaccgctatgacctggcctcgggagccaccgagcagttgcctctcaccgggctacgggcagcagtggccctggactttgactatgagcacaactgtttgtattggtccgacctggccttggacgtcatccagcgcctctgtttgaatggaagcacagggcaagaggtgatcatcaattctggcctggagacagtagaagctttggcttttgaacccctcagccagctgctttactgggtagatgcaggcttcaaaaagattgaggtagctaatccagatggcgacttccgactcacaatcgtcaattcctctgtgcttgatcgtcccagggctctggtcctcgtgccccaagagggggtgatgttctggacagactggggagacctgaagcctgggatttatcggagcaatatggatggttctgctgcctatcacctggtgtctgaggatgtgaagtggcccaatggcatctctgtggacgaccagtggatttactggacggatgcctacctggagtgcatagagcggatcacgttcagtggccagcagcgctctgtcattctggacaacctcccgcacccctatgccattgctgtctttaagaatgaaatctactgggatgactggtcacagctcagcatattccgagcttccaaatacagtgggtcccagatggagattctggcaaaccagctcacggggctcatggacatgaagattttctacaaggggaagaacactggaagcaatgcctgtgtgcccaggccatgcagcctgctgtgcctgcccaaggccaacaacagtagaagctgcaggtgtccagaggatgtgtccagcagtgtgcttccatcaggggacctgatgtgtgactgccctcagggctatcagctcaagaacaatacctgtgtcaaagaagagaacacctgtcttcgcaaccagtatcgctgcagcaacgggaactgtatcaacagcatttggtggtgtgactttgacaacgactgtggagacatgagcgatgagagaaactgccctaccaccatctgtgacctggacacccagtttcgttgccaggagtctgggacttgtatcccactgtcctataaatgtgaccttgaggatgactgtggagacaacagtgatgaaagtcattgtgaaatgcaccagtgccggagtgacgagtacaactgcagttccggcatgtgcatccgctcctcctgggtatgtgacggggacaacgactgcagggactggtctgatgaagccaactgtaccgccatctatcacacctgtgaggcctccaacttccagtgccgaaacgggcactgcatcccccagcggtgggcgtgtgacggggatacggactgccaggatggttccgatgaggatccagtcaactgtgagaagaagtgcaatggattccgctgcccaaacggcacttgcatcccatccagcaaacattgtgatggtctgcgtgattgctctgatggctccgatgaacagcactgcgagcccctctgtacgcacttcatggactttgtgtgtaagaaccgccagcagtgcctgttccactccatggtctgtgacggaatcatccagtgccgcgacgggtccgatgaggatgcggcgtttgcaggatgctcccaagatcctgagttccacaaggtatgtgatgagttcggtttccagtgtcagaatggagtgtgcatcagtttgatttggaagtgcgacgggatggatgattgcggcgattattctgatgaagccaactgcgaaaaccccacagaagccccaaactgctcccgctacttccagtttcggtgtgagaatggccactgcatccccaacagatggaaatgtgacagggagaacgactgtggggactggtctgatgagaaggattgtggagattcacatattcttcccttctcgactcctgggccctccacgtgtctgcccaattactaccgctgcagcagtgggacctgcgtgatggacacctgggtgtgcgacgggtaccgagattgtgcagatggctctgacgaggaagcctgccccttgcttgcaaacgtcactgctgcctccactcccacccaacttgggcgatgtgaccgatttgagttcgaatgccaccaaccgaagacgtgtattcccaactggaagcgctgtgacggccaccaagattgccaggatggccgggacgaggccaattgccccacacacagcaccttgacttgcatgagcagggagttccagtgcgaggacggggaggcctgcattgtgctctcggagcgctgcgacggcttcctggactgctcggacgagagcgatgaaaaggcctgcagtgatgagttgactgtgtacaaagtacagaatcttcagtggacagctgacttctctggggatgtgactttgacctggatgaggcccaaaaaaatgccctctgcttcttgtgtatataatgtctactacagggtggttggagagagcatatggaagactctggagacccacagcaataagacaaacactgtattaaaagtcttgaaaccagataccacgtatcaggttaaagtacaggttcagtgtctcagcaaggcacacaacaccaatgactttgtgaccctgaggaccccagagggattgccagatgcccctcgaaatctccagctgtcactccccagggaagcagaaggtgtgattgtaggccactgggctcctcccatccacacccatggcctcatccgtgagtacattgtagaatacagcaggagtggttccaagatgtgggcctcccagagggctgctagtaactttacagaaatcaagaacttattggtcaacactctatacaccgtcagagtggctgcggtgactagtcgtggaataggaaactggagcgattctaaatccattaccaccataaaaggaaaagtgatcccaccaccagatatccacattgacagctatggtgaaaattatctaagcttcaccctgaccatggagagtgatatcaaggtgaatggctatgtggtgaaccttttctgggcatttgacacccacaagcaagagaggagaactttgaacttccgaggaagcatattgtcacacaaagttggcaatctgacagctcatacatcctatgagatttctgcctgggccaagactgacttgggggatagccctctggcatttgagcatgttatgaccagaggggttcgcccacctgcacctagcctcaaggccaaagccatcaaccagactgcagtggaatgtacctggaccggcccccggaatgtggtttatggtattttctatgccacgtcctttcttgacctctatcgcaacccgaagagcttgactacttcactccacaacaagacggtcattgtcagtaaggatgagcagtatttgtttctggtccgtgtagtggtaccctaccaggggccatcctctgactacgttgtagtgaagatgatcccggacagcaggcttccaccccgtcacctgcatgtggttcatacgggcaaaacctccgtggtcatcaagtgggaatcaccgtatgactctcctgaccaggacttgttgtatgcaattgcagtcaaagatctcataagaaagactgacaggagctacaaagtaaaatcccgtaacagcactgtggaatacacccttaacaagttggagcctggcgggaaataccacatcattgtccaactggggaacatgagcaaagattccagcataaaaattaccacagtttcattatcagcacctgatgccttaaaaatcataacagaaaatgatcatgttcttctgttttggaaaagcctggctttaaaggaaaagcattttaatgaaagcaggggctatgagatacacatgtttgatagtgccatgaatatcacagcttaccttgggaatactactgacaatttctttaaaatttccaacctgaagatgggtcataattacacgttcaccgtccaagcaagatgcctttttggcaaccagatctgtggggagcctgccatcctgctgtacgatgagctggggtctggtgcagatgcatctgcaacgcaggctgccagatctacggatgttgctgctgtggtggtgcccatcttattcctgatactgctgagcctgggggtggggtttgccatcctgtacacgaagcaccggaggctgcagagcagcttcaccgccttcgccaacagccactacagctccaggctggggtccgcaatcttctcctctggggatgacctgggggaagatgatgaagatgcccctatgataactggattttcagatgacgtccccatggtgatagcctgaaagagctttcctcactagaaaccaaatggtgtaaatattttatttgataaagatagttgatggtttattttaaaagatgcactttgagttgcaatatgttatttttatatgggccaaaaacaaaaaacaaaaaaaaaaaaaaggaaagaaaggaatgaataaactttgtagtaatcaactgtgaacttcaaaccaggttgattttagtaacccaattgctttgatttgacattaatgtagtcttacagggctgtgcttgctgggcatgcttttacgtctgtgagataatttcggttcagtaaattggccaatctttttatttttctaagacacagaaatgtatttaataaaaacctcgagagagtgatgggtggaaccccttctccttgaaagtgtgtacagatattccattttgtttggatatagtttataggaaagtgtgtggatgtattatggcggaaggtttctttatgttattttgttaatttattgggactctgtgtaaggccaggctttagtggtcattagacaccacatgtgttatgagccccttacccatagggttgggggtgggaagagaagcatatttttttgccattccggaagcaatccatttttattcacttgtgtgtcatgtaatggtctttggcaggagagagcactgagtcattgctggagttcagttcaacagagctgcagcttgggaagccctgtaagcccacagcttcctctcttatattaattgatggaattttactgtatgtgcctctgtacaagatgtagctttgagagctacaaaatgataacactgctttattacacactggtttcattgtcattgcaaaaacttaccctggttgtgggggagagttctagatctgtgccatgatccatacactggctaatagagtacataatttttccattttccattttttgtttttacttactactgaaggatctcagatgtaaaattatgtatttggtttgagatggccacttattgtccttaaaaatccatactgatatatgcagtcattttgaattggacagtgccttctctttttttttctcctcttcttccatctccctcacccatgcccccacccaatctaaagagacagtgctgtacattctcatagagatagagaagatctaaaaagttgagactactcaatccagttaacaacagcaggagcactagagtttgttcatttattctctctgtaaaacaagctgtgctttttttcttctgcctttaaaatgccacccgtgtattcaaaccatggccacttgatacttatgtagaatccatcgtgggctgatgcaagccctttatttaggcttagtgttgtgggcaccaatgtcgagcatcgttgtgacttgtgctgtatgattctcactgaagaatttcctttcagccaagaagcagtgaggtctgggaatattccaaagtcatgtctctgaatatgtgtccttgacgtgcaagctttgtaaaaccccatccccgcttaggtgcgaggcatcaccttctcacaagtgtttagtttcttttaaccacaagtatcattcttgggtgataatatagtttcattctacttagggattgtttagaaaacaaagaaagagccaattaaattttttagtttttgaaatttttatttatatgtatacttagatgagtattttaagctgtcgacctttagtttgccatacgggtaggactgtatttcatgttaacaactggtggtaatgataagccttcttctagcgtattttctcttctttcctgtcactttcctaagtttttttttttaaagactggaattttttttggctttatcttgtcttaccgtagagatttgttcaaaactctaagccctaccacctcccctttaataagctctttaaatagttgaatcattaacaacctggtgggaggcaagtcatttaattgaaccactaggaagtgtattttcttttctttttctgccaactttttggtggcatttgtaaaagctgatataaaaggctctgagatgttattttcagttattccataggcaagcctttttacagagcatatgtctccagttggcagcttgagatatttccgagcatccggttctagctaccagtgcctcccaatgcttagtgcacagtactgtagactggccatcacccctctccttggaaaatgccactgtgctgtttgaaaaaaagcagccttttagggctagagtattttatataaacagaagagctaagttcctgaagactaagctagatagctgcagctatatgtaaattgtatatttttatgaacttttgaagcacacactcctgtttccctctgtgtagctttgtggggatttcatgtatatatgctgtctgaaagaatccagaggttggagtgccaatagaaaatgaaaacaaatgccttgtactacaggcagcctctgaaggtgaccacataactgtcttcactgtgaccaatcggagtccctgcttgcttgtgaagaaggggcttttgtaccttgttggagatgccacctcagaagttcacactgtgcaggaaaaaggttttattctctcctggcatacattagaatgtcagatgcttgcatccatgtggaccacgatgggcctctaaaaattggtgggcagggggtttgcttatgagttttctctggaaaccgattttactcctggatgtattgaatgccccttgagctttatgagatacgagtccacatggataaaatgttagagagtggagttctacagaggattccaggaagaggccatgtctgtgcagtcctagttccagacaggtgagaagctccaggaactactggctaccttgacaagctgggtaaatagttatcattctgggtaactggttgaaactctgacttttggacaagtaattcctggggttctgtctttggtagcatcaccagggatatttgggtgggacagacagaagacacacagctgcctgttctctcctgcccatcatgtttggcccactagatgaagctgtactcagcaatttagggaatgtaacccttctcagaactggccattttcaggggaagcttgggagagcaatagtatggtgagccccttagagatgagcgcctactccttcttggcgaatgctgccttcagatgcttaccaagtggtcactgcatctagtaagattatatttccagtacacttccttagggcagaaacaccatcctatcaggtttggtcagtcccttcttcatgaagggagtcatggggaattcctgaaaattttcttccttctgcagacagttggatgagtcccttagagaaggcatccagagacataactaaactgaatatcatcccatattgattttaggaattgactctaaaactctgtgcagaatcttgtgttgggattgtatcttgacattcctgttgtgttatttttcttaactggagtgtgtgctgcctttcaggtacaatttttgtgtaataaaagccagtgcattaagtttatatagactactttctatgcaagactgagatatggaatagataggaagagatatgtactgctgggtacatggacagtaagtgtgttttcagatggagtaccagcaccgaaaatgggttgagggaggatgggttgtatgtatgtttctgcccactaattttgagcagccatattatgaattaaatcgtcacagccaagtaataacccaagaatggtatgagtttcatgtgtaatagctcaaatggaataagcatgaatgctggagtggaccattatcctcaaatattctatgtcacttctcatttaaagactcttgttatgaactattagaaactttaggcaaaatcaaaagtatttgcggcaaaataaaggcctattctactcttatttaaagtgaaacactgtatacttgtttctctccaaagcgaaattaagtatttataatttcaattgcctcgataagtttccaagtcactgaaatctgctgaaggttttactgtattgttgcacaactttaagataatttttgtctcaatgtcaacttttttcactgaataaaaatttaactgggtcaagaaaacacctctttgaaaatccactgtctctgtgtgtctcgagctgttctttagagcgcaataaagatggctgacgcagtctccaaacccc

SEQ ID NO: 2 - protein, 2208 aa, Homo sapiens
MATRSSRRESRLPFLFTLVALLPPGALCEVWTQRLHGGSAPLPQDRGFLVVQGDPRELRLWARGDARGASRADEKPLRRKRSAALQPEPIKVYGQVSLNDSHNQMVVHWAGEKSNVIVALARDSLALARPKSSDVYVSYDYGKSFKKISDKLNFGLGNRSEAVIAQFYHSPADNKRYIFADAYAQYLWITFDFCNTLQGFSIPFRAADLLLHSKASNLLLGFDRSHPNKQLWKSDDFGQTWIMIQEHVKSFSWGIDPYDKPNTIYIERHEPSGYSTVFRSTDFFQSRENQEVILEEVRDFQLRDKYMFATKVVHLLGSEQQSSVQLWVSFGRKPMRAAQFVTRHPINEYYIADASEDQVFVCVSHSNNRTNLYISEAEGLKFSLSLENVLYYSPGGAGSDTLVRYFANEPFADFHRVEGLQGVYIATLINGSMNEENMRSVITFDKGGTWEFLQAPAFTGYGEKINCELSQGCSLHLAQRLSQLLNLQLRRMPILSKESAPGLIIATGSVGKNLASKTNVYISSSAGARWREALPGPHYYTWGDHGGIITAIAQGMETNELKYSTNEGETWKTFIFSEKPVFVYGLLTEPGEKSTVFTIFGSNKENVHSWLILQVNATDALGVPCTENDYKLWSPSDERGNECLLGHKTVFKRRTPHATCFNGEDFDRPVVVSNCSCTREDYECDFGFKMSEDLSLEVCVPDPEFSGKSYSPPVPCPVGSTYRRTRGYRKISGDTCSGGDVEARLEGELVPCPLAEENEFILYAVRKSIYRYDLASGATEQLPLTGLRAAVALDFDYEHNCLYWSDLALDVIQRLCLNGSTGQEVIINSGLETVEALAFEPLSQLLYWVDAGFKKIEVANPDGDFRLTIVNSSVLDRPRALVLVPQEGVMFWTDWGDLKPGIYRSNMDGSAAYHLVSEDVKWPNGISVDDQWIYWTDAYLECIERITFSGQQRSVILDNLPHPYAIAVFKNEIYWDDWSQLSIFRASKYSGSQMEILANQLTGLMDMKIFYKGKNTGSNACVPRPCSLLCLPKANNSRSCRCPEDVSSSVLPSGDLMCDCPQGYQLKNNTCVKEENTCLRNQYRCSNGNCINSIWWCDFDNDCGDMSDERNCPTTICDLDTQFRCQESGTCIPLSYKCDLEDDCGDNSDESHCEMHQCRSDEYNCSSGMCIRSSWVCDGDNDCRDWSDEANCTAIYHTCEASNFQCRNGHCIPQRWACDGDTDCQDGSDEDPVNCEKKCNGFRCPNGTCIPSSKHCDGLRDCSDGSDEQHCEPLCTHFMDFVCKNRQQCLFHSMVCDGIIQCRDGSDEDAAFAGCSQDPEFHKVCDEFGFQCQNGVCISLIWKCDGMDDCGDYSDEANCENPTEAPNCSRYFQFRCENGHCIPNRWKCDRENDCGDWSDEKDCGDSHILPFSTPGPSTCLPNYYRCSSGTCVMDTWVCDGYRDCADGSDEEACPLLANVTAASTPTQLGRCDRFEFECHQPKTCIPNWKRCDGHQDCQDGRDEANCPTHSTLTCMFQCEDGEACIVLSERCDGFLDCSDESDEKACSDELTVYKVQNLQWTADFSGDVTLTWMRPKKMPSASCVYNVYYRVVGESIWKTLETHSNKTNTVLKVLKPDTTYQVKVQVQCLSKAHNTNDFVTLRTPEGLPDAPRNLQLSLPREAEGVIVGHWAPPIHTHGLIREYIVEYSRSGSKMWASQRAASNFTEIKNLLVNTLYTVRVAAVTSRGIGNWSDSKSITTIKGVIPPPDIHIDSYGENYLSFTLTMESDIKVNGYVVNLFWAFDTHKQERRTLNFRGSILSHKVGNLTAHTSYEISAWAKTDLGDSPLAFEHVMTRGVRPPAPSLKAKAIQTAVECTWTGPRNVVYGIFYATSFLDLYRNPKSLTTSLHNKTVIVSKDEQYLFLVRVVVPYQGPSSDYVVVKMIPDSRLPPRHLHVVHTGKTSVVIKWESPYDSPDQDLLYAIAVKDLIRKTDRSYKVKSRNSTVEYTLNKLEPGGKYHIIVQLGNMSKDSSIKITTVSLSAPDALKIITENDHVLLFWKSLALKEKHFNESRGYEIHMFDSAMNITAYLGNTTDNFFKISNLKMGHNYTFTVQARCLFGNQICGEPAILLYDELGSGADASATQAARSTVAAVVVPILFLILLSLGVGFAILYTKHRRLQSSFTAFANSHYSSRLGSAIFSSGDDLGEDDEDAPMITGFSDDVPMVIA

SEQ ID NO: 3 - DNA, 3203 bp, Homo sapiens
aaactcacacaacaactcttccccgctgagaggagacagccagtgcgactccaccctccagctcgacggcagccgccccggccgacagccccgagacgacagcccggcgcgtcccggtccccacctccgaccaccgccagcgctccaggccccgccgctccccgctcgccgccaccgcgccctccgctccgcccgcagtgccaaccatgaccgccgccagtatgggccccgtccgcgtcgccttcgtggtcctcctcgccctctgcagccgggtaagcgccgggagcccccgctgcggccggcggctgccagggagggactcggggccggccggggagggcgtgcgcgccgaccgagcgccgctgaccgccctgtcctccctgcagccggccgtcggccagaactgcagcgggccgtgccggtgcccggacgagccggcgccgcgctgcccggcgggcgtgagcctcgtgctggacggctgcggctgctgccgcgtctgcgccaagcagctgggcgagctgtgcaccgagcgcgacccatgcgacccgcacaagggcctattctgtcacttcggctccccggccaaccgcaagatcggcgtgtgcaccggtaagacccgcagcccccaccgctaggtgtccggccgcctcctccctcacgcccacccgcccgctggaaaaagaaaccgctcggactgagtttctttctccagctgctgccagcccgccccctgcagcccagatcccaactcgcatccctgacgctctggatgtgagagtgccccaatgcctgacctctgcatcccccacccctctcttcccttcctcttctccagccaaagatggtgctccctgcatcttcggtggtacggtgtaccgcagcggagagtccttccagagcagctgcaagtaccagtgcacgtgcctggacggggcggtgggctgcatgcccctgtgcagcatggacgttcgtctgcccagccctgactgccccttcccgaggagggtcaagctgcccgggaaatgctgcgaggagtgggtgtgtgacgagcccaaggaccaaaccgtggttgggcctgccctcgcgggtgagtcgagtcttcctctaagtcagggtcgtgattctctcccagggagggagtcctaactgtgccgaccgaacgggggaaataccttatccaggcgttttacatggtgtttgtgtgctctgctctcgcagcttaccgactggaagacacgtttggcccagacccaactatgattagagccaactgcctggtccagaccacagagtggagcgcctgttccaagacctgtgggatgggcatctccacccgggttaccaatgacaacgcctcctgcaggctagagaagcagagccgcctgtgcatggtcaggccttgcgaagctgacctggaagagaacattaaggtacatgttctgctcctattaactatttttcacaggaaaaacagtggataggacccaacttagggctcttgccacgcttgttagtataagcccgttatctccaaaactatctaaccattgagctgttttgctggaatgagagcttgtgtaatagcaaccaccagttttccactacgaaatcttccacagggttagttaattcaagacattccaagagaggctctggctatttttggacatagcaaatgagactcaaacttcctcccctcaaaatataaacagaagtcagacaacagaagactaaaacacagagggttgaagaaagccactcctcttgtagagtcgctgatttttttttttcctctctcttttcccttgtcttccttagaagggcaaaaagtgcatccgtactcccaaaatctccaagcctatcaagtttgagctttctggctgcaccagcatgaagacataccgagctaaattctgtggagtatgtaccgacggccgatgctgcaccccccacagaaccaccaccctgccggtggagttcaagtgccctgacggcgaggtcatgaagaagaacatgatgttcatcaagacctgtgcctgccattacaactgtcccggagacaatgacatctttgaatcgctgtactacaggaagatgtacggagacatggcatgaagccagagagtgagagacattaactcattagactggaacttgaactgattcacatctcatttttccgtaaaaatgatttcagtagcacaagttatttaaatctgtttttctaactgggggaaaagattcccacccaattcaaaacattgtgccatgtcaaacaaatagtctatcaaccccagacactggtttgaagaatgttaagacttgacagtggaactacattagtacacagcaccagaatgtatattaaggtgtggctttaggagcagtgggagggtaccagcagaaaggttagtatcatcagatagcatcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttgcctgtaacaagccagattttttaaaatttatattgtaaatattgtgtgtgtgtgtgtgtgtgtatatatatatatatgtacagttatctaagttaatttaaagttgtttgtgcctttttatttttgtttttaatgctttgatatttcaatgttagcctcaatttctgaacaccataggtagaatgtaaagcttgtctgatcgttcaaagcatgaaatggatacttatatggaaattctgctcagatagaatgacagtccgtcaaaacagattgtttgcaaaggggaggcatcagtgtccttggcaggctgatttctaggtaggaaatgtggtagcctcacttttaatgaacaaatggcctttattaaaaactgagtgactctatatagctgatcagttttttcacctggaagcatttgtttctactttgatatgactgtttttcggacagtttatttgttgagagtgtgaccaaaagttacatgtttgcacctttctagttgaaaataaagtgtatattttttctataaa

SEQ ID NO: 4 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCHFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 5 - protein, 323 aa, Homo sapiens
QNCSGPCRCPDEPAPRCPAGVSLVLDGCGCCRVCAKQLGELCTERDPCDPHKGLFCHFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 6 - DNA, 2418 bp, Mus musculus
agttctttggcgagccggctcccgggagcgtataaaagccagcgccgcccgcctagtctcacacagctcttctctccaagaagactcagccagatccactccagctccgaccccaggagaccgacctcctccagacggcagcagccccagcccagccgacaaccccagacgccaccgcctggagcgtccagacaccaacctccgcccctgtccgaatccaggctccggccgcgcctctcgtcgcctctgcaccctgctgtgcatcctcctaccgcgtcccgatcatgctcgcctccgtcgcaggtcccatcagcctcgccttggtgctcctcgctctctgcacccggcctgctatgggccaggactgcagcgcgcaatgtcagtgcgcagccgaagcagcgccgcactgccccgccggcgtgagcctggtgctggacggctgcggctgctgccgcgtctgcgccaagcagctgggagaactgtgtacggagcgtgacccctgcgacccacacaagggcctcttctgcgatttcggctcccccgccaaccgcaagatcggagtgtgcactgccaaagatggtgcaccctgtgtcttcggtgggtcggtgtaccgcagcggtgagtccttccaaagcagctgcaaataccaatgcacttgcctggatggggccgtgggctgcgtgcccctgtgcagcatggacgtgcgcctgcccagccctgactgccccttcccgagaagggtcaagctgcctgggaaatgctgcgaggagtgggtgtgtgacgagcccaaggaccgcacagcagttggccctgccctagctgcctaccgactggaagacacatttggcccagacccaactatgatgcgagccaactgcctggtccagaccacagagtggagcgcctgttctaagacctgtgggatgggcatctccacccgagttaccaatgacaataccttctgcagactggagaagcagagccgcctctgcatggtcaggccctgcgaagctgacctggaggaaaacattaagaagggcaaaaagtgcatccggacacctaaaatcgccaagcctgtcaagtttgagctttctggctgcaccagtgtgaagacatacagggctaagttctgcggggtgtgcacagacggccgctgctgcacaccgcacagaaccaccactctgccagtggagttcaaatgccccgatggcgagatcatgaaaaagaatatgatgttcatcaagacctgtgcctgccattacaactgtcctggggacaatgacatctttgagtccctgtactacaggaagatgtacggagacatggcgtaaagccaggaagtaagggacacgaactcattagactataacttgaactgagttgcatctcattttcttctgtaaaaacaattacagtagcacattaatttaaatctgtgtttttaactaccgtgggaggaactatcccaccaaagtgagaacgttatgtcatggccatacaagtagtctgtcaacctcagacactggtttcgagacagtttacacttgacagttgttcattagcgcacagtgccagaatgcacactgaggtgagtctcctggaacagtggagatgccaggagaaagaaagacaggtactagctgaggttattttaaaagcagcagtgtgcctactttttggagtgtaaccggggagggaaattatagcatgcttgcagacagacctgctctagcgagagctgagcatgtgtcctccactagatgaggctgagtccagctgttctttaagaacagcagtttcagctctgaccattctgattccagtgacacttgtcaggagtcagagccttgtctgttagactggacagcttgtggcaagtaagtttgcctgtaacaagccagatttttattgatattgtaaatattgtggatatatatatatatatatatatatatatttgtacagttatctaagttaatttaaagtcatttgtttttgttttaagtgcttttgggattttaaactgatagcctcaaactccaaacaccataggtaggacacgaagcttatctgtgattcaaaacaaaggagatactgcagtgggaattgtgacctgagtgactctctgtcagaacaaatgctgtgcaggtgataaagctatgtattggaagtcagatttctagtaggaaatgtggtcaaatccctgttggtgaacaaatggcctttattaagaaatggctggctcagggtaaggtccgattcctaccaggaagtgcttgctgcttctttgattatgactggtttggggtggggggcagtttatttgttgagagtgtgaccaaaagttacatgtttgcacctttctagttgaaaataaagtatatatatattttttatatgaaaaaaaaaaaaaaaaaa

SEQ ID NO: 7 - protein, 348 aa, Mus musculus
MLASVAGPISLALVLLALCTRPATGQDCSAQCQCAAEAAPHCPAGVSLVLDGCGCCRVCAKQLGELCTERDPCDPHKGLFCDFGSPANRKIGVCTAKDGAPCVFGGSVYRSGESFQSSCKYQCTCLDGAVGCVPLCSMDVRLPSPDCPFPRRVKLPGKCCEEWVCDEPKDRTAVGPALAAYRLEDTFGPDPTMMRANCLVQTTEWSACSKTCGMGISTRVTNDNTFCRLEKQSRLCMVRPCEADLEENIKKGKKCIRTPKIAKPVKFELSGCTSVKTYRAKFCGVCTDGRCCTPHRTTTLPVEFKCPDGEIMKKNMMFIKTCACHYNCPGDNDIFESLYYRKMYGDMA

SEQ ID NO: 8 - protein, 323 aa, Mus musculus
QDCSAQCQCAAEAAPHCPAGVSLVLDGCGCCRVCAKQLGELCTERDPCDPHKGLFCDFGSPANRKIGVCTAKDGAPCVFGGSVYRSGESFQSSCKYQCTCLDGAVGCVPLCSMDVRLPSPDCPFPRRVKLPGKCCEEWVCDEPKDRTAVGPALAAYRLEDTFGPDPTMMRANCLVQTTEWSACSKTCGMGISTRVTNDNTFCRLEKQSRLCMVRPCEADLEENIKKGKKCIRTPKIAKPVKFELSGCTSVKTYRAKFCGVCTDGRCCTPHRTTTLPVEFKCPDGEIMKKNMMFIKTCACHYNCPGDNDIFESLYYRKMYGDMA

SEQ ID NO: 9 - DNA, 2338 bp, Rattus norvegicus
ctccaagaagactcagccagacccactccagctccgaccctaggagaccgacctcctccagacggcagcagccccagcccagtggacaaccccaggagccaccacctggagcgtccggacaccaacctccgccccgagaccgagtccaggctccggccgcgcccctcgtcgcctctgcaccccgctgtgcgtcctcctgccgcgccccgaccatgctcgcctccgtcgcgggtcccgttagcctcgccttggtgctcctcctctgcacccggcctgccaccggccaggactgcagcgcgcagtgtcagtgcgcacgtgaagcggcgccgcgctgccccgccggcgtgagcctggtgctggacggctgcggctgctgccgcgtctgcgccaagcagctgggagaactgtgcacggagcgtgatccctgcgacccacacaagggtctcttctgcgacttcggctcccccgccaaccgcaagattggcgtgtgccctgccaaagatggtgcaccctgtgtcttcggtgggtccgtgtaccgcagcggcgagtccttccaaagcagttgcaaataccagtgcacttgcctggatggggccgtgggctgtgtgcccctgtgcagcatggacgtgcgcctgcccagccctgactgccccttcccgagaagggtcaagctgcccgggaaatgctgtgaggagtgggtgtgtgatgagcccaaggaccgcacagtggttggccctgccctagctgcctaccgactggaagacacatttggccctgacccaactatgatgcgagccaactgcctggtccagaccacagagtggagcgcctgttctaagacctgtgggatgggcatctccacccgggttaccaatgacaataccttctgcaggctggagaagcagagtcgtctctgcatggtcaggccctgtgaagctgacctagaggaaaacattaagaagggcaaaaagtgcatccggacgcctaaaattgccaagcctgtcaagtttgagctttctggctgcaccagtgtgaagacctaccgggctaagttctgtggggtgtgcacggacggccgctgctgcacaccgcacagaaccaccacactgccggtggagttcaagtgccccgatggcgagatcatgaaaaagaacatgatgttcatcaagacctgtgcctgccattacaactgtcccggggacaatgacatctttgagtccttgtactacaggaagatgtatggagacatggcgtaaagccagggagtaagggacacgaactcatttagactataacttgaactgagttacatctcattttcttctgtaaaaaaaacaaaaagggttacagtagcacattaatttaaatctgggttcctaactgctgtgggagaaaacaccccaccgaagtgagaaccgtgtgtcattgtcatgcaaatagcctgtcaatctcagacactggtttcgagacagtttagacttgacagttgttcactagcgcacagtgacagaacgcacactaaggtgagcctcctggaagagtggagatgccaggagaaagacaggtactagctgaggtcattttaaaagcagcgatatgcctactttttggagtgtgacaggggagggacattatagcttgcttgcagacagacctgctctagcaagagctgggtgtgtgtcctccactcggtgaggctgaagccagctattctttcagtaagaacagcagtttcagcgctgacattctgattccagtgacactggtcgggagtcagaaccttgtctattagactggacagcttgtggcaagtgaatttgccggtaacaagccagatttttatggatcttgtaaatattgtggataaatatatatatttgtacagttatctaagttaatttaaagacgtttgtgcctattgttcttgttttaagtgcttttggaatttttaaactgatagcctcaaactccaaacaccatcgataggacataaagcttgtctgtgattcaaaacaaaggagatactgcagtggaaactgtaacctgagtgactgtctgtcagaacatatggtacgtagacggtaaagcaatggatcagaagtcagatttctagtaggaaatgtaaaatcactgttggcgaacaaatggcctttattaagaaatggcttgctcagggtaactggtcagatttccacgaggaagtgtttgctgcttctttgactatgactggtttgggaggcagtttatttgttgagagtgtgaccaaaagttacatgtttgcacctttctagttgaaaataaagtatatatattttttatatgaaaaaaaaaaaaaaaa

SEQ ID NO: 10 - protein, 347 aa, Rattus norvegicus
MLASVAGPVSLALVLLLCTRPATGQDCSAQCQCAAEAAPRCPAGVSLVLDGCGCCRVCAKQLGELCTERDPCDPHKGLFCDFGSPANRKIGVCTAKDGAPCVFGGSVYRSGESFQSSCKYQCTCLDGAVGCVPLCSMDVRLPSPDCPFPRRVKLPGKCCEEWVCDEPKDRTVVGPALAAYRLEDTFGPDPTMMRANCLVQTTEWSACSKTCGMGISTRVTNDNTFCRLEKQSRLCMVRPCEADLEENIKKGKKCIRTPKIAKPVKFELSGCTSVKTYRAKFCGVCTDGRCCTPHRTTTLPVEFKCPDGEIMKKNMMFIKTCACHYNCPGDNDIFESLYYRKMYGDMA

SEQ ID NO: 11 - protein, 323 aa, Rattus norvegicus
QDCSAQCQCAAEAAPRCPAGVSLVLDGCGCCRVCAKQLGELCTERDPCDPHKGLFCDFGSPANRKIGVCTAKDGAPCVFGGSVYRSGESFQSSCKYQCTCLDGAVGCVPLCSMDVRLPSPDCPFPRRVKLPGKCCEEWVCDEPKDRTVVGPALAAYRLEDTFGPDPTMMRANCLVQTTEWSACSKTCGMGISTRVTNDNTFCRLEKQSRLCMVRPCEADLEENIKKGKKCIRTPKIAKPVKFELSGCTSVKTYRAKFCGVCTDGRCCTPHRTTTLPVEFKCPDGEIMKKNMMFIKTCACHYNCPGDNDIFESLYYRKMYGDMA

SEQ ID NO: 12 - DNA, 2330 bp, Bos taurus
gaggcagccagtgcgagtaggcagccagtgcgactcccaccgccggcgacccacctcctccagtccgacggcagccgccccggccgacagctcccgagacagcagcccggcgctccccggcccacgcctccggcccaaacctgcgccgcccgcccggccagccgcctctcgccgcccgccgccctccggacacagcgccccgaccatgtcagccaccggcctgggcccggtccgctgcgccttcgtgctcctgctcgccctctgcagccggcccgcctccagccaggactgctgcagcgccccgtgccagtgccctgccggcccggcgccgcgctgccccgccggcgtcagcttggtgctggacggctgcggctgctgcgtgtgcgccaagcagctgagcgagttgtgtaccgagcgcgacccctgcgatccgcacaagggcctcttctgcgacttcggctccccaaccaaccgcaagatcggcgtgtgcaccgctaaagatggtgccccctacattttcggaggaactgtgtaccagagcggagagtctttccagagcagctgcaaataccagtgcacgtgtctggacgggtcggtgggctgcgtgcccctatgcagcgtggacgtccgcctgcccagccccgactgccccttcccacggagggtcaaactgcccgggaaatgctgcgaggaatgggtgtcccgtgatgagaaggagcacaccgtggtcggccctgcgctggcagcttaccggctggaagacacgtttggcccagacccaaccatgatccgagccaactgccaggtccagaccacagagtggagtgcctattccaagacctgcggaatgggcatctccacccgggttaccaatgacaacgcattctgcaggctggagaagcagagccgcctctgcatggtcaggccttgcgaagctgacctggaggagaacattaagaaaggcaaaaagtgcatccggactcccaaaatctccaagcctatcaagtttcagctttctggctgcaccagcatgaagacataccgagctaaattcttcggagtgtgcacagacgggcggtgctgcaccccccacagaaccaccacccttcccgtggagttcaagtgtcctgatggggaggtcatgaagaagagcatgatgttcatcaagacctgtgcctgccattacaactgccccggagacaatgacatcttcgagtcactgtactacaggaagatgtatggagacatggcctaaagccagagacagtgagacacgtgaacattttaggctgtcacttgaatcgattcacatctcatttttgtgtacacgtgatttcagtggcacaagttatttaaatctgtgcttctaactggggaaaagaaaaattcccaccaaattcaaaatactgtgccatgtgatattcaaacgaatagtccgtcaaccccagacactggtttgaagaaattgagacttgatcataggactgtattagtgcacagcaccagcatgtatgctaggagcagtgggaggaggccagtagaaagccttgtcatctttaggggtagtgatgtgactgctatttggagtgtcactgaaaaggaaaactttagcatgctcactgatctgcctatagctccagcaacagctcggatgtgcgttctccagccatcatgaggctgagtcaagttcgtctctaagtcagaacagcagattcagctatgacattctgattcaaggacattgttcaggaatcagaattctgtctattagactggacagcttgtggcaagctaatttgcctgtaacaagccagatttttttttattgatactgtaaatattgtgtgtatatatatatatttgtacagttatctaagttaatttaaagttgtttgtgcctttttgtttatgtttttaatgctttgatatttcaagcgttagcctcaattctgaacaccataggtaggacggaaagcttgtctgataattcaaagcatgaaatggatattcaaatagaatttctgtgcagttggagcaacagtccatccaaacaggttgtttgctgaaggtgaggcagtgatgtccttcaaagtctgatctgtaggttggaaacgtggtagcctccttttaacgaacaaatgacctttattaaaaatgagtagctctgtatagctgattggtttttccacctggaagcatttgtctctactttgactatgactgttttttggacagatttatttgttgagagtgtgaccaaaagttacatgtttgcacctttctagttgaaaataaagtatatattttttctataaaaaaaaaaaaaaaaa

SEQ ID NO: 13 - protein, 349 aa, Bos taurus
MSATGLGPVRCAFVLLLALCSRPASSQDCSAPCQCPAGPAPRCPAGVSLVLDGCGCCRVCAKQLSELCTERDPCDPHKGLFCDFGSPANRKIGVCTAKDGAPCVFGGTVYQSGESFQSSCKYQCTCLDGSVGCVPLCSVDVRLPSPDCPFPRRVKLPGKCCEEWVCDEPKEHTVVGPALAAYRPEDTFGPDPTMIRANCLVQTTEWSACSKTCGMGISTRVTNDNAFCRLEKQSRLCMVRPCEADLEENIKKGKKCIRTPKISKPIKFELSGCTSMKTYRAKFCGVCTDGRCCTPHRTTTLPVEFKCPDGEVMKKSMMFIKTCACHYNCPGDNDIFESLYYRKMYGDMA

SEQ ID NO: 14 - protein, 323 aa, Bos taurus
QDCSAPCQCPAGPAPRCPAGVSLVLDGCGCCRVCAKQLSELCTERDPCDPHKGLFCDFGSPANRKIGVCTAKDGAPCVFGGTVYQSGESFQSSCKYQCTCLDGSVGCVPLCSVDVRLPSPDCPFPRRVKLPGKCCEEWVCDEPKEHTVVGPALAAYRPEDTFGPDPTMIRANCLVQTTEWSACSKTCGMGISTRVTNDNAFCRLEKQSRLCMVRPCEADLEENIKKGKKCIRTPKISKPIKFELSGCTSMKTYRAKFCGVCTDGRCCTPHRTTTLPVEFKCPDGEVMKKSMMFIKTCACHYNCPGDNDIFESLYYRKMYGDMA

SEQ ID NO: 15 - DNA, 1598 bp, Sus scrofa
gcacgagctgagaggaggcagtcagcccgactcccaccgccgacgacccaccgccttcagtccgacggcagccgccccggccgacagctcccgagacagccgcccggcgcgttccaggctcccgcctccggcccgaacccgcgccgcccgctcggcccgccacgccaccccagccccgccgccagccgcccgcagcgccccgaccatgtccgccaccggcctgagcccggtccgctgcgccttcgtgctcctgctcgccctctgcagccggcccgcctctggccaggactgcagcggccaatgccagtgcgcggccgggaagcgccgcgcttgccccgccggcgtcagctcgttgctggaaggctgcggctgctgccgattgtgcgccaagcacttgggtgacttgtgcacggagcgcgcaccctgcgacccgcacaagggcctcttctgtgacttcggctccccggccaaccgcaagatcggagtgtgcacagccaaagattgtgccccctgcgtctttggaggaacggtgtaccggagcggagagtccttccagagcagctgcaaataccagtgcacttgcctggacggggccgtgggctgcgtgcccctgtgcagcatggacgtccgcctgcccagccccgactgccccttcccacggagggtcaagctgcccgggaaatgctgcgaggagtgggtgtgtgacgagcccaaggaccacaccgtggtcgggcctgccctggcggcttaccgactggaagacacgtttggcccagacccaactatgatgcgagccaactgcctggtccagaccacagagtggagtgcctgttccaagacctgtgggatgggcatctccacccgggttaccaatgacaacgcttcttgcagactggagaagcagagccgcctctgcatggtcaggccttgtgaagctgacctggaagagaacattaagaagggcaaaaagtgcatccgtacccccaaaatctccaagcccgtcaagtttgagctttccggctgcaccagtgtgaagacataccgggctaagttctgcggggtatgcacagacggccgctgctgcacccctcacagaaccaccacccttcctgtggagttcaagtgtcccgacggtgaggtcatgaagaagagcatgatgttcatcaagacttgtgcctgccattacaactgccccggggacaatgacatcttcgagtccctgtactacaggaagatgtatggagacatggcctaaagccagagagagtgagaccatgaacacttgagactgtcactttgaacttgattcacatctcatttttgcgtaaacatgattttcagtagcacaggttatttaaagccagagagagtgagacccatgaacacttgagactgtcacttgaactgattcacatctcatttttgcgtaaacatgatttcagtagcacaggttatttaaatctgtttttctaatgggggacaaaggaaaattcccaccaacattcaaaatcgtgtgccatgtgacattcaaaaaaagatctatcaaacccagacactggtttgaagaaagttcagacttgaccgtggggacggtatta

SEQ ID NO: 16 - protein, 349 aa, Sus scrofa
MSATGLSPVRCAFVLLLALCSRPASGQDCSGQCQCAAGKRRACPAGVSSLLEGCGCCRLCAKHLGDLCTERAPCDPHKGLFCDFGSPANRKIGVCTAKDCAPCVFGGTVYRSGESFQSSCKYQCTCLDGAVGCVPLCSMDVRLPSPDCPFPRRVKLPGKCCEEWVCDEPKDHTVVGPALAAYRLEDTFGPDPTMMRANCLVQTTEWSACSKTCGMGISTRVTNDNASCRLEKQSRLCMVRPCEADLEENIKKGKKCIRTPKISKPVKFELSGCTSVKTYRAKFCGVCTDGRCCTPHRTTTLPVEFKCPDGEVMKKSMMFIKTCACHYNCPGDNDIFESLYYRKMYGDMA

SEQ ID NO: 17 - protein, 323 aa, Sus scrofa
QDCSGQCQCAAGKRRACPAGVSLVLDGCGCCRLCAKQLGELCTERDPCDPHKGLFCDFGSPANRKIGVCTAKDGAPCVFGGTVYRSGESFQSSCKYQCTCLDGAVGCVPLCSMDVRLPSPDCPFPRRVKLPGKCCEEWVCDEPKDHTVVGPALAAYRLEDTFGPDPTMMRANCLVQTTEWSACSKTCGMGISTRVTNDNAFCRLEKQSRLCMVRPCEADLEENIKKGKKCIRTPKISKPVKFELSGCTSVKTYRAKFCGVCTDGRCCTPHRTTTLPVEFKCPDGEVMKKSMMFIKTCACHYNCPGDNDIFESLYYRKMYGDMA

SEQ ID NO: 18 - DNA, 2361 bp, Xenopus laevis
tcccaagacaaggaaccaaatacagcgaacgagggaaaaggaaagaggcttgagagaaaagaaactaagggaactacacaatcgccaacgagatcactttgagatcaagacttgagagaacgacttttctttggagtagaagagaactaatctgctgctgtacaatgtctgcaggaaaagtgacagctgtgctcctctttgctctcttctgctgggtgtctgatgcccaggagtgtaatggggaatgccagtgcccaaataaagtgcctgtgtgtgatcctggagtcagcctggtgcaggatggctgtggctgctgcaaggtgtgctccaagcagctaggggagctgtgcaccgaaagagatgtgtgcgacccacacaaagggctcttctgtgacttcggatccagagtgaacaggaaaattggagtttgcactgccagggaaggtgccccttgtgtgttcggaggcactgtgtatagaagtggggagtctttccagagcagctgcaagtaccaatgtacttgtatcgatgggggtgtgggatgtgttccactttgcagcatggacatccgcctgcccagccccgagtgccccttcccacgaagagtcaaactgcctggcaagtgctgtgaagaatgggtctgtgatcaacctcaagagagaaccttagtcggacctgctttgcctgctttcagaatggaagaaacctatggtcctgatccatccctaatccgcgccaactgcctagtacagactactgaatggagtgcttgctcaaagacctgtggcatgggaatctccaccagggtcactaatgacaatgagcactgcagactggagaaacagagcagactctgcatggtcaggccctgtgaagctgacctagaagaaaacatcaagaaagggaaaaagtgcatccgtacaccaaaaatttcaaaaccagtgaagtttgaattttccggctgcaccagcgtaaaaacctacagagccaagttctgcggtgtgtgcactgatggtcgttgttgcactcctcacagaaccgctaccctcccagttgagttcaagtgccctgatggtgaggtcatgaagaagaacatgatgttcatcaagacatgcgcatgccatttcaactgtccaggagacaacgacatctttgaggccatgtactatcggaaaatgtatggagacatggcatagaggcagagaaacacacttcaaagactttttcacttgaaataactttgcatctcattgctaaacataaatgtatagcacaagagcatatttaaatgtaattaatgaaaagtctttattttgaagacttaaaacgttaaatgaccccagtcgttggtttaaagaaattggcacaaaacaagccacagccactgacttgagtgtaatgtgctgtcactgtactcccaaaattccggtacagtatagggatgtccatcatgtgacctttacctgaaactgtcatgtcatgagagcatcacatattggccacaggttggacatatctggtaatctgcattgcacccagtcaagctactggaaaaaaatttctatttacagtatatgatgaactggcgctatgtcagtgaccgctgaagttttatagcccaactaccttattgttctgaagaaagccgtccttgtgggacagctgttttagaaacccctaagtacaaattgggaggaactttaactacgtctgttacaagactggaaagattgtagcatatcaatgtacatgctgtaatcttgttttattattattgtatataatgtatatatatttttgtacagttatctaacttaacttaaatttgtttcgttcttcattttttacaatgctttaatatgtgtatgttaaccgtttttatttttgtttgtttgcgataaatctatgcttatctgaaaaagaaaccattcaaagcacgaaatagattctcaaatagaaattgttctcagttaaagagagggacagtccagtgactgcttacagttctgtttattacgagccatccggcagtgttgattgtactggaaacattgtattaacttgacttcctttaggtgccagagaggtatccagtgcatttcacaagaaagcaagggcaacttctctggtacatatggggtgaatgaacaaataaccagttgagtgtatatcctgaaacggatccatgtattttgaaccagatcatctgtttcctcctttgaccatgactgttctattttcaacagtttgttacgtgttgagaagtgtgaccaaatggtacatgtttgcacttttctagatgaaaaataaaattactttatttttatataaaaaaaaaaaaaaaaaaaaaaaaaaaaaaaa

SEQ ID NO: 19 - protein, 340 aa, Xenopus laevis
MSAGKVTAVLLFALFCWVSDAQECNGECQCPNKVPVCDPGVSLVQDGCGCCKVCSKQLGELCTERDVCDPHKGLFCDFGSRVNRKIGVCTAREGAPCVFGGTVYRSGESFQSSCKYQCTCIDGGVGCVPLCSMDIRLPSPECPFPRVKLPGKCCEEWVCDQPQERTLVGPALPAFRMEETYGPDPSLIRANCLVQTTEWSACSKTCGMGISTRVTNDNEHCRLEKQSRLCMVRPCEADLEENIKKGKKCIRTPKISKVKFEFSGCTSVKTRAKFCGVCTDGRCCTPHRTATLPVEFKCPDGEVMKKNMMFIKTCACHFNCPGDNDIFEAMYYRKMYGDMA

SEQ ID NO: 20 - protein, 322 aa, Xenopus laevis
QECNGECQCPNKVPVCDPGVSLVQDGCGCCKVCSKQLGELCTERDVCDPHKGLFCDFGSRVNRKIGVCTAREGAPCVFGGTVYRSGESFQSSCKYQCTCIDGGVGCVPLCSMDIRLPSPECPFPRRVKLPGKCCEEWVCDQPQERTLVGPALPAFRMEETYGPDPSLIRANCLVQTTEWSACSKTCGMGISTRVTNDNEHCRLEKQSRLCMVRPCEADLEENIKKGKKCIRTPKISKPVKFEFSGCTSVKTYRAKFCGVCTDGRCCTPHRTATLPVEFKCPDGEVMKKNMMFIKTCACHFNCPGDNDIFEAMYYRKMYGDMA

SEQ ID NO: 21 - DNA, 2119 bp, Danio rerio
gtcactcgactcggctcagcctaaacttcttaccaaagctaaactctagctttcactcaacacgatcaggaggaactggattcaaagactacacaacgagatcgacttgatcaaatctgaagagaagactttgtaggatctcaaaagaagatttttctttttgctacaaactttaaagaagtcatcatgttttctggaatgactcaaagtgtgattgctctgctgttcctgactttcttaagatgggctgtggctcaagagtgcagtggacaatgccactgccctgaagtgccgccccagtgttcacctggtgtaagcctagttctggacacctgtgggtgctgccgggtgtgtgccaagcagctgggcgagctgtgcacagaacgagatgtttgcgacccccacaaaggtctttactgtgactacggctccccaagtaaccgtcgtattggtgtctgcacagccagagatggtgccacttgtgtgtttggtggaatggtgtaccgcagtggagagtctttccagagcagttgtaaataccagtgcacgtgtctggacggtgctgtaggttgcgtgcccctctgtggaatggacatcaggctccccagcccagactgcccaatgccccgcagggtgaaagtgcctgggaagtgctgcgaggagtgggtgtgtgactcccctcgtcaaaacacctttgtgggatcagctttagcagcttacagagaggaggagacatacggtcccgatccctccatgatgagagagaactgcctggttcagactacagaatggagcgcatgctcaaaaacctgtggcttgggaatctctactcgtgtgaccaatgacaaccgtgagtgccgccttgagaagcagtctcgcctttgcatggtccgcccttgcgagtcacacctggaggagaaaataaggaaagggaaaaagtgcatccgcacaccacgagtctccaaacccatgaagtttgagatttccggctgtaccactaccaagtcttacagacccaagttctgcggcgtttgcacagatggtcgctgttgcactcctcacagaaccgccaccttgcccatggagttcaagtgccccgatggccaagtcatgaagaagcagatgatgttcatcaagacctgcgcatgccactacaactgccctggggagaacgacatctttgaatccatgtactacaagaagatggttggcgacatggcgtgaaaagtcatgacctgacaaggagttccatgcagtgactgtccacttgaattgaacagatatccatctcatatctcagcacaaattacttgtttgtctttttatggtttgttgctttcattttacgtctttttgcgtgtatgtgtttgcatcttgttggtggactctggatttctatgctgtatgtgtattcatttgtcatggtggatgtctaagcctagggggtggggatcgtaaaatgaccgggccctgttcggacgcccctaaaaaacaatactgcactatagtccgatgctgtgacgcccaatgtcactgattggcagatgacgccaagcccctcctctaaactgtggtgagcagattcagattctgagagcaaaaatagtaagaaatggatggcagctttaaccccagaagctaatcgcctcaccaagctaagaaggcggaaaaaagagacagagagaaattgatgtgctcttatgtttagttgatattgtcagctcagagtttgattgagctgatccaagacatgaagtttggccaagaactatgaaggttaatatagcactgagtgtttgactgtctggcccctcctgttgggaaagagccagtatccctgtcaaaaagactggatgaattgtagcttcattcttttttatgacaatatctatttttgaaagctgtaaataatgtacatattttgtacagcttaagttaatttaaattgaaagcattttgtcttcttatttccttgcttcgtcatgtttaatgatagcttcatgtgctgacttgactattcttggtacagcttgtccaatattttattgagaagtgtgaccaaaatgttacatgttttcacttttgtagtttacaataaaatattatatttttatacaaa

SEQ ID NO: 22 - protein, 345 aa, Danio rerio
MFSGMTQSVIALLFLTFLRWAVAQECSGQCHCPEVPPQCSPGVSLVLDTCGCCRVCAKQLGELCTERDVCDPHKGLYCDYGSPSNRRIGVCTARDGATCVFGGMVYRSGESFQSSCKYQCTCLDGAVGCVPLCGMDIRLPSPDCPMPRRVKVPGKCCEEWVCDSPRQNTFVGSALAAYREEETYGPDPSMMRENCLVQTTEWSACSKTCGLGISTRVTNDNRECRLEKQSRLCMVRPCESHLEEKIRKGKKCIRTPRVSKPMKFEISGCTTTKSYRPKFCGVCTDGRCCTPHRTATLPMEFKCPDGQVMKKQMMFIKTCACHYNCPGENDIFESMYYKKMVGDMA

SEQ ID NO: 23 - protein, 322 aa, Danio rerio
QECSGQCHCPEVPPQCSPGVSLVLDTCGCCRVCAKQLGELCTERDVCDPHKGLYCDYGSPSNRRIGVCTARDGATCVFGGMVYRSGESFQSSCKYQCTCLDGAVGCVPLCGMDIRLPSPDCPMPRRVKVPGKCCEEWVCDSPRQNTFVGSALAAYREEETYGPDPSMMRENCLVQTTEWSACSKTCGLGISTRVTNDNRECRLEKQSRLCMVRPCESHLEEKIRKGKKCIRTPRVSKPMKFEISGCTTTKSYRPKFCGVCTDGRCCTPHRTATLPMEFKCPDGQVMKKQMMFIKTCACHYNCPGENDIFESMYYKKMVGDMA

SEQ ID NO: 24 - DNA, 212 bp, Homo sapiens
cttaccgactggaagacacgtttggcccagacccaactatgattagagccaactgcctggtccagaccacagagtggagcgcctgttccaagacctgtgggatgggcatctccacccgggttaccaatgacaacgcctcctgcaggctagagaagcagagccgcctgtgcatggtcaggccttgcgaagctgacctggaagagaacattaag

SEQ ID NO: 25 - DNA, 1385 bp, Homo sapiens
aagggcaaaaagtgcatccgtactcccaaaatctccaagcctatcaagtttgagctttctggctgcaccagcatgaagacataccgagctaaattctgtggagtatgtaccgacggccgatgctgcaccccccacagaaccaccaccctgccggtggagttcaagtgccctgacggcgaggtcatgaagaagaacatgatgttcatcaagacctgtgcctgccattacaactgtcccggagacaatgacatctttgaatcgctgtactacaggaagatgtacggagacatggcatgaagccagagagtgagagacattaactcattagactggaacttgaactgattcacatctcatttttccgtaaaaatgatttcagtagcacaagttatttaaatctgtttttctaactgggggaaaagattcccacccaattcaaaacattgtgccatgtcaaacaaatagtctatcaaccccagacactggtttgaagaatgttaagacttgacagtggaactacattagtacacagcaccagaatgtatattaaggtgtggctttaggagcagtgggagggtaccagcagaaaggttagtatcatcagatagcatcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttgcctgtaacaagccagattttttaaaatttatattgtaaatattgtgtgtgtgtgtgtgtgtgtatatatatatatatgtacagttatctaagttaatttaaagttgtttgtgcctttttatttttgtttttaatgctttgatatttcaatgttagcctcaatttctgaacaccataggtagaatgtaaagcttgtctgatcgttcaaagcatgaaatggatacttatatggaaattctgctcagatagaatgacagtccgtcaaaacagattgtttgcaaaggggaggcatcagtgtccttggcaggctgatttctaggtaggaaatgtggtagcctcacttttaatgaacaaatggcctttattaaaaactgagtgactctatatagctgatcagttttttcacctggaagcatttgtttctactttgatatgactgtttttcggacagtttatttgttgagagtgtgaccaaaagttacatgtttgcacctttctagttgaaaataaagtgtatattttttctataaa

SEQ ID NO: 26 - DNA, 4247 bp, Homo sapiens
gttccccaactgctgttttattgtgctattcatgcctagacatcacatagctagaaaggcccatcagacccctcaggccactgctgttcctgtcacacattcctgcaaaggaccatgttgctaacttgaaaaaaattactattaattacacttgcagttgttgcttagtaacatttatgattttgtgtttctcgtgacagcatgagcagagatcattaaaaattaaacttacaaagctgctaaagtgggaagaaggagaacttgaagccacaatttttgcacttgcttagaagccatctaatctcaggtttatatgctagatcttgggggaaacactgcatgtctctggtttatattaaaccacatacagcacactactgacactgatttgtgtctggtgcagctggagtttatcaccaagacataaaaaaaccttgaccctgcagaatggcctggaattacaatcagatgggccacatggcatcccggtgaaagaaagccctaaccagttttctgtcttgtttctgctttctccctacagttccaccaggtgagaagagtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataaactgtcctctttcagaaaacagaaaaaaaacacacacacacacaacaaaaatttgaaccaaaacattccgtttacattttagacagtaagtatcttcgttcttgttagtactatatctgttttactgcttttaacttctgatagcgttggaattaaaacaatgtcaaggtgctgttgtcattgctttactggcttaggggatgggggatggggggtatatttttgtttgttttgtgtttttttttcgtttgtttgttttgttttttagttcccacagggagtagagatggggaaagaattcctacaatatatattctggctgataaaagatacatttgtatgttgtgaagatgtttgcaatatcgatcagatgactagaaagtgaataaaaattaaggcaactgaacaaaaaaatgctcacactccacatcccgtgatgcacctcccaggccccgctcattctttgggcgttggtcagagtaagctgcttttgacggaaggacctatgtttgctcagaacacattctttccccccctccccctctggtctcctctttgttttgttttaaggaagaaaaatcagttgcgcgttctgaaatattttaccactgctgtgaacaagtgaacacattgtgtcacatcatgacactcgtataagcatggagaacagtgatttttttttagaacagaaaacaacaaaaaataaccccaaaatgaagattattttttatgaggagtgaacatttgggtaaatcatggctaagcttaaaaaaaactcatggtgaggcttaacaatgtcttgtaagcaaaaggtagagccctgtatcaacccagaaacacctagatcagaacaggaatccacattgccagtgacatgagactgaacagccaaatggaggctatgtggagttggcattgcatttaccggcagtgcgggaggaatttctgagtggccatcccaaggtctaggtggaggtggggcatggtatttgagacattccaaaacgaaggcctctgaaggacccttcagaggtggctctggaatgacatgtgtcaagctgcttggacctcgtgctttaagtgcctacattatctaactgtgctcaagaggttctcgactggaggaccacactcaagccgacttatgcccaccatcccacctctggataattttgcataaaattggattagcctggagcaggttgggagccaaatgtggcatttgtgatcatgagattgatgcaatgagatagaagatgtttgctacctgaacacttattgctttgaaactagacttgaggaaaccagggtttatcttttgagaacttttggtaagggaaaagggaacaggaaaagaaaccccaaactcaggccgaatgatcaaggggacccataggaaatcttgtccagagacaagacttcgggaaggtgtctggacattcagaacaccaagacttgaaggtgccttgctcaatggaagaggccaggacagagctgacaaaattttgctccccagtgaaggccacagcaaccttctgcccatcctgtctgttcatggagagggtccctgcctcacctctgccattttgggttaggagaagtcaagttgggagcctgaaatagtggttcttggaaaaatggatccccagtgaaaactagagctctaagcccattcagcccatttcacacctgaaaatgttagtgatcaccacttggaccagcatccttaagtatcagaaagccccaagcaattgctgcatcttagtagggtgagggataagcaaaagaggatgttcaccataacccaggaatgaagataccatcagcaaagaatttcaatttgttcagtctttcatttagagctagtctttcacagtaccatctgaatacctctttgaaagaaggaagactttacgtagtgtagatttgttttgtgttgtttgaaaatattatctttgtaattatttttaatatgtaaggaatgcttggaatatctgctatatgtcaactttatgcagcttccttttgagggacaaatttaaaacaaacaaccccccatcacaaacttaaaggattgcaagggccagatctgttaagtggtttcataggagacacatccagcaattgtgtggtcagtggctcttttacccaataagatacatcacagtcacatgcttgatggtttatgttgacctaagatttattttgttaaaatctctctctgttgtgttcgttcttgttctgttttgttttgttttttaaagtcttgctgtggtctctttgtggcagaagtgtttcatgcatggcagcaggcctgttgcttttttatggcgattcccattgaaaatgtaagtaaatgtctgtggccttgttctctctatggtaaagatattattcaccatgtaaaacaaaaaacaatatttattgtattttagtatatttatataattatgttattgaaaaaaattggcattaaaacttaaccgcatcagaacctattgtaaatacaagttctatttaagtgtactaattaacatataatatatgttttaaatatagaatttttaatgtttttaaatatattttcaaagtacataaaaaaaaaaaaaaaaaa

SEQ ID NO: 27 - DNA, 4084 bp, Homo sapiens
gtgtgtaatccgggcgataggagtccattcagcaccttggacagagccaacggatttgtccgaggtggcggtacccccaggtagtcttcttggccccgctgtaaagccaaccctgtgtcgcccttaaaaagcgtcttttctgaggttcggctcacactgagatcggggctggagagagagtcagattttggagcggagcgtttggaaagcgagccccagtttggtcccctcattgagctcgctgaagttggcttcctagcggtgtaggctggaatagactcttggcaagctccgggttggtatactgggttaactttgggaaatgcaagtgtttatctccaggatctagccaccggggtggtgtaagccgcaaagaagttccaccaggtgagaagagtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataaactgtcctctttcagaaaacagaaaaaaaacacacacacacacaacaaaaatttgaaccaaaacattccgtttacattttagacagtaagtatcttcgttcttgttagtactatatctgttttactgcttttaacttctgatagcgttggaattaaaacaatgtcaaggtgctgttgtcattgctttactggcttaggggatgggggatggggggtatatttttgtttgttttgtgtttttttttcgtttgtttgttttgttttttagttcccacagggagtagagatggggaaagaattcctacaatatatattctggctgataaaagatacatttgtatgttgtgaagatgtttgcaatatcgatcagatgactagaaagtgaataaaaattaaggcaactgaacaaaaaaatgctcacactccacatcccgtgatgcacctcccaggccccgctcattctttgggcgttggtcagagtaagctgcttttgacggaaggacctatgtttgctcagaacacattctttccccccctccccctctggtctcctctttgttttgttttaaggaagaaaaatcagttgcgcgttctgaaatattttaccactgctgtgaacaagtgaacacattgtgtcacatcatgacactcgtataagcatggagaacagtgatttttttttagaacagaaaacaacaaaaaataaccccaaaatgaagattattttttatgaggagtgaacatttgggtaaatcatggctaagcttaaaaaaaactcatggtgaggcttaacaatgtcttgtaagcaaaaggtagagccctgtatcaacccagaaacacctagatcagaacaggaatccacattgccagtgacatgagactgaacagccaaatggaggctatgtggagttggcattgcatttaccggcagtgcgggaggaatttctgagtggccatcccaaggtctaggtggaggtggggcatggtatttgagacattccaaaacgaaggcctctgaaggacccttcagaggtggctctggaatgacatgtgtcaagctgcttggacctcgtgctttaagtgcctacattatctaactgtgctcaagaggttctcgactggaggaccacactcaagccgacttatgcccaccatcccacctctggataattttgcataaaattggattagcctggagcaggttgggagccaaatgtggcatttgtgatcatgagattgatgcaatgagatagaagatgtttgctacctgaacacttattgctttgaaactagacttgaggaaaccagggtttatcttttgagaacttttggtaagggaaaagggaacaggaaaagaaaccccaaactcaggccgaatgatcaaggggacccataggaaatcttgtccagagacaagacttcgggaaggtgtctggacattcagaacaccaagacttgaaggtgccttgctcaatggaagaggccaggacagagctgacaaaattttgctccccagtgaaggccacagcaaccttctgcccatcctgtctgttcatggagagggtccctgcctcacctctgccattttgggttaggagaagtcaagttgggagcctgaaatagtggttcttggaaaaatggatccccagtgaaaactagagctctaagcccattcagcccatttcacacctgaaaatgttagtgatcaccacttggaccagcatccttaagtatcagaaagccccaagcaattgctgcatcttagtagggtgagggataagcaaaagaggatgttcaccataacccaggaatgaagataccatcagcaaagaatttcaatttgttcagtctttcatttagagctagtctttcacagtaccatctgaatacctctttgaaagaaggaagactttacgtagtgtagatttgttttgtgttgtttgaaaatattatctttgtaattatttttaatatgtaaggaatgcttggaatatctgctatatgtcaactttatgcagcttccttttgagggacaaatttaaaacaaacaaccccccatcacaaacttaaaggattgcaagggccagatctgttaagtggtttcataggagacacatccagcaattgtgtggtcagtggctcttttacccaataagatacatcacagtcacatgcttgatggtttatgttgacctaagatttattttgttaaaatctctctctgttgtgttcgttcttgttctgttttgttttgttttttaaagtcttgctgtggtctctttgtggcagaagtgtttcatgcatggcagcaggcctgttgcttttttatggcgattcccattgaaaatgtaagtaaatgtctgtggccttgttctctctatggtaaagatattattcaccatgtaaaacaaaaaacaatatttattgtattttagtatatttatataattatgttattgaaaaaaattggcattaaaacttaaccgcatcagaacctattgtaaatacaagttctatttaagtgtactaattaacatataatatatgttttaaatatagaatttttaatgtttttaaatatattttcaaagtacataaaaaaaaaaaaaaaaaa

SEQ ID NO: 28 - DNA, 4084 bp, Homo sapiens; identical to SEQ ID NO: 27

SEQ ID NO: 29 - DNA, 3972 bp, Homo sapiens
gctgccgccgccgcgcccgggcgcacccgcccgctcgctgtcccgcgcaccccgtagcgcctcgggctcccgggccggacagaggagccagcccggtgcgcccctccacctcctgctcggggggctttaatgagacacccaccgctgctgtggggccggcggggagcagcaccgcgacggggaccggggctgggcgctggagccagaatcggaaccacgatgtgactccgccgccggggacccgtgaggtttgtgtggaccccgagttccaccaggtgagaagagtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataaactgtcctctttcagaaaacagaaaaaaaacacacacacacacaacaaaaatttgaaccaaaacattccgtttacattttagacagtaagtatcttcgttcttgttagtactatatctgttttactgcttttaacttctgatagcgttggaattaaaacaatgtcaaggtgctgttgtcattgctttactggcttaggggatgggggatggggggtatatttttgtttgttttgtgtttttttttcgtttgtttgttttgttttttagttcccacagggagtagagatggggaaagaattcctacaatatatattctggctgataaaagatacatttgtatgttgtgaagatgtttgcaatatcgatcagatgactagaaagtgaataaaaattaaggcaactgaacaaaaaaatgctcacactccacatcccgtgatgcacctcccaggccccgctcattctttgggcgttggtcagagtaagctgcttttgacggaaggacctatgtttgctcagaacacattctttccccccctccccctctggtctcctctttgttttgttttaaggaagaaaaatcagttgcgcgttctgaaatattttaccactgctgtgaacaagtgaacacattgtgtcacatcatgacactcgtataagcatggagaacagtgatttttttttagaacagaaaacaacaaaaaataaccccaaaatgaagattattttttatgaggagtgaacatttgggtaaatcatggctaagcttaaaaaaaactcatggtgaggcttaacaatgtcttgtaagcaaaaggtagagccctgtatcaacccagaaacacctagatcagaacaggaatccacattgccagtgacatgagactgaacagccaaatggaggctatgtggagttggcattgcatttaccggcagtgcgggaggaatttctgagtggccatcccaaggtctaggtggaggtggggcatggtatttgagacattccaaaacgaaggcctctgaaggacccttcagaggtggctctggaatgacatgtgtcaagctgcttggacctcgtgctttaagtgcctacattatctaactgtgctcaagaggttctcgactggaggaccacactcaagccgacttatgcccaccatcccacctctggataattttgcataaaattggattagcctggagcaggttgggagccaaatgtggcatttgtgatcatgagattgatgcaatgagatagaagatgtttgctacctgaacacttattgctttgaaactagacttgaggaaaccagggtttatcttttgagaacttttggtaagggaaaagggaacaggaaaagaaaccccaaactcaggccgaatgatcaaggggacccataggaaatcttgtccagagacaagacttcgggaaggtgtctggacattcagaacaccaagacttgaaggtgccttgctcaatggaagaggccaggacagagctgacaaaattttgctccccagtgaaggccacagcaaccttctgcccatcctgtctgttcatggagagggtccctgcctcacctctgccattttgggttaggagaagtcaagttgggagcctgaaatagtggttcttggaaaaatggatccccagtgaaaactagagctctaagcccattcagcccatttcacacctgaaaatgttagtgatcaccacttggaccagcatccttaagtatcagaaagccccaagcaattgctgcatcttagtagggtgagggataagcaaaagaggatgttcaccataacccaggaatgaagataccatcagcaaagaatttcaatttgttcagtctttcatttagagctagtctttcacagtaccatctgaatacctctttgaaagaaggaagactttacgtagtgtagatttgttttgtgttgtttgaaaatattatctttgtaattatttttaatatgtaaggaatgcttggaatatctgctatatgtcaactttatgcagcttccttttgagggacaaatttaaaacaaacaaccccccatcacaaacttaaaggattgcaagggccagatctgttaagtggtttcataggagacacatccagcaattgtgtggtcagtggctcttttacccaataagatacatcacagtcacatgcttgatggtttatgttgacctaagatttattttgttaaaatctctctctgttgtgttcgttcttgttctgttttgttttgttttttaaagtcttgctgtggtctctttgtggcagaagtgtttcatgcatggcagcaggcctgttgcttttttatggcgattcccattgaaaatgtaagtaaatgtctgtggccttgttctctctatggtaaagatattattcaccatgtaaaacaaaaaacaatatttattgtattttagtatatttatataattatgttattgaaaaaaattggcattaaaacttaaccgcatcagaacctattgtaaatacaagttctatttaagtgtactaattaacatataatatatgttttaaatatagaatttttaatgtttttaaatatattttcaaagtacataaaaaaaaaaaaaaaaaa

SEQ ID NO: 30 - DNA, 4016 bp, Homo sapiens
ggctgctctcgctgccgctccccccggcgaactagcatgaaatctccctgcctctgccgagatcaaatggagcttctcgctgatggggtgcgagtattacctccgccatgcaatttccactatcaataatttaacttctttgctgcagaacagaaggagtacataccgggcaccaaagactcgcgccccctcccccctttaattaagcgaagggaacgtgaaaaaataatagagtgtgggagttttggggccgaagtctttcccggagcagctgccttgatggttactttgacaagtagtgactgaaaagttccaccaggtgagaagagtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataaactgtcctctttcagaaaacagaaaaaaaacacacacacacacaacaaaaatttgaaccaaaacattccgtttacattttagacagtaagtatcttcgttcttgttagtactatatctgttttactgcttttaacttctgatagcgttggaattaaaacaatgtcaaggtgctgttgtcattgctttactggcttaggggatgggggatggggggtatatttttgtttgttttgtgtttttttttcgtttgtttgttttgttttttagttcccacagggagtagagatggggaaagaattcctacaatatatattctggctgataaaagatacatttgtatgttgtgaagatgtttgcaatatcgatcagatgactagaaagtgaataaaaattaaggcaactgaacaaaaaaatgctcacactccacatcccgtgatgcacctcccaggccccgctcattctttgggcgttggtcagagtaagctgcttttgacggaaggacctatgtttgctcagaacacattctttccccccctccccctctggtctcctctttgttttgttttaaggaagaaaaatcagttgcgcgttctgaaatattttaccactgctgtgaacaagtgaacacattgtgtcacatcatgacactcgtataagcatggagaacagtgatttttttttagaacagaaaacaacaaaaaataaccccaaaatgaagattattttttatgaggagtgaacatttgggtaaatcatggctaagcttaaaaaaaactcatggtgaggcttaacaatgtcttgtaagcaaaaggtagagccctgtatcaacccagaaacacctagatcagaacaggaatccacattgccagtgacatgagactgaacagccaaatggaggctatgtggagttggcattgcatttaccggcagtgcgggaggaatttctgagtggccatcccaaggtctaggtggaggtggggcatggtatttgagacattccaaaacgaaggcctctgaaggacccttcagaggtggctctggaatgacatgtgtcaagctgcttggacctcgtgctttaagtgcctacattatctaactgtgctcaagaggttctcgactggaggaccacactcaagccgacttatgcccaccatcccacctctggataattttgcataaaattggattagcctggagcaggttgggagccaaatgtggcatttgtgatcatgagattgatgcaatgagatagaagatgtttgctacctgaacacttattgctttgaaactagacttgaggaaaccagggtttatcttttgagaacttttggtaagggaaaagggaacaggaaaagaaaccccaaactcaggccgaatgatcaaggggacccataggaaatcttgtccagagacaagacttcgggaaggtgtctggacattcagaacaccaagacttgaaggtgccttgctcaatggaagaggccaggacagagctgacaaaattttgctccccagtgaaggccacagcaaccttctgcccatcctgtctgttcatggagagggtccctgcctcacctctgccattttgggttaggagaagtcaagttgggagcctgaaatagtggttcttggaaaaatggatccccagtgaaaactagagctctaagcccattcagcccatttcacacctgaaaatgttagtgatcaccacttggaccagcatccttaagtatcagaaagccccaagcaattgctgcatcttagtagggtgagggataagcaaaagaggatgttcaccataacccaggaatgaagataccatcagcaaagaatttcaatttgttcagtctttcatttagagctagtctttcacagtaccatctgaatacctctttgaaagaaggaagactttacgtagtgtagatttgttttgtgttgtttgaaaatattatctttgtaattatttttaatatgtaaggaatgcttggaatatctgctatatgtcaactttatgcagcttccttttgagggacaaatttaaaacaaacaaccccccatcacaaacttaaaggattgcaagggccagatctgttaagtggtttcataggagacacatccagcaattgtgtggtcagtggctcttttacccaataagatacatcacagtcacatgcttgatggtttatgttgacctaagatttattttgttaaaatctctctctgttgtgttcgttcttgttctgttttgttttgttttttaaagtcttgctgtggtctctttgtggcagaagtgtttcatgcatggcagcaggcctgttgcttttttatggcgattcccattgaaaatgtaagtaaatgtctgtggccttgttctctctatggtaaagatattattcaccatgtaaaacaaaaaacaatatttattgtattttagtatatttatataattatgttattgaaaaaaattggcattaaaacttaaccgcatcagaacctattgtaaatacaagttctatttaagtgtactaattaacatataatatatgttttaaatatagaatttttaatgtttttaaatatattttcaaagtacataaaaaaaaaaaaaaaaaa

SEQ ID NO: 31 - DNA, 3958 bp, Homo sapiens
agtggacttacaagtccgaagccaatgtagcttggaaaacttgggaggcggaattcctaccgctgggaactgaaagggtctgcgacactctcgggcaggccgaacccacatctctacccatcctgcgcccctcttctgaagcgccctccagggaagttaagagttttgactttcggggagtggttgggatgtacgtgggggattcttgactcgggttagtctctggggatgcagagccgggaagaggaatggttccaccaggtgagaagagtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataaactgtcctctttcagaaaacagaaaaaaaacacacacacacacaacaaaaatttgaaccaaaacattccgtttacattttagacagtaagtatcttcgttcttgttagtactatatctgttttactgcttttaacttctgatagcgttggaattaaaacaatgtcaaggtgctgttgtcattgctttactggcttaggggatgggggatggggggtatatttttgtttgttttgtgtttttttttcgtttgtttgttttgttttttagttcccacagggagtagagatggggaaagaattcctacaatatatattctggctgataaaagatacatttgtatgttgtgaagatgtttgcaatatcgatcagatgactagaaagtgaataaaaattaaggcaactgaacaaaaaaatgctcacactccacatcccgtgatgcacctcccaggccccgctcattctttgggcgttggtcagagtaagctgcttttgacggaaggacctatgtttgctcagaacacattctttccccccctccccctctggtctcctctttgttttgttttaaggaagaaaaatcagttgcgcgttctgaaatattttaccactgctgtgaacaagtgaacacattgtgtcacatcatgacactcgtataagcatggagaacagtgatttttttttagaacagaaaacaacaaaaaataaccccaaaatgaagattattttttatgaggagtgaacatttgggtaaatcatggctaagcttaaaaaaaactcatggtgaggcttaacaatgtcttgtaagcaaaaggtagagccctgtatcaacccagaaacacctagatcagaacaggaatccacattgccagtgacatgagactgaacagccaaatggaggctatgtggagttggcattgcatttaccggcagtgcgggaggaatttctgagtggccatcccaaggtctaggtggaggtggggcatggtatttgagacattccaaaacgaaggcctctgaaggacccttcagaggtggctctggaatgacatgtgtcaagctgcttggacctcgtgctttaagtgcctacattatctaactgtgctcaagaggttctcgactggaggaccacactcaagccgacttatgcccaccatcccacctctggataattttgcataaaattggattagcctggagcaggttgggagccaaatgtggcatttgtgatcatgagattgatgcaatgagatagaagatgtttgctacctgaacacttattgctttgaaactagacttgaggaaaccagggtttatcttttgagaacttttggtaagggaaaagggaacaggaaaagaaaccccaaactcaggccgaatgatcaaggggacccataggaaatcttgtccagagacaagacttcgggaaggtgtctggacattcagaacaccaagacttgaaggtgccttgctcaatggaagaggccaggacagagctgacaaaattttgctccccagtgaaggccacagcaaccttctgcccatcctgtctgttcatggagagggtccctgcctcacctctgccattttgggttaggagaagtcaagttgggagcctgaaatagtggttcttggaaaaatggatccccagtgaaaactagagctctaagcccattcagcccatttcacacctgaaaatgttagtgatcaccacttggaccagcatccttaagtatcagaaagccccaagcaattgctgcatcttagtagggtgagggataagcaaaagaggatgttcaccataacccaggaatgaagataccatcagcaaagaatttcaatttgttcagtctttcatttagagctagtctttcacagtaccatctgaatacctctttgaaagaaggaagactttacgtagtgtagatttgttttgtgttgtttgaaaatattatctttgtaattatttttaatatgtaaggaatgcttggaatatctgctatatgtcaactttatgcagcttccttttgagggacaaatttaaaacaaacaaccccccatcacaaacttaaaggattgcaagggccagatctgttaagtggtttcataggagacacatccagcaattgtgtggtcagtggctcttttacccaataagatacatcacagtcacatgcttgatggtttatgttgacctaagatttattttgttaaaatctctctctgttgtgttcgttcttgttctgttttgttttgttttttaaagtcttgctgtggtctctttgtggcagaagtgtttcatgcatggcagcaggcctgttgcttttttatggcgattcccattgaaaatgtaagtaaatgtctgtggccttgttctctctatggtaaagatattattcaccatgtaaaacaaaaaacaatatttattgtattttagtatatttatataattatgttattgaaaaaaattggcattaaaacttaaccgcatcagaacctattgtaaatacaagttctatttaagtgtactaattaacatataatatatgttttaaatatagaatttttaatgtttttaaatatattttcaaagtacataaaaaaaaaaaaaaaaaa

SEQ ID NO: 32 - protein, 247 aa, Homo sapiens
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 33 - protein, 229 aa, Homo sapiens
APMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 34 - protein, 119 aa, Homo sapiens
HSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 35 - DNA, 4302 bp, Mus musculus
taaagcagtagccggctggtgcagaaaagcaacaagttccccagcggtcttcccgccctagcttgacaaggcgaagggtttcttacctggcgacagggaaatctcctgagccgagctcatctttgccagagccccaggtgtgacctgagcagtgggcaaaggatcggcgtgcaaattggattatttttatgggggtactctgaaactccctcactttctctgggaactttttgtgctagggctcagtgacaggcgttgagaaagctgcttcaggaaacgcccgctatatagcagggcaattggacagtcattggtaacctcgctcattcattagaatcacgtaagaactcaaagggaaacgtgtctctcagaatgagggcgtttgcgtaaatctataggtttttcaacatcgatgccagttgctttgtcttctgtagtcgccaaggtggatgagagttgaagctttgcggatattgcgaagggttattagattcataagtcacaccaagtggtgggcgatccactgagcaaagccgaacttctcacatgatgacttcaaacaagacacattaccttcctgcatctgttggggagacaagattttaagacactgagtctccaggacagcaaagccacaatgttccaccaggtgagaagagtgatgaccatccttttccttactatggttatttcatacttcggttgcatgaaggcggcgcccatgaaagaagtaaacgtccacggacaaggcaacttggcctacccaggtgtgcggacccatgggactctggagagcgtgaatgggcccagggcaggttcgagaggtctgacgacgacatcactggctgacacttttgagcacgtcatcgaagagctgctggatgaggaccagaaggttcggcccaacgaagaaaaccataaggacgcggacttgtacacttcccgggtgatgctcagcagtcaagtgcctttggagcctcctctactctttctgctggaggaatacaaaaattacctggatgccgcaaacatgtctatgagggttcggcgccactccgaccctgcccgccgtggggagctgagcgtgtgtgacagtattagcgagtgggtcacagcggcagataaaaagactgcagtggacatgtctggcgggacggtcacagtcctagagaaagtcccggtatccaaaggccaactgaagcagtatttctacgagaccaagtgtaatcccatgggttacaccaaggaaggctgcaggggcatagacaaaaggcactggaactcgcaatgccgaactacccaatcgtatgttcgggcccttactatggatagcaaaaagagaattggctggcgattcataaggatagacacttcctgtgtatgtacactgaccattaaaaggggaagatagtggatttatgttgtatagattatattgagacaaaattatctatttgtatatatacataacagggtaaattattcagttaagaaaaaataattttatgaactgcatgtataaatgaagtttatacagtacagtggttctacaatctatttattggacatatccatgacctgaaaggaaacagtcatttgcgcacaactttaaaagtctgcattacattcctcgataatgttgtggtttgttgccgttgccaagaattgaaaacaaaaagtttaaaaaaaataataataaattgcatgctgctttaattgtgaattgataataaactgtccctctttcagaaaacagattaaaaaaacaaaaaacaaaaaaaaaaaaacaaaaaacaaaaacaaaaattggaaccaaaacattccgtttacattttagacactaagtatcttcgttcttgttagtactctgttttactgctttcgacttctgatagcgttggaattaaaacaatgtcaaggtgctgttgtcattgctttactggcgtaagggacggggaatgggaggggtagatttctgtttgttttgtgttttattttgtttgtttgtttgttttgttttttagttccacccggagtagggatggagaaaatttcttcactatccattctggttgataaagcgttacatttgtatgttgtaaagatgtttgcaaaatccaatcagatgactggaaaacaaataaaaattaaggcaactgaataaaatgctcacactccactgcccatgatgtatctccctggtccccctcagctcactcttctggcatgggtcagggaaaattgcttttattggaaagaccagcatttgttcaaagcatactctttccctccctcctcccattttggtcccttctttttgttttgttttaagaaagaaaattaagttgcgcgctttaaaatattttactactgctacaaacagatgaacaatgtgtgtcattttatgacactcatggaaaacagtgatttttttttaccctaaagaaaaacaaataaaaataacccaaaatattctttttttaaaaggcataaatattgggtaaattgtaatatggcctaacagtgtttgcagataaaagttattgtatacacccagatacttagataagagcagggatccacactgccattgaaataggactgaatggccctgcggaggctaagtggagctgacatactatttcctggcagtgcaggaggaatttctgagtggccatcctaaggtctaggatggaggtggggaatggtacttgagacattcctaaaggaaggctcggaagcacccttcagagcaggctctggaatgatgtgtcaagttgcttaggccttctgctttaagtgcctacattacctaacagtgctcaagaggttctcgattggagaaccacactcaaatccatttatagcctccatcccatttctaaataattgtgtataaagttggattaacctggagcaactttggatccaaatatggcacagcaataatgatattaatgcagcatgatgggaaatgtttgctgtgaagagaattgatttgctttgagcttagacttcaggaagcctaggttttttatttttttatttttgagacattttggtaaaaggaaaaaaagaaaacaaacaaacaaacaaacaaaaccagaaaaagcatcaaaactcaggcagaatgagcaatgtctgaaagggctagaaaaacaagacatagcaaggtgctttcactgtgaaagagacaagaacacaggaggaaatattgcttcagtgaagagcacagacggctcctgccaatttattacaagagtcccgtctgtactttaccctttggggttagaagtcaagttggaagcctgaatgaatggacccaatgagaactagtgttaagcccatttccctagtcaggtttttttcaagcgtgaatgtgttagtggttactctcctgggttcctgagcatcagaaaaaaaaaaaaaaagaggcaaacaatcgcttcatcttaggagtggaaaggaaacagaagtggacgtccgctgtgactcagggagtgaagataccatcagcaaatagtttcttttttgttcattcgttcctttcgagttagcctgtcttttggaataccactgaatatgctgtttttgaaagacttcatgtagcatagattgttttgtgccgtttaccaaattaacctttgtcatcgttttttaacctattcaggaatgcttggaatatctgctctatgttaactttttgcagcttcattctgagagacattagtcaaacaaacaaaaggatccccatcacaatcttacagtactgcaagggccaggtctgttaatcggcttcacaggagacatcagcaattgtgtggtcagtggctggctctcttacccactaagatacatcatagctacatgttggtggtttatgttgacctgagatttatttgttaaaatctcttcttcgtttctgttcgttctggttctgttctgttctgttctgttctgttttggttttaaagtcttgctgtggtctcttgttggcagaaatgttttatgcatggcagcaggcctgttgcttttttatagtgattcccattgaaactgtaagtaaatgtctgtggccttgttctctctatggtaaagatattattcaccatgtaaaacaagaaaaaatatttattgtattttagtatatttatataattatgttattgaaaaaattggcattaaaacttaaccacatcagaagcctattgtaaatacaggttctatttaagtgtaccaattaacatataatatatgttttaaatatagaatttttaatgtttttaaatatattttcaaagt

SEQ ID NO: 36 - DNA, 4161 bp, Mus musculus
gctttggcaaagccatccacacgtgacaaaacgtaaggaagtggaagaaaccgtctagagcaatatcaagtaccacttaattagagaatatttttttaaccttttcctcctcctgcgccgggtgtgtgatcccggagagcagagtccattcagcaccttggacagagccagcggatttgtccgaggtggtagtacttcatccaggtattcttttcctcgctgtcaagccaacccggtgtcgcccttaaaaagcgtcttttccgaggttcggctcacaccgagatcggggctggagagagagtcagattttggagcggagcgtttggagagcgagccccagtttggtcccctcattgagctcgctgaagttggcttcctagcggtgtaggctggaatagactcttggcaagctccgggttggtatactgggttaactttgggaaatgcaagtgtttatcaccaggatctagccaccggggtggtgtaagccgcaaagaagttccaccaggtgagaagagtgatgaccatccttttccttactatggttatttcatacttcggttgcatgaaggcggcgcccatgaaagaagtaaacgtccacggacaaggcaacttggcctacccaggtgtgcggacccatgggactctggagagcgtgaatgggcccagggcaggttcgagaggtctgacgacgacatcactggctgacacttttgagcacgtcatcgaagagctgctggatgaggaccagaaggttcggcccaacgaagaaaaccataaggacgcggacttgtacacttcccgggtgatgctcagcagtcaagtgcctttggagcctcctctactctttctgctggaggaatacaaaaattacctggatgccgcaaacatgtctatgagggttcggcgccactccgaccctgcccgccgtggggagctgagcgtgtgtgacagtattagcgagtgggtcacagcggcagataaaaagactgcagtggacatgtctggcgggacggtcacagtcctagagaaagtcccggtatccaaaggccaactgaagcagtatttctacgagaccaagtgtaatcccatgggttacaccaaggaaggctgcaggggcatagacaaaaggcactggaactcgcaatgccgaactacccaatcgtatgttcgggcccttactatggatagcaaaaagagaattggctggcgattcataaggatagacacttcctgtgtatgtacactgaccattaaaaggggaagatagtggatttatgttgtatagattatattgagacaaaattatctatttgtatatatacataacagggtaaattattcagttaagaaaaaataattttatgaactgcatgtataaatgaagtttatacagtacagtggttctacaatctatttattggacatatccatgacctgaaaggaaacagtcatttgcgcacaactttaaaagtctgcattacattcctcgataatgttgtggtttgttgccgttgccaagaattgaaaacaaaaagtttaaaaaaaataataataaattgcatgctgctttaattgtgaattgataataaactgtccctctttcagaaaacagattaaaaaaacaaaaaacaaaaaaaaaaaaacaaaaaacaaaaacaaaaattggaaccaaaacattccgtttacattttagacactaagtatcttcgttcttgttagtactctgttttactgctttcgacttctgatagcgttggaattaaaacaatgtcaaggtgctgttgtcattgctttactggcgtaagggacggggaatgggaggggtagatttctgtttgttttgtgttttattttgtttgtttgtttgttttgttttttagttccacccggagtagggatggagaaaatttcttcactatccattctggttgataaagcgttacatttgtatgttgtaaagatgtttgcaaaatccaatcagatgactggaaaacaaataaaaattaaggcaactgaataaaatgctcacactccactgcccatgatgtatctccctggtccccctcagctcactcttctggcatgggtcagggaaaattgcttttattggaaagaccagcatttgttcaaagcatactctttccctccctcctcccattttggtcccttctttttgttttgttttaagaaagaaaattaagttgcgcgctttaaaatattttactactgctacaaacagatgaacaatgtgtgtcattttatgacactcatggaaaacagtgatttttttttaccctaaagaaaaacaaataaaaataacccaaaatattctttttttaaaaggcataaatattgggtaaattgtaatatggcctaacagtgtttgcagataaaagttattgtatacacccagatacttagataagagcagggatccacactgccattgaaataggactgaatggccctgcggaggctaagtggagctgacatactatttcctggcagtgcaggaggaatttctgagtggccatcctaaggtctaggatggaggtggggaatggtacttgagacattcctaaaggaaggctcggaagcacccttcagagcaggctctggaatgatgtgtcaagttgcttaggccttctgctttaagtgcctacattacctaacagtgctcaagaggttctcgattggagaaccacactcaaatccatttatagcctccatcccatttctaaataattgtgtataaagttggattaacctggagcaactttggatccaaatatggcacagcaataatgatattaatgcagcatgatgggaaatgtttgctgtgaagagaattgatttgctttgagcttagacttcaggaagcctaggttttttatttttttatttttgagacattttggtaaaaggaaaaaaagaaaacaaacaaacaaacaaacaaaaccagaaaaagcatcaaaactcaggcagaatgagcaatgtctgaaagggctagaaaaacaagacatagcaaggtgctttcactgtgaaagagacaagaacacaggaggaaatattgcttcagtgaagagcacagacggctcctgccaatttattacaagagtcccgtctgtactttaccctttggggttagaagtcaagttggaagcctgaatgaatggacccaatgagaactagtgttaagcccatttccctagtcaggtttttttcaagcgtgaatgtgttagtggttactctcctgggttcctgagcatcagaaaaaaaaaaaaaaagaggcaaacaatcgcttcatcttaggagtggaaaggaaacagaagtggacgtccgctgtgactcagggagtgaagataccatcagcaaatagtttcttttttgttcattcgttcctttcgagttagcctgtcttttggaataccactgaatatgctgtttttgaaagacttcatgtagcatagattgttttgtgccgtttaccaaattaacctttgtcatcgttttttaacctattcaggaatgcttggaatatctgctctatgttaactttttgcagcttcattctgagagacattagtcaaacaaacaaaaggatccccatcacaatcttacagtactgcaagggccaggtctgttaatcggcttcacaggagacatcagcaattgtgtggtcagtggctggctctcttacccactaagatacatcatagctacatgttggtggtttatgttgacctgagatttatttgttaaaatctcttcttcgtttctgttcgttctggttctgttctgttctgttctgttctgttttggttttaaagtcttgctgtggtctcttgttggcagaaatgttttatgcatggcagcaggcctgttgcttttttatagtgattcccattgaaactgtaagtaaatgtctgtggccttgttctctctatggtaaagatattattcaccatgtaaaacaagaaaaaatatttattgtattttagtatatttatataattatgttattgaaaaaattggcattaaaacttaaccacatcagaagcctattgtaaatacaggttctatttaagtgtaccaattaacatataatatatgttttaaatatagaatttttaatgtttttaaatatattttcaaagt

SEQ ID NO: 37 - DNA, 4001 bp, Mus musculus
acccactttcccattcaccgaggagaggactgctctcgctgccgctccccccacccacccccggcgagctagcatgaaatctcccagcctctgcctagatcaaatggagcttctcgctgaaggcgtgcgagtattacctccgccatgcaatttccactatcaataatttaacttctttgctgcagaacaggagtacatatcggccaccaaagactcgccccctcccccttttaactgaagagaaggggaaatatatagtaagagtctagaaccttggggaccggtcttccccagagcagctgccttgatgtttactttgacaagtagtgactgaaaaagttccaccaggtgagaagagtgatgaccatccttttccttactatggttatttcatacttcggttgcatgaaggcggcgcccatgaaagaagtaaacgtccacggacaaggcaacttggcctacccaggtgtgcggacccatgggactctggagagcgtgaatgggcccagggcaggttcgagaggtctgacgacgacatcactggctgacacttttgagcacgtcatcgaagagctgctggatgaggaccagaaggttcggcccaacgaagaaaaccataaggacgcggacttgtacacttcccgggtgatgctcagcagtcaagtgcctttggagcctcctctactctttctgctggaggaatacaaaaattacctggatgccgcaaacatgtctatgagggttcggcgccactccgaccctgcccgccgtggggagctgagcgtgtgtgacagtattagcgagtgggtcacagcggcagataaaaagactgcagtggacatgtctggcgggacggtcacagtcctagagaaagtcccggtatccaaaggccaactgaagcagtatttctacgagaccaagtgtaatcccatgggttacaccaaggaaggctgcaggggcatagacaaaaggcactggaactcgcaatgccgaactacccaatcgtatgttcgggcccttactatggatagcaaaaagagaattggctggcgattcataaggatagacacttcctgtgtatgtacactgaccattaaaaggggaagatagtggatttatgttgtatagattatattgagacaaaattatctatttgtatatatacataacagggtaaattattcagttaagaaaaaataattttatgaactgcatgtataaatgaagtttatacagtacagtggttctacaatctatttattggacatatccatgacctgaaaggaaacagtcatttgcgcacaactttaaaagtctgcattacattcctcgataatgttgtggtttgttgccgttgccaagaattgaaaacaaaaagtttaaaaaaaataataataaattgcatgctgctttaattgtgaattgataataaactgtccctctttcagaaaacagattaaaaaaacaaaaaacaaaaaaaaaaaaacaaaaaacaaaaacaaaaattggaaccaaaacattccgtttacattttagacactaagtatcttcgttcttgttagtactctgttttactgctttcgacttctgatagcgttggaattaaaacaatgtcaaggtgctgttgtcattgctttactggcgtaagggacggggaatgggaggggtagatttctgtttgttttgtgttttattttgtttgtttgtttgttttgttttttagttccacccggagtagggatggagaaaatttcttcactatccattctggttgataaagcgttacatttgtatgttgtaaagatgtttgcaaaatccaatcagatgactggaaaacaaataaaaattaaggcaactgaataaaatgctcacactccactgcccatgatgtatctccctggtccccctcagctcactcttctggcatgggtcagggaaaattgcttttattggaaagaccagcatttgttcaaagcatactctttccctccctcctcccattttggtcccttctttttgttttgttttaagaaagaaaattaagttgcgcgctttaaaatattttactactgctacaaacagatgaacaatgtgtgtcattttatgacactcatggaaaacagtgatttttttttaccctaaagaaaaacaaataaaaataacccaaaatattctttttttaaaaggcataaatattgggtaaattgtaatatggcctaacagtgtttgcagataaaagttattgtatacacccagatacttagataagagcagggatccacactgccattgaaataggactgaatggccctgcggaggctaagtggagctgacatactatttcctggcagtgcaggaggaatttctgagtggccatcctaaggtctaggatggaggtggggaatggtacttgagacattcctaaaggaaggctcggaagcacccttcagagcaggctctggaatgatgtgtcaagttgcttaggccttctgctttaagtgcctacattacctaacagtgctcaagaggttctcgattggagaaccacactcaaatccatttatagcctccatcccatttctaaataattgtgtataaagttggattaacctggagcaactttggatccaaatatggcacagcaataatgatattaatgcagcatgatgggaaatgtttgctgtgaagagaattgatttgctttgagcttagacttcaggaagcctaggttttttatttttttatttttgagacattttggtaaaaggaaaaaaagaaaacaaacaaacaaacaaacaaaaccagaaaaagcatcaaaactcaggcagaatgagcaatgtctgaaagggctagaaaaacaagacatagcaaggtgctttcactgtgaaagagacaagaacacaggaggaaatattgcttcagtgaagagcacagacggctcctgccaatttattacaagagtcccgtctgtactttaccctttggggttagaagtcaagttggaagcctgaatgaatggacccaatgagaactagtgttaagcccatttccctagtcaggtttttttcaagcgtgaatgtgttagtggttactctcctgggttcctgagcatcagaaaaaaaaaaaaaaagaggcaaacaatcgcttcatcttaggagtggaaaggaaacagaagtggacgtccgctgtgactcagggagtgaagataccatcagcaaatagtttcttttttgttcattcgttcctttcgagttagcctgtcttttggaataccactgaatatgctgtttttgaaagacttcatgtagcatagattgttttgtgccgtttaccaaattaacctttgtcatcgttttttaacctattcaggaatgcttggaatatctgctctatgttaactttttgcagcttcattctgagagacattagtcaaacaaacaaaaggatccccatcacaatcttacagtactgcaagggccaggtctgttaatcggcttcacaggagacatcagcaattgtgtggtcagtggctggctctcttacccactaagatacatcatagctacatgttggtggtttatgttgacctgagatttatttgttaaaatctcttcttcgtttctgttcgttctggttctgttctgttctgttctgttctgttttggttttaaagtcttgctgtggtctcttgttggcagaaatgttttatgcatggcagcaggcctgttgcttttttatagtgattcccattgaaactgtaagtaaatgtctgtggccttgttctctctatggtaaagatattattcaccatgtaaaacaagaaaaaatatttattgtattttagtatatttatataattatgttattgaaaaaattggcattaaaacttaaccacatcagaagcctattgtaaatacaggttctatttaagtgtaccaattaacatataatatatgttttaaatatagaatttttaatgtttttaaatatattttcaaagt

SEQ ID NO: 38 - DNA, 4009 bp, Mus musculus
ccaatcgaagctcaaccgaagagctaaataatgtctgaccccagtgcctggctctggctgagctctgggtgcccgtcgctgctgccgtgccggggcgcacccgctggctggctgtcgcacggttcccagtgcgcccgggactcccgggcttggagaaggaaaccgcctggggcggcgcgccacctccgcctgggaggctttgatgagacccggttccttcaactgccaccactgccttggggcagacgagaaagcgcacggggcccagggcagggcgcagggaccagaagcgtgacaacaatgtgactccactgccggggatccgagagctttgtgtggaccctgagttccaccaggtgagaagagtgatgaccatccttttccttactatggttatttcatacttcggttgcatgaaggcggcgcccatgaaagaagtaaacgtccacggacaaggcaacttggcctacccaggtgtgcggacccatgggactctggagagcgtgaatgggcccagggcaggttcgagaggtctgacgacgacatcactggctgacacttttgagcacgtcatcgaagagctgctggatgaggaccagaaggttcggcccaacgaagaaaaccataaggacgcggacttgtacacttcccgggtgatgctcagcagtcaagtgcctttggagcctcctctactctttctgctggaggaatacaaaaattacctggatgccgcaaacatgtctatgagggttcggcgccactccgaccctgcccgccgtggggagctgagcgtgtgtgacagtattagcgagtgggtcacagcggcagataaaaagactgcagtggacatgtctggcgggacggtcacagtcctagagaaagtcccggtatccaaaggccaactgaagcagtatttctacgagaccaagtgtaatcccatgggttacaccaaggaaggctgcaggggcatagacaaaaggcactggaactcgcaatgccgaactacccaatcgtatgttcgggcccttactatggatagcaaaaagagaattggctggcgattcataaggatagacacttcctgtgtatgtacactgaccattaaaaggggaagatagtggatttatgttgtatagattatattgagacaaaattatctatttgtatatatacataacagggtaaattattcagttaagaaaaaataattttatgaactgcatgtataaatgaagtttatacagtacagtggttctacaatctatttattggacatatccatgacctgaaaggaaacagtcatttgcgcacaactttaaaagtctgcattacattcctcgataatgttgtggtttgttgccgttgccaagaattgaaaacaaaaagtttaaaaaaaataataataaattgcatgctgctttaattgtgaattgataataaactgtccctctttcagaaaacagattaaaaaaacaaaaaacaaaaaaaaaaaaacaaaaaacaaaaacaaaaattggaaccaaaacattccgtttacattttagacactaagtatcttcgttcttgttagtactctgttttactgctttcgacttctgatagcgttggaattaaaacaatgtcaaggtgctgttgtcattgctttactggcgtaagggacggggaatgggaggggtagatttctgtttgttttgtgttttattttgtttgtttgtttgttttgttttttagttccacccggagtagggatggagaaaatttcttcactatccattctggttgataaagcgttacatttgtatgttgtaaagatgtttgcaaaatccaatcagatgactggaaaacaaataaaaattaaggcaactgaataaaatgctcacactccactgcccatgatgtatctccctggtccccctcagctcactcttctggcatgggtcagggaaaattgcttttattggaaagaccagcatttgttcaaagcatactctttccctccctcctcccattttggtcccttctttttgttttgttttaagaaagaaaattaagttgcgcgctttaaaatattttactactgctacaaacagatgaacaatgtgtgtcattttatgacactcatggaaaacagtgatttttttttaccctaaagaaaaacaaataaaaataacccaaaatattctttttttaaaaggcataaatattgggtaaattgtaatatggcctaacagtgtttgcagataaaagttattgtatacacccagatacttagataagagcagggatccacactgccattgaaataggactgaatggccctgcggaggctaagtggagctgacatactatttcctggcagtgcaggaggaatttctgagtggccatcctaaggtctaggatggaggtggggaatggtacttgagacattcctaaaggaaggctcggaagcacccttcagagcaggctctggaatgatgtgtcaagttgcttaggccttctgctttaagtgcctacattacctaacagtgctcaagaggttctcgattggagaaccacactcaaatccatttatagcctccatcccatttctaaataattgtgtataaagttggattaacctggagcaactttggatccaaatatggcacagcaataatgatattaatgcagcatgatgggaaatgtttgctgtgaagagaattgatttgctttgagcttagacttcaggaagcctaggttttttatttttttatttttgagacattttggtaaaaggaaaaaaagaaaacaaacaaacaaacaaacaaaaccagaaaaagcatcaaaactcaggcagaatgagcaatgtctgaaagggctagaaaaacaagacatagcaaggtgctttcactgtgaaagagacaagaacacaggaggaaatattgcttcagtgaagagcacagacggctcctgccaatttattacaagagtcccgtctgtactttaccctttggggttagaagtcaagttggaagcctgaatgaatggacccaatgagaactagtgttaagcccatttccctagtcaggtttttttcaagcgtgaatgtgttagtggttactctcctgggttcctgagcatcagaaaaaaaaaaaaaaagaggcaaacaatcgcttcatcttaggagtggaaaggaaacagaagtggacgtccgctgtgactcagggagtgaagataccatcagcaaatagtttcttttttgttcattcgttcctttcgagttagcctgtcttttggaataccactgaatatgctgtttttgaaagacttcatgtagcatagattgttttgtgccgtttaccaaattaacctttgtcatcgttttttaacctattcaggaatgcttggaatatctgctctatgttaactttttgcagcttcattctgagagacattagtcaaacaaacaaaaggatccccatcacaatcttacagtactgcaagggccaggtctgttaatcggcttcacaggagacatcagcaattgtgtggtcagtggctggctctcttacccactaagatacatcatagctacatgttggtggtttatgttgacctgagatttatttgttaaaatctcttcttcgtttctgttcgttctggttctgttctgttctgttctgttctgttttggttttaaagtcttgctgtggtctcttgttggcagaaatgttttatgcatggcagcaggcctgttgcttttttatagtgattcccattgaaactgtaagtaaatgtctgtggccttgttctctctatggtaaagatattattcaccatgtaaaacaagaaaaaatatttattgtattttagtatatttatataattatgttattgaaaaaattggcattaaaacttaaccacatcagaagcctattgtaaatacaggttctatttaagtgtaccaattaacatataatatatgttttaaatatagaatttttaatgtttttaaatatattttcaaagt

SEQ ID NO: 39 - protein, 249 aa, Mus musculus
MTILFLTMVISYFGCMKAAPMKEVNVHGQGNLAYPGVRTHGTLESVNGPRAGSRGLTTTSLADTFEHVIEELLDEDQKVRPNEENHKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 40 - protein, 221 aa, Mus musculus
APMKEVNVHGQGNLAYPGVRTHGTLESVNGPRAGSRGLTTTSLADTFEHVIEELLDEDQKVRPNEENHKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWCTLTIKRGR

SEQ ID NO: 41 - protein, 119 aa, Mus musculus; identical to SEQ ID NO: 34

SEQ ID NO: 42 - DNA, 4252 bp, Rattus norvegicus
taaagcggtagccggctggtgcaggaaagcaacaagttccccagcggtcttcccgccctagcctgacaaggcgaaggttttcttacctggcgacagggaaatctcctgagccgagctcatctttgccacagccccaggtgtgacctgagcagtgggcaaaggagcggcgtgcaaattggattatttgtatgggggtactctgaaactccctcactttttctgggaactttttgtgctagggcgcagtgacaggcgttgagaaagctgcttcaggaaacgcccgctatatagcagggcagttggacagtcattggtaacctcgctcattcattagaatcacgtaagaactcaaagggaaacgtgtctctcagaatgagggcgtttgcgtaaatctataggtttttcaacatcgatgccagttgctttgtcttctgtaatcgccaaggtggatgagagttgaagcttgcggatattgcaaagggttattagattcataagtcacaccaagtggtgggcgatccactgagcaaagccgaacttctcacatgatgacttcaaacaagacacattaccttccagcatctgttggggagacgagattttaagacactgagtctccaggacagcaaagccacaatgttccaccaggtgagaagagtgatgaccatccttttccttactatggttatttcatacttcggttgcatgaaggctgcgcccatgaaagaagcaaacgtccacggacaaggcaacttggcctacccagctgtgcggacccatgggactctggagagcgtgaatgggcccagggcaggttcgagaggtctgacgacgacgtccctggctgacacttttgagcacgtgatcgaagagctgctggatgaggaccagaaggttcggcccaacgaagaaaaccataaggacgcggacttgtacacttcccgggtgatgctcagcagtcaagtgcctttggagcctcctctgctctttctgctggaggaatacaaaaattacctggatgccgcaaacatgtctatgagggttcggcgccactccgaccccgcccgccgtggggagctgagcgtgtgtgacagtattagcgagtgggtcacagcggcagataaaaagactgcagtggacatgtccggtgggacggtcacagtcctggagaaagtcccggtatcaaaaggccaactgaagcaatatttctacgagaccaagtgtaatcccatgggttacacgaaggaaggctgcaggggcatagacaaaaggcactggaactcgcaatgccgaactacccaatcgtatgttcgggcccttactatggatagcaaaaagagaattggctggcggttcataaggatagacacttcctgtgtatgtacactgaccattaaaaggggaagatagtggatttatgttgtatagattatattgagacaaaaattatctatttgtatatatacataacagggtaaattattcagttaagaaaaagataattttatgaactgcatgtataaatgaagtttatacagtacagtggttctacaatctatttattggacatatccatgaccagaaagaaacagtcatttgcgcacaactttaaaagtctgcattacattcctcgataatgttgtggtttgttgccgttgccaagaattgaaaacaaaaagttaaaaaaaataataaattgcatgctgctttaattgtgaattgataataaactgtccctctttcagaaaacagacaaaaaaacaaaaaacaaaaaaaagcaaaaacaaaaatttgaaccaaaacattccgtttacattttagacactaagtatcttcgttcttgttagtactctgttctactgctttcaacttctcatagcgttggaattaaaactatgtcaaggtgctgttgtcattgctttactggcttaggggatggggaacgggaggggtagatttctgtttgttttgtgttttatttcgtttgtttgtttgttttgtttttttagttccacccggagtagggatggagaaaatttcttcactctccattctggttgataaagcgttacatttgtatgttgtaaaaaatgtttgcaaaatccaatcagatgactggaaaacgaataaaaattaaggcaactgaataaaatgctcacacaacactgcccatgatgtatctccctggtcccccaggtcactcttctggcatgggtcagggaaagctgcttttattggaaagaccagcatttgtttaaagcacattctttccctccctcctcccattttggtccctttcttttttgttttgttttaagaaagaaaattaagttgcgcgctttgaaatattttatcactgctgtgaacagatgaacaatgtgtgtcatttcatgacactcgtggaaaacagtgattttttttttattttttgccctaaggagaaacaagtaagaataaccgaaaatgttctttttttttaaaggcataaacagtggataagttataatatggcctaacaatgtttgcagataaaagatattgcatacagccagatactagagcagggatccacactgccactgaaatgcgactgaatggccctgtggaggctaagtggagctgacatactatttcctggcagagcaggaggaatttctgagtggccatcctgaggtctagatggaggtggggaatggtacttgagacattcctaaaggaaggctcggaagcacccttcagagcaggctctggaatgatgtgtcaagtttcttaggccttctgctttaagtgcctacgttacctaacagtgctcaagaggttctcaattggagaaccacactcaaatccatttatggcctccatcccattttaaataattatggataaagttggattaacctggagcagctttggatccaaatatggcatagcagtgatgctatcagtgcagcatgatgggaaatgtttgctgtgaagagacttaactttctttgcgcttagacttcaggaagcctaggttttatttatttatttttttgagacattttggtgaaaggaaaaaagaaagaagaaaacaaacaaacaaaaccagaaaaagcaccaaaacttaggcagaatgagcaatgtctgtctgtaagggctagaatgacaaggcataggaaggtgctttcactgtgaaagagacaagaacacaggaggaaatactgcttaagtgaagagcacagaaagctcctgatagttctgtccattcagcacaagggtcccttctacactttacctcttggggttaggagaagtcaagctggaagcctgaatgaatggccccaatgagaactagtgttaagcccatttccctagtgaggttttccgccagcgcgaatgtgttagtggttacctgactgggctcctgggcatcagaaaaagaggcaaacaattgcttcatcttaggagtggaaagggtgaaacaaagtggctgtccactgtgactcagggagtgaagataccatcagcaaatagtttctttttgttcaactgttcctttagaactagtctgtcttctggagtcccactgaatcccctgtttttggaagacttcacgtagcctagattgttttgtgccgtttgacaacattaatctctgtcatcatttttaacctattaaggaatgctttgaatatctgctatatgctaactttttgcagcttcattctgagagacgttagtcaaacaaataaaaggagccccatcacaatctcacggtattcgaagggccaggtcgattaggtggcttcataggagaccctccgcaactgtgtggtcagtggctggctctcatacccactaagatacatcatagctccatgtcggtggtttatgttgacctgagattgatttgttaaaatctctcctctgtttctgttcgttctgtttccgtcctgttctgttctgttctgttctgaaagtcttgctgtggtctctttttggcagaagtgtttcatgcatggcagcaggcctgatgctttttatagtgattcccattgaaactgtaagtaaatgtctgtggccttgttctctctatggtaaagatattattcaccatgtaaaacaagaaaaatatttattgtattttagtatatttatataattatgttattgaaaaaaattggcattaaaacttaaccacatcagaagcctattgtaaatacaggttctatttaagtgtaccaattaacatataatatatgttttaaat

SEQ ID NO: 43 - protein, 249 aa, Rattus norvegicus
MTILFLTMVISYFGCMKAAPMKEANVHGQGNLAYPAVRTHGTLESVNGPRAGSRGLTTTSLADTFEHVIEELLDEDQKVRPNEENHKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 44 - protein, 231 aa, Rattus norvegicus
APMKEANVHGQGNLAYPAVRTHGTLESVNGPRAGSRGLTTTSLADTFEHVIEELLDEDQKVRPNEENHKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 45 - protein, 119 aa, Rattus norvegicus; identical to SEQ ID NO: 34

SEQ ID NO: 46 - DNA, 1207 bp, Bos taurus
ggacgcttgaactgcagctgccaccgctgctgtggggccggcggcgagcggctccgcgacggggacctgggctgggcgcaggggccaggagcgggacgacgatgtgactccgccgccggggacccgtgagctttgtgtggaccccgagttccaccaggtgagaagagtgatgaccatccttttccttactatggttatttcatacttcggttgcatgaaggctgcccccatgaaagaagccaacctccgagcccaaggcagcttggcctacccaggtgtgcggacccatgggactctggagagcatgaatgggcccaaggtgggttcaagaggcctgacgtcctcgtcgtcgttggctgacacttttgaacacgtgatcgaagagctgttggacgaggaccagaaagttcggcccagcgaggaaaacaataaagacgcggacatgtacacgtcccgggtgatgctcagcagtcaagtgcctttggagccccctctcctcttcctgctcgaggaatacaaaaattacctggatgccgcaaacatgtccatgagggtccggcgccactcggaccccgcccgccgcggggagctgagcgtgtgtgacagcatcagcgagtgggtgaccgcagcggataaaaagactgcagtggacatgtcgggcgggacggtcacggtccttgaaaaagtccccgtctcaaaaggccagctgaagcagtacttctacgagaccaagtgcaatcccatgggttacacgaaggagggctgcaggggcatagacaagaggcattggaactcccagtgccgaactacccagtcgtacgtgcgggccctcaccatggatagcaaaaagcgtattggctggcggttcatacggatagacacttcttgtgtatgtacactgaccattaagaggggaagatagtggctttatgttgtatagattatattgagacaaaaatgatctatttgtatatatacataacagggtaaattattcagttaagaaaaaaaataattttatgaactgcatgtataaatgaagtttatacagtacagtggttctacaatctatttattggacatttccatgaccagaagggaaacagtcattttttgcgcacaactttaaaaagtctgctttacattcctcaatgttgtggtttgttgccgttgccaagaattgaaaaacgtaaaaaaaaaaaaaaaa

SEQ ID NO: 47 - protein, 250 aa, Bos taurus
MTILFLTMVISYFGCMKAAPMKEANLRAQGSLAYPGVRTHGTLESMNGPKVGSRGLTSSSSLADTFEHVIEELLDEDQKVRPSEENNKDADMYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 48 - protein, 232 aa, Bos taurus
APMKEANLRAQGSLAYPGVRTHGTLESMNGPKVGSRGLTSSSSLADTFEHVIEELLDEDQKVRPSEENNKDADMYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 49 - protein, 119 aa, Bos taurus; identical to SEQ ID NO: 34

SEQ ID NO: 50 - DNA, 1184 bp, Sus scrofa
aaaccgggcaccaaagattcccccctaccccttctttttgaccaaagggaacgtgaaaaaataatagagtctggggatttcggggccgaagtcttccccagagcagctgccttgatgtttactttgacaagtagtgactgaaaagttccaccaggtgagaagagtgatgaccatccttttccttactatggttatttcatacttcggttgcatgaaggctgcccccatgaaagaagccaacgtccgaggacaaggcagcttggcctacccaggtgtgcggacccatgggactctggagagcgtgaatgggcccaaggcaggttcaagaggcctgacatcgtcgtcatcgtcgtcgttggcggacacttttgaacacgtgatcgaggagctgttggacgaggaccagaaagttcggcccaatgaggaaaacaataaggacgcggacatgtatacgtcccgagtcatgctcagcagtcaagtgcctttggagcctcctcttctctttctgctggaggaatacaaaaattacctggatgctgcaaacatgtccatgagggtccggcgccactcggaccccgcccgccgcggggagctgagcgtgtgcgacagcattagcgagtgggtgacggcggcggataaaaagacggcagtggacatgtcgggtggcacggtcacggtcctcgaaaaagtccccgtctcgaaaggccaactgaagcagtacttctacgagaccaagtgcaatcctatggggtacacaaaggagggctgcaggggcatagacaagaggcactggaactcccagtgccgaactacccagtcgtatgtgcgggccctcaccatggatagcaaaaaacgaattggctggcggttcataaggatagacacttcctgtgtatgtactttgaccattaagaggggaagatagtggctttatgttgtatagattatattgagacaaaaattatctatttgtatatatacataacagggtaaattattcagttaagaaaaaaaataattttatgaactgcatgtataaatgaagtttatacagtacagtggttctacaatctatttattggacatttccatgaccagagggaaacagtcattttttgcgcacaactttaaaaaaaaagtctgcattacattcctcgataatgttgtggtttgttgccgttgct

SEQ ID NO: 51 - protein, 252 aa, Sus scrofa
MTILFLTMVISYFGCMKAAPMKEANVRGQGSLAYPGVRTHGTLESVNGPKAGSRGLTSSSSSSLADTFEHVIEELLDEDQKVRPNEENNKDADMYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 52 - protein, 234 aa, Sus scrofa
APMKEANVRGQGSLAYPGVRTHGTLESVNGPKAGSRGLTSSSSSSLADTFEHVIEELLDEDQKVRPNEENNKDADMYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 53 - protein, 119 aa, Sus scrofa; identical to SEQ ID NO: 34

SEQ ID NO: 54 - DNA, 6042 bp, Xenopus laevis
caaatggagccatttaatgaagagatgccattacgtccaccatgcagcttccactatcaataatttaaatatcactcgctgagagcccacgaagagcctgaattattttttttttcctcccgttgtttattgtttgtagccttttaattttttcccaaggaaagctggaaagaaaacttcaccccaacaaacaaaaaaacccaaaacctatattatccagagtttcagctgagatccccaatacaagtgtgttgatgtgttctttgacaagtactagctaaaataagttccatcaggtgagaagagtgatgaccatccttttccttactatggttatttcatacttcagttgcatgaaagctgcccccatgaaagaagccagtgtcagaggacaaaatggcctggcctatccgggtcttcggacccatggtactcttgagagcataggtggtcccagtggctcaagaggaggtggacttccttcactgacagatacttttgagcaagtcatagaagaactcctggaagaggaacaaagcataaggcaaagtgaggaaagcaaggactctgacttgtattcatctagagtaatgctaagcagtcaagtacctttggagccaccattgcttttcctacttgaggagtacaaaaactacttggatgcggcaaacatgtccatgagggtccggcgccactctgacccagccaggcgtggagagctgagtgtgtgtgacagtattagtgaatgggttacagcagcaaacaagaaaactgcagtggacatgtcggggcagacagttactgtcctagaaaaagtcccagtatccaaaggccaactgaagcaatatttctacgagaccaaatgcaaccctatgggttacatgaaagaaggctgcagaggcatagacaaaaggtactggaactctcagtgccgaactactcagtcttacgtgcgggctttcaccatggatagcaaaaaaaaagttggttggcgctttataagaatagacacttcttgtgtatgtacactgaccattaaaaggggaagatagtgaatttatcttgtatagattatattgagaagaaaatatctatttgtatatatacataacagggtaaattattccgtaagaaaaagaaaattaattttatggactgcatgtagaaaaaaaaaaagaagtttatacagtaaagtggttctacaatctatttattgaacatatccataccattaaaaaaacagagtcttctgcgcacaatgtaacctgtttcccgctgaagtatcttggaacccattgccgtgcaatgttttcctggttcctccagccggttatgggttaaagggaaaatattcccccagtttctcttccatttcttttctttcacatgaacagtaaactatgataatgcatattgttgggtgtagagaagaccctttctctagcatgtatgagcacagccgccatattgctgatcttgactgaggcatctccatagatttcagcagacggtgttgtttctgccggtaaacaccttggctcagatggttctaccgaagattggcaaacatttaagtgttggacttcttctagatggtgatgctctctgaaagtctatggaggactcccagttcccagttgtagggtttaaatgcaaatatgcagtaggctatattatcatccttcactgtctgtttggggaacaagaaaatggaagagggtatattctcccttaaagaattaagcctgcgttatgttcctcaataacattgtggtttgttgctgttgccaagaattgaaaagcataaaaggtaaaaaccaagaattgcatgctgcttcagttgtgaattgacaatatgtcttctattcagacaaaatttgaaccaaaacattccgtttacatttcacacagtatactttcccgtcggtattatatctgtttactgcttttaaacttctggtcgcgttggaattaaacaatgtcaaggtgctgttacagctttcactgttttaatttaatttttatttttattgggggtttttttttttttgtttttttaactcctgaaaaaagttgcaacgagcttgtcatgaatacatgtgttctggttaaaaaaaaaaaaaaaattttgtatgttgtaaaggtgtttgcaaactcgaatcaaactactgatcaaaaaaataaaataaaaaaaaaaaacagcaaggcaaacaagaaaatgatgctcgtggtacacttttgggactcgcaccggcaaccaaagtgtaagcttcatgtgtaacagaggtaattgctttataaatctgctctttcaacaggcttctgtgtgctgctttaaatgtgtatggccatttttagggggggaaatggaggtaaatggtgtcctcaacctaatgaaaggctgttgcaccaatctatatataaaataaagtctgacagctcatatataaaggaaattgtaagtcagcaccagaataatgctggtattagtcagtatttccaatgcaaatgctgcctgaatattgtttacctacgtcccattggccccgtaacagtgtcctcaaaatgctaattggaaaacaaatcagatctccagtgatcacagcatttccaaggaaaatggaaattggcaatgaaatttgttttggaatgaagaagcgtaggagaacatgggccggttgtgtgtcctaagtcaataaaggagccattaacacgtgaatggcacgtaggctggcatcttgattaaatagcacatctgaccattaggagatcttattctgttgatagtgaaagagagatggaagtgtgctgtagctgaacataaaagccacatatacctcagaaccacttggaaggaaaagctgtgaatggctttaaagggatactgtcgtctcaaattcctcccttttatgtatataaattgatataatcaaaagctacattacagttgtgttattcagaaggtacacctttatcagtgcttctgttcattctacctctgccaagcttttgtatatctttttagtctcattaataatcagccacatacatatccaataagcagtgcagtaaagattgggccatcaaaatcaagagtaattcattctactttgccttgtgggtcaggaaactgataatttaaaaaaaaaaaattacattacatgatacaataactactacagtgtccctttaaaggagaactaaacccgtgcaatgttaagtccccactggcccccctccgttgaccccccttcctgcttcccccttaccccagaattcttttccctcttgaaaaagtgaccgcacatgcagagtgagcgcagcggagctcacgggcgccattttattctcttcggtaatcttcttgtcttcttcctccacttcggcaatttccgtctctttcagcgcatgcgcagttgtcgcataccggaagattgctccaacagctcatgcgccattaagccgctcacttcacgaatattaacaaagagaagaagatggcgtctgtgagcttctctgctctcacgctgggagggggccaacggagtgggccagtgggaacttaacatcagctttagttctcctttaaggccacacttttatacttcatcaagcagcctagtacccccatgaggttctgcagtagctcaggcctaatgtggtagaacaaatattttgtgctgttcccaaatttgctaaatcttaacttgcctttctattgttcataattggcttcaggctccattttagggttgggcatgaatttgtgaaattagaagaatcagacgggaaaccaaaaaaaacagctaatctgaaaaccaagctatttaaaggtcagctaggaaaatgatgcttaggaccttgccatcacttcttgacagtggaaaattcaattacatctgataagtaggataatgtttggagtggcatttaccccagcattgttcattaaagattccctctaacaatctgtcagcggaaagtgaaataagacatggtagtcccttactgcaccactgtaagccttagaaggaggctcagatttgggaatactggtcctgaaccttactttacttgccttagcaggagggttggataaagtggccatgttttcaggtcatggtggtcatgttttaggtgccaggtgcaaccctgttgctgaaatgcaactccttagtcttagtttagcaacagctagagagttagtatgtgagctatcaaaagttctgggcatcttaagtcatcatctatagtttacaccaattttactaggtaaagaagacctcatcaagactaaatgagctatactggacatagagcacaactggccccaaaatccacagtataatgccattcactgtatcaaaaattaaatgatgtaatgaagctaaggaacaagaatgtctcgcccagcggatctagccaaagatgtgctaggagaaagctgaaggcaatgtcaattgaagatcaatatgagttaatcacatgtctagggaatggacaattaaagtggaaacaacagcagctacacaatgagcgtggatttcatattactatctacagtagagctgaaattctctagagtgtaccataatatctttgtttactttatttaatctagtgatgtgtatctttatctactgcccaaagccagatataatattaataacgtccaaagcctaaattgttggttccttctcaacaagttggggaagccttctaatcattgaacaaagcttgagttttgagaaggcatgaccttgattagtattggtttcactgtttgctttgtcatatatgggtgtgcgcatgtgtatatatagacatataggctcatcggtctgtgaccgcgctcttatttctccatgtacacgtggacacaaatatatgtataaacccctacttgatttcttttatatggagtatgagaaggctccctcactcgcaggagaaatataaatatatttttttttcaactaacactattcagtctatgcatttacgctaggatgatcaacgagtcatctggatgccctaacttttgccttgaacctttgcctagaaaaaaaaggcgtgtatgtgtgtttgtctgtaaaaagaagttaaaaaaaaattccatcagtcaatgagaagatgttcttgaagcttaaagctcccttaaatatgggttgcagctcatagacttttttgtttcctacacccagatctacaaatctatatgataaagcagggggagaactcatgagagccggagaagtcaagaagtggccttccttagctgtcagatatatagcggccatgaccctctatcatgtcccccaatactgtggtctaaaaaaagaaatgtcctcctatatgtttgtgtttgtctgtatatacaagtacgcaatcagtgtatacatgactttaaaaaatatgcaaaactattaagtggaaacaatatgaggcagttagaatagattcagcagtgcgaggatatattgtctgttctgttggacgcaaagcgacgtaacgtacaaaaaaagctactacatgccactgaatatacctgtcggatactaggcttggcagttttagctgaactgagctgcaacacagacttttttttattttatttattttcgttatttttttctttgtctttttcgataattttgttttgccgtggtctgtttgtggcaaacgtatattctgcatgtcacctgcctaccgccttgttaaaaaaagtgagacactttgaaaatgtaagttttatgtatgtgaccttgttctttctggaaaaaaaaaggtatatattttcacgatgtgtaagaaatatttattgtatgttaagtatatttatataattatgttattgaaacatggagattggcatttaaaaatggaagcatttgaaacatattgtaaataggattacctctatttaagtgtactacataacatataatatatgttgaaaaatagaaatttttaatgtttttaaatatattttcaaaatatataaactaaaaaaaaaaaaaaaaaaaaaaaaaaaaaaaaaaa

SEQ ID NO: 55 - protein, 247 aa, Xenopus laevis
MTILFLTMVISYFSCMKAAPMKEASVRGQNGLAYPGLRTHGTLESIGGPSGSRGGGLPSLTDTFEQVIEELLEEEQSIRQSEESKDSDLYSSRVMLSSQVPLEPPLLFLLEEYKNYLDAANMSMRVRRHSDPARRGELSVCDSISEWVTAANKKTAVDMSGQTVTVLEKVPVSKGQLKQYFYETKCNPMGYMKEGCRGIDKRYWNSQCRTTQSYVRAFTMDSKKKVGWRFIRIDTSCVCTLTIKRGR

SEQ ID NO: 56 - protein, 114 aa, Xenopus laevis
RHSDPARRGELSVCDSISEWVTAANKKTAVDMSGATVTVLEKVPVSKGQLKQYFYETKCNPMGYMKEGCRGIEKRYWNSQCRTTQSYVRAFTMDSKKKVGWRFIRIDTSCVCTL

SEQ ID NO: 57 - protein, 114 aa, Xenopus laevis; identical to SEQ ID NO: 56

SEQ ID NO: 58 - protein, 119 aa, Gallus gallus
HSDPARRGELSVCDSTSEWVTAAEKKTAVDMSGATVTVLEKVPVPKGQLKQYFYETKCNPKGYTKEGCRGIDKRHWNSQCRTTQSYVRALTMDNKKRVGWRFIRIDTSCVCTLTIKRGR

SEQ ID NO: 59 - protein, 119 aa, Canis familiaris; identical to SEQ ID NO: 34

SEQ ID NO: 60 - protein, 113 aa, Macaca mulatta
HSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTL

SEQ ID NO: 61 - protein, 119 aa, Felis catus
HSDPARRGELSVCDGISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 62 - protein, 10 aa, Homo sapiens
CVPVSKGQLC

SEQ ID NO: 63 - protein, 10 aa, Homo sapiens
CVCVSKGQLC

SEQ ID NO: 64 - protein, 10 aa, Homo sapiens
CVPCSKGQLC

SEQ ID NO: 65 - protein, 10 aa, Homo sapiens
CVPVCKGQLC

SEQ ID NO: 66 - protein, 11 aa, Homo sapiens
CVPVSKGQLCE

SEQ ID NO: 67 - protein, 11 aa, Homo sapiens
CVPVCKGQLCE

(19) United States
(12) Patent Application Publication
(10) Pub. No.: US 2011/0104165 A1
(43) Pub. Date: May 5, 2011

(54) METHOD OF UPREGULATING SORLA FOR THE TREATMENT OF ALZHEIMER'S DISEASE

(75) Inventor: Thomas Willnow, Berlin (DE)

(73) Assignee: H. LUNDBECK A/S, Valby-Copenhagen (DK)

(21) Appl. No.: 13/001,157

(22) PCT Filed: Sep. 10, 2009
(86) PCT No.: PCT/DK09/50233

§ 371 Date: Dec. 23, 2010

Related U.S. Application Data
(60) Provisional application No. 61/096,218, filed on Sep. 11, 2008.
(60) Provisional application No. 61/185,159, filed on Jun. 8, 2009.

(30) Foreign Application Priority Data
Sep. 11, 2008 (DK) PA200801273

(51) Int. Cl. A61K 39/395; A61K 31/7088; A61K 38/18; A61P 25/28

(52) U.S. Cl. 424/135.1; 514/44 R; 424/133.1; 424/158.1; 424/172.1; 424/143.1; 514/7.6; 514/8.3

(57) ABSTRACT

The present invention is directed to use of an agent capable of upregulating the Vps10p-domain receptor SorLA. In particular, the present invention relates to use of CTGF and/or BDNF and/or fragments and/or variants thereof for the inhibition of plaque formation thereby being useful in the treatment of Alzheimer's Disease or other disorders associated with elevated of amyloid beta or amyloid plaque.

All patent and non-patent references cited in the application, or in the present application, are hereby incorporated by reference in their entirety.

### TECHNICAL FIELD OF INVENTION

The present invention relates to the field of therapeutic use of proteins, genes and cells, in particular to the therapy based on the biological function of a secreted therapeutic protein, CTGF, alone or as combination treatment with BDNF, in particular for the treatment of disorders of the nervous system.

### BACKGROUND OF INVENTION

Extracellular proteins play important roles in, among other things, the formation, differentiation and maintenance of multicellular organisms. The fate of many individual cells, e.g., growth including survival, proliferation, migration, differentiation, or interaction with other cells, is typically governed by information received from other cells and/or the immediate environment. This information is often transmitted by secreted polypeptides (for instance, mitogenic factors, survival factors, cytotoxic factors, differentiation factors, neuropeptides, and hormones) which are, in turn, received and interpreted by diverse cell receptors or membrane-bound proteins. These secreted polypeptides or signaling molecules normally pass through the cellular secretory pathway to reach their site of action in the extracellular environment.

Disorders such as Parkinson's disease, Alzheimer's disease, Huntington's disease, multiple sclerosis, amyotrophic lateral sclerosis, stroke, schizophrenia, epilepsy and peripheral neuropathy and associated pain affect millions of people. It is the loss of normal neuronal function, which produces the behavioral and physical deficits which are characteristic of each of the different neurological disorders. In addition to chronic and acute neurodegenerative disorders, the aging process, physical trauma to the nervous system, and metabolic disorders may result in the loss, dysfunction, or degeneration of neural cells accompanied by the associated behavioral and physical deficits. Many of these diseases are today incurable, highly debilitating, and traditional drug therapies often fail. There is thus a great medical need for new therapeutic proteins that are disease modifying or for symptomatic use or both.

Several secreted factors with expression in the nervous system or associated target areas have important therapeutic uses in various neurological indications associated with reduction or loss of neuronal functions. E.g. NGF is a candidate for treatment of Alzheimer's disease, Artemin a candidate for treatment of peripheral neuropathy, and GDNF is a candidate for treatment of Parkinson's Disease.

Alzheimer's disease is the most common age-related dementia, affecting millions of patients worldwide. Due to an overall increase in life expectancy, this disease will reach epidemic proportions in ageing societies of industrialized nations. Central to the pathology of Alzheimer's disease is the amyloid precursor protein (APP), a membrane protein in neurons. APP is proteolytic processed to a 40 to 42 amino acid amyloid β-peptide (Aβ) that acts as neurotoxin. Accelerated processing of APP to Aβ in individuals is the underlying cause of neurodegenerative processes resulting in Alzheimer's disease [1]. The molecular mechanisms that control APP processing and Aβ formation are poorly understood, but pharmacological blockade of Aβ production is considered a preferred goal in treatment of this disease. Thus, intensive research efforts have been directed towards identification of factors (and mechanisms) that control APP processing and that may represent novel drug targets for therapeutic intervention.

VPS10P domain-containing receptors are a novel class of sorting proteins that regulate the intracellular transport of target proteins in neurons. SorLA is a member of this gene family recently implicated in processes related to Alzheimer's disease [2]. Studies have demonstrated that this receptor acts as intracellular sorting protein for APP that regulates transport of the precursor protein into compartments less favourable for proteolytic processing [3]. Consequently, genetic overexpression of SorLA in neurons decreases APP processing and Aβ production, while loss of SorLA expression in genetically engineered knockout mouse models results in enhanced Aβ formation and Alzheimer's disease like phenotypes [3-5]. A central role for SorLA in Alzheimer's disease was recently substantiated by demonstrating loss of receptor expression in the brain of patients with sporadic Alzheimer's disease [6, 7]. Finally, association of certain SorLA gene variants with occurrence of neurodegenerative diseases in several ethnic populations strongly suggests its relevance as major genetic risk factor in Alzheimer's disease [8-10].

Currently, a number of therapeutic strategies are contemplated to treat patients with Alzheimer's disease. However, none of these therapies has had any major beneficial impact on disease progression, warranting the need for new conceptual approaches [11]. The major drugs on the market are cholinesterase inhibitors such as donepezil (Eisai/Pfizer), rivastigmine (Exelon; Novartis), galantamine (Razadyne; Johnson & Johnson) and tacrine (Cognex; First Horizon Pharmaceuticals), as well as the N-methyl-D-aspartate (NMDA)-receptor modulator memantine (Namenda; Forest/Lundbeck). These drugs do not address processes specific to Alzheimer's disease, but unspecific modulate neuronal activities (aimed at improving cognitive functions in affected individuals). In addition to being only modestly efficacious, none of these drugs prevents or reverses the disease progression.

An alternative disease modifying strategy aims at modulation of the metabolism of Aβ, the peptide that is causative of neurodegenerative disease processes. Therapeutic strategies seek prevention of Aβ formation, blockade of its aggregation into plaques, or lowering its levels in the brain [12]. The most advanced disease-modifying drug candidate in development is Neurochem's tramiprosate. Tramiprosate is a glycosaminoglycan mimetic designed to bind to Aβ peptides, thereby stopping the formation of amyloid plaques. No significant improvement of cognition was observed in the tramiprosate Phase II trial, raising doubts about the efficacy of drugs interfering with plaque formation. A third group of therapeutic drugs aims at interfering with the activity of secretases, the proteases responsible for proteolytic cleavage of APP to Aβ (beta-secretase, gamma-secretase). Myriad Genetics' taren-flurbil—a modulator of gamma-secretase activity—is the most advanced agent in clinical development. However, recent data from animal experimentation have uncovered important biological activities for secretases in normal neuronal function (such as formation of the myelin sheet surrounding axons) [13]. Because loss of secretase activity in genetically engineered mouse models results in severe neuronal dysfunction, therapeutic strategies aimed at blockade of secretase activities in patients are unlikely to reach the clinics [13].

It has been shown that SorLA acts as negative modulator of APP processing and that SorLA expression levels are inversely correlated with Aβ production rates (FIG. 1). These findings suggest pharmacological strategies to raise SorLA expression in neurons as a major novel therapeutic concept to combat neurodegenerative disease processes. Accordingly, compounds were screened for their ability to raise SorLA levels in neurons (FIG. 2). Connective Tissue Growth Factor (CTGF) and brain-derived neurotrophic factor (BDNF) were able to raise SorLA mRNA and protein levels by up to 10-fold when applied to primary neurons (FIG. 2). This effect of CTGF and BDNF was specific for induction of SorLA expression and not seen for related receptors of the VPS10P domain receptor gene family such as sortilin (FIG. 3). Raising SorLA levels by treatment with CTGF and BDNF significantly impaired Aβ formation in neurons as shown be determination of Aβ concentrations in treated neuronal cell cultures using ELISA (FIG. 4A). Furthermore, treatment of wild type mice with recombinant BDNF by intracranial injection reduced Aβ levels in the brain in vivo (FIG. 4B). BDNF application did not affect Aβ levels in mice genetically deficient for SORLA, demonstrating that this receptor is the unique drug target for the beneficial action of trophic factors in Alzheimer's disease (FIG. 4B). A function for BDNF as inductor of SORLA expression was further substantiated by demonstrating significantly reduced levels of SORLA protein in the brain of mice genetically deficient for BNDF (FIG. 5)

### SorLA

The present inventors have demonstrated that the loss of the Vps10p-domain receptor SorLA increases processing of Amyloid Precursor Protein (APP) to generate Aβ and to form amyloid plaques considered by those skilled in the art to be a major contributor to the onset of Alzheimer's disease. The inventors have furthermore demonstrated that an increased expression of SorLA blocks processing of APP, and that such an increase can be achieved by administering CTGF and/or BDNF to neurons. Accordingly, in a main aspect, the present invention relates to the use of CTGF and/or BDNF for the preparation of a medicament for the treatment of a neurodegenerative disease associated with elevated levels of Aβ and amyloid plaque, said disease including but not being limited to Alzheimer's Disease (AD).

Sorting protein-related receptor abbreviated SorLA (Swiss prot ID no Q92673), also known as LR11, is a 250-kDa type-1 membrane protein and the second member identified in the Vps10p-domain receptor family. SorLA, like sortilin, whose lumenal domain consists of a Vps10p domain only, is synthesized as a proreceptor that is cleaved by furin in late Golgi compartments. It has been demonstrated [33] that the truncation conditions the Vps10p domain for propeptide inhabitable binding of neuropeptides and the receptor-associated protein. In transfected cells, about 10% of full-length SorLA is expressed on the cell surface capable of mediating endocytosis. The major pool of receptors is found in late Golgi compartments, where interaction with newly synthesized ligands has been suggested to occur.

### CTGF

Connective Tissue Growth Factor (CTGF) is a cysteine-rich, matrix-associated, heparin-binding protein. In vitro, CTGF mirrors some of the effects of TGF beta on skin fibroblasts, such as stimulation of extracellular matrix production, chemotaxis, proliferation and integrin expression. CTGF can promote endothelial cell growth, migration, adhesion and survival and is thus implicated in endothelial cell function and angiogenesis (Brigstock D R (2002) Angiogenesis 5:153-165).

CTGF binds to perlecan (Nishida T et al. (2003) J Cell Physiol 196:265-275), a proteoglycan which has been localised in synovium, cartilage and numerous other tissues.

CTGF has been implicated in extracellular matrix remodelling in wound healing, scleroderma and other fibrotic processes, as it is capable of upregulating both matrix metalloproteinases (MMPs) and their inhibitors (TIMPs). Therefore, CTGF has the potential to activate both the synthesis and degradation of the extracellular matrix.

Knockout mice which have had the gene for CTGF removed do not develop normally. Impaired chondrocyte proliferation, angiogenesis, extracellular matrix production and turnover leads to abnormal skeletal growth (Ivkovic S et al. (2003) Development 130:2779-2791).

### BDNF

Brain-derived neurotrophic factor (BDNF) is a neurotrophic factor found in the brain and the periphery. It is a protein that acts on certain neurons of the central nervous system and the peripheral nervous system, helping to support the survival of existing neurons and encourage the growth and differentiation of new neurons and synapses. In the brain, it is active in the hippocampus, cortex, and basal forebrain—areas vital to learning, memory, and higher thinking. BDNF was the second neurotrophic factor to be characterized after nerve growth factor (NGF).

Although the vast majority of neurons in the mammalian brain are formed prenatally, parts of the adult brain retain the ability to grow new neurons from neural stem cells in a process known as neurogenesis. Neurotrophins are chemicals that help to stimulate and control neurogenesis, BDNF being one of the most active. Mice born without the ability to make BDNF suffer developmental defects in the brain and sensory nervous system, and usually die soon after birth, suggesting that BDNF plays an important role in normal neural development.

Despite its name, BDNF is actually found in a range of tissue and cell types, not just in the brain. It is also expressed in the retina, the CNS, motor neurons, the kidneys, and the prostate.

Exposure to stress and the stress hormone corticosterone has been shown to decrease the expression of BDNF in rats, and leads to an eventual atrophy of the hippocampus if exposure is persistent. Similar atrophy has been shown to take place in humans suffering from chronic depression (Hamilton et al, Mol. Psychiatry, May 2008 and Pezawas et al, Nat. Neurosci. 8:828-34, 2005). In addition, mice bred to be heterozygous for the BDNF gene defect, therefore reducing its expression, have been observed to exhibit similar hippocampal atrophy, suggesting that an etiological link between the development of depressive illness and regulation of BDNF exists. On the other hand, the excitatory neurotransmitter glutamate, voluntary exercise, (Russo-Neustadt A A, Beard R C, Huang Y M, Cotman C W (2000). Neuroscience 101 (2): 305-12) caloric restriction, intellectual stimulation, curcumin and various treatments for depression (such as antidepressants and electroconvulsive therapy) strongly increase expression of BDNF in the brain of rats, and have been shown to protect against this atrophy (Brunoni et al, Int J Neuropsychopharmacol. August 28:1-12, 2008).

BDNF binds at least two receptors on the surface of cells which are capable of responding to this growth factor, TrkB (pronounced “Track B”) and the LNGFR (for “low affinity nerve growth factor receptor”, also known as p75). It has also been reported to bind to nicotinic acetylcholine receptor alpha7 (Catarina C. Fernandes et al. (2008) J. Neuroscience 28 (21): 5611-5618).

TrkB is a receptor tyrosine kinase, accordingly it mediates its actions by causing the addition of phosphate molecules on certain tyrosines in the cell and activating cellular signaling. Other neurotrophins structurally related to BDNF includes NGF (Nerve Growth Factor), NT-3 (Neurotrophin-3) and NT-4/5 (Neurotrophin-4/5). While TrkB mediates the effects of BDNF and NT-4/5, TrkA binds and is activated by NGF, and TrkC binds and is activated by NT-3. NT-3 binds to TrkA and TrkB as well, but with less affinity.

The other BDNF receptor, the p75^{NTR} acts as co-receptor to TrKs, modulating their specificity for certain neurotrophins. It has been demonstrated that the p75^{NTR} in complex with proNGF and the Vps10p-domain receptor Sortilin signals to cells to go into apoptosis (Nykjær et al (2004) Nature)

### BDNF and Alzheimer's Disease

BDNF has been the focus of intense interest in the Alzheimer's field for a number of years. BDNF belongs to the neurotrophin family of growth factors and affects the survival and function of neurons in the central nervous system, particularly in brain regions susceptible to degeneration in AD. BDNF improves survival of cholinergic neurons of the basal forebrain, as well as neurons in the hippocampus and cortex. This discovery kindled hope in the early 1990s that Alzheimer's could be slowed or halted if brain levels of BDNF could be increased. The idea gained support with the observation that BDNF gene activity and protein levels are reduced in AD brains.

Further research on BDNF in the mid-90s revealed additional exciting functions of this molecule in the brain. Beyond promoting neuronal survival and resilience to injury, BDNF also has a powerful role in facilitating activity-dependent plasticity, which underlies the capacity for learning and memory. Brain regions where plasticity is particularly important include the hippocampus and cortex, critical centers for learning and memory. The hippocampus is a central component for encoding new information, and damage there severely impairs learning. Hippocampal function is compromised early on in the course of AD, and this is considered the principal cause of the memory problems that characterize this disease. The reduction of BDNF seen in AD could cripple the hippocampus in two ways: From a plasticity point of view, insufficient BDNF would weaken synaptic encoding strength or capacity, while from the neurotrophic angle, reduced BDNF makes hippocampal neurons more vulnerable to insult and degeneration.

BDNF is an unusual neurotrophic factor. Its widespread functions in the brain go beyond the traditional role of a growth factor to promote growth, survival, and maintenance of cells. Recently, a third role for BDNF has emerged, in that it appears to be an important factor in psychiatric conditions such as epilepsy, depression, obsessive compulsive disorder, and possibly bipolar disorder. While unlikely to be causally related to Alzheimer's, these mood disorders, particularly depression, often coexist with Alzheimer's and may have a common link through BDNF.

Below, we discuss evidence supporting a role for BDNF in learning and memory, followed by recent genetic data demonstrating a link between BDNF and AD.

### BDNF in Learning and Memory

BDNF is produced by neurons, particularly in the hippocampus and cortex. Neuronal activity, i.e., during encoding of information, stimulates BDNF gene transcription, transport of BDNF mRNA into dendritic spines, and BDNF protein release into the synaptic cleft (Hartmann M et al. (2001) EMBO J. 20(21):5887-97). BDNF can be transported into the dendrite and may also be synthesized locally in the spine. It has been speculated that one or both of these mechanisms may be able to target active synapses within dendrites. BDNF acts on neurons at both presynaptic and postsynaptic sites by binding to its tyrosine kinase receptor TrkB, and subsequent internalization of the BDNF-TrkB complex. Interestingly, internalization does not lead to termination of the BDNF signal, such as occurs for most other growth factor receptors. Rather, the internalized TrkB receptor remains phosphorylated and activated. It resides in a specialized compartment called a “signaling endosome,” which seems to be critical for downstream signaling effects of BDNF on the cell body. (For an excellent review on BDNF regulation and plasticity (Lu B. (2003) Learn Mem. 10(2):86-98).

By enhancing synaptic transmission and neuronal excitability (Korte M et al. (1996) Proc Natl Acad Sci 93(22):12547-52), BDNF modulates synaptic change, including hippocampal long-term potentiation (LTP), a neural mechanism associated with learning and adaptive behaviors in adult animals (Poo M M. (2001) Nat Rev Neurosci. 2(1):24-32; Tyler W J et al. (2002) Learn Mem. 9(5):224-37). A critical role for BDNF/TrkB signaling in plasticity mechanisms is evidenced by in vivo studies where BDNF/TrkB signaling has been impaired by genetic or immunopharmacological means. Mice deficient in BDNF/TrkB signaling have impaired learning and LTP and, importantly, restoring BDNF levels reverses both the electrophysiological and learning deficits (Levine E S et al. (1995) Proc Natl Acad Sci USA 92(17):8074-7; Korte M et al. (1996) Proc Natl Acad Sci 93(22):12547-52; Patterson S L et al. (1996) Neuron. 16(6):1137-45). In addition, BDNF-deficient mice show decreased synaptic innervation and reduced levels of synaptic vesicle proteins (Martinez A et al. (1998) J. Neurosci. 18(18):7336-50; Pozzo-Miller L D et al. (1999) J. Neurosci. 19(12):4972-83), demonstrating that BDNF is important for normal synaptic signaling (Martinez A et al. (1998) J. Neurosci. 18(18):7336-50).

Other genetic studies have established a decisive role for BDNF in human cognition. Polymorphisms in the DNA sequence of a gene can result in seemingly subtle differences in the final protein product, which nevertheless can profoundly change the functionality of the resulting protein. One polymorphism in the BDNF gene that does just that is caused by a single amino acid substitution in the coding region of the BDNF gene (val/met substitution at codon 66). This substitution derails trafficking of the BDNF protein within the cell such that it is no longer released in response to appropriate cellular cues. The effect of this is seen at the level of hippocampal function, as the polymorphism is associated with impaired memory and abnormal hippocampal activation. Remarkably, these cognitive decrements were revealed in a cohort of 641 cognitively intact adults aged 25-45 (Egan M F et al. (2003) Cell 112(2):257-69; Hariri A R et al. (2003) J. Neurosci. 23(17):6690-4)

Having made it clear that deficiencies in BDNF function has serious cognitive consequences even in young people, these studies prompt the question of what the relationship is between abnormal BDNF and AD.

### BDNF Polymorphisms are Risk Factors for AD

Three different BDNF polymorphisms have been proposed as possible risk factors for AD based on genetic association studies. The val/met polymorphism (position 196, codon 66) described above conferred increased susceptibility to AD that appeared to be independent of ApoE genotype (Ventriglia M et al. (2002) Mol Psychiatry. 7(2):136-7).

The single nucleotide polymorphism C270T has been associated with late-onset but not early-onset AD in a Japanese population (51 early onset; 119 late onset; 498 controls (Kunugi H et al. (2001) Mol Psychiatry 6(1):83-6).

Another study of the C-270T polymorphism in a German population (210 AD cases, 188 controls) found its frequency increased in AD, and risk appeared to be higher in AD patients lacking the ApoE4 allele (Riemenschneider M et al. (2002) Mol Psychiatry. 7(7):782-5). Except for the met-BDNF polymorphism, little is known about how the polymorphisms affect BDNF function. These questions are currently under investigation, and are likely to expand our understanding of the role of BDNF in AD, as well as in learning, memory, and cognitive function throughout life.

### Increased Levels of BDNF in the Brain

Animal studies demonstrate that brain levels of BDNF are modified in response to certain types of stimulation that occur normally in our daily lives. Remarkably, two potent stimuli that rapidly increase BDNF levels in the hippocampus are exercise and learning. In rodents, voluntary daily wheel running consistently increases BDNF mRNA and protein levels in the hippocampus and other brain regions, including parts of the cortex (for review on exercise and BDNF, (Cotman C W et al. (2002) Trends Neurosci. 25(6):295-301). In addition, learning itself increases brain BDNF levels, particularly in the hippocampus. Interestingly, in humans, regular exercise is associated with benefits to brain health and cognitive function, which may in part be due to increased availability of BDNF. Indeed, physically active adults not only have a lower risk of cognitive impairment, but also a lower risk of depression and of developing AD or dementia of any type (Friedland R P et al. (2001) Proc Natl Acad Sci USA. 98(6):3440-5; Laurin D et al. (2001) Arch Neurol. 58(3):498-504). Furthermore, exercise improves depression not only in normal adults, but also in people with moderate to severe AD, demonstrating that exercise can be an effective intervention when the course of neurodegeneration/neuropathology has already progressed. In addition, there is evidence that mental activity/learning may also be protective against AD. An association between BDNF and these positive effects of exercise (and learning) on depression and dementia has not yet been definitely established; however, BDNF may serve as a common molecular mechanism. Increasing BDNF availability in the brain (stimulated, for example, by exercise or learning) is rapidly gaining strength as an important approach to improving cognitive function throughout life and offsetting depression and dementia.

### SUMMARY OF THE INVENTION

In a main aspect, the present invention is directed to the use of at least one isolated polypeptide capable of upregulating the Vps10p-domain receptor SorLA, for the preparation of a medicament for inhibiting formation of amyloid plaque in an individual.

In a further main aspect, the present invention relates to use of at least one isolated Brain Derived Neurotrophic Factor (BDNF) and/or Connective Tissue Growth Factor (CTGF) polypeptide for the preparation of a medicament for inhibiting formation of amyloid plaque in an individual.

In an important aspect, the present invention relates to use of at least one isolated nucleotide encoding the polypeptide as defined herein above, for the preparation of a medicament for inhibiting formation of amyloid plaque in an individual.

In another aspect, the present invention relates to the use of the isolated Brain Derived Neurotrophic Factor (BDNF) and/or Connective Tissue Growth Factor (CTGF) polypeptide defined herein above wherein said polypeptide comprises an amino acid sequence selected from the group consisting of:
a) the amino acid sequence selected from the group consisting of SEQ ID NO. 5, 8, 11, 14, 17, 20, 23, 34, 41, 45, 49, 53, 57, 58, 59, 60, 61 and 62; and
b) a sequence variant of the amino acid sequence selected from the group consisting of SEQ ID NO. 5, 8, 11, 14, 17, 20, 23, 34, 41, 45, 49, 53, 57, 58, 59, 60, 61 and 62, wherein the sequence variant has at least 70% sequence identity to said SEQ ID NO. 5, 8, 11, 14, 17, 20, 23, 34, 41, 45, 49, 53, 57, 58, 59, 60, 61 or 62; or a biologically active fragment thereof, said fragment comprising at least 50 contiguous amino acids, wherein any amino acid specified in the selected sequence is altered to a different amino acid, provided that no more than 15 of the amino acid residues in the sequence are so altered.

In a main aspect, the present invention relate to the use of at least one isolated peptide for the preparation of a medicament for inhibiting formation of amyloid plaque, said peptide comprising a fragment comprising at least 8 contiguous amino acid residues of an amino acid sequence selected from the group consisting of
a) SEQ ID NO. 5, 8, 11, 14, 17, 20, 23, 34, 41, 45, 49, 53, 57, 58, 59, 60, 61 and 62; and
   a sequence variant of the amino acid sequence of a), wherein the sequence variant has at least 70% sequence identity to said SEQ ID NO. 5, 8, 11, 14, 17, 20, 23, 34, 41, 45, 49, 53, 57, 58, 59, 60, 61 or 62.

In a main aspect, the present invention comprises at least one isolated Brain Derived Neurotrophic Factor (BDNF) and/or Connective Tissue Growth Factor (CTGF) nucleotide for the preparation of a medicament for inhibiting formation of amyloid plaque in an individual.

In one main aspect the present invention relates to the use of
a) the polypeptide as defined herein above; or
b) the isolated nucleic acid sequence as defined herein above; or
c) the expression vector as defined herein; or
d) a composition of host cells as defined herein;
e) a packaging cell line according as defined herein;
for the manufacture of a medicament for inhibiting formation of amyloid plaque in an individual in need thereof.

In a main aspect, the present invention relate to the use of at least one isolated peptide for the preparation of a medicament for inhibiting formation of amyloid plaque, said peptide comprising a fragment comprising at least 8 contiguous amino acid residues of an amino acid sequence selected from the group consisting of
b) SEQ ID NO. 5, 8, 11, 14, 17, 20, 23, 34, 41, 45, 49, 53, 57, 58, 59, 60, 61 and 62; and
   a sequence variant of the amino acid sequence of a), wherein the sequence variant has at least 70% sequence identity to said SEQ ID NO. 5, 8, 11, 14, 17, 20, 23, 34, 41, 45, 49, 53, 57, 58, 59, 60, 61 or 62.

In one important aspect the present invention relates to an isolated polypeptide selected from the group consisting of:
a) A polypeptide having an amino acid sequence as set forth in SEQ ID NO. 5, and polypeptides having from one to five extra amino acids;
b) A polypeptide having an amino acid sequence as set forth in SEQ ID NO. 8, and polypeptides having from one to five extra amino acids;
c) A polypeptide having an amino acid sequence as set forth in SEQ ID NO. 11, and polypeptides having from one to five extra amino acids;
d) A polypeptide having an amino acid sequence as set forth in SEQ ID NO. 14, and polypeptides having from one to five extra amino acids;
e) A polypeptide having an amino acid sequence as set forth in SEQ ID NO. 17, and polypeptides having from one to five extra amino acids;
f) A polypeptide having an amino acid sequence as set forth in SEQ ID NO. 20, and polypeptides having from one to five extra amino acids;
g) A polypeptide having an amino acid sequence as set forth in SEQ ID NO. 23, and polypeptides having from one to five extra amino acids; A polypeptide having an amino acid sequence as set forth in SEQ ID NO 34, and polypeptides having from one to five extra amino acids;
h) A polypeptide having an amino acid sequence as set forth in SEQ ID NO 41, and polypeptides having from one to five extra amino acids;
i) A polypeptide having an amino acid sequence as set forth in SEQ ID NO 45, and polypeptides having from one to five extra amino acids;
j) A polypeptide having an amino acid sequence as set forth in SEQ ID NO 49, and polypeptides having from one to five extra amino acids;
k) A polypeptide having an amino acid sequence as set forth in SEQ ID NO 53, and polypeptides having from one to five extra amino acids;
l) A polypeptide having an amino acid sequence as set forth in SEQ ID NO 57, and polypeptides having from one to five extra amino acids;
m) A polypeptide having an amino acid sequence as set forth in SEQ ID NO 58, and polypeptides having from one to five extra amino acids;
n) A polypeptide having an amino acid sequence as set forth in SEQ ID NO 59, and polypeptides having from one to five extra amino acids;
o) A polypeptide having an amino acid sequence as set forth in SEQ ID NO 60, and polypeptides having from one to five extra amino acids;
p) A polypeptide having an amino acid sequence as set forth in SEQ ID NO 61, and polypeptides having from one to five extra amino acids;
q) A polypeptide having an amino acid sequence as set forth in SEQ ID NO 62, and polypeptides having from one to five extra amino acids; and
r) variants of said polypeptides, wherein any amino acid specified in the chosen sequence is changed to a different amino acid, provided that no more than 15 of the amino acid residues in the sequence are so changed.

In another aspect, the present invention relates to at least one isolated polypeptide for use in a method of treatment of disease resulting from formation of amyloid plaque, said polypeptide comprising an amino acid sequence selected from the group consisting of:
a) the amino acid sequence selected from the group consisting of SEQ ID NO. 5, 8, 11, 14, 17, 20, 23, 34, 41, 45, 49, 53, 57, 58, 59, 60, 61 and 62; and
b) a sequence variant of the amino acid sequence selected from the group consisting of SEQ ID NO. 5, 8, 11, 14, 17, 20, 23, 34, 41, 45, 49, 53, 57, 58, 59, 60, 61 and 62, wherein the sequence variant has at least 70% sequence identity to said SEQ ID NO. 5, 8, 11, 14, 17, 20, 23, 34, 41, 45, 49, 53, 57, 58, 59, 60, 61 or 62; or a biologically active fragment of at least 30 contiguous amino acids thereof wherein any amino acid specified in the selected sequence is altered to a different amino acid, provided that no more than 15 of the amino acid residues in the sequence are so altered.

In another aspect, the invention relates to use of at least one isolated polypeptide for the preparation of a medicament for inhibiting formation of amyloid plaque, said polypeptide comprising an amino acid sequence selected from the group consisting of:
a) the amino acid sequence selected from the group consisting of SEQ ID NO. 5, 8, 11, 14, 17, 20 and 23; and
b) a sequence variant of the amino acid sequence selected from the group consisting of SEQ ID NO. 5, 8, 11, 14, 17, 20 and 23, wherein the sequence variant has at least 70% sequence identity to said SEQ ID NO. 5, 8, 11, 14, 17, 20 and 23; and
c) a biologically active fragment of at least 50 contiguous amino acids of any of a) wherein any amino acid specified in the selected sequence is altered to a different amino acid, provided that no more than 15 of the amino acid residues in the sequence are so altered.

In another aspect, the invention relates to use of at least one isolated nucleic acid molecule for the preparation of a medicament for the treatment of Alzheimer's Disease, said nucleic acid molecule comprising an nucleic acid sequence encoding upon expression, a polypeptide selected from the group consisting of:
a) the amino acid sequence selected from the group consisting of SEQ ID NO. 5, 8, 11, 14, 17, 20 and 23 or a naturally occurring precursor protein thereof; and
b) a sequence variant or a naturally occurring precursor protein of the amino acid sequence selected from the group consisting of SEQ ID NO. 5, 8, 11, 14, 17, 20 and 23, wherein the sequence variant has at least 70% sequence identity to said SEQ ID NO. 5, 8, 11, 14, 17, 20 and 23; and
c) a biologically active fragment of at least 50 contiguous amino acids of any of a) wherein any amino acid specified in the selected sequence is altered to a different amino acid, provided that no more than 50 of the amino acid residues in the sequence are so altered.

In a main aspect the present invention comprises use of a vector comprising at least one nucleic acid molecule as defined herein above, for the preparation of a medicament for inhibiting formation of amyloid plaque.

In a main aspect, the present invention relate to the use of an isolated host cell transformed or transduced with at least one vector as defined herein above, for the preparation of a medicament for inhibiting formation of amyloid plaque.

In one aspect the present invention relates to the use of an antibody capable of binding specifically to a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin, thereby exhibiting the same physiological response as the polypeptide of the present invention, for the manufacture of a medicament for inhibiting formation of amyloid plaque in an individual in need thereof.

In a main aspect, the present invention relate to a pharmaceutical composition comprising a combination of:
a) an isolated polypeptide selected from the group consisting of a sequence variant of the amino acid sequence selected from the group consisting of SEQ ID NO. 5, 8, 11, 14, 17, 20 and 23, wherein the sequence variant has at least 70% sequence identity to said SEQ ID NO. 5, 8, 11, 14, 17, 20 and 23; or
b) a biologically active fragment of at least 50 contiguous amino acids of any of a) wherein any amino acid specified in the selected sequence is altered to a different amino acid, provided that no more than 15 of the amino acid residues in the sequence are so altered;
   and
c) an isolated polypeptide selected from the group consisting of a sequence variant of the amino acid sequence selected from the group consisting of SEQ ID NO. 34, 41, 45, 49, 53, 57, 58, 59, 60, 61 and 62, wherein the sequence variant has at least 70% sequence identity to said SEQ ID NO. 34, 41, 45, 49, 53, 57, 58, 59, 60, 61 or 62; or
d) a biologically active fragment of at least 50 contiguous amino acids of any of a) wherein any amino acid specified in the selected sequence is altered to a different amino acid, provided that no more than 15 of the amino acid residues in the sequence are so altered.
   for the preparation of a combination medicament for inhibiting formation of amyloid plaque.

In another main aspect, the invention relate to the use of a pharmaceutical composition comprising a combination of:
a) an isolated polypeptide selected from the group consisting of a sequence variant of the amino acid sequence selected from the group consisting of SEQ ID NO. 5, 8, 11, 14, 17, 20 and 23, wherein the sequence variant has at least 70% sequence identity to said SEQ ID NO. 5, 8, 11, 14, 17, 20 and 23; or
b) a biologically active fragment of at least 50 contiguous amino acids of any of a) wherein any amino acid specified in the selected sequence is altered to a different amino acid, provided that no more than 15 of the amino acid residues in the sequence are so altered;
   and
c) an isolated polypeptide selected from the group consisting of a sequence variant of the amino acid sequence selected from the group consisting of SEQ ID NO. 34, 41, 45, 49, 53, 57, 58, 59, 60, 61 and 62, wherein the sequence variant has at least 70% sequence identity to said SEQ ID NO. 34, 41, 45, 49, 53, 57, 58, 59, 60, 61 or 62; or
d) a biologically active fragment of at least 50 contiguous amino acids of any of a) wherein any amino acid specified in the selected sequence is altered to a different amino acid, provided that no more than 15 of the amino acid residues in the sequence are so altered.
   for the preparation of a combination medicament for inhibiting formation of amyloid plaque.

In yet another aspect, the present invention relate to a pharmaceutical composition for use in a method of treatment of a disease or disorder resulting from amyloid plaque said composition comprising a combination of:
a) an isolated polypeptide selected from the group consisting of a sequence variant of the amino acid sequence selected from the group consisting of SEQ ID NO. 5, 8, 11, 14, 17, 20 and 23, wherein the sequence variant has at least 70% sequence identity to said SEQ ID NO. 5, 8, 11, 14, 17, 20 and 23; or
b) a biologically active fragment of at least 50 contiguous amino acids of any of a) wherein any amino acid specified in the selected sequence is altered to a different amino acid, provided that no more than 15 of the amino acid residues in the sequence are so altered;
   and
c) an isolated polypeptide selected from the group consisting of a sequence variant of the amino acid sequence selected from the group consisting of SEQ ID NO. 34, 41, 45, 49, 53, 57, 58, 59, 60, 61 and 62, wherein the sequence variant has at least 70% sequence identity to said SEQ ID NO. 34, 41, 45, 49, 53, 57, 58, 59, 60, 61 or 62; or
d) a biologically active fragment of at least 50 contiguous amino acids of any of a) wherein any amino acid specified in the selected sequence is altered to a different amino acid, provided that no more than 15 of the amino acid residues in the sequence are so altered;
   for the preparation of a combination medicament for inhibiting formation of amyloid plaque.

In a further aspect, the present invention relate to a pharmaceutical composition comprising a combination of:
a) an isolated polypeptide selected from the group consisting of a sequence variant of the amino acid sequence selected from the group consisting of SEQ ID NO. 5, 8, 11, 14, 17, 20 and 23, wherein the sequence variant has at least 70% sequence identity to said SEQ ID NO. 5, 8, 11, 14, 17, 20 and 23; or
b) a biologically active fragment of at least 50 contiguous amino acids of any of a) wherein any amino acid specified in the selected sequence is altered to a different amino acid, provided that no more than 15 of the amino acid residues in the sequence are so altered;
   and
c) an isolated polypeptide selected from the group consisting of a sequence variant of the amino acid sequence selected from the group consisting of SEQ ID NO. 34, 41, 45, 49, 53, 57, 58, 59, 60, 61 and 62, wherein the sequence variant has at least 70% sequence identity to said SEQ ID NO. 34, 41, 45, 49, 53, 57, 58, 59, 60, 61 or 62; or
d) a biologically active fragment of at least 50 contiguous amino acids of any of a) wherein any amino acid specified in the selected sequence is altered to a different amino acid, provided that no more than 15 of the amino acid residues in the sequence are so altered.
   for the preparation of a combination medicament for inhibiting formation of amyloid plaque.

In another main aspect, the present invention relates to the use of a pharmaceutical composition comprising a combination of:
a) an isolated polypeptide selected from the group consisting of a sequence variant of the amino acid sequence selected from the group consisting of SEQ ID NO. 5, 8, 11, 14, 17, 20 and 23, wherein the sequence variant has at least 70% sequence identity to said SEQ ID NO. 5, 8, 11, 14, 17, 20 and 23; or
b) a biologically active fragment of at least 50 contiguous amino acids of any of a) wherein any amino acid specified in the selected sequence is altered to a different amino acid, provided that no more than 15 of the amino acid residues in the sequence are so altered;
   and
c) an isolated polypeptide selected from the group consisting of a sequence variant of the amino acid sequence selected from the group consisting of SEQ ID NO. 34, 41, 45, 49, 53, 57, 58, 59, 60, 61 and 62, wherein the sequence variant has at least 70% sequence identity to said SEQ ID NO. 34, 41, 45, 49, 53, 57, 58, 59, 60, 61 or 62; or
d) a biologically active fragment of at least 50 contiguous amino acids of any of a) wherein any amino acid specified in the selected sequence is altered to a different amino acid, provided that no more than 15 of the amino acid residues in the sequence are so altered.
   for the preparation of a combination medicament for inhibiting formation of amyloid plaque.

In an important aspect, the present invention relate to a pharmaceutical composition for use in a method of treatment of a disease or disorder resulting from amyloid plaque said composition comprising a combination of:
a) an isolated polypeptide selected from the group consisting of a sequence variant of the amino acid sequence selected from the group consisting of SEQ ID NO. 5, 8, 11, 14, 17, 20 and 23, wherein the sequence variant has at least 70% sequence identity to said SEQ ID NO. 5, 8, 11, 14, 17, 20 and 23; or
b) a biologically active fragment of at least 50 contiguous amino acids of any of a) wherein any amino acid specified in the selected sequence is altered to a different amino acid, provided that no more than 15 of the amino acid residues in the sequence are so altered;
   and
c) an isolated polypeptide selected from the group consisting of a sequence variant of the amino acid sequence selected from the group consisting of SEQ ID NO. 34, 41, 45, 49, 53, 57, 58, 59, 60, 61 and 62, wherein the sequence variant has at least 70% sequence identity to said SEQ ID NO. 34, 41, 45, 49, 53, 57, 58, 59, 60, 61 or 62; or
d) a biologically active fragment of at least 50 contiguous amino acids of any of a) wherein any amino acid specified in the selected sequence is altered to a different amino acid, provided that no more than 15 of the amino acid residues in the sequence are so altered;
   for the preparation of a combination medicament for inhibiting formation of amyloid plaque.

In an important aspect the present invention relates to a method of inhibiting formation of amyloid plaque in a patient in need thereof, said method comprising administering to the patient the
a) at least one polypeptide of the invention; and/or
b) the at least one isolated nucleic acid sequence as defined herein above; and/or
c) the at least one expression vector as defined herein above; and/or
d) a composition of host cells as defined herein above; and/or;
e) a packaging cell line according as defined herein above, or
f) an antibody capable of binding specifically to a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin, or
g) a small organic molecule, or
h) a combination of two or more of any of a) through g).

In another important aspect the present invention relate to a method of inhibiting cleavage of APP to Aβ and APPα in an individual in need thereof, said method comprising administering to the individual the
a) at least one polypeptide as defined herein above; and/or
b) the at least one isolated nucleic acid sequence as defined herein above; and/or
c) the at least one expression vector as defined herein above; and/or
d) a composition of host cells as defined herein above; and/or;
e) a packaging cell line as defined herein above, or
f) an antibody capable of binding specifically to a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin, or
g) a small organic molecule, or
h) or a combination of two or more of any of a) through g).

In one aspect, the present invention relate to a method of inhibiting formation of Aβ and APPα plaque in a patient in need thereof, said method comprising administering to the patient:
a) the at least one polypeptide as defined herein above; and/or
b) the at least one isolated nucleic acid sequence as defined herein above; and/or
c) the at least one expression vector as defined herein above; and/or
d) a composition of host cells as defined herein above; and/or;
e) a packaging cell line as defined herein above, or
f) an antibody capable of binding specifically to a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin, or
g) a small organic molecule, or
h) a combination of two or more of any of a) through g).

In a highly preferred aspect the present invention relate to a method of upregulating SorLA (SEQ ID NO. 2) or a fragment or variant thereof, in a patient in need thereof, said method comprising administering to the patient:
a) the at least one polypeptide as defined herein above; and/or
b) the at least one isolated nucleic acid sequence as defined herein above; and/or
c) the at least one expression vector as defined herein above; and/or
d) a composition of host cells as defined herein above; and/or;
e) a packaging cell line as defined herein above, or
f) an antibody capable of binding specifically to a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin, or
g) a small organic molecule, or
h) a combination of two or more of any of a) through g)

In one aspect the present invention relate to an in vitro method of upregulating SorLA (SEQ ID NO. 2), said method comprising administering to the patient:
a) the at least one polypeptide as defined herein above; and/or
b) the at least one isolated nucleic acid sequence as defined herein above; and/or
c) the at least one expression vector as defined herein above; and/or
d) a composition of host cells as defined herein above; and/or;
e) a packaging cell line as defined herein above, or
f) an antibody capable of binding specifically to a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin, or
g) a small organic molecule, or
h) a combination of two or more of any of a) through g).

In one aspect the present invention relate to the use of the isolated polypeptide as defined herein above, for the preparation of a medicament for inhibiting cleavage of amyloid precursor protein (APP), said polypeptide comprising an amino acid sequence selected from the group consisting of:
a) the amino acid sequence selected from the group consisting of SEQ ID NO. 5, 8, 11, 14, 17, 20, 23, 34, 41, 45, 49, 53, 57, 58, 59, 60, 61 and 62 or a naturally occurring precursor protein thereof; and
b) a sequence variant or a naturally occurring precursor protein of the amino acid sequence selected from the group consisting of SEQ ID NO. 5, 8, 11, 14, 17, 20, 23, 34, 41, 45, 49, 53, 57, 58, 59, 60, 61 and 62, wherein the sequence variant has at least 70% sequence identity to said SEQ ID NO. 5, 8, 11, 14, 17, 20, 23, 34, 41, 45, 49, 53, 57, 58, 59, 60, 61 or 62; and
c) a biologically active fragment of at least 50 contiguous amino acids of any of a) wherein any amino acid specified in the selected sequence is altered to a different amino acid, provided that no more than 15 of the amino acid residues in the sequence are so altered.

In one aspect the present invention relate to the use of at least one agonist of a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin for the preparation of a medicament or a combination medicament for inhibiting formation of amyloid plaque.

In one aspect the present invention relate to the use of at least one agonist of a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin for the preparation of a medicament or a combination medicament for inhibiting cleavage of APP to Aβ and soluble APPα in an individual in need thereof.

In one aspect the present invention relate to the use of at least one agonist of a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin for the preparation of a medicament or a combination medicament for inhibiting cleavage of APP to Aβ and soluble APPα in an individual suffering from Alzheimer's Disease.

In one aspect the present invention relate to the use In one aspect the present invention relate to the use of at least one agonist of a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin for use in a method of treatment of abnormal levels of amyloid plaque, Aβ and soluble APPα in an individual.

In one aspect the invention relates to a kit in parts comprising:
a pharmaceutical composition as defined herein,
a medical instrument or other means for administering said pharmaceutical composition,
instructions on how to use the kit in parts and optionally
a second active ingredient.

### OVERVIEW OF THE DRAWINGS

FIG. 1: Overexpression of SorLA reduces Aβ production in neurons.

FIG. 2: Treatment with BDNF and CTGF induces SorLA mRNA and protein expression in neurons.

FIG. 3: Treatment with BDNF and CTGF does not induce Sortilin mRNA expression in neurons.

FIG. 4: A: Treatment with BDNF and CTGF reduces Aβ production in primary neurons. B: Treatment with BDNF in vivo reduces Aβ production in a SORLA dependent manner.

FIG. 5: Loss of BDNF in the mouse brain impairs SORLA expression

FIG. 6: Sequence alignment CTGF

FIG. 7: Sequence alignment BDNF

### DEFINITIONS

Addition: An addition or insertion, as used herein, refers to a change in an amino acid or nucleotide sequence resulting in the addition of one or more amino acid residues or nucleotides, respectively, as compared to the naturally occurring molecule.

Adjuvant: Any substance whose admixture with an administered immunogenic determinant/antigen increases or otherwise modifies the immune response to said determinant.

Affinity: The interaction of most ligands with their binding sites can be characterized in terms of a binding affinity. In general, high affinity ligand binding results from greater intermolecular force between the ligand and its receptor while low affinity ligand binding involves less intermolecular force between the ligand and its receptor. In general, high affinity binding involves a longer residence time for the ligand at its receptor binding site than is the case for low affinity binding. High affinity binding of ligands to receptors is often physiologically important when some of the binding energy can be used to cause a conformational change in the receptor, resulting in altered behavior of an associated ion channel or enzyme.

A ligand that can bind to a receptor, alter the function of the receptor and trigger a physiological response is called an agonist for that receptor. Agonist binding to a receptor can be characterized both in terms of how much physiological response can be triggered and the concentration of the agonist that is required to produce the physiological response. High affinity ligand binding implies that a relatively low concentration of a ligand is adequate to maximally occupy a ligand binding site and trigger a physiological response. Low affinity binding implies that a relatively high concentration of a ligand is required before the binding site is maximally occupied and the maximum physiological response to the ligand is achieved. Ligand binding is often characterized in terms of the concentration of ligand at which half of the receptor binding sites are occupied, known as the dissociation constant (k_{d}). Affinity is also the strength of binding between receptors and their ligands, for example between an antibody and its antigen.

Alcohol: A class of organic compounds containing one or more hydroxyl groups (OH). In this context a saturated or unsaturated, branched or unbranched hydrocarbon group sitting as a substituent on a larger molecule.

Alicyclic group: the term “alicyclic group” means a cyclic hydrocarbon group having properties resembling those of aliphatic groups.

Aliphatic group: in the context of the present invention, the term “aliphatic group” means a saturated or unsaturated linear or branched hydrocarbon group. This term is used to encompass alkyl, alkenyl, and alkynyl groups, for example.

Alkyl group: the term “alkyl group” means a saturated linear or branched hydrocarbon group including, for example, methyl, ethyl, isopropyl, t-butyl, heptyl, dodecyl, octadecyl, amyl, 2-ethylhexyl, and the like.

Alkenyl group: the term “alkenyl group” means an unsaturated, linear or branched hydrocarbon group with one or more carbon-carbon double bonds, such as a vinyl group.

Alkynyl group: the term “alkynyl group” means an unsaturated, linear or branched hydrocarbon group with one or more carbon-carbon triple bonds.

Amphiphil: substance containing both polar, water-soluble and nonpolar, water-insoluble groups.

Agonist: An agonist is a compound capable of increasing or effecting the activity of a receptor. Specifically, a Vps10p-domain receptor agonist is a compound capable of binding to one or more of binding sites of a Vps10p-domain receptor thereby inducing the same physiological response as a given endogenous agonist ligand compound.

Antagonist: An antagonist is in this case synonymous with an inhibitor. An antagonist is a compound capable of decreasing the activity of an effector such as a receptor. Specifically, a Vps10p-domain receptor antagonist is a compound capable of binding to one or more of binding sites of Vps10p-domain receptor thereby inhibiting binding of another ligand thus inhibiting a physiological response.

antisense-RNA: an RNA molecule capable of causing gene silencing by specifically binding to an mRNA molecule of interest.

antisense-DNA: a DNA molecule capable of causing gene silencing by specifically binding to an mRNA molecule of interest.

Antibody: The term “antibody” as referred to herein includes whole antibodies and any antigen binding fragment (i.e., “antigen-binding portion”) or single chain thereof.

“A whole antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region (abbreviated herein as C_{H}). Each light chain is comprised of a light chain variable region (abbreviated herein as V_{L}) and a light chain constant region (abbreviated herein as C_{L}). The V_{H} and V_{L} regions can be further subdivided into regions of hypervariability, termed complementarity determining regions (CDRs), interspersed with regions that are more conserved, termed framework regions (FRs). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR), and (vii) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody.

A further example of an antigen binding-domain is immunoglobulin fusion proteins comprising (i) a binding domain polypeptide that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The binding domain polypeptide can be a heavy chain variable region or a light chain variable region. Such binding-domain immunoglobulin fusion proteins are further disclosed in US 2003/0118592 and US 2003/0133939.

These antibody fragments are obtained using conventional techniques known to those with skill in the art, and the fragments are screened for utility in the same manner as are intact antibodies.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The term “discontinuous epitope”, as used herein, means a conformational epitope on a protein antigen which is formed from at least two separate regions in the primary sequence of the protein. A discontinuous epitope may also be formed by at least two regions of one or more proteins, in such a case the antigen may be formed by one or more proteins.

The term “bispecific molecule” is intended to include any agent, e.g., a protein, peptide, or protein or peptide complex, which has two different binding specificities. For example, the molecule may bind to, or interact with, (a) a cell surface antigen and (b) an Fc receptor on the surface of an effector cell. The term “multispecific molecule” or “heterospecific molecule” is intended to include any agent, e.g., a protein, peptide, or protein or peptide complex, which has more than two different binding specificities. For example, the molecule may bind to, or interact with, (a) a cell surface antigen, (b) an Fc receptor on the surface of an effector cell, and (c) at least one other component. Accordingly, the invention includes, but is not limited to, bispecific, trispecific, tetraspecific, and other multispecific molecules which are directed to the CaOU-1 epitope, and to other cell surface antigens or targets, such as Fc receptors on effector cells.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library, and wherein the selected human antibody is at least 90%, more preferably at least 95%, even more preferably at least 96%, 97%, 98%, or 99% identical in amino acid sequence to the amino acid sequence encoded by the germline immunoglobulin gene. Typically, a human anti-body derived from a particular human germline sequence will display no more than 10 amino acid differences, more preferably, no more than 5, or even more preferably, no more than 4, 3, 2, or 1 amino acid difference from the amino acid sequence encoded by the germline immunoglobulin gene.

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “recombinant human antibody”, as used herein, includes all human anti-bodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable and constant regions derived from human germline immunoglobulin sequences.

In certain embodiments, however, such recombinant human antibodies can be subjected to in vitro mutagenesis (or, when an animal transgenic for human Ig sequences is used, in vivo somatic mutagenesis) also called affinity maturation and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, a “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

An “isolated antibody”, as used herein, is intended to refer to an antibody which is substantially free of other antibodies having different antigenic specificities (e.g., an isolated antibody that specifically binds to the CaOU-1 epitope is substantially free of antibodies that specifically bind antigens other than the CaOU-1 epitope). An isolated antibody that specifically binds to an epitope, isoform or variant of the human CaOU-1 epitope may, however, have cross-reactivity to other related antigens, e.g., from other species (e.g., CaOU-1 epitope species homologs). Moreover, an isolated antibody may be substantially free of other cellular material and/or chemicals.

As used herein, “specific binding” refers to antibody binding to a predetermined antigen. Typically, the antibody binds with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹M or even less, when measured as apparent affinities based on IC₅₀ values in FACS, and binds to the predetermined antigen with an affinity corresponding to a K_{D} that is at least ten-fold lower, such as at least 100-fold lower than its affinity for binding to a non-specific antigen (e.g., BSA, casein) other than the predetermined antigen or a closely-related antigen.

Avidity: The functional combining strength of an antibody with its antigen which is related to both the affinity of the reaction between the epitopes and paratopes, and the valencies of the antibody and antigen

Antibody Classes: Depending on the amino acid sequences of the constant domain of their heavy chains, immunoglobulins can be assigned to different classes. There are at least five (5) major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2. The heavy chains constant domains that correspond to the different classes of immunoglobulins are called alpha (α), delta (δ), epsilon (ε), gamma (γ) and mu (μ), respectively. The light chains of antibodies can be assigned to one of two clearly distinct types, called kappa (κ) and lambda (λ), based on the amino sequences of their constant domain. The subunit structures and three-dimensional configurations of different classes of immunoglobulins are well known.

Antibody Combining Site: An antibody combining site is that structural portion of an antibody molecule comprised of a heavy and light chain variable and hypervariable regions that specifically binds (immunoreacts with) an antigen. The term immunoreact in its various forms means specific binding between an antigenic determinant-containing molecule and a molecule containing an antibody combining site such as a whole antibody molecule or a portion thereof. Alternatively, an antibody combining site is known as an antigen binding site.

Chimeric antibody: An antibody in which the variable regions are from one species of animal and the constant regions are from another species of animal. For example, a chimeric antibody can be an antibody having variable regions which derive from a mouse monoclonal antibody and constant regions which are human.

Complementarity determining region or CDR: Regions in the V-domains of an anti-body that together form the antibody recognizing and binding domain.

Constant Region or constant domain or C-domain: Constant regions are those structural portions of an antibody molecule comprising amino acid residue sequences within a given isotype which may contain conservative substitutions therein. Exemplary heavy chain immunoglobulin constant regions are those portions of an immunoglobulin molecule known in the art as CH1, CH2, CH3, CH4 and CH5. An exemplary light chain immunoglobulin constant region is that portion of an immunoglobulin molecule known in the art as C_{L}.

Diabodies: This term refers to a small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90: 6444-6448 (1993).

Fv: dual chain antibody fragment containing both a V_{H} and a V_{L}.

Human antibody framework: A molecule having an antigen binding site and essentially all remaining immunoglobulin-derived parts of the molecule derived from a human immunoglobulin.

Humanised antibody framework: A molecule having an antigen binding site derived from an immunoglobulin from a non-human species, whereas some or all of the remaining immunoglobulin-derived parts of the molecule is derived from a human immunoglobulin. The antigen binding site may comprise: either a complete variable domain from the non-human immunoglobulin fused onto one or more human constant domains; or one or more of the complementarity determining regions (CDRs) grafted onto appropriate human framework regions in the variable domain. In a humanized antibody, the CDRs can be from a mouse monoclonal antibody and the other regions of the antibody are human.

Immunoglobulin: The serum antibodies, including IgG, IgM, IgA, IgE and IgD.

Immunoglobulin isotypes: The names given to the Ig which have different H chains, the names are IgG (IgG_{1,2,3,4}), IgM, IgA (IgA_{1,2}), sIgA, IgE, IgD.

Immunologically distinct: The phrase immunologically distinct refers to the ability to distinguish between two polypeptides on the ability of an antibody to specifically bind one of the polypeptides and not specifically bind the other polypeptide.

Monoclonal Antibody: The phrase monoclonal antibody in its various grammatical forms refers to a population of antibody molecules that contains only one species of antibody combining site capable of immunoreacting with a particular antigen. A monoclonal antibody thus typically displays a single binding affinity for any antigen with which it immunoreacts. A monoclonal antibody may contain an antibody molecule having a plurality of antibody combining sites, each immunospecific for a different antigen, e.g., a bispecific monoclonal antibody.

Polyclonal antibody: Polyclonal antibodies are a mixture of antibody molecules recognising a specific given antigen, hence polyclonal antibodies may recognise different epitopes within said antigen.

Single Chain Antibody or scFv: The phrase single chain antibody refers to a single polypeptide comprising one or more antigen binding sites, most commonly one antigen binding site. Furthermore, although the H and L chains of an Fv fragment are encoded by separate genes, they may be linked either directly or via a peptide, for example a synthetic linker can be made that enables them to be made as a single protein chain (known as single chain antibody, sAb; Bird et al. 1988 Science 242:423-426; and Huston et al. 1988 PNAS 85:5879-5883) by recombinant methods. Such single chain antibodies are also encompassed within the term “antibody”, and may be utilized as binding determinants in the design and engineering of a multispecific binding molecule.

Valency: The term valency refers to the number of potential antigen binding sites, i.e. binding domains, in a polypeptide. A polypeptide may be monovalent and contain one antigen binding site or a polypeptide may be bivalent and contain two antigen binding sites. Additionally, a polypeptide may be tetravalent and contain four antigen binding sites. Each antigen binding site specifically binds one antigen. When a polypeptide comprises more than one antigen binding site, each antigen binding site may specifically bind the same or different antigens. Thus, a polypeptide may contain a plurality of antigen binding sites and therefore be multivalent and a polypeptide may specifically bind the same or different antigens.

V-domain: Variable domain are those structural portions of an antibody molecule comprising amino acid residue sequences forming the antigen binding sites. An exemplary light chain immunoglobulin variable region is that portion of an immunoglobulin molecule known in the art as V_{L}.

V_{L}: Variable domain of the light chain.

V_{H}: Variable domain of the heavy chain.

Apoptosis: Apoptosis is a process of suicide by a cell in a multi-cellular organism. It is one of the main types of programmed cell death (PCD), and involves an orchestrated series of biochemical events leading to a characteristic cell morphology and death.

Apoptosis inhibitor: Any compound capable of decreasing the process of apoptosis.

Aromatic group: the term “aromatic group” or “aryl group” means a mono- or polycyclic aromatic hydrocarbon group.

Binding: The term “binding” or “associated with” refers to a condition of proximity between chemical entities or compounds, or portions thereof. The association may be non-covalent-wherein the juxtaposition is energetically favoured by hydrogen bonding or van der Waals or electrostatic interactions—or it may be covalent.

Binding site: The term “binding site” or “binding pocket”, as used herein, refers to a region of a molecule or molecular complex that, as a result of its shape, favourably associates with another molecule, molecular complex, chemical entity or compound. As used herein, the pocket comprises at least a deep cavity and, optionally a shallow cavity.

Bioreactive agent: The term “bioactive agent” as used herein refers to any a substance which may be used in connection with an application that is therapeutic or diagnostic, such as, for example, in methods for diagnosing the presence or absence of a disease in a patient and/or methods for the treatment of a disease in a patient. “Bioactive agent” refers to substances, which are capable of exerting a biological effect in vitro and/or in vivo. The bioactive agents may be neutral, positively or negatively charged. Suitable bioactive agents include, for example, prodrugs, diagnostic agents, therapeutic agents, pharmaceutical agents, drugs, oxygen delivery agents, blood substitutes, synthetic organic molecules, polypeptides, peptides, vitamins, steroids, steroid analogues and genetic determinants, including nucleosides, nucleotides and polynucleotides.

Cationic group: A chemical group capable of functioning as a proton donor when a compound comprising the chemical group is dissolved in a solvent, preferably when dissolved in water.

Complex: As used herein the term “complex” refers to the combination of a molecule or a protein, conservative analogues or truncations thereof associated with a chemical entity.

Cyclic group: the term “cyclic group” means a closed ring hydrocarbon group that is classified as an alicyclic group, aromatic group, or heterocyclic group.

Cycloalkenyl: means a monovalent unsaturated carbocyclic radical consisting of one, two or three rings, of three to eight carbons per ring, which can optionally be substituted with one or two substituents selected from the group consisting of hydroxy, cyano, lower alkenyl, lower alkoxy, lower haloalkoxy, alkenylthio, halo, haloalkenyl, hydroxyalkenyl, nitro, alkoxycarbonenyl, amino, alkenylamino, alkenylsulfonyl, arylsulfonyl, alkenylaminosulfonyl, arylaminosulfonyl, alkylsulfonylamino, arylsulfonylamino, alkenylaminocarbonyl, arylaminocarbonyl, alkenylcarbonylamino and arylcarbonylamino.

Cycloalkyl: means a monovalent saturated carbocyclic radical consisting of one, two or three rings, of three to eight carbons per ring, which can optionally be substituted with one or two substituents selected from the group consisting of hydroxy, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino and arylcarbonylamino.

Deletion: A deletion, as used herein, refers to a change in the amino acid or nucleotide sequence and results in the absence of one or more amino acid residues or nucleotides.

Dipole-dipole interaction: The term “dipole-dipole interaction” as used herein refers to the attraction which can occur among two or more polar molecules. Thus, “dipole-dipole interaction” refers to the attraction of the uncharged, partial positive end of a first polar molecule to the uncharged, partial negative end of a second polar molecule. “Dipole-dipole interaction” also refers to intermolecular hydrogen bonding.

Down-regulation of expression: a process leading to decreased expression of genes, preferably of endogenous genes. Specifically, agents such as BDNF and/or CTGF of the present invention are capable of downregulating or preventing expression of the Vps10p-domain receptor SorLA.

Electrostatic interaction: The term “electrostatic interaction” as used herein refers to any interaction occurring between charged components, molecules or ions, due to attractive forces when components of opposite electric charge are attracted to each other. Examples include, but are not limited to: ionic interactions, covalent interactions, interactions between a ion and a dipole (ion and polar molecule), interactions between two dipoles (partial charges of polar molecules), hydrogen bonds and London dispersion bonds (induced dipoles of polarizable molecules). Thus, for example, “ionic interaction” or “electrostatic interaction” refers to the attraction between a first, positively charged molecule and a second, negatively charged molecule. Ionic or electrostatic interactions include, for example, the attraction between a negatively charged bioactive agent.

Form a ring: means that the atoms mentioned are connected through a bond when the ring structure is formed.

Fragments: The polypeptide fragments according to the present invention, including any functional equivalents thereof, may in one embodiment comprise less than 500 amino acid residues, such as less than 450 amino acid residues, for example less than 400 amino acid residues, such as less than 350 amino acid residues, for example less than 300 amino acid residues, for example less than 250 amino acid residues, such as less than 240 amino acid residues, for example less than 225 amino acid residues, such as less than 200 amino acid residues, for example less than 180 amino acid residues, such as less than 160 amino acid residues, for example less than 150 amino acid residues, such as less than 140 amino acid residues, for example less than 130 amino acid residues, such as less than 120 amino acid residues, for example less than 110 amino acid residues, such as less than 100 amino acid residues, for example less than 90 amino acid residues, such as less than 85 amino acid residues, for example less than 80 amino acid residues, such as less than 75 amino acid residues, for example less than 70 amino acid residues, such as less than 65 amino acid residues, for example less than 60 amino acid residues, such as less than 55 amino acid residues, for example less than 50 amino acid residues. Fragments of neurotensin include but is not limited to the C-terminal amino acids of neurotensin PYIL and YIL.

Functional equivalency: “Functional equivalency” as used in the present invention is, according to one preferred embodiment, established by means of reference to the corresponding functionality of a predetermined fragment of the sequence.

Functional equivalents or variants of a Vps10p-domain receptor modulator will be understood to exhibit amino acid sequences gradually differing from the preferred predetermined proneurotrophin activity modulator sequence, as the number and scope of insertions, deletions and substitutions including conservative substitutions increase. This difference is measured as a reduction in homology between the preferred predetermined sequence and the fragment or functional equivalent.

A functional variant obtained by substitution may well exhibit some form or degree of native proneurotrophin activity modulator activity, and yet be less homologous, if residues containing functionally similar amino acid side chains are substituted. Functionally similar in this respect refers to dominant characteristics of the side chains such as hydrophobic, basic, neutral or acidic, or the presence or absence of steric bulk. Accordingly, in one embodiment of the invention, the degree of identity is not a principal measure of a fragment being a variant or functional equivalent of a preferred predetermined fragment according to the present invention.

Gene “silencing”: a process leading to reduced expression of endogenous genes. Gene silencing is preferably the result of post-transcriptional reduction of gene expression.

Group: (Moiety/substitution) as is well understood in this technical area, a large degree of substitution is not only tolerated, but is often advisable. Substitution is anticipated on the materials of the present invention. As a means of simplifying the discussion and recitation of certain terminology used throughout this application, the terms “group” and “moiety” are used to differentiate between chemical species that allow for substitution or that may be substituted and those that do not allow or may not be so substituted. Thus, when the term “group” is used to describe a chemical substituent, the described chemical material includes the unsubstituted group and that group with O, N, or S atoms, for example, in the chain as well as carbonyl groups or other conventional substitution. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, t-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, sulfoalkyls, etc. On the other hand, the phrase “alkyl moiety” is limited to the inclusion of only pure open chain saturated hydrocarbon alkyl substituents, such as methyl, ethyl, propyl, t-butyl, and the like. The same definitions apply to “alkenyl group” and “alkenyl moiety”; to “alkynyl group” and “alkynyl moiety”; to “cyclic group” and “cyclic moiety; to “alicyclic group” and “alicyclic moiety”; to “aromatic group” or “aryl group” and to “aromatic moiety” or “aryl moiety”; as well as to “heterocyclic group” and “heterocyclic moiety”.

Heterocyclic group: the term “heterocyclic group” means a closed ring hydrocarbon in which one or more of the atoms in the ring is an element other than carbon (e.g., nitrogen, oxygen, sulphur, etc.).

Heterocyclyl means a monovalent saturated cyclic radical, consisting of one to two rings, of three to eight atoms per ring, incorporating one or two ring heteroatoms (chosen from N, O or S(O)₀₋₂, and which can optionally be substituted with one or two substituents selected from the group consisting of hydroxyl, oxo, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminofarbonyl, arylaminocarbonyl, alkylcarbonylamino, or arylcarbonylamino.

Heteroaryl means a monovalent aromatic cyclic radical having one to three rings, of four to eight atoms per ring, incorporating one or two heteroatoms (chosen from nitrogen, oxygen, or sulphur) within the ring which can optionally be substituted with one or two substituents selected from the group consisting of hydroxy, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonlamino and arylcarbonylamino.

Homology: The homology between amino acid sequences may be calculated using well known scoring matrices such as any one of BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, and BLOSUM 90.

Fragments sharing homology with fragments of SEQ ID NO:1 to 13, respectively, are to be considered as falling within the scope of the present invention when they are preferably at least about 60 percent homologous, for example at least 65 percent homologous, for example at least 70 percent homologous, for example at least 75 percent homologous, for example at least 80 percent homologous, for example at least 85 percent homologous, for example at least 90 percent homologous, for example at least 92 percent homologous, such as at least 94 percent homologous, for example at least 95 percent homologous, such as at least 96 percent homologous, for example at least 97 percent homologous, such as at least 98 percent homologous, for example at least 99 percent homologous with said predetermined fragment sequences, respectively. According to one embodiment of the invention, the homology percentages refer to identity percentages.

A further suitably adaptable method for determining structure and function relationships of peptide fragments is described in U.S. Pat. No. 6,013,478, which is herein incorporated by reference. Also, methods of assaying the binding of an amino acid sequence to a receptor moiety are known to the skilled artisan.

In addition to conservative substitutions introduced into any position of a preferred predetermined proneurotrophin activity modulator, or a fragment thereof, it may also be desirable to introduce non-conservative substitutions in any one or more positions of such a proneurotrophin activity modulator.

A non-conservative substitution leading to the formation of a functionally equivalent fragment of proneurotrophin activity modulator would for example i) differ substantially in polarity, for example a residue with a non-polar side chain (Ala, Leu, Pro, Trp, Val, Ile, Leu, Phe or Met) substituted for a residue with a polar side chain such as Gly, Ser, Thr, Cys, Tyr, Asn, or Gln or a charged amino acid such as Asp, Glu, Arg, or Lys, or substituting a charged or a polar residue for a non-polar one; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Variants obtained by substitution of amino acids may in one preferred embodiment be made based upon the hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include: arginine and lysine; glutamate and aspartate; serine and threonine; glutamine and asparagine; and valine, leucine and isoleucine.

In addition to the variants described herein, sterically similar variants may be formulated to mimic the key portions of the variant structure and that such compounds may also be used in the same manner as the variants of the invention. This may be achieved by techniques of modelling and chemical designing known to those of skill in the art. It will be understood that all such sterically similar constructs fall within the scope of the present invention.

In a further embodiment the present invention relates to functional variants comprising substituted amino acids having hydrophilic values or hydropathic indices that are within +/−4.9, for example within +/−4.7, such as within +/−4.5, for example within +/−4.3, such as within +/−4.1, for example within +/−3.9, such as within +/−3.7, for example within +/−3.5, such as within +/−3.3, for example within +/−3.1, such as within +/−2.9, for example within +/−2.7, such as within +/−2.5, for example within +/−2.3, such as within +/−2.1, for example within +/−2.0, such as within +/−1.8, for example within +/−1.6, such as within +/−1.5, for example within +/−1.4, such as within +/−1.3 for example within +/−1.2, such as within +/−1.1, for example within +/−1.0, such as within +/−0.9, for example within +/−0.8, such as within +/−0.7, for example within +/−0.6, such as within +/−0.5, for example within +/−0.4, such as within +/−0.3, for example within +/−0.25, such as within +/−0.2 of the value of the amino acid it has substituted.

The importance of the hydrophilic and hydropathic amino acid indices in conferring interactive biologic function on a protein is well understood in the art (Kyte & Doolittle, 1982 and Hopp, U.S. Pat. No. 4,554,101, each incorporated herein by reference).

The amino acid hydropathic index values as used herein are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5) (Kyte & Doolittle, 1982).

The amino acid hydrophilicity values are: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4) (U.S. Pat. No. 4,554,101).

In addition to the peptidyl compounds described herein, sterically similar compounds may be formulated to mimic the key portions of the peptide structure and that such compounds may also be used in the same manner as the peptides of the invention.

This may be achieved by techniques of modelling and chemical designing known to those of skill in the art. For example, esterification and other alkylations may be employed to modify the amino terminus of, e.g., a di-arginine peptide backbone, to mimic a tetra peptide structure. It will be understood that all such sterically similar constructs fall within the scope of the present invention.

Peptides with N-terminal alkylations and C-terminal esterifications are also encompassed within the present invention. Functional equivalents also comprise glycosylated and covalent or aggregative conjugates formed with the same or other proneurotrophin activity modulator fragments and/or proneurotrophin activity modulator molecules, including dimers or unrelated chemical moieties. Such functional equivalents are prepared by linkage of functionalities to groups which are found in fragment including at any one or both of the N- and C-termini, by means known in the art.

Functional equivalents may thus comprise fragments conjugated to aliphatic or acyl esters or amides of the carboxyl terminus, alkylamines or residues containing carboxyl side chains, e.g., conjugates to alkylamines at aspartic acid residues; O-acyl derivatives of hydroxyl group-containing residues and N-acyl derivatives of the amino terminal amino acid or amino-group containing residues, e.g. conjugates with fMet-Leu-Phe or immunogenic proteins. Derivatives of the acyl groups are selected from the group of alkyl-moieties (including C3 to C10 normal alkyl), thereby forming alkanoyl species, and carbocyclic or heterocyclic compounds, thereby forming aroyl species. The reactive groups preferably are difunctional compounds known per se for use in cross-linking proteins to insoluble matrices through reactive side groups.

Covalent or aggregative functional equivalents and derivatives thereof are useful as reagents in immunoassays or for affinity purification procedures. For example, a fragment of proneurotrophin activity modulator according to the present invention may be insolubilized by covalent bonding to cyanogen bromide-activated Sepharose by methods known per se or adsorbed to polyolefin surfaces, either with or without glutaraldehyde cross-linking, for use in an assay or purification of anti-neurotrophin activity modulator antibodies or cell surface receptors. Fragments may also be labelled with a detectable group, e.g., radioiodinated by the chloramine T procedure, covalently bound to rare earth chelates or conjugated to another fluorescent moiety for use in e.g. diagnostic assays.

Mutagenesis of a preferred predetermined fragment of proneurotrophin activity modulator can be conducted by making amino acid insertions, usually on the order of about from 1 to 10 amino acid residues, preferably from about 1 to 5 amino acid residues, or deletions of from about from 1 to 10 residues, such as from about 2 to 5 residues.

In one embodiment the ligand of binding site 1, 2 or 3 is an oligopeptide synthesised by automated synthesis. Any of the commercially available solid-phase techniques may be employed, such as the Merrifield solid phase synthesis method, in which amino acids are sequentially added to a growing amino acid chain (see Merrifield, J. Am. Chem. Soc. 85:2149-2146, 1963).

Equipment for automated synthesis of polypeptides is commercially available from suppliers such as Applied Biosystems, Inc. of Foster City, Calif., and may generally be operated according to the manufacturer's instructions. Solid phase synthesis will enable the incorporation of desirable amino acid substitutions into any fragment of proneurotrophin activity modulator according to the present invention. It will be understood that substitutions, deletions, insertions or any subcombination thereof may be combined to arrive at a final sequence of a functional equivalent. Insertions shall be understood to include amino-terminal and/or carboxyl-terminal fusions, e.g. with a hydrophobic or immunogenic protein or a carrier such as any polypeptide or scaffold structure capable as serving as a carrier.

Oligomers including dimers including homodimers and heterodimers of fragments of sortilin inhibitors according to the invention are also provided and fall under the scope of the invention. Proneurotrophin activity modulator functional equivalents and variants can be produced as homodimers or heterodimers with other amino acid sequences or with native sortilin inhibitor sequences. Heterodimers include dimers containing immunoreactive sortilin inhibiting fragments as well as sortilin inhibiting fragments that need not have or exert any biological activity.

Vpas10p-domain receptor antagonists including but not limited to Sortilin inhibiting peptide fragments may be synthesised both in vitro and in vivo. Method for in vitro synthesis are well known, and methods being suitable or suitably adaptable to the synthesis in vivo of sortilin inhibitors are also described in the prior art. When synthesized in vivo, a host cell is transformed with vectors containing DNA encoding a sortilin peptide inhibitor or a fragment thereof. A vector is defined as a replicable nucleic acid construct. Vectors are used to mediate expression of proneurotrophin activity modulator. An expression vector is a replicable DNA construct in which a nucleic acid sequence encoding the predetermined sortilin inhibiting fragment, or any functional equivalent thereof that can be expressed in vivo, is operably linked to suitable control sequences capable of effecting the expression of the fragment or equivalent in a suitable host. Such control sequences are well known in the art. Both prokaryotic and eukaryotic cells may be used for synthesising ligands. Cultures of cells derived from multicellular organisms however represent preferred host cells. In principle, any higher eukaryotic cell culture is workable, whether from vertebrate or invertebrate culture. Examples of useful host cell lines are VERO and HeLa cells, Chinese hamster ovary (CHO) cell lines, and WI38, BHK, COS-7, 293 and MDCK cell lines. Preferred host cells are eukaryotic cells known to synthesize endogenous sortilin inhibitors. Cultures of such host cells may be isolated and used as a source of the fragment, or used in therapeutic methods of treatment, including therapeutic methods aimed at promoting or inhibiting a growth state, or diagnostic methods carried out on the human or animal body.

Hydrophobic bond: The term “hydrogen bond” as used herein refers to an attractive force, or bridge, which may occur between a hydrogen atom which is bonded covalently to an electronegative atom, for example, oxygen, sulphur, or nitrogen, and another electronegative atom. The hydrogen bond may occur between a hydrogen atom in a first molecule and an electronegative atom in a second molecule (intermolecular hydrogen bonding). Also, the hydrogen bond may occur between a hydrogen atom and an electronegative atom which are both contained in a single molecule (intramolecular hydrogen bonding).

Hydrophobic interaction: The term “hydrophobic interaction” as used herein refers to any interaction occurring between essentially non-polar (hydrophobic) components located within attraction range of one another in a polar environment (e.g. water). As used herein, attraction range is on the scale of from 0.1 up to 2 nm. A particular type of hydrophobic interaction is exerted by “Van der Waal's forces”, i.e. the attractive forces between non-polar molecules that are accounted for by quantum mechanics. Van der Waal's forces are generally associated with momentary dipole moments which are induced by neighbouring molecules and which involve changes in electron distribution.

Inhibiting: The term inhibiting as used herein refers to the prevention of binding between two or more components of a Vps10p-domain receptor: TrpV receptor binary complex, and/or a Vps10p-domain receptor: TrkA:TrpV receptor ternary complex.

Insertion: An insertion or addition, as used herein, refers to a change in an amino acid or nucleotide sequence resulting in the addition of one or more amino acid residues or nucleotides, respectively, as compared to the naturally occurring molecule.

In vitro/in vivo: the terms are used in their normal meaning.

Lesions: are caused by any process that damages tissues. A cancerous tumor is an example of a lesion, however the surrounding tissue damaged by a tumor is also a lesion. Trauma, including electrocution and chemical burns can also cause lesions. Certain diseases present lesions, for example the skin deformities caused by chicken pox. Lesions can also be caused by metabolic processes, like an ulcer or autoimmune activity, as in the case with many forms of arthritis. Lesions are sometimes intentionally inflicted during neurosurgery, such as the carefully-placed brain lesion used to treat epilepsy and other brain disorders.

Ligand: a substance, compound or biomolecule such as a protein including receptors, that is able to bind to and form a complex with (a second) biomolecule to serve a biological purpose. In a narrower sense, it is a signal triggering molecule binding to a site on a target protein, by intermolecular forces such as ionic bonds, hydrogen bonds and Van der Waals forces. The docking (association) is usually reversible (dissociation). Actual irreversible covalent binding between a ligand and its target molecule is rare in biological systems. As opposed to the meaning in metalorganic and inorganic chemistry, it is irrelevant, whether or not the ligand actually binds at a metal site, as it is the case in hemoglobin. Ligand binding to receptors may alter the chemical conformation, i.e. the three dimensional shape of the receptor protein. The conformational state of a receptor protein determines the functional state of a receptor. The tendency or strength of binding is called affinity. Ligands include substrates, inhibitors, activators, non-self receptors, co-receptors and neurotransmitters. Radioligands are radioisotope labeled compounds and used in vivo as tracers in PET studies and for in vitro binding studies.

Moieties of a particular compound cover group(s) or part(s) of said particular compound.

Neuropathic pain: is a chronic pain initiated or caused by a primary lesion or dysfunction in the nervous system.

Pharmaceutical agent: The terms “pharmaceutical agent” or “drug” or “medicament” refer to any therapeutic or prophylactic agent which may be used in the treatment (including the prevention, diagnosis, alleviation, or cure) of a malady, affliction, condition, disease or injury in a patient. Therapeutically useful genetic determinants, peptides, polypeptides and polynucleotides may be included within the meaning of the term pharmaceutical or drug. As defined herein, a “therapeutic agent”, “pharmaceutical agent” or “drug” or “medicament” is a type of bioactive agent.

Pharmaceutical composition: or drug, medicament or agent refers to any chemical or biological material, compound, or composition capable of inducing a desired therapeutic effect when properly administered to a patient. Some drugs are sold in an inactive form that is converted in vivo into a metabolite with pharmaceutical activity. For purposes of the present invention, the terms “pharmaceutical composition” and “medicament” encompass both the inactive drug and the active metabolite.

Polypeptide: The term “polypeptide” as used herein refers to a molecule comprising at least two amino acids. The amino acids may be natural or synthetic. “Oligopeptides” are defined herein as being polypeptides of length not more than 100 amino acids. The term “polypeptide” is also intended to include proteins, i.e. functional biomolecules comprising at least one polypeptide; when comprising at least two polypeptides, these may form complexes, be covalently linked or may be non-covalently linked. The polypeptides in a protein can be glycosylated and/or lipidated and/or comprise prosthetic groups.

Polynucleotide: “Polynucleotide” as used herein refers to a molecule comprising at least two nucleic acids. The nucleic acids may be naturally occurring or modified, such as locked nucleic acids (LNA), or peptide nucleic acids (PNA). Polynucleotide as used herein generally pertains to
i) a polynucleotide comprising a predetermined coding sequence, or
ii) a polynucleotide encoding a predetermined amino acid sequence, or
iii) a polynucleotide encoding a fragment of a polypeptide encoded by polynucleotides (i) or (ii), wherein said fragment has at least one predetermined activity as specified herein; and
iv) a polynucleotide the complementary strand of which hybridizes under stringent conditions with a polynucleotide as defined in any one of (i), (ii) and (iii), and encodes a polypeptide, or a fragment thereof, having at least one predetermined activity as specified herein; and
v) a polynucleotide comprising a nucleotide sequence which is degenerate to the nucleotide sequence of polynucleotides (iii) or (iv);
   or the complementary strand of such a polynucleotide.

Precursor polypeptide: also called protein precursor or pro-protein or pro-peptide, is a protein or peptide that can be structurally modified by posttranslational modification. The name of the precursor for a protein is often prefixed by pro. Examples include proBDNF and proNGF. Protein precursors are often used by an organism when the subsequent protein is potentially harmful, but needs to be available on short notice and/or in large quantities. Some protein precursors are secreted from the cell. Many of these are synthesized with an N-terminal signal peptide that targets them for secretion. Like other proteins that contain a signal peptide, their name is prefixed by pre. They are thus called pre-proteins (e.g. preCTGF), pre-peptides, pre-pro-proteins (e.g. pre-pro-BDNF) or pre-pro-peptides. The signal peptide is normally cleaved off in the endoplasmic reticulum.

Purified antibody: The term a “purified antibody” is an antibody at least 60 weight percent of which is free from the polypeptides and naturally-occurring organic molecules with which it is naturally associated. Preferably, the preparation comprises antibody in an amount of at least 75 weight percent, more preferably at least 90 weight percent, and most preferably at least 99 weight percent.

Root mean square deviation: The term “root mean square deviation” (rmsd) is used as a mean of comparing two closely related structures and relates to a deviation in the distance between related atoms of the two structures after structurally minimizing this distance in an alignment. Related proteins with closely related structures will be characterized by relatively low RMSD values whereas larger differences will result in an increase of the RMSD value.

Sequence identity: Sequence identity is determined in one embodiment by utilising fragments of proneurotrophin activity modulator peptides comprising at least 25 contiguous amino acids and having an amino acid sequence which is at least 80%, such as 85%, for example 90%, such as 95%, for example 99% identical to the amino acid sequence of any of SEQ ID NO: 1, SEQ ID NO: 2 and SEQ ID NO: 3 respectively, wherein the percent identity is determined with the algorithm GAP, BESTFIT, or FASTA in the Wisconsin Genetics Software Package Release 7.0, using default gap weights.

The following terms are used to describe the sequence relationships between two or more polynucleotides: “predetermined sequence”, “comparison window”, “sequence identity”, “percentage of sequence identity”, and “substantial identity”.

A “predetermined sequence” is a defined sequence used as a basis for a sequence comparison; a predetermined sequence may be a subset of a larger sequence, for example, as a segment of a full-length DNA or gene sequence given in a sequence listing, such as a polynucleotide sequence of SEQ ID NO:1, or may comprise a complete DNA or gene sequence. Generally, a predetermined sequence is at least 20 nucleotides in length, frequently at least 25 nucleotides in length, and often at least 50 nucleotides in length.

Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a predetermined sequence of at least 20 contiguous nucleotides and wherein the portion of the polynucleotide sequence in the comparison window may comprise additions or deletions (i.e., gaps) of 20 percent or less as compared to the predetermined sequence (which does not comprise additions or deletions) for optimal alignment of the two sequences.

Optimal alignment of sequences for aligning a comparison window may be conducted by the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Natl. Acad. Sci. (U.S.A.) 85: 2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a predetermined sequence over a comparison window of at least 20 nucleotide positions, frequently over a window of at least 25-50 nucleotides, wherein the percentage of sequence identity is calculated by comparing the predetermined sequence to the polynucleotide sequence which may include deletions or additions which total 20 percent or less of the predetermined sequence over the window of comparison. The predetermined sequence may be a subset of a larger sequence, for example, as a segment of the full-length SEQ ID NO:1 polynucleotide sequence illustrated herein.

As applied to polypeptides, a degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences.

An “unrelated” or “non-homologous” sequence shares less than 40% identity, though preferably less than 25% identity, with the polypeptides of the present invention. The term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine, a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine.

Additionally, variants are also determined based on a predetermined number of conservative amino acid substitutions as defined herein below. Conservative amino acid substitution as used herein relates to the substitution of one amino acid (within a predetermined group of amino acids) for another amino acid (within the same group), wherein the amino acids exhibit similar or substantially similar characteristics.

Within the meaning of the term “conservative amino acid substitution” as applied herein, one amino acid may be substituted for another within the groups of amino acids indicated herein below:
i) Amino acids having polar side chains (Asp, Glu, Lys, Arg, His, Asn, Gln, Ser, Thr, Tyr, and Cys,)
ii) Amino acids having non-polar side chains (Gly, Ala, Val, Leu, Ile, Phe, Trp, Pro, and Met)
iii) Amino acids having aliphatic side chains (Gly, Ala Val, Leu, Ile)
iv) Amino acids having cyclic side chains (Phe, Tyr, Trp, His, Pro)
v) Amino acids having aromatic side chains (Phe, Tyr, Trp)
vi) Amino acids having acidic side chains (Asp, Glu)
vii) Amino acids having basic side chains (Lys, Arg, His)
viii) Amino acids having amide side chains (Asn, Gln)
ix) Amino acids having hydroxy side chains (Ser, Thr)
x) Amino acids having sulphur-containing side chains (Cys, Met),
xi) Neutral, weakly hydrophobic amino acids (Pro, Ala, Gly, Ser, Thr)
xii) Hydrophilic, acidic amino acids (Gln, Asn, Glu, Asp), and
xiii) Hydrophobic amino acids (Leu, Ile, Val)

Accordingly, a variant or a fragment thereof according to the invention may comprise, within the same variant of the sequence or fragments thereof, or among different variants of the sequence or fragments thereof, at least one substitution, such as a plurality of substitutions introduced independently of one another.

It is clear from the above outline that the same variant or fragment thereof may comprise more than one conservative amino acid substitution from more than one group of conservative amino acids as defined herein above.

The addition or deletion of at least one amino acid may be an addition or deletion of from preferably 2 to 250 amino acids, such as from 10 to 20 amino acids, for example from 20 to 30 amino acids, such as from 40 to 50 amino acids. However, additions or deletions of more than 50 amino acids, such as additions from 50 to 100 amino acids, addition of 100 to 150 amino acids, addition of 150-250 amino acids, are also comprised within the present invention. The deletion and/or the addition may—independently of one another—be a deletion and/or an addition within a sequence and/or at the end of a sequence.

siRNA: “small interfering RNA” (siRNA) is a short (often, but not restricted to, less than 30 nucleotides long) double-stranded RNA molecule capable of causing gene-specific silencing in mammalian cells.

Specific binding: The terms “specific binding” or “specifically binding”, as used herein, refers to the high affinity interaction between a protein or peptide and a binding molecule such as an antibody and a receptor or fragments thereof. The interaction is dependent upon the presence of a particular structure (i.e., the antigenic determinant or epitope) of the protein recognized by the binding molecule. For example, if an antibody is specific for epitope “A”, the presence of a protein containing epitope A (or free, unlabeled A) in a reaction containing labeled “A” and the antibody will reduce the amount of labeled A bound to the antibody.

Substantially purified: The term “substantially purified”, as used herein, refers to nucleic or amino acid sequences that are removed from their natural environment, isolated or separated, and are at least 60% free, preferably 75% free, and most preferably 90% free from other components with which they are naturally associated.

Substituted lower alkyl means a lower alkyl having one to three substituents selected from the group consisting of hydroxyl, alkoxy, amino, amido, carboxyl, acyl, halogen, cyano, nitro and thiol.

Substitution: A “substitution”, as used herein, refers to the replacement of one or more amino acids or nucleotides by different amino acids or nucleotides, respectively.

Treatment: The term “treatment” as used herein refers to a method involving therapy including surgery of a clinical condition in an individual including a human or animal body. The therapy may be ameliorating, curative or prophylactic, i.e. reducing pain symptoms.

Variants: The term “variants” as used herein refers to amino acid sequence variants said variants preferably having at least 60% identity, for example at least 63% identity, such as at least 66% identity, for example at least 70% sequence identity, for example at least 72% sequence identity, for example at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with any of the predetermined sequences.

Up-regulation of expression: a process leading to increased expression of genes, preferably of endogenous genes.

### DETAILED DESCRIPTION OF THE INVENTION

The present invention relates to the medical use of polypeptides and polynucleotides being identified as CTGF and/or BDNF. The CTGF protein has been identified in human beings (SEQ ID NO. 5), mouse (SEQ ID NO. 8), rat (SEQ ID NO. 11), cow (SEQ ID NO. 14), pig (SEQ ID NO. 17), Frog (SEQ ID NO. 21) and Zebra fish (SEQ ID NO 23) while the BDNF has been identified in in human beings (SEQ ID NO. 34), mouse (SEQ ID NO. 41), rat (SEQ ID NO. 45), cow (SEQ ID NO. 49), pig (SEQ ID NO. 53), Frog (SEQ ID NO. 57), Chick (SEQ ID NO. 58), Dog (SEQ ID NO. 59), Rhesus monkey (SEQ ID NO. 60), Cat (SEQ ID NO. 61) and Zebra fish (SEQ ID NO 62).

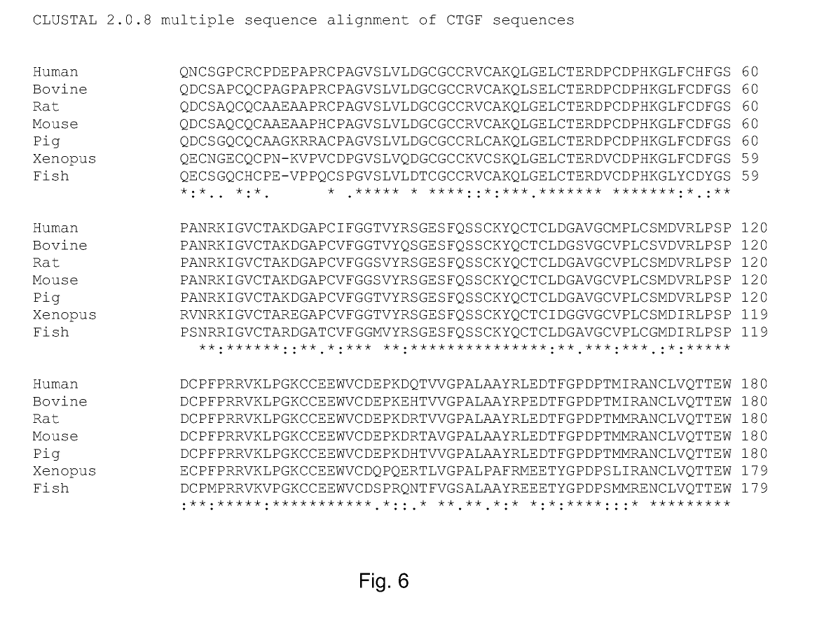

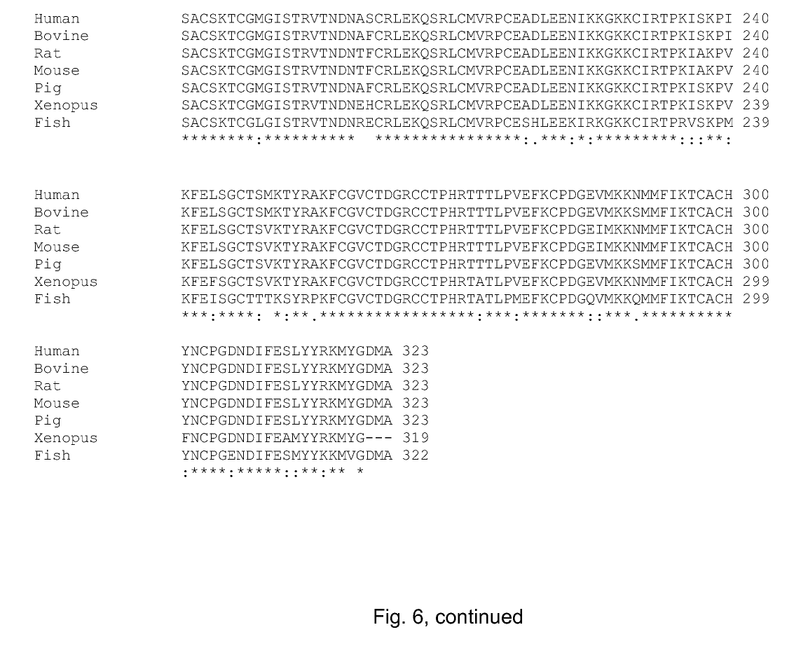

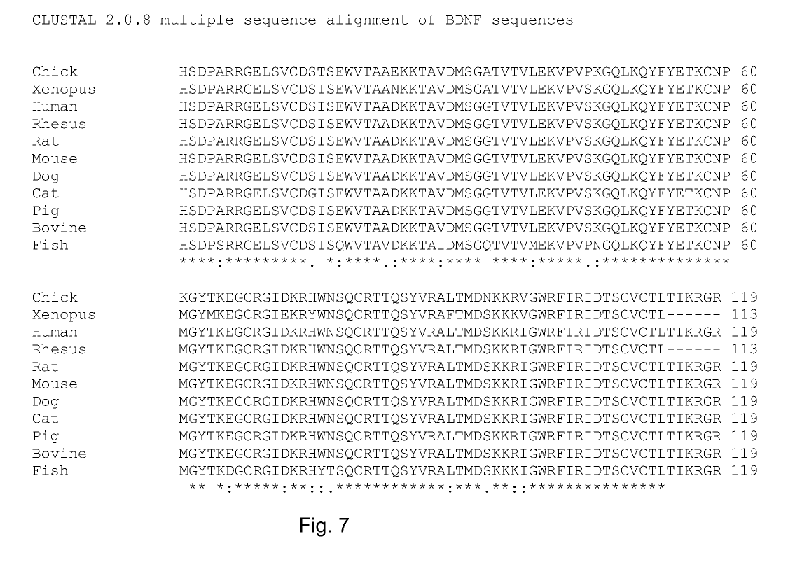

Sequence alignments of the mature CTGF and BDNF proteins from the above mentioned species is displayed in FIGS. 6 and 7 and the sequence alignment statistics are displayed in Tables 1 and 2 below.

TABLE 1
CTGF
Score of similarity (% of human sequence):

| SEQ | | Len | | | | |
|---|---|---|---|---|---|---|
| ID NO. | Name | (aa) | SEQ ID NO. | Name | Len (aa) | Score (%) |
| 5 | Human | 323 | 11 | Rat | 323 | 94 |
| 5 | Human | 323 | 8 | Mouse | 323 | 93 |
| 5 | Human | 323 | 14 | Bovine | 323 | 94 |
| 5 | Human | 323 | 17 | Pig | 323 | 93 |
| 5 | Human | 323 | 20 | Xenopus | 319 | 85 |
| 5 | Human | 323 | 23 | Fish | 322 | 81 |

TABLE 2
BDNF
Score of similarity (% of human sequence):

| SEQ | | Len | | | | |
|---|---|---|---|---|---|---|
| ID NO. | Name | (aa) | SEQ ID NO. | Name | Len (aa) | Score (%) |
| 34 | Human | 119 | 45 | Rat | 119 | 100 |
| 34 | Human | 119 | 41 | Mouse | 119 | 100 |
| 34 | Human | 119 | 58 | Chick | 119 | 94 |
| 34 | Human | 119 | 49 | Bovine | 119 | 100 |
| 34 | Human | 119 | 59 | Dog | 119 | 100 |
| 34 | Human | 119 | 60 | Rhesus | 113 | 100 |
| 34 | Human | 119 | 53 | Pig | 119 | 100 |
| 34 | Human | 119 | 57 | Xenopus | 113 | 92 |
| 34 | Human | 119 | 61 | Cat | 119 | 99 |
| 34 | Human | 119 | 62 | Fish | 119 | 89 |

The present inventor has demonstrated that CTGF and BDNF polypeptides may be used to upregulate the Vps10p-domain receptor SorLA (SEQ ID NO. 2) and thereby increase binding of amyloid precursor protein (APP) thereby inhibiting dissociation of APP to form Aβ and APPα amyloid plaque thereby being useful for the preparation of a medicament for the treatment of disease and disorders resulting from the accumulation of said amyloid plaque (Aβ and APPα amyloid plaque).

Both factors acts through Trk receptors to upregulate SORLA and prevent Aβ formation as outlined above (US 2006-0275797; Wahab et al (2005) J Am Soc Nephrol 16:340-351; Reichardt, L. F. (2006) Philos Trans R Soc Lond B Biol Sci 361, 1545-64)

### I. CTGF and BDNF Polypeptides and Uses Thereof

In a main aspect, the present invention relates to use of at least one isolated Brain Derived Neurotrophic Factor (BDNF) and/or Connective Tissue Growth Factor (CTGF) polypeptide for the preparation of a medicament for inhibiting formation of amyloid plaque in an individual.

In a further main aspect, the present invention relates to the use of the isolated Brain Derived Neurotrophic Factor (BDNF) and/or Connective Tissue Growth Factor (CTGF) polypeptide defined herein above wherein said polypeptide comprises an amino acid sequence selected from the group consisting of:
c) the amino acid sequence selected from the group consisting of SEQ ID NO. 5, 8, 11, 14, 17, 20, 23, 34, 41, 45, 49, 53, 57, 58, 59, 60, 61 and 62; and
d) a sequence variant of the amino acid sequence selected from the group consisting of SEQ ID NO. 5, 8, 11, 14, 17, 20, 23, 34, 41, 45, 49, 53, 57, 58, 59, 60, 61 and 62, wherein the sequence variant has at least 70% sequence identity to said SEQ ID NO. 5, 8, 11, 14, 17, 20, 23, 34, 41, 45, 49, 53, 57, 58, 59, 60, 61 or 62; or a biologically active fragment thereof, said fragment comprising at least 50 contiguous amino acids, wherein any amino acid specified in the selected sequence is altered to a different amino acid, provided that no more than 15 of the amino acid residues in the sequence are so altered.

In one embodiment, the polypeptide of the present invention is a naturally occurring allelic variant of the sequence selected from the group consisting of SEQ ID NO. 5, 8, 11, 14, 17, 20, 23, 34, 41, 45, 49, 53, 57, 58, 59, 60, 61 and 62, wherein said allelic variant comprises an amino acid sequence that is the translation of a nucleic acid sequence differing by a single nucleotide from a nucleic acid sequence selected from the group consisting of SEQ ID NO 3, 6, 9, 12, 15, 18, 21, 26, 27, 28, 29, 30, 31, 35, 36, 37, 38, 42, 46, 50 and 54.

In another embodiment, the polypeptide of the present invention is a variant polypeptide described therein, wherein any amino acid specified in the selected sequence is altered to provide a conservative substitution.

In another embodiment, the signal peptide of the polypeptide of the present invention has been replaced by a heterologous signal peptide.

In one embodiment, the polypeptide of the present invention has at least 70% sequence identity to a protein having a sequence selected from the group consisting of SEQ ID No. 5, 8, 11, 14, 17, 20, 23, 34, 41, 45, 49, 53, 57, 58, 59, 60, 61 and 62.

In another embodiment the polypeptide of the present invention has at least 75% sequence identity to a protein having a sequence selected from the group consisting of SEQ ID No. 5, 8, 11, 14, 17, 20, 23, 34, 41, 45, 49, 53, 57, 58, 59, 60, 61 and 62.

In a preferred embodiment, the polypeptide of the present invention has at least 80% sequence identity to a protein having a sequence selected from the group consisting of SEQ ID No. 5, 8, 11, 14, 17, 20, 23, 34, 41, 45, 49, 53, 57, 58, 59, 60, 61 and 62.

In an even more preferred embodiment the polypeptide of the present invention has at least 85% sequence identity to a protein having a sequence selected from the group consisting of SEQ ID No. 5, 8, 11, 14, 17, 20, 23, 34, 41, 45, 49, 53, 57, 58, 59, 60, 61 and 62.

In another preferred embodiment the polypeptide has at least 90% sequence identity to a protein having a sequence selected from the group consisting of SEQ ID No. 5, 8, 11, 14, 17, 20, 23, 34, 41, 45, 49, 53, 57, 58, 59, 60, 61 and 62.

In highly preferred embodiment the polypeptide of the present invention has at least 95% sequence identity to a protein having a sequence selected from the group consisting of SEQ ID No. 5, 8, 11, 14, 17, 20, 23, 34, 41, 45, 49, 53, 57, 58, 59, 60, 61 and 62.

In a further highly preferred embodiment the polypeptide of the present invention has at least 98% sequence identity to a protein having a sequence selected from the group consisting of SEQ ID No. 5, 8, 11, 14, 17, 20, 23, 34, 41, 45, 49, 53, 57, 58, 59, 60, 61 and 62.

In a most preferred embodiment the polypeptide of the present invention has at least 99% sequence identity to a protein having a sequence selected from the group consisting of SEQ ID No. 5, 8, 11, 14, 17, 20, 23, 34, 41, 45, 49, 53, 57, 58, 59, 60, 61 and 62.

In one embodiment the polypeptide of the present invention has at least 70% sequence identity to a protein having a sequence selected from the group consisting of SEQ ID No. 5, 8 and 11, more preferably at least 75%, more preferably at least 80%, more preferably at least 95%, more preferably at least 98%, more preferably a protein having the sequence selected from the group consisting of SEQ ID No. 5, 8 and 11.

In one embodiment the polypeptide of the present invention has at least 70% sequence identity to a protein having a sequence selected from the group consisting of SEQ ID No. 11, 14 and 17, more preferably at least 75%, more preferably at least 80%, more preferably at least 95%, more preferably at least 98%, more preferably a protein having the sequence selected from the group consisting of SEQ ID No. 11, 14 and 17.

In one embodiment the polypeptide of the present invention has at least 70% sequence identity to a protein having a sequence selected from the group consisting of SEQ ID No. 17, 20 and 23, more preferably at least 75%, more preferably at least 80%, more preferably at least 95%, more preferably at least 98%, more preferably a protein having the sequence selected from the group consisting of SEQ ID No. 17, 20 and 23.

In one embodiment the polypeptide of the present invention has at least 70% sequence identity to a protein having a sequence selected from the group consisting of SEQ ID No. 23, 34 and 41, more preferably at least 75%, more preferably at least 80%, more preferably at least 95%, more preferably at least 98%, more preferably a protein having the sequence selected from the group consisting of SEQ ID No. 23, 34 and 41.

In one embodiment the polypeptide of the present invention has at least 70% sequence identity to a protein having a sequence selected from the group consisting of SEQ ID No. 41, 45 and 49, more preferably at least 75%, more preferably at least 80%, more preferably at least 95%, more preferably at least 98%, more preferably a protein having the sequence selected from the group consisting of SEQ ID No. 41, 45 and 49.

In one embodiment the polypeptide of the present invention In one embodiment the polypeptide of the present invention has at least 70% sequence identity to a protein having a sequence selected from the group consisting of SEQ ID No. 49, 53 and 57, more preferably at least 75%, more preferably at least 80%, more preferably at least 95%, more preferably at least 98%, more preferably a protein having the sequence selected from the group consisting of SEQ ID No. 41, 45 and 49.

In one embodiment the polypeptide of the present invention has at least 70% sequence identity to a protein having a sequence selected from the group consisting of SEQ ID No. 49, 53 and 57, more preferably at least 75%, more preferably at least 80%, more preferably at least 95%, more preferably at least 98%, more preferably a protein having the sequence selected from the group consisting of SEQ ID No. 49, 53 and 57.

In one embodiment the polypeptide of the present invention has at least 70% sequence identity to a protein having a sequence selected from the group consisting of SEQ ID No. 57, 58 and 59, more preferably at least 75%, more preferably at least 80%, more preferably at least 95%, more preferably at least 98%, more preferably a protein having the sequence selected from the group consisting of SEQ ID No. 57, 58 and 59.

In one embodiment the polypeptide of the present invention has at least 70% sequence identity to a protein having a sequence selected from the group consisting of SEQ ID No. 59, 60, 61 and 62, more preferably at least 75%, more preferably at least 80%, more preferably at least 95%, more preferably at least 98%, more preferably a protein having the sequence selected from the group consisting of SEQ ID No. 59, 60, 61 and 62.

In one embodiment the polypeptide of the present invention has at least 70% sequence identity to the protein having the sequence of SEQ ID No. 5, more preferably at least 75%, more preferably at least 80%, more preferably at least 95%, more preferably at least 98%, more preferably a protein having the sequence of SEQ ID No. 5.

In one embodiment the polypeptide of the present invention has at least 70% sequence identity to the protein having the sequence of SEQ ID No. 8, more preferably at least 75%, more preferably at least 80%, more preferably at least 95%, more preferably at least 98%, more preferably a protein having the sequence of SEQ ID No. 8.

In one embodiment the polypeptide of the present invention has at least 70% sequence identity to the protein having the sequence of SEQ ID No. 11, more preferably at least 75%, more preferably at least 80%, more preferably at least 95%, more preferably at least 98%, more preferably a protein having the sequence of SEQ ID No. 11.

In one embodiment the polypeptide of the present invention has at least 70% sequence identity to the protein having the sequence of SEQ ID No. 14, more preferably at least 75%, more preferably at least 80%, more preferably at least 95%, more preferably at least 98%, more preferably a protein having the sequence of SEQ ID No. 14.

In one embodiment the polypeptide of the present invention has at least 70% sequence identity to the protein having the sequence of SEQ ID No. 17, more preferably at least 75%, more preferably at least 80%, more preferably at least 95%, more preferably at least 98%, more preferably a protein having the sequence of SEQ ID No. 17.

In one embodiment the polypeptide of the present invention has at least 70% sequence identity to the protein having the sequence of SEQ ID No. 20, more preferably at least 75%, more preferably at least 80%, more preferably at least 95%, more preferably at least 98%, more preferably a protein having the sequence of SEQ ID No. 20.

In one embodiment the polypeptide of the present invention has at least 70% sequence identity to the protein having the sequence of SEQ ID No. 23, more preferably at least 75%, more preferably at least 80%, more preferably at least 95%, more preferably at least 98%, more preferably a protein having the sequence of SEQ ID No. 23.

In one embodiment the polypeptide of the present invention has at least 70% sequence identity to the protein having the sequence of SEQ ID No. 34, more preferably at least 75%, more preferably at least 80%, more preferably at least 95%, more preferably at least 98%, more preferably a protein having the sequence of SEQ ID No. 34.

In one embodiment the polypeptide of the present invention has at least 70% sequence identity to the protein having the sequence of SEQ ID No. 41, more preferably at least 75%, more preferably at least 80%, more preferably at least 95%, more preferably at least 98%, more preferably a protein having the sequence of SEQ ID No. 41.

In one embodiment the polypeptide of the present invention has at least 70% sequence identity to the protein having the sequence of SEQ ID No. 45, more preferably at least 75%, more preferably at least 80%, more preferably at least 95%, more preferably at least 98%, more preferably a protein having the sequence of SEQ ID No. 45.

In one embodiment the polypeptide of the present invention has at least 70% sequence identity to the protein having the sequence of SEQ ID No. 49, more preferably at least 75%, more preferably at least 80%, more preferably at least 95%, more preferably at least 98%, more preferably a protein having the sequence of SEQ ID No. 49.

In one embodiment the polypeptide of the present invention has at least 70% sequence identity to the protein having the sequence of SEQ ID No. 53, more preferably at least 75%, more preferably at least 80%, more preferably at least 95%, more preferably at least 98%, more preferably a protein having the sequence of SEQ ID No. 53.

In one embodiment the polypeptide of the present invention has at least 70% sequence identity to the protein having the sequence of SEQ ID No. 57, more preferably at least 75%, more preferably at least 80%, more preferably at least 95%, more preferably at least 98%, more preferably a protein having the sequence of SEQ ID No. 57.

In one embodiment the polypeptide of the present invention has at least 70% sequence identity to the protein having the sequence of SEQ ID No. 58, more preferably at least 75%, more preferably at least 80%, more preferably at least 95%, more preferably at least 98%, more preferably a protein having the sequence of SEQ ID No. 58.

In one embodiment the polypeptide of the present invention has at least 70% sequence identity to the protein having the sequence of SEQ ID No. 59, more preferably at least 75%, more preferably at least 80%, more preferably at least 95%, more preferably at least 98%, more preferably a protein having the sequence of SEQ ID No. 59.

In one embodiment the polypeptide of the present invention has at least 70% sequence identity to the protein having the sequence of SEQ ID No. 60, more preferably at least 75%, more preferably at least 80%, more preferably at least 95%, more preferably at least 98%, more preferably a protein having the sequence of SEQ ID No. 60.

In one embodiment the polypeptide of the present invention has at least 70% sequence identity to the protein having the sequence of SEQ ID No. 61 or 62, more preferably at least 75%, more preferably at least 80%, more preferably at least 95%, more preferably at least 98%, more preferably a protein having the sequence of SEQ ID No. 61 or 62.

In another embodiment the polypeptide of the present invention is capable of forming at least one intramolecular cystine bridge.

In one embodiment the polypeptide of the present invention comprises a dimer of said protein linked through at least one intermolecular cystine bridge.

In a further embodiment the polypeptide of the present invention further comprises an affinity tag, such as a polyhis tag, a GST tag, a HA tag, a Flag tag, a C-myc tag, a HSV tag, a V5 tag, a maltose binding protein tag, a cellulose binding domain tag.

### II. CTGF and BDNF Nucleotide Sequences and Uses Thereof

In a main aspect, the present invention comprises at least one isolated Brain Derived Neurotrophic Factor (BDNF) and/or Connective Tissue Growth Factor (CTGF) nucleotide for the preparation of a medicament for inhibiting formation of amyloid plaque in an individual.

In one embodiment the at least one isolated nucleic acid molecule of the present invention comprises a nucleic acid sequence encoding upon expression, a polypeptide selected from the group consisting of:
a) the amino acid sequence selected from the group consisting of SEQ ID NO. 5, 8, 11, 14, 17, 20, 23, 34, 41, 45, 49, 53, 57, 58, 59, 60, 61 and 62 or a naturally occurring precursor protein thereof; and
b) a sequence variant of the amino acid sequence selected from the group consisting of SEQ ID NO. 5, 8, 11, 14, 17, 20, 23, 34, 41, 45, 49, 53, 57, 58, 59, 60, 61 and 62, wherein the sequence variant has at least 70% sequence identity to said SEQ ID NO. 5, 8, 11, 14, 17, 20, 23, 34, 41, 45, 49, 53, 57, 58, 59, 60, 61 or 62; or a biologically active fragment thereof, said fragment comprising at least 50 contiguous amino acids, wherein any amino acid specified in the selected sequence is altered to a different amino acid, provided that no more than 15 of the amino acid residues in the sequence are so altered.

In one embodiment the nucleic acid molecule of the present invention comprises the nucleotide sequence of a naturally occurring allelic nucleic acid variant, wherein the variant polypeptide has the polypeptide sequence of a naturally occurring polypeptide variant.

In one embodiment, the nucleic acid molecule of the present invention is selected from the group consisting of SEQ ID NO. 3, 6, 9, 12, 15, 18, 21, 26, 27, 28, 29, 30, 31, 35, 36, 37, 38, 42, 46, 50 and 54.

In one embodiment, the nucleic acid molecule of the present invention differs by a single nucleotide from a nucleic acid sequence selected from the group consisting of SEQ ID NO. 3, 6, 9, 12, 15, 18, 21, 26, 27, 28, 29, 30, 31, 35, 36, 37, 38, 42, 46, 50 and 54.

In one embodiment, the nucleic acid molecule of the present invention encodes a polypeptide having at least 70% sequence identity to a sequence selected from the group consisting of SEQ ID NO. SEQ ID No. 5, 8, 11, 14, 17, 20, 23, 34, 41, 45, 49, 53, 57, 58, 59, 60, 61 and 62.

In one embodiment, the nucleic acid molecule of the present invention encodes a polypeptide having at least 75% sequence identity to a sequence selected from the group consisting of SEQ ID NO. SEQ ID No. 5, 8, 11, 14, 17, 20, 23, 34, 41, 45, 49, 53, 57, 58, 59, 60, 61 and 62.

In one embodiment, the nucleic acid molecule of the present invention encodes a polypeptide having at least 80% sequence identity to a sequence selected from the group consisting of SEQ ID NO. SEQ ID No. 5, 8, 11, 14, 17, 20, 23, 34, 41, 45, 49, 53, 57, 58, 59, 60, 61 and 62.

In one embodiment, the nucleic acid molecule of the present invention encodes a polypeptide having at least 85% sequence identity to a sequence selected from the group consisting of SEQ ID NO. SEQ ID No. 5, 8, 11, 14, 17, 20, 23, 34, 41, 45, 49, 53, 57, 58, 59, 60, 61 and 62.

In one embodiment, the nucleic acid molecule of the present invention encodes a polypeptide having at least 90% sequence identity to a sequence selected from the group consisting of SEQ ID NO. SEQ ID No. 5, 8, 11, 14, 17, 20, 23, 34, 41, 45, 49, 53, 57, 58, 59, 60, 61 and 62.

In one embodiment, the nucleic acid molecule of the present invention encodes a polypeptide having at least 95% sequence identity to a sequence selected from the group consisting of SEQ ID NO. SEQ ID No. 5, 8, 11, 14, 17, 20, 23, 34, 41, 45, 49, 53, 57, 58, 59, 60, 61 and 62.

In one embodiment, the nucleic acid molecule of the present invention encodes a polypeptide having at least 98% sequence identity to a sequence selected from the group consisting of SEQ ID NO. SEQ ID No. 5, 8, 11, 14, 17, 20, 23, 34, 41, 45, 49, 53, 57, 58, 59, 60, 61 and 62.

In one embodiment, the nucleic acid molecule of the present invention encodes a polypeptide having at least 99% sequence identity to a sequence selected from the group consisting of SEQ ID NO. SEQ ID No. 5, 8, 11, 14, 17, 20, 23, 34, 41, 45, 49, 53, 57, 58, 59, 60, 61 and 62.

In one embodiment, the nucleic acid molecule of the present invention comprises a nucleotide sequence selected from the group consisting of
a) the nucleotide sequence selected from the group consisting of SEQ ID NO. 3, 6, 9, 12, 15, 18, 21, 26, 27, 28, 29, 30, 31, 35, 36, 37, 38, 42, 46, 50 and 54;
b) a nucleotide sequence having at least 70% sequence identity to a nucleotide sequence selected from the group consisting of SEQ ID NO. 3, 6, 9, 12, 15, 18, 21, 26, 27, 28, 29, 30, 31, 35, 36, 37, 38, 42, 46, 50 and 54;
c) a nucleic acid sequence of at least 150 contiguous nucleotides of a sequence selected from the group consisting of SEQ ID NO. 3, 6, 9, 12, 15, 18, 21, 26, 27, 28, 29, 30, 31, 35, 36, 37, 38, 42, 46, 50 and 54;
c) the complement of a nucleic acid capable of hybridising with nucleic acid having the sequence selected from the group consisting of SEQ ID NO.: 3, 6, 9, 12, 15, 18, 21, 26, 27, 28, 29, 30, 31, 35, 36, 37, 38, 42, 46, 50 and 54 under conditions of high stringency; and
d) the nucleic acid sequence of the complement of any of the above.

The use of the nucleic acid molecule as defined herein above, comprising a nucleotide sequence having at least 70% sequence identity to a nucleotide sequence selected from the group consisting of SEQ ID NO. 3, 6 and 9, more preferably at least 75%, more preferably at least 80%, more preferably at least 95%, more preferably at least 98%, more preferably a nucleic acid having the sequence of SEQ ID NO. 3, 6 and 9.

In one embodiment, the nucleic acid molecule of the present invention comprises a nucleotide sequence having at least 70% sequence identity to a nucleotide sequence selected from the group consisting of SEQ ID NO. 9, 12 and 15, more preferably at least 75%, more preferably at least 80%, more preferably at least 95%, more preferably at least 98%, more preferably a nucleic acid having the sequence of SEQ ID NO. 9, 12, and 15.

In one embodiment, the nucleic acid molecule of the present invention comprises a nucleotide sequence having at least 70% sequence identity to a nucleotide sequence selected from the group consisting of SEQ ID NO. 15, 18 and 21, more preferably at least 75%, more preferably at least 80%, more preferably at least 95%, more preferably at least 98%, more preferably a nucleic acid having the sequence of SEQ ID NO. 15, 18 and 21.

In one embodiment, the nucleic acid molecule of the present invention comprises a nucleotide sequence having at least 70% sequence identity to a nucleotide sequence selected from the group consisting of SEQ ID NO. 21, 26 and 27, more preferably at least 75%, more preferably at least 80%, more preferably at least 95%, more preferably at least 98%, more preferably a nucleic acid having the sequence of SEQ ID NO. 21, 26 and 27.

In one embodiment, the nucleic acid molecule of the present invention comprises a nucleotide sequence having at least 70% sequence identity to a nucleotide sequence selected from the group consisting of SEQ ID NO. 27, 28 and 29, more preferably at least 75%, more preferably at least 80%, more preferably at least 95%, more preferably at least 98%, more preferably a nucleic acid having the sequence of SEQ ID NO. 27, 28 and 29.

In one embodiment, the nucleic acid molecule of the present invention comprises a nucleotide sequence having at least 70% sequence identity to a nucleotide sequence selected from the group consisting of SEQ ID NO. 29, 30 and 31, more preferably at least 75%, more preferably at least 80%, more preferably at least 95%, more preferably at least 98%, more preferably a nucleic acid having the sequence of SEQ ID NO. 29, 30 and 31.

In one embodiment, the nucleic acid molecule of the present invention comprises a nucleotide sequence having at least 70% sequence identity to a nucleotide sequence selected from the group consisting of SEQ ID NO. 31, 35 and 36, more preferably at least 75%, more preferably at least 80%, more preferably at least 95%, more preferably at least 98%, more preferably a nucleic acid having the sequence of SEQ ID NO. 31, 35 and 36.

In one embodiment, the nucleic acid molecule of the present invention comprises a nucleotide sequence having at least 70% sequence identity to a nucleotide sequence selected from the group consisting of SEQ ID NO. 36, 37 and 38, more preferably at least 75%, more preferably at least 80%, more preferably at least 95%, more preferably at least 98%, more preferably a nucleic acid having the sequence of SEQ ID NO. 36, 37, and 38.

In one embodiment, the nucleic acid molecule of the present invention comprises a nucleotide sequence having at least 70% sequence identity to a nucleotide sequence selected from the group consisting of SEQ ID NO. 38, 42 and 46, more preferably at least 75%, more preferably at least 80%, more preferably at least 95%, more preferably at least 98%, more preferably a nucleic acid having the sequence of SEQ ID NO. 38, 42 and 46.

In one embodiment, the nucleic acid molecule of the present invention comprises a nucleotide sequence having at least 70% sequence identity to a nucleotide sequence selected from the group consisting of SEQ ID NO. 46, 50 and 54, more preferably at least 75%, more preferably at least 80%, more preferably at least 95%, more preferably at least 98%, more preferably a nucleic acid having the sequence of SEQ ID NO. 46, 50 and 54.

In one embodiment, the nucleic acid molecule of the present invention has at least 70% sequence identity to the nucleic acid molecule having the sequence of SEQ ID No. 3, more preferably at least 75%, more preferably at least 80%, more preferably at least 95%, more preferably at least 98%, more preferably a nucleic acid having the sequence of SEQ ID No. 3.

In one embodiment, the nucleic acid molecule of the present invention has at least 70% sequence identity to the nucleic acid molecule having the sequence of SEQ ID No. 6, more preferably at least 75%, more preferably at least 80%, more preferably at least 95%, more preferably at least 98%, more preferably a nucleic acid having the sequence of SEQ ID No. 6.

In one embodiment, the nucleic acid molecule of the present invention has at least 70% sequence identity to the nucleic acid molecule having the sequence of SEQ ID No. 9, more preferably at least 75%, more preferably at least 80%, more preferably at least 95%, more preferably at least 98%, more preferably a nucleic acid having the sequence of SEQ ID No. 9.

In one embodiment, the nucleic acid molecule of the present invention has at least 70% sequence identity to the nucleic acid molecule having the sequence of SEQ ID No. 12, more preferably at least 75%, more preferably at least 80%, more preferably at least 95%, more preferably at least 98%, more preferably a nucleic acid having the sequence of SEQ ID No. 12.

In one embodiment, the nucleic acid molecule of the present invention has at least 70% sequence identity to the nucleic acid molecule having the sequence of SEQ ID No. 15, more preferably at least 75%, more preferably at least 80%, more preferably at least 95%, more preferably at least 98%, more preferably a nucleic acid having the sequence of SEQ ID No. 15.

In one embodiment, the nucleic acid molecule of the present invention has at least 70% sequence identity to the nucleic acid molecule having the sequence of SEQ ID No. 18, more preferably at least 75%, more preferably at least 80%, more preferably at least 95%, more preferably at least 98%, more preferably a nucleic acid having the sequence of SEQ ID No. 18.

In one embodiment, the nucleic acid molecule of the present invention has at least 70% sequence identity to the nucleic acid molecule having the sequence of SEQ ID No. 21, more preferably at least 75%, more preferably at least 80%, more preferably at least 95%, more preferably at least 98%, more preferably a nucleic acid having the sequence of SEQ ID No. 21.

In one embodiment, the nucleic acid molecule of the present invention has at least 70% sequence identity to the nucleic acid molecule having the sequence of SEQ ID No. 26, more preferably at least 75%, more preferably at least 80%, more preferably at least 95%, more preferably at least 98%, more preferably a nucleic acid having the sequence of SEQ ID No. 26.

In one embodiment, the nucleic acid molecule of the present invention has In one embodiment, the nucleic acid molecule of the present invention has at least 70% sequence identity to the nucleic acid molecule having the sequence of SEQ ID No. 27, more preferably at least 75%, more preferably at least 80%, more preferably at least 95%, more preferably at least 98%, more preferably a nucleic acid having the sequence of SEQ ID No. 27.

In one embodiment, the nucleic acid molecule of the present invention has at least 70% sequence identity to the nucleic acid molecule having the sequence of SEQ ID No. 28, more preferably at least 75%, more preferably at least 80%, more preferably at least 95%, more preferably at least 98%, more preferably a nucleic acid having the sequence of SEQ ID No. 28.

In one embodiment, the nucleic acid molecule of the present invention has at least 70% sequence identity to the nucleic acid molecule having the sequence of SEQ ID No. 29, more preferably at least 75%, more preferably at least 80%, more preferably at least 95%, more preferably at least 98%, more preferably a nucleic acid having the sequence of SEQ ID No. 29.

In one embodiment, the nucleic acid molecule of the present invention has at least 70% sequence identity to the nucleic acid molecule having the sequence of SEQ ID No. 30, more preferably at least 75%, more preferably at least 80%, more preferably at least 95%, more preferably at least 98%, more preferably a nucleic acid having the sequence of SEQ ID No. 30.

In one embodiment, the nucleic acid molecule of the present invention has at least 70% sequence identity to the nucleic acid molecule having the sequence of SEQ ID No. 31, more preferably at least 75%, more preferably at least 80%, more preferably at least 95%, more preferably at least 98%, more preferably a nucleic acid having the sequence of SEQ ID No. 31.

In one embodiment, the nucleic acid molecule of the present invention has at least 70% sequence identity to the nucleic acid molecule having the sequence of SEQ ID No. 35, more preferably at least 75%, more preferably at least 80%, more preferably at least 95%, more preferably at least 98%, more preferably a nucleic acid having the sequence of SEQ ID No. 35.

In one embodiment, the nucleic acid molecule of the present invention has at least 70% sequence identity to the nucleic acid molecule having the sequence of SEQ ID No. 36, more preferably at least 75%, more preferably at least 80%, more preferably at least 95%, more preferably at least 98%, more preferably a nucleic acid having the sequence of SEQ ID No. 36.

In one embodiment, the nucleic acid molecule of the present invention has In one embodiment, the nucleic acid molecule of the present invention has In one embodiment, the nucleic acid molecule of the present invention has at least 70% sequence identity to the nucleic acid molecule having the sequence of SEQ ID No. 37, more preferably at least 75%, more preferably at least 80%, more preferably at least 95%, more preferably at least 98%, more preferably a nucleic acid having the sequence of SEQ ID No. 38.

In one embodiment, the nucleic acid molecule of the present invention has at least 70% sequence identity to the nucleic acid molecule having the sequence of SEQ ID No. 38, more preferably at least 75%, more preferably at least 80%, more preferably at least 95%, more preferably at least 98%, more preferably a nucleic acid having the sequence of SEQ ID No. 38.

In one embodiment, the nucleic acid molecule of the present invention has at least 70% sequence identity to the nucleic acid molecule having the sequence of SEQ ID No. 42, more preferably at least 75%, more preferably at least 80%, more preferably at least 95%, more preferably at least 98%, more preferably a nucleic acid having the sequence of SEQ ID No. 42.

In one embodiment, the nucleic acid molecule of the present invention has at least 70% sequence identity to the nucleic acid molecule having the sequence of SEQ ID No. 46, more preferably at least 75%, more preferably at least 80%, more preferably at least 95%, more preferably at least 98%, more preferably a nucleic acid having the sequence of SEQ ID No. 46.

In one embodiment, the nucleic acid molecule of the present invention has at least 70% sequence identity to the nucleic acid molecule having the sequence of SEQ ID No. 50, more preferably at least 75%, more preferably at least 80%, more preferably at least 95%, more preferably at least 98%, more preferably a nucleic acid having the sequence of SEQ ID No. 50.

In one embodiment, the nucleic acid molecule of the present invention has at least 70% sequence identity to the nucleic acid molecule having the sequence of SEQ ID No. 54, more preferably at least 75%, more preferably at least 80%, more preferably at least 95%, more preferably at least 98%, more preferably a nucleic acid having the sequence of SEQ ID No. 54.

In one embodiment, the nucleic acid molecule of the present invention has been codon optimised for expression in E. coli, Chinese Hamster, Baby Hamster, Yeast, insect and/or fungus.

In one main aspect the present invention relates to the use of
f) the polypeptide as defined herein above; or
g) the isolated nucleic acid sequence as defined herein above; or
h) the expression vector as defined herein; or
i) a composition of host cells as defined herein;
j) a packaging cell line according as defined herein; for the manufacture of a medicament for inhibiting formation of amyloid plaque in an individual in need thereof.

In one embodiment, the production of Aβ and formation of amyloid plaque as used herein above results in onset of Alzheimer's Disease.

In one embodiment, the formation of amyloid plaque as used herein above results in onset of Alzheimer's Disease. In a main aspect, the present invention relate to the use of at least one isolated peptide for the preparation of a medicament for inhibiting formation of amyloid plaque, said peptide comprising a fragment comprising at least 8 contiguous amino acid residues of an amino acid sequence selected from the group consisting of
c) SEQ ID NO. 5, 8, 11, 14, 17, 20, 23, 34, 41, 45, 49, 53, 57, 58, 59, 60, 61 and 62; and
d) a sequence variant of the amino acid sequence of a), wherein the sequence variant has at least 70% sequence identity to said SEQ ID NO. 5, 8, 11, 14, 17, 20, 23, 34, 41, 45, 49, 53, 57, 58, 59, 60, 61 or 62.

In one embodiment, the peptide as defined herein above has been modified to form a dimer.

In a further embodiment of the present invention said dimer is cyclised.

In one embodiment of the present invention the peptide is selected from the group consisting of SEQ ID NO. 63, 64, 65, 66, 67 and 68.

In one embodiment, the peptide of the present invention is encoded by a nucleic acid molecule selected from the group consisting of SEQ ID NO. 24 and 25.

In one important aspect the present invention relates to an isolated polypeptide selected from the group consisting of:
a) A polypeptide having an amino acid sequence as set forth in SEQ ID NO. 5, and polypeptides having from one to five extra amino acids;
b) A polypeptide having an amino acid sequence as set forth in SEQ ID NO. 8, and polypeptides having from one to five extra amino acids;
c) A polypeptide having an amino acid sequence as set forth in SEQ ID NO. 11, and polypeptides having from one to five extra amino acids;
d) A polypeptide having an amino acid sequence as set forth in SEQ ID NO. 14, and polypeptides having from one to five extra amino acids;
e) A polypeptide having an amino acid sequence as set forth in SEQ ID NO. 17, and polypeptides having from one to five extra amino acids;
f) A polypeptide having an amino acid sequence as set forth in SEQ ID NO. 20, and polypeptides having from one to five extra amino acids;
g) A polypeptide having an amino acid sequence as set forth in SEQ ID NO. 23, and polypeptides having from one to five extra amino acids; A polypeptide having an amino acid sequence as set forth in SEQ ID NO 34, and polypeptides having from one to five extra amino acids;
h) A polypeptide having an amino acid sequence as set forth in SEQ ID NO 41, and polypeptides having from one to five extra amino acids;
i) A polypeptide having an amino acid sequence as set forth in SEQ ID NO 45, and polypeptides having from one to five extra amino acids;
j) A polypeptide having an amino acid sequence as set forth in SEQ ID NO 49, and polypeptides having from one to five extra amino acids;
k) A polypeptide having an amino acid sequence as set forth in SEQ ID NO 53, and polypeptides having from one to five extra amino acids;
l) A polypeptide having an amino acid sequence as set forth in SEQ ID NO 57, and polypeptides having from one to five extra amino acids;
m) A polypeptide having an amino acid sequence as set forth in SEQ ID NO 58, and polypeptides having from one to five extra amino acids;
n) A polypeptide having an amino acid sequence as set forth in SEQ ID NO 59, and polypeptides having from one to five extra amino acids;
o) A polypeptide having an amino acid sequence as set forth in SEQ ID NO 60, and polypeptides having from one to five extra amino acids;
p) A polypeptide having an amino acid sequence as set forth in SEQ ID NO 61, and polypeptides having from one to five extra amino acids;
q) A polypeptide having an amino acid sequence as set forth in SEQ ID NO 62, and polypeptides having from one to five extra amino acids; and
r) variants of said polypeptides, wherein any amino acid specified in the chosen sequence is changed to a different amino acid, provided that no more than 15 of the amino acid residues in the sequence are so changed.

In one embodiment the changed amino acids of the polypeptide defined in r) above are selected from those designated as unconserved in FIG. 6 or 7.

In another aspect, the present invention relates to at least one isolated polypeptide for use in a method of treatment of disease resulting from formation of amyloid plaque, said polypeptide comprising an amino acid sequence selected from the group consisting of:
c) the amino acid sequence selected from the group consisting of SEQ ID NO. 5, 8, 11, 14, 17, 20, 23, 34, 41, 45, 49, 53, 57, 58, 59, 60, 61 and 62; and
d) a sequence variant of the amino acid sequence selected from the group consisting of SEQ ID NO. 5, 8, 11, 14, 17, 20, 23, 34, 41, 45, 49, 53, 57, 58, 59, 60, 61 and 62, wherein the sequence variant has at least 70% sequence identity to said SEQ ID NO. 5, 8, 11, 14, 17, 20, 23, 34, 41, 45, 49, 53, 57, 58, 59, 60, 61 or 62; or a biologically active fragment of at least 30 contiguous amino acids thereof wherein any amino acid specified in the selected sequence is altered to a different amino acid, provided that no more than 15 of the amino acid residues in the sequence are so altered.

In another aspect, the invention relates to use of at least one isolated polypeptide for the preparation of a medicament for inhibiting formation of Aβ peptide and of amyloid plaque, said polypeptide comprising an amino acid sequence selected from the group consisting of:
a) the amino acid sequence selected from the group consisting of SEQ ID NO. 5, 8, 11, 14, 17, 20 and 23; and
b) a sequence variant of the amino acid sequence selected from the group consisting of SEQ ID NO. 5, 8, 11, 14, 17, 20 and 23, wherein the sequence variant has at least 70% sequence identity to said SEQ ID NO. 5, 8, 11, 14, 17, 20 and 23; and
c) a biologically active fragment of at least 50 contiguous amino acids of any of a) wherein any amino acid specified in the selected sequence is altered to a different amino acid, provided that no more than 15 of the amino acid residues in the sequence are so altered.

In another aspect, the invention relates to use of at least one isolated nucleic acid molecule for the preparation of a medicament for the treatment of Alzheimer's Disease, said nucleic acid molecule comprising an nucleic acid sequence encoding upon expression, a polypeptide selected from the group consisting of:
a) the amino acid sequence selected from the group consisting of SEQ ID NO. 5, 8, 11, 14, 17, 20 and 23 or a naturally occurring precursor protein thereof; and
b) a sequence variant or a naturally occurring precursor protein of the amino acid sequence selected from the group consisting of SEQ ID NO. 5, 8, 11, 14, 17, 20 and 23, wherein the sequence variant has at least 70% sequence identity to said SEQ ID NO. 5, 8, 11, 14, 17, 20 and 23; and
c) a biologically active fragment of at least 50 contiguous amino acids of any of a) wherein any amino acid specified in the selected sequence is altered to a different amino acid, provided that no more than 50 of the amino acid residues in the sequence are so altered.

In one embodiment the nucleic acid molecule as defined herein above is selected from the group consisting of SEQ ID NO. 3, 6, 9, 12, 15, 18, and 21.

In one embodiment, the polypeptide of the present invention is not BDNF.

In one embodiment, the polypeptide of the present invention is not CTGF.

### III. Gene Therapy Applications

To form a CTGF, BDNF or a CTGF/BDNF combination composition for gene therapy use in the invention, a CTGF and/or BDNF encoding expression viral vector may be placed into a pharmaceutically acceptable suspension, solution or emulsion. Suitable mediums include saline and liposomal preparations.

More specifically, pharmaceutically acceptable carriers may include sterile aqueous of non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils.

Intravenous vehicles include fluid and nutrient replenishers, electrolyte replenishers (such as those based on Ringer's dextrose), and the like.

Preservatives and other additives may also be present such as, for example, antimicrobials, antioxidants, chelating agents, and inert gases and the like. Furthermore, a composition of CTGF and/or BDNF transgenes may be lyophilized using means well known in the art, for subsequent reconstitution and use according to the invention.

A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, and liposoms. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macro molecules. RNA, DNA and intact virions can be encapsulated within the aqueous interior and be delivered to cells in a biologically active form (Fraley, et al., Trends Biochem. Sci., 6: 77, 1981). In addition to mammalian cells, liposomes have been used for delivery of operatively encoding transgenes in plant, yeast and bacterial cells. In order for a liposome to be an efficient gene transfer vehicle, the following characteristics should be present: (1) encapsulation of the genes encoding the CTGF and/or BDNF at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6: 682, 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides.

Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries.

Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted gene delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

An important parameter is the dosage of the CTGF and/or BDNF gene therapy vector to be delivered into the target tissue. For viral vectors, the concentration may be defined by the number of transducing units/ml. Optimally, for delivery using a viral expression vector, each unit dosage will comprise 2.5 to 25 μL of a composition, wherein the composition includes a viral expression vector in a pharmaceutically acceptable fluid and provides from 10⁸ up to 10¹⁰ CTGF or BDNF transducing units per ml.

Importantly, specific in vivo gene delivery sites are selected so as to cluster in an area of accumulation of amyloid plaque. Such areas may be identified clinically using a number of known techniques, including magnetic resonance imaging (MRI) and biopsy. In humans, non-invasive, in vivo imaging methods such as MRI will be preferred. Once areas of neuronal loss are identified, delivery sites are selected for stereotaxic distribution so each unit dosage of CTGF and/or BDNF is delivered into the brain at, or within 500 μm from, a targeted cell, and no more than about 10 mm from another delivery site.

Within a given target site, the vector system may transduce a target cell. The target cell may be a cell found in nervous tissue, such as a neuron, astrocyte, oligodendrocyte, microglia, stem cells, neural precursor cells, or ependymal cell.

The vector system is preferably administered by direct injection. Methods for injection into the brain are well known in the art (Bilang-Bleuel et al (1997) Proc. Acad. Natl. Sci. USA 94:8818-8823; Choi-Lundberg et al (1998) Exp. Neurol. 154:261-275; Choi-Lundberg et al (1997) Science 275:838-841; and Mandel et al (1997)) Proc. Acad. Natl. Sci. USA 94:14083-14088). Stereotaxic injections may be given.

For transduction in tissues such as the brain, it is necessary to use very small volumes, so the viral preparation is concentrated by ultracentrifugation. The resulting preparation should have at least 10⁸ TU/ml, preferably from 10⁸ to 10¹⁰ TU/ml, more preferably at least 10⁹ TU./ml. (The titer is expressed in transducing units per ml (TU./ml)). It has been found that improved dispersion of transgene expression can be obtained by increasing the number of injection sites and decreasing the rate of injection (Horellou and Mallet (1997) as above). Usually between 1 and 10 injection sites are used, more commonly between 2 and 6. For a dose comprising 1−5×10⁹ TU./ml, the rate of injection is commonly between 0.1 and 10 μl/min, usually about 1 μl/min.

The virus composition is delivered to each delivery cell site in the target tissue by microinjection, infusion, scrape loading, electroporation or other means suitable to directly deliver the composition directly into the delivery site tissue through a surgical incision. The delivery is accomplished slowly, such as over a period of about 5-10 minutes (depending on the total volume of virus composition to be delivered).

Broadly, gene therapy seeks to transfer new genetic material to the cells of a patient with resulting therapeutic benefit to the patient. Such benefits include treatment or prophylaxis of a broad range of diseases, disorders and other conditions.

Ex vivo gene therapy approaches involve modification of isolated cells (including but not limited to stem cells, neural and glial precursor cells, and foetal stem cells), which are then infused, grafted or otherwise transplanted into the patient. See, e.g., U.S. Pat. Nos. 4,868,116, 5,399,346 and 5,460,959. In vivo gene therapy seeks to directly target host patient tissue.

Viruses useful as gene transfer vectors include papovavirus, adenovirus, vaccinia virus, adeno-associated virus, herpesvirus, and retroviruses. Suitable retroviruses include the group consisting of HIV, SIV, FIV, EIAV, MoMLV. A further group of suitable retroviruses includes the group consisting of HIV, SIV, FIV, EAIV, CIV. Another group of preferred virus vectors includes the group consisting of alphavirus, adenovirus, adeno associated virus, baculovirus, HSV, coronavirus, Bovine papilloma virus, Mo-MLV, preferably adeno associated virus.

Methods for preparation of AAV are described in the art, e.g. U.S. Pat. No. 5,677,158. U.S. Pat. No. 6,309,634 and U.S. Pat. No. 6,683,058 describe examples of delivery of AAV to the central nervous system.

Preferably, a lentivirus vector is a replication-defective lentivirus particle. Such a lentivirus particle can be produced from a lentiviral vector comprising a 5′ lentiviral LTR, a tRNA binding site, a packaging signal, a promoter operably linked to a polynucleotide signal encoding said fusion protein, an origin of second strand DNA synthesis and a 3′ lentiviral LTR. Methods for preparation and in vivo administration of lentivirus to neural cells are described in US 20020037281 (Methods for transducing neural cells using lentiviral vectors).

Retroviral vectors are the vectors most commonly used in human clinical trials, since they carry 7-8 kb and since they have the ability to infect cells and have their genetic material stably integrated into the host cell with high efficiency. See, e.g., WO 95/30761; WO 95/24929. Oncovirinae require at least one round of target cell proliferation for transfer and integration of exogenous nucleic acid sequences into the patient. Retroviral vectors integrate randomly into the patient's genome. Retroviruses can be used to target stem cells of the nervous system as very few cell divisions take place in other cells of the nervous system (in particular the CNS).

Three classes of retroviral particles have been described; ecotropic, which can infect murine cells efficiently, and amphotropic, which can infect cells of many species. The third class includes xenotrophic retrovirus which can infect cells of another species than the species which produced the virus. Their ability to integrate only into the genome of dividing cells has made retroviruses attractive for marking cell lineages in developmental studies and for delivering therapeutic or suicide genes to cancers or tumors.

For use in human patients, the retroviral vectors must be replication defective. This prevents further generation of infectious retroviral particles in the target tissue. Instead the replication defective vector becomes a “captive” transgene stable incorporated into the target cell genome. Typically in replication defective vectors, the gag, env, and pol genes have been deleted (along with most of the rest of the viral genome). Heterologous DNA is inserted in place of the deleted viral genes. The heterologous genes may be under the control of the endogenous heterologous promoter, another heterologous promoter active in the target cell, or the retroviral 5′LTR (the viral LTR is active in diverse tissues). Typically, retroviral vectors have a transgene capacity of about 7-8 kb.

Replication defective retroviral vectors require provision of the viral proteins necessary for replication and assembly in trans, from, e.g., engineered packaging cell lines. It is important that the packaging cells do not release replication competent virus and/or helper virus. This has been achieved by expressing viral proteins from RNAs lacking the ψ signal, and expressing the gag/pol genes and the env gene from separate transcriptional units. In addition, in some 2. and 3. generation retriviruses, the 5′ LTR's have been replaced with non-viral promoters controlling the expression of these genes, and the 3′ promoter has been minimised to contain only the proximal promoter. These designs minimize the possibility of recombination leading to production of replication competent vectors, or helper viruses.

In embodiment, the vector of the present invention is selected from the group consisting of vectors derived from the Retroviridae family including lentivirus, HIV, SIV, FIV, EAIV, CIV.

In yet another embodiment, the vector of the present invention is selected from the group consisting of alphavirus, adenovirus, adeno associated virus, baculovirus, HSV, coronavirus, Bovine papilloma virus, Mo-MLV, preferably adeno associated virus.

### IV. Expression Vectors

Construction of vectors for recombinant expression of CTGF and/or BDNF polypeptides for use in the invention may be accomplished using conventional techniques which do not require detailed explanation to one of ordinary skill in the art. For review, however, those of ordinary skill may wish to consult Maniatis et al., in Molecular Cloning: A Laboratory Manual, Cold Spring Harbor Laboratory, (NY 1982). Expression vectors may be used for generating producer cells for recombinant production of CTGF and/or BDNF polypeptides for medical use, and for generating therapeutic cells secreting CTGF and/or BDNF polypeptides for naked or encapsulated therapy.

Briefly, construction of recombinant expression vectors employs standard ligation techniques. For analysis to confirm correct sequences in vectors constructed, the genes are sequenced using, for example, the method of Messing, et al., (Nucleic Acids Res., 9: 309-, 1981), the method of Maxam, et al., (Methods in Enzymology, 65: 499, 1980), or other suitable methods which will be known to those skilled in the art.

Size separation of cleaved fragments is performed using conventional gel electrophoresis as described, for example, by Maniatis, et al., (Molecular Cloning, pp. 133-134, 1982).

For generation of efficient expression vectors, these should contain regulatory sequences necessary for expression of the encoded gene in the correct reading frame. Expression of a gene is controlled at the transcription, translation or posttranslation levels. Transcription initiation is an early and critical event in gene expression. This depends on the promoter and enhancer sequences and is influenced by specific cellular factors that interact with these sequences. The transcriptional unit of many genes consists of the promoter and in some cases enhancer or regulator elements (Banerji et al. 1981, Cell 27: 299; Corden et al. 1980, Science 209: 1406; and Breathnach and Chambon 1981, Ann. Rev. Biochem. 50: 349). For retroviruses, control elements involved in the replication of the retroviral genome reside in the long terminal repeat (LTR) (Weiss et al., eds., The molecular biology of tumor viruses: RNA tumor viruses, Cold Spring Harbor Laboratory, (NY 1982)). Moloney murine leukemia virus (MLV) and Rous sarcoma virus (RSV) LTRs contain promoter and enhancer sequences (Jolly et al. 1983, Nucleic Acids Res. 11: 1855; Capecchi et al., In: Enhancer and eukaryotic gene expression, Gulzman and Shenk, eds., pp. 101-102, Cold Spring Harbor Laboratories (NY 1991). Other potent promoters include those derived from cytomegalovirus (CMV) and other wild-type viral promoters.

Promoter and enhancer regions of a number of non-viral promoters have also been described (Schmidt et al. 1985, Nature 314: 285; Rossi and deCrombrugghe 1987, Proc. Natl. Acad. Sci. USA 84: 5590-5594). Methods for maintaining and increasing expression of transgenes in quiescent cells include the use of promoters including collagen type I (1 and 2) (Prockop and Kivirikko, 1984 N. Eng. J. Med. 311: 376; Smith and Niles 1980, Biochem. 19: 1820; de Wet et al. 1983, J. Biol. Chem., 258: 14385), SV40 and LTR promoters.

According to one embodiment of the invention, the promoter is a constitutive promoter selected from the group consisting of: ubiquitin promoter, CMV promoter, JeT promoter (U.S. Pat. No. 6,555,674), SV40 promoter, Chicken beta-action promoter, Elongation Factor 1 alpha promoter (EF1-alpha), RSV, Mo-MLV-LTR. Examples of inducible/repressible promoters include: Tet-On, Tet-Off, Rapamycin-inducible promoter, Mx1.

In a main aspect the present invention comprises use of a vector comprising at least one nucleic acid molecule as defined herein above, for the preparation of a medicament for inhibiting formation of amyloid plaque.

In one embodiment, the vector of the present invention further comprising a promoter operably linked to the nucleic acid molecule, said promoter selected from the group consisting of: CMV, human UbiC, RSV, Tet-regulatable promoter, MoMLV-LTR, Mx1, EF-1alpha, PDGF beta and CaMK II.

In addition to using viral and non-viral promoters to drive transgene expression, an enhancer sequence may be used to increase the level of transgene expression. Enhancers can increase the transcriptional activity not only of their native gene but also of some foreign genes (Armelor 1973, Proc. Natl. Acad. Sci. USA 70: 2702). For example, in the present invention collagen enhancer sequences may be used with the collagen promoter 2 (I) to increase transgene expression. In addition, the enhancer element found in SV40 viruses may be used to increase transgene expression. This enhancer sequence consists of a 72 base pair repeat as described by Gruss et al. 1981, Proc. Natl. Acad. Sci. USA 78: 943; Benoist and Chambon 1981, Nature 290: 304, and Fromm and Berg 1982, J. Mol. Appl. Genetics, 1: 457, all of which are incorporated by reference herein. This repeat sequence can increase the transcription of many different viral and cellular genes when it is present in series with various promoters (Moreau et al. 1981, Nucleic Acids Res. 9: 6047).

Further expression enhancing sequences include but are not limited to Woodchuck hepatitis virus post-transcriptional regulation element, WPRE, SP163, CMV enhancer, and Chicken [beta]-globin insulator or other insulators.

Transgene expression may also be increased for long term stable expression using cytokines to modulate promoter activity. Several cytokines have been reported to modulate the expression of transgene from collagen 2 (I) and LTR promoters (Chua et al., connective Tissue Res., 25: 161-170 (1990); Elias et al., Annals N.Y. Acad. Sci., 580: 233-244 (1990)); Seliger et al., J. Immunol. 141: 2138-2144 (1988) and Seliger et al., J. Virology 62: 619-621 (1988)). For example, transforming growth factor (TGF), interleukin (IL)-I, and interferon (INF) down regulate the expression of transgenes driven by various promoters such as LTR. Tumor necrosis factor (TNF) and TGF 1 up regulate, and may be used to control, expression of transgenes driven by a promoter. Other cytokines that may prove useful include basic fibroblast growth factor (bFGF) and epidermal growth factor (EGF).

Collagen promoter with the collagen enhancer sequence (Coll (E)) may also be used to increase transgene expression by suppressing further any immune response to the vector which may be generated in a treated brain notwithstanding its immune-protected status. In addition, anti-inflammatory agents including steroids, for example dexamethasone, may be administered to the treated host immediately after vector composition delivery and continued, preferably, until any cytokine-mediated inflammatory response subsides. An immunosuppression agent such as cyclosporin may also be administered to reduce the production of interferons, which downregulates LTR promoter and Coll (E) promoter-enhancer, and reduces transgene expression.

The vector may comprise further sequences such as a sequence coding for the Cre-recombinase protein, and LoxP sequences. A further way of ensuring temporary expression of the CTGF and/or BDNF polypeptides is through the use of the Cre-LoxP system which results in the excision of part of the inserted DNA sequence either upon administration of Cre-recombinase to the cells (Daewoong et al, Nature Biotechnology 19:929-933) or by incorporating a gene coding for the recombinase into the virus construct (Pluck, Int J Exp Path, 77:269-278). Incorporating a gene for the recombinase in the virus construct together with the LoxP sites and a structural gene (an CTGF, BDNF or a combination thereof in the present case) often results in expression of the structural gene for a period of approximately five days.

### V. Cells

In one aspect the invention relates to isolated host cells genetically modified with the vector according to the invention.

According to one embodiment, the host cells are prokaryotic cells such as E. coli which are capable producing recombinant protein in high quantities and which can easily be scaled up to industrial scale. The use of prokaryotic producer cells may require refolding and glycosylation of CTGF and/or BDNF in order to obtain a biologically active protein. In another embodiment, the host cells are eukaryotic producer cells from non-mammals, including but not limited to known producer cells such as yeast (Saccharomyces cerevisiae), filamentous fungi such as aspergillus, and insect cells, such as Sf9

In a main aspect, the present invention relate to the use of an isolated host cell transformed or transduced with at least one vector as defined herein above, for the preparation of a medicament for inhibiting formation of amyloid plaque.

In one embodiment, the host cell of the present invention is selected from the group consisting of Saccharomyces cerevisiae, E. coli, Aspergillus and Sf9 insect cells.

In one embodiment, the host cell of the present invention is selected from the group consisting of mammalian cells selected from the group consisting of human, feline, porcine, simian, canine, murine and rat cells.

In one embodiment, the host cell of the present invention is selected from the group consisting of immortalised retinal pigmented epithelial cells selected from the group consisting of immortalised human fibroblasts, ARPE-19 cells and immortalised human astrocytes.

In one embodiment, the host cell of the present invention is selected from the group consisting of stem cells, selected from the group consisting of human neural stem or precursor cells, human glial stem or precursor cells, and foetal stem cells.

In one embodiment, the host cell of the present invention is selected from the group consisting of CHO, CHO-K1, HEI193T, HEK293, COS, PC12, HiB5, RN33b, BHK cells.

In yet another aspect, the present invention relate to the use of a packaging cell line capable of producing an infective virus particle for the preparation of a medicament for inhibiting formation of amyloid plaque, said virus particle comprising a Retroviridae derived genome comprising a 5′ retroviral LTR, a tRNA binding site, a packaging signal, a promoter operably linked to a polynucleotide sequence encoding the polypeptide as defined herein above, an origin of second strand DNA synthesis, and a 3′ retroviral LTR.

In one embodiment the genome of the packaging cell line as defined herein above, is lentivirally derived.

In one embodiment the LTR of the packaging cell line as defined herein above, is lentiviral.

### VI. Antibodies

The preparation of polyclonal and monoclonal antibodies is well known in the art. Polyclonal antibodies may in particular be obtained as described by, e.g., Green et al.,: “Production of Polyclonal Antisera” in Immunochemical Protocols (Manson, Ed.); Humana Press, 1992, pages 1-5; by Coligan et al.,: “Production of Polyclonal Antisera in Rabbits, Rats, Mice and Hamsters” in Current Protocols in Immunology, 1992, Section 2.4.1, and by Ed Harlow and David Lane (Eds.) in “Antibodies; A laboratory manual” Cold Spring Harbor Lab. Press 1988. Monoclonal antibodies may in particular be obtained as described by, e.g., Kohler & Milstein, Nature, 1975, 256:495; Coligan et al., in Current Protocols in Immunology, 1992, Sections 2.5.1-2.6.7; and Harlow et al., in Antibodies: A Laboratory Manual; Cold Spring Harbor, Pub., 1988, page 726.

Briefly, monoclonal antibodies may be obtained by injecting, e.g., mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce the antibodies to the antigen, and isolating the antibodies from the hybridoma cultures.

Monoclonal antibodies can be isolated and purified from hybridoma cultures by a variety of well-established techniques, including affinity chromatography with protein A Sepharose, size-exclusion chromatography, and ion-exchange chromatography, see. e.g. Coligan et al. in Current Protocols in Immunology, 1992, Sections 2.7.1-2.7.12, and Sections 2.9.1-2.9.3; and Barnes et al.: “Purification of Immunoglobulin G (IgG)” in Methods in Molecular Biology; Humana Press, 1992, Vol. 10, Pages 79-104. Polyclonal or monoclonal antibodies may optionally be further purified, e.g. by binding to and elution from a matrix to which the polypeptide, to which the antibodies were raised, is bound.

Antibodies which bind to the same receptor targets as CTGF and BDNF polypeptide of the invention can be prepared using an intact polypeptide or fragments containing small peptides of interest as the immunising antigen. The polypeptide used to immunise an animal may be obtained by recombinant DNA techniques or by chemical synthesis, and may optionally be conjugated to a carrier protein.

In one aspect the present invention relates to the use of an antibody capable of binding specifically to a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin, thereby exhibiting the same physoplogical response as the polypeptide of the present invention, for the manufacture of a medicament for inhibiting formation of amyloid plaque in an individual in need thereof.

In one embodiment, the antibody as defined herein above, is selected from the group consisting of: polyclonal antibodies, monoclonal antibodies, humanised antibodies, single chain antibodies, recombinant antibodies.

In one embodiment, the present invention relates to an immunoconjugate comprising the antibody as defined herein above and a conjugate selected from the group consisting of: a cytotoxic agent such as a chemotherapeutic agent, a toxin, or a radioactive isotope; a member of a specific binding pair, such as avidin or streptavidin or an antigen; an enzyme capable of producing a detectable product for use in the inhibition of formation of amyloid plaque.

### VII. Pharmaceutical Composition and Administration Forms

The main routes of drug delivery, in the treatment method are intravenous, oral, and topical. Other drug-administration methods, such as subcutaneous injection or via inhalation, which are effective to deliver the drug to a target site or to introduce the drug into the bloodstream, are also contemplated.

The mucosal membrane to which the pharmaceutical preparation of the invention is administered may be any mucosal membrane of the mammal to which the biologically active substance is to be given, e.g. in the nose, vagina, eye, mouth, genital tract, lungs, gastrointestinal tract, or rectum, preferably the mucosa of the nose, mouth or vagina.

Compounds of the invention may be administered parenterally, that is by intravenous, intramuscular, subcutaneous intranasal, intrarectal, intravaginal or intraperitoneal administration. The subcutaneous and intramuscular forms of parenteral administration are generally preferred. Appropriate dosage forms for such administration may be prepared by conventional techniques. The compounds may also be administered by inhalation, which is by intranasal and oral inhalation administration. Appropriate dosage forms for such administration, such as an aerosol formulation or a metered dose inhaler, may be prepared by conventional techniques.

The compounds according to the invention may be administered with at least one other compound. The compounds may be administered simultaneously, either as separate formulations or combined in a unit dosage form, or administered sequentially.

### Formulations

Whilst it is possible for the compounds or salts of the present invention to be administered as the raw chemical, it is preferred to present them in the form of a pharmaceutical formulation. Accordingly, the present invention further provides a pharmaceutical formulation, for medicinal application, which comprises a compound of the present invention or a pharmaceutically acceptable salt thereof, as herein defined, and a pharmaceutically acceptable carrier therefore.

The compounds of the present invention may be formulated in a wide variety of oral administration dosage forms. The pharmaceutical compositions and dosage forms may comprise the compounds of the invention or its pharmaceutically acceptable salt or a crystal form thereof as the active component. The pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, wetting agents, tablet disintegrating agents, or an encapsulating material.

Preferably, the composition will be about 0.5% to 75% by weight of a compound or compounds of the invention, with the remainder consisting of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like.

In powders, the carrier is a finely divided solid which is a mixture with the finely divided active component. In tablets, the active component is mixed with the carrier having the necessary binding capacity in suitable proportions and compacted in the shape and size desired. Powders and tablets preferably contain from one to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container aseptically. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Also included are solid form preparations which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilizing agents, and the like.

Other forms suitable for oral administration include liquid form preparations including emulsions, syrups, elixirs, aqueous solutions, aqueous suspensions, toothpaste, gel dentrifrice, chewing gum, or solid form preparations which are intended to be converted shortly before use to liquid form preparations. Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavours, stabilizing and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavours, stabilizers, buffers, artificial and natural sweeteners, dispersants, thickeners, solubilizing agents, and the like.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides; (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-.beta.-aminopropionates, and 2-alkyl-imidazoline quaternary ammonium salts, and (e) mixtures thereof.

The parenteral formulations typically will contain from about 0.5 to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The compounds of the invention can also be delivered topically. Regions for topical administration include the skin surface and also mucous membrane tissues of the vagina, rectum, nose, mouth, and throat. Compositions for topical administration via the skin and mucous membranes should not give rise to signs of irritation, such as swelling or redness.

The topical composition may include a pharmaceutically acceptable carrier adapted for topical administration. Thus, the composition may take the form of a suspension, solution, ointment, lotion, sexual lubricant, cream, foam, aerosol, spray, suppository, implant, inhalant, tablet, capsule, dry powder, syrup, balm or lozenge, for example. Methods for preparing such compositions are well known in the pharmaceutical industry.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or colouring agents. Formulations suitable for topical administration in the mouth include lozenges comprising active agents in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

### Transdermal Delivery

The pharmaceutical agent-chemical modifier complexes described herein can be administered transdermally. Transdermal administration typically involves the delivery of a pharmaceutical agent for percutaneous passage of the drug into the systemic circulation of the patient. The skin sites include anatomic regions for transdermally administering the drug and include the forearm, abdomen, chest, back, buttock, mastoidal area, and the like.

Transdermal delivery is accomplished by exposing a source of the complex to a patient's skin for an extended period of time. Transdermal patches have the added advantage of providing controlled delivery of a pharmaceutical agent-chemical modifier complex to the body. See Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987). Such dosage forms can be made by dissolving, dispersing, or otherwise incorporating the pharmaceutical agent-chemical modifier complex in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

### Passive Transdermal Drug Delivery

A variety of types of transdermal patches will find use in the methods described herein. For example, a simple adhesive patch can be prepared from a backing material and an acrylate adhesive. The pharmaceutical agent-chemical modifier complex and any enhancer are formulated into the adhesive casting solution and allowed to mix thoroughly. The solution is cast directly onto the backing material and the casting solvent is evaporated in an oven, leaving an adhesive film. The release liner can be attached to complete the system.

Alternatively, a polyurethane matrix patch can be employed to deliver the pharmaceutical agent-chemical modifier complex. The layers of this patch comprise a backing, a polyurethane drug/enhancer matrix, a membrane, an adhesive, and a release liner. The polyurethane matrix is prepared using a room temperature curing polyurethane prepolymer. Addition of water, alcohol, and complex to the prepolymer results in the formation of a tacky firm elastomer that can be directly cast only the backing material.

A further embodiment of this invention will utilize a hydrogel matrix patch. Typically, the hydrogel matrix will comprise alcohol, water, drug, and several hydrophilic polymers. This hydrogel matrix can be incorporated into a transdermal patch between the backing and the adhesive layer.

The liquid reservoir patch will also find use in the methods described herein. This patch comprises an impermeable or semipermeable, heat sealable backing material, a heat sealable membrane, an acrylate based pressure sensitive skin adhesive, and a siliconized release liner. The backing is heat sealed to the membrane to form a reservoir which can then be filled with a solution of the complex, enhancers, gelling agent, and other excipients.

Foam matrix patches are similar in design and components to the liquid reservoir system, except that the gelled pharmaceutical agent-chemical modifier solution is constrained in a thin foam layer, typically a polyurethane. This foam layer is situated between the backing and the membrane which have been heat sealed at the periphery of the patch.

For passive delivery systems, the rate of release is typically controlled by a membrane placed between the reservoir and the skin, by diffusion from a monolithic device, or by the skin itself serving as a rate-controlling barrier in the delivery system. See U.S. Pat. Nos. 4,816,258; 4,927,408; 4,904,475; 4,588,580, 4,788,062; and the like. The rate of drug delivery will be dependent, in part, upon the nature of the membrane. For example, the rate of drug delivery across membranes within the body is generally higher than across dermal barriers. The rate at which the complex is delivered from the device to the membrane is most advantageously controlled by the use of rate-limiting membranes which are placed between the reservoir and the skin. Assuming that the skin is sufficiently permeable to the complex (i.e., absorption through the skin is greater than the rate of passage through the membrane), the membrane will serve to control the dosage rate experienced by the patient.

Suitable permeable membrane materials may be selected based on the desired degree of permeability, the nature of the complex, and the mechanical considerations related to constructing the device. Exemplary permeable membrane materials include a wide variety of natural and synthetic polymers, such as polydimethylsiloxanes (silicone rubbers), ethylenevinylacetate copolymer (EVA), polyurethanes, polyurethane-polyether copolymers, polyethylenes, polyamides, polyvinylchlorides (PVC), polypropylenes, polycarbonates, polytetrafluoroethylenes (PTFE), cellulosic materials, e.g., cellulose triacetate and cellulose nitrate/acetate, and hydrogels, e.g., 2-hydroxyethylmethacrylate (HEMA).

Other items may be contained in the device, such as other conventional components of therapeutic products, depending upon the desired device characteristics. For example, the compositions according to this invention may also include one or more preservatives or bacteriostatic agents, e.g., methyl hydroxybenzoate, propyl hydroxybenzoate, chlorocresol, benzalkonium chlorides, and the like. These pharmaceutical compositions also can contain other active ingredients such as antimicrobial agents, particularly antibiotics, anesthetics, analgesics, and antipruritic agents.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The active compound may be formulated into a suppository comprising, for example, about 0.5% to about 50% of a compound of the invention, disposed in a polyethylene glycol (PEG) carrier (e.g., PEG 1000 [96%] and PEG 4000 [4%].

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example with a dropper, pipette or spray. The formulations may be provided in a single or multidose form. In the latter case of a dropper or pipette this may be achieved by the patient administering an appropriate, predetermined volume of the solution or suspension. In the case of a spray this may be achieved for example by means of a metering atomizing spray pump.

The compounds of the present invention may be formulated for aerosol administration, particularly to the respiratory tract and including intranasal administration. The compound will generally have a small particle size for example of the order of 5 microns or less. Such a particle size may be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

### Pharmaceutically Acceptable Salts

Pharmaceutically acceptable salts of the instant compounds, where they can be prepared, are also intended to be covered by this invention. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

Accordingly, in one aspect the invention relates to a pharmaceutical composition-comprising an agent as defined herin above.

In one embodiment the pharmaceutical composition as defined herein above comprises a pharmaceutically acceptable carrier.

In one embodiment of the present invention the pH of the pharmaceutical composition as defined herein above is between pH 5 and pH 9.

In one embodiment the pharmaceutical composition as defined herein above is formulated for administration by injection, suppository, oral administration, sublingual tablet or spray, cutaneous administration, inhalation or for local administration using an implantable biocompatible capsule.

In a further embodiment the injection is intravenous, intramuscular, intraspinal, intraperitoneal, subcutaneous, a bolus or a continuous administration.

In one embodiment the pharmaceutical composition according to the present invention is administered at intervals of 30 minutes to 24 hours.

In a further embodiment the pharmaceutical composition according to the present invention is administered at intervals of 1 to 6 hours.

In a further embodiment the pharmaceutical composition according to the present invention is administered at intervals of 6 to 72 hours.

In another embodiment the pharmaceutical composition comprising the antagonist/inhibitor to the Vps10p-domain receptor according to the present invention is administered at a dosage of between 10 μg to 500 mg per kg body mass.

A target tissue for CTGF, BDNF and CTGF/BDNF combination therapy is e.g. a region of the brain selected for its retained responsiveness to CTGF and/or BDNF. In humans, neurons, which retain responsiveness to growth factors into adulthood include the cholinergic basal forebrain neurons, the entorhinal cortical neurons, the thalamic neurons, the locus coeruleus neurons, the spinal sensory neurons, the spinal motor neurons, neurons of substantia nigra, sympathetic neurons, dorsal root ganglia, retina neurons, otic neurons, cerebellar neurons, and ciliary ganglia. Stem cells, such as stem cells of the subventricular zone, and neural and glial progenitor cells also retain responsiveness to growth factors into adulthood. Also myelinating oligodendrocytes retain responsiveness to growth factors into adulthood.

The polypeptides of the present invention may be administered in any manner, which is medically acceptable. This may include injections, by parenteral routes such as intravenous, intravascular, intraarterial, subcutaneous, intramuscular, intratumor, intraperitoneal, intraventricular, intraepidural, intertracheal, intrathecal, intracerebroventricular, intercerebral, interpulmonary, or others as well as nasal, ophthalmic, rectal, or topical. Sustained release administration is also specifically included in the invention, by such means as depot injections or erodible implants. Peroral administration is also conceivable provided the protein is protected against degradation in the stomach.

Administration of CTGF, BDNF or a combination thereof according to this invention may be achieved using any suitable delivery means, including: pump (see, e.g., Annals of Pharmacotherapy, 27:912 (1993); Cancer, 41:1270 (1993); Cancer Research, 44:1698 (1984), incorporated herein by reference), microencapsulation (see, e.g., U.S. Pat. Nos. 4,352,883; 4,353,888; and 5,084,350, herein incorporated by reference), continuous release polymer implants (see, e.g., Sabel, U.S. Pat. No. 4,883,666, incorporated herein by reference), encapsulated cells expressing CTGF and/or BDNF, naked or unencapsulated cell grafts expressing CTGF and/or BDNF to the CNS (see, e.g., U.S. Pat. Nos. 5,082,670 and 5,618,531, each incorporated herein by reference); injection, either subcutaneously, intravenously, intra-arterially, intramuscularly, or to other suitable site; inhalation; and oral administration, in capsule, liquid, tablet, pill, or prolonged release formulation.

Administration may be by periodic injections of a bolus of the preparation, or may be made more continuous by intravenous or intraperitoneal administration from a reservoir which is external (e.g., an IV bag) or internal (e.g., a bioerodable implant, a bioartificial organ, a biocompatible capsule of CTGF and/or BDNF producing cells, or a colony of implanted CTGF and/or BDNF production cells). See, e.g., U.S. Pat. Nos. 4,407,957, 5,798,113, and 5,800,828, each incorporated herein by reference. Intrapulmonary delivery methods and apparatus are described, for example, in U.S. Pat. Nos. 5,654,007, 5,780,014, and 5,814,607, each incorporated herein by reference. Apart from systemic delivery, delivery directly to the CNS behind the blood-brain or blood-retina barriers is also contemplated. Localised delivery may be by such means as delivery via a catheter to one or more arteries, such as the cerebral artery to the CNS. Methods for local pump-based delivery of protein formulations to the CNS are described in U.S. Pat. No. 6,042,579 (Medtronic). Another type of localised delivery comprises delivery using encapsulated cells. A further type of localised delivery comprises local delivery of gene therapy vectors, which are normally injected. In subjects with neurodegenerative diseases such as AD, neurons in the Ch4 region (nucleus basalis of Meynert) which have nerve growth factor (NGF) receptors undergo marked atrophy as compared to normal controls (see, e.g., Kobayashi, et al., Mol. Chem. Neuropathol., 15: 193-206 (1991)). In normal subjects, neurotrophins prevent sympathetic and sensory neuronal death during development and prevents cholinergic neuronal degeneration in adult rats and primates (Tuszynski, et al., Gene Therapy, 3: 305314 (1996)). The resulting loss of functioning neurons in this region of the basal forebrain is believed to be causatively linked to the cognitive decline experienced by subjects suffering from neurodegenerative conditions such as AD (Tuszynski, et al., supra; Lehericy, et al., J. Comp. Neurol., 330: 15-31 (1993)).

In general it is contemplated, that AD can be treated with a formulation comprising CTGF, BDNF or a combination said formulations delivered intracerebroventricularly, or intraparenchymally. Within the intraparenchymal area, delivery is preferably to the basal forebrain, and to the hippocampus.

Gene therapy vector, encapsulated or naked cells secreting CTGF and/or BDNF can also be administered to the basal forebrain or the hippocampus.

The term “pharmaceutically acceptable carrier” means one or more organic or inorganic ingredients, natural or synthetic, with which CTGF and/or BDNF polypeptides is combined to facilitate its application. A suitable carrier includes sterile saline although other aqueous and non-aqueous isotonic sterile solutions and sterile suspensions known to be pharmaceutically acceptable are known to those of ordinary skill in the art. An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

A liposome system may be any variety of unilamellar vesicles, multilamellar vesicles, or stable plurilamellar vesicles, and may be prepared and administered according to methods well known to those of skill in the art, for example in accordance with the teachings of U.S. Pat. Nos. 5,169,637, 4,762,915, 5,000,958 or 5,185,154. In addition, it may be desirable to express the novel polypeptides of this invention, as well as other selected polypeptides, as lipoproteins, in order to enhance their binding to liposomes. A recombinant CTGF or BDNF is purified, for example, from CHO cells by immunoaffinity chromatography or any other convenient method, then mixed with liposomes and incorporated into them at high efficiency. The liposome-encapsulated protein may be tested in vitro for any effect on stimulating cell growth.

Any of CTGF, BDNF or the combination of the two polypeptides of this invention may be used in the form of a pharmaceutically acceptable salt. Suitable acids and bases which are capable of forming salts with CTGF and BDNF are well known to those of skill in the art, and include inorganic and organic acids and bases.

In addition to the active ingredients, the pharmaceutical compositions may comprise suitable ingredients. Further details on techniques for formulation and administration may be found in the latest edition of Remington's Pharmaceutical Sciences (Maack Publishing Co., Easton, Pa.).

Various dosing regimes for systemic administration are contemplated. In one embodiment, methods of administering to a subject a formulation comprising CTGF, BDNF or a combination of the polypeptides include administering said polypeptide at a dosage of between 1 μg/kg to 30,000 μg/kg body weight of the subject, per dose. In another embodiment, the dosage is between 10 μg/kg to 30,000 μg/kg body weight of the subject, per dose. In a further embodiment, the dosage is between 10 μg/kg to 10,000 μg/kg body weight of the subject, per dose. In a different embodiment, the dosage is between 25 μg/kg to 10,000 μg/kg body weight of the subject, per dose. In yet another embodiment, the dosage is between 25 μg/kg to 3,000 μg/kg body weight of the subject, per dose. In a most preferable embodiment, the dosage is between 50 μg/kg to 3,000 μg/kg body weight of the subject, per dose.

Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue.

Where sustained-release administration of CTGF, BDNF its combination is desired in a formulation with release characteristics suitable for the treatment of any disease or disorder requiring administration of CTGF, BDNF its combination thereof, microencapsulation of CTGF, BDNF or its combination is contemplated. Microencapsulation of recombinant proteins for sustained release has been successfully performed with human growth hormone (rhGH), interferon-(rhIFN-), interleukin-2, and MN rgp120. Johnson et al., Nat. Med., 2:795-799 (1996); Yasuda, Biomed. Ther., 27:1221-1223 (1993); Hora et al., Bio/Technology, 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010.

The sustained-release formulations of these proteins were developed using poly-lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41.

The dose administered must be carefully adjusted to the age, weight and condition of the individual being treated, as well as the route of administration, dosage form and regimen, and the result desired, and the exact dosage should be determined by the practitioner.

In a main aspect, the present invention relate to a pharmaceutical composition comprising a combination of:
a) an isolated polypeptide selected from the group consisting of a sequence variant of the amino acid sequence selected from the group consisting of SEQ ID NO. 5, 8, 11, 14, 17, 20 and 23, wherein the sequence variant has at least 70% sequence identity to said SEQ ID NO. 5, 8, 11, 14, 17, 20 and 23; or
b) a biologically active fragment of at least 50 contiguous amino acids of any of a) wherein any amino acid specified in the selected sequence is altered to a different amino acid, provided that no more than 15 of the amino acid residues in the sequence are so altered;
   and
c) an isolated polypeptide selected from the group consisting of a sequence variant of the amino acid sequence selected from the group consisting of SEQ ID NO. 34, 41, 45, 49, 53, 57, 58, 59, 60, 61 and 62, wherein the sequence variant has at least 70% sequence identity to said SEQ ID NO. 34, 41, 45, 49, 53, 57, 58, 59, 60, 61 or 62; or
d) a biologically active fragment of at least 50 contiguous amino acids of any of a) wherein any amino acid specified in the selected sequence is altered to a different amino acid, provided that no more than 15 of the amino acid residues in the sequence are so altered.
   for the preparation of a combination medicament for inhibiting formation of amyloid plaque.

In another main aspect, the invention relate to the use of a pharmaceutical composition comprising a combination of:
a) an isolated polypeptide selected from the group consisting of a sequence variant of the amino acid sequence selected from the group consisting of SEQ ID NO. 5, 8, 11, 14, 17, 20 and 23, wherein the sequence variant has at least 70% sequence identity to said SEQ ID NO. 5, 8, 11, 14, 17, 20 and 23; or
b) a biologically active fragment of at least 50 contiguous amino acids of any of a) wherein any amino acid specified in the selected sequence is altered to a different amino acid, provided that no more than 15 of the amino acid residues in the sequence are so altered;
   and
c) an isolated polypeptide selected from the group consisting of a sequence variant of the amino acid sequence selected from the group consisting of SEQ ID NO. 34, 41, 45, 49, 53, 57, 58, 59, 60, 61 and 62, wherein the sequence variant has at least 70% sequence identity to said SEQ ID NO. 34, 41, 45, 49, 53, 57, 58, 59, 60, 61 or 62; or
d) a biologically active fragment of at least 50 contiguous amino acids of any of a) wherein any amino acid specified in the selected sequence is altered to a different amino acid, provided that no more than 15 of the amino acid residues in the sequence are so altered.
   for the preparation of a combination medicament for inhibiting formation of amyloid plaque.

In yet another aspect, the present invention relate to a pharmaceutical composition for use in a method of treatment of a disease or disorder resulting from amyloid plaque said composition comprising a combination of:
a) an isolated polypeptide selected from the group consisting of a sequence variant of the amino acid sequence selected from the group consisting of SEQ ID NO. 5, 8, 11, 14, 17, 20 and 23, wherein the sequence variant has at least 70% sequence identity to said SEQ ID NO. 5, 8, 11, 14, 17, 20 and 23; or
b) a biologically active fragment of at least 50 contiguous amino acids of any of a) wherein any amino acid specified in the selected sequence is altered to a different amino acid, provided that no more than 15 of the amino acid residues in the sequence are so altered;
   and
c) an isolated polypeptide selected from the group consisting of a sequence variant of the amino acid sequence selected from the group consisting of SEQ ID NO. 34, 41, 45, 49, 53, 57, 58, 59, 60, 61 and 62, wherein the sequence variant has at least 70% sequence identity to said SEQ ID NO. 34, 41, 45, 49, 53, 57, 58, 59, 60, 61 or 62; or
d) a biologically active fragment of at least 50 contiguous amino acids of any of a) wherein any amino acid specified in the selected sequence is altered to a different amino acid, provided that no more than 15 of the amino acid residues in the sequence are so altered;
   for the preparation of a combination medicament for inhibiting formation of amyloid plaque.

In a further aspect, the present invention relate to a pharmaceutical composition comprising a combination of:
a) an isolated polypeptide selected from the group consisting of a sequence variant of the amino acid sequence selected from the group consisting of SEQ ID NO. 5, 8, 11, 14, 17, 20 and 23, wherein the sequence variant has at least 70% sequence identity to said SEQ ID NO. 5, 8, 11, 14, 17, 20 and 23; or
b) a biologically active fragment of at least 50 contiguous amino acids of any of a) wherein any amino acid specified in the selected sequence is altered to a different amino acid, provided that no more than 15 of the amino acid residues in the sequence are so altered;
   and
c) an isolated polypeptide selected from the group consisting of a sequence variant of the amino acid sequence selected from the group consisting of SEQ ID NO. 34, 41, 45, 49, 53, 57, 58, 59, 60, 61 and 62, wherein the sequence variant has at least 70% sequence identity to said SEQ ID NO. 34, 41, 45, 49, 53, 57, 58, 59, 60, 61 or 62; or
d) a biologically active fragment of at least 50 contiguous amino acids of any of a) wherein any amino acid specified in the selected sequence is altered to a different amino acid, provided that no more than 15 of the amino acid residues in the sequence are so altered.
   for the preparation of a combination medicament for inhibiting formation of amyloid plaque.

In another main aspect, the present invention relates to the use of a pharmaceutical composition comprising a combination of:
a) an isolated polypeptide selected from the group consisting of a sequence variant of the amino acid sequence selected from the group consisting of SEQ ID NO. 5, 8, 11, 14, 17, 20 and 23, wherein the sequence variant has at least 70% sequence identity to said SEQ ID NO. 5, 8, 11, 14, 17, 20 and 23; or
b) a biologically active fragment of at least 50 contiguous amino acids of any of a) wherein any amino acid specified in the selected sequence is altered to a different amino acid, provided that no more than 15 of the amino acid residues in the sequence are so altered;
   and
c) an isolated polypeptide selected from the group consisting of a sequence variant of the amino acid sequence selected from the group consisting of SEQ ID NO. 34, 41, 45, 49, 53, 57, 58, 59, 60, 61 and 62, wherein the sequence variant has at least 70% sequence identity to said SEQ ID NO. 34, 41, 45, 49, 53, 57, 58, 59, 60, 61 or 62; or
d) a biologically active fragment of at least 50 contiguous amino acids of any of a) wherein any amino acid specified in the selected sequence is altered to a different amino acid, provided that no more than 15 of the amino acid residues in the sequence are so altered.
   for the preparation of a combination medicament for inhibiting formation of amyloid plaque.

In an important aspect, the present invention relate to a pharmaceutical composition for use in a method of treatment of a disease or disorder resulting from amyloid plaque said composition comprising a combination of:
a) an isolated polypeptide selected from the group consisting of a sequence variant of the amino acid sequence selected from the group consisting of SEQ ID NO. 5, 8, 11, 14, 17, 20 and 23, wherein the sequence variant has at least 70% sequence identity to said SEQ ID NO. 5, 8, 11, 14, 17, 20 and 23; or
b) a biologically active fragment of at least 50 contiguous amino acids of any of a) wherein any amino acid specified in the selected sequence is altered to a different amino acid, provided that no more than 15 of the amino acid residues in the sequence are so altered;
   and
c) an isolated polypeptide selected from the group consisting of a sequence variant of the amino acid sequence selected from the group consisting of SEQ ID NO. 34, 41, 45, 49, 53, 57, 58, 59, 60, 61 and 62, wherein the sequence variant has at least 70% sequence identity to said SEQ ID NO. 34, 41, 45, 49, 53, 57, 58, 59, 60, 61 or 62; or
d) a biologically active fragment of at least 50 contiguous amino acids of any of a) wherein any amino acid specified in the selected sequence is altered to a different amino acid, provided that no more than 15 of the amino acid residues in the sequence are so altered;
   for the preparation of a combination medicament for inhibiting formation of amyloid plaque.

In an important embodiment, the present invention relates to a pharmaceutical composition comprising:
a) the at least one polypeptide as defined herein above; and/or
b) the at least one isolated nucleic acid sequence as defined herein above; and/or
c) the at least one expression vector as defined herein above; and/or
d) a composition of host cells as defined herein; and/or;
e) a packaging cell line as defined herein, and/or
f) a monoclonal or polyclonal antibody as defined herein.

In one embodiment, the invention relates to a method of treating a disease resulting from formation of amyloid plaque, in a subject comprising administering to an individual in need thereof a therapeutically effective amount of:
a) at least one polypeptide of the present invention; or
b) at least one isolated nucleic acid sequence of the present invention; or
c) at least one expression vector of the present invention; or
d) a composition of host cells according to the present invention; or;
e) a packaging cell line according to the present invention.

In one embodiment the pharmaceutical composition of the present invention comprises a pharmaceutically acceptable carrier.

In a further embodiment the pharmaceutical composition of the present invention comprises a second active ingredient.

In one embodiment the pH of the pharmaceutical composition according to the present invention is between pH 4 and pH 10.

In another embodiment, the pharmaceutical composition according to the present invention is formulated for administration by injection, suppository, oral administration, sublingual tablet or spray, cutaneous administration, inhalation or for local administration using an implantable biocompatible capsule.

In a further embodiment, the injection of the pharmaceutical composition is intravenous, intramuscular, intraspinal, intraperitoneal, subcutaneous, a bolus or a continuous administration.

In one embodiment, the administration of the pharmaceutical composition occurs at intervals of 30 minutes to 24 hours.

In one embodiment, the administration of the pharmaceutical composition occurs at intervals of 1 to 6 hours.

In one embodiment, the duration of the treatment with the pharmaceutical composition of the present invention is from 6 to 72 hours.

In one embodiment, the dosage of the active ingredient in the pharmaceutical composition according to the present invention is between 10 μg to 500 mg per kg body mass.

### VIII. Second Active Ingredient

In one embodiment the pharmaceutical composition as defined herein above comprises a second active ingredient such as a conventional medicament useful in the treatment of AD.

### IX. Further Novel Methods of the Invention

In an important aspect the present invention relates to a method of inhibiting formation of amyloid plaque in a patient in need thereof, said method comprising administering to the patient the
a) at least one polypeptide of the invention; and/or
b) the at least one isolated nucleic acid sequence as defined herein above; and/or
c) the at least one expression vector as defined herein above; and/or
d) a composition of host cells as defined herein above; and/or;
e) a packaging cell line according as defined herein above, or
f) an antibody capable of binding specifically to a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin, or
g) a small organic molecule, or
h) a combination of two or more of any of a) through g).

In another important aspect the present invention relate to a method of inhibiting cleavage of APP to Aβ and soluble APPα in an individual in need thereof, said method comprising administering to the individual the
a) at least one polypeptide as defined herein above; and/or
b) the at least one isolated nucleic acid sequence as defined herein above; and/or
c) the at least one expression vector as defined herein above; and/or
d) a composition of host cells as defined herein above; and/or;
e) a packaging cell line as defined herein above, or
f) an antibody capable of binding specifically to a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin, or
g) a small organic molecule, or
h) or a combination of two or more of any of a) through g).

In one aspect, the present invention relate to a method of inhibiting formation of Aβ and soluble APPα plaque in a patient in need thereof, said method comprising administering to the patient:
a) the at least one polypeptide as defined herein above; and/or
b) the at least one isolated nucleic acid sequence as defined herein above; and/or
c) the at least one expression vector as defined herein above; and/or
d) a composition of host cells as defined herein above; and/or;
e) a packaging cell line as defined herein above, or
f) an antibody capable of binding specifically to a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin, or
g) a small organic molecule, or
h) a combination of two or more of any of a) through g).

In a highly preferred aspect the present invention relate to a method of upregulating SorLA (SEQ ID NO. 2) or a fragment or variant thereof, in a patient in need thereof, said method comprising administering to the patient:
a) the at least one polypeptide as defined herein above; and/or
b) the at least one isolated nucleic acid sequence as defined herein above; and/or
c) the at least one expression vector as defined herein above; and/or
d) a composition of host cells as defined herein above; and/or;
e) a packaging cell line as defined herein above, or
f) an antibody capable of binding specifically to a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin, or
g) a small organic molecule, or
h) a combination of two or more of any of a) through g)

In one aspect the present invention relate to an in vitro method of upregulating SorLA (SEQ ID NO. 2), said method comprising administering to the patient:
a) the at least one polypeptide as defined herein above; and/or
b) the at least one isolated nucleic acid sequence as defined herein above; and/or
c) the at least one expression vector as defined herein above; and/or
d) a composition of host cells as defined herein above; and/or;
e) a packaging cell line as defined herein above, or
f) an antibody capable of binding specifically to a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin, or
g) a small organic molecule, or
h) a combination of two or more of any of a) through g).

In one aspect the present invention relate to the use of the isolated polypeptide as defined herein above, for the preparation of a medicament for inhibiting cleavage of amyloid precursor protein (APP), said polypeptide comprising an amino acid sequence selected from the group consisting of:
a) the amino acid sequence selected from the group consisting of SEQ ID NO. 5, 8, 11, 14, 17, 20, 23, 34, 41, 45, 49, 53, 57, 58, 59, 60, 61 and 62 or a naturally occurring precursor protein thereof; and
b) a sequence variant or a naturally occurring precursor protein of the amino acid sequence selected from the group consisting of SEQ ID NO. 5, 8, 11, 14, 17, 20, 23, 34, 41, 45, 49, 53, 57, 58, 59, 60, 61 and 62, wherein the sequence variant has at least 70% sequence identity to said SEQ ID NO. 5, 8, 11, 14, 17, 20, 23, 34, 41, 45, 49, 53, 57, 58, 59, 60, 61 or 62; and
c) a biologically active fragment of at least 50 contiguous amino acids of any of a) wherein any amino acid specified in the selected sequence is altered to a different amino acid, provided that no more than 15 of the amino acid residues in the sequence are so altered.

In one aspect the present invention relate to the use of at least one agonist of a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin for the preparation of a medicament or a combination medicament for inhibiting formation of amyloid plaque.

In one aspect the present invention relate to the use of at least one agonist of a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin for the preparation of a medicament or a combination medicament for inhibiting cleavage of APP to Aβ and soluble APPα in an individual in need thereof.

In one aspect the present invention relate to the use of at least one agonist of a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin for the preparation of a medicament or a combination medicament for inhibiting cleavage of APP to Aβ and soluble APPα in an individual suffering from Alzheimer's Disease.

In one aspect the present invention relate to the use In one aspect the present invention relate to the use of at least one agonist of a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin for use in a method of treatment of abnormal levels of amyloid plaque, Aβ and soluble APPα in an individual.

### X. Kit of Parts

In one aspect the invention relates to a kit in parts comprising:
a pharmaceutical composition as defined herein,
a medical instrument or other means for administering said pharmaceutical composition,
instructions on how to use the kit in parts and optionally
a second active ingredient.

### DETAILED DESCRIPTION OF THE DRAWINGS

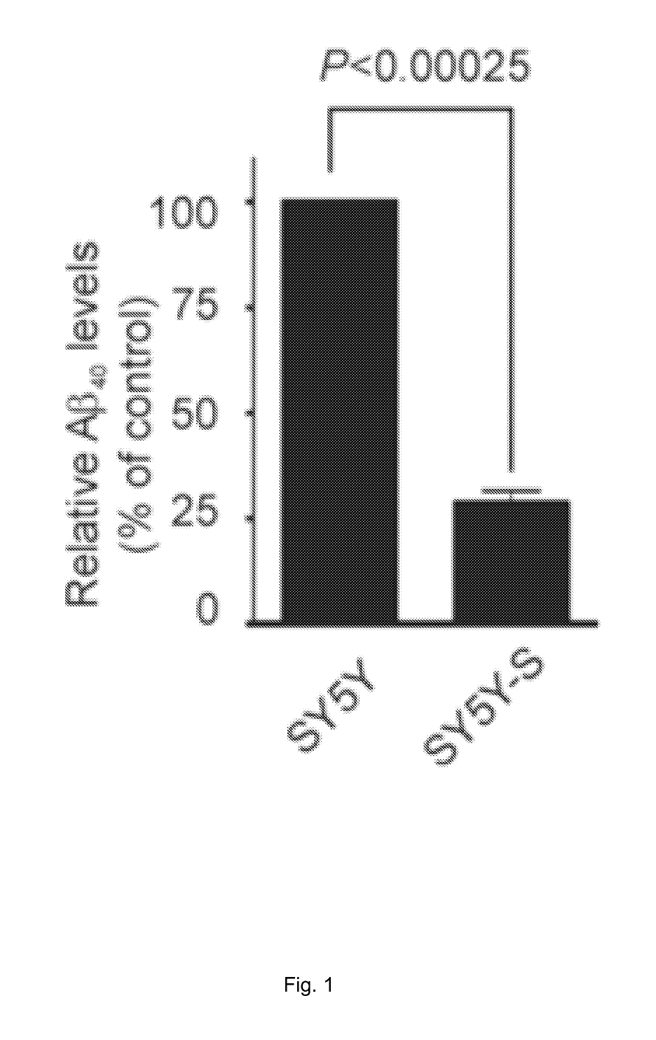

FIG. 1: Overexpression of SorLA reduces Aβ production in neurons: Neuronal SY5Y cells were stably transfected with an expression construct for SorLA (SY5Y-S) and the amount of Aβ produced and secreted into the medium was determined by ELISA. Overexpression of SorLA significantly reduces the amount of Aβ produced in SY5Y-S cells compared to the parental SY5Y cells not overexpressing SorLA.

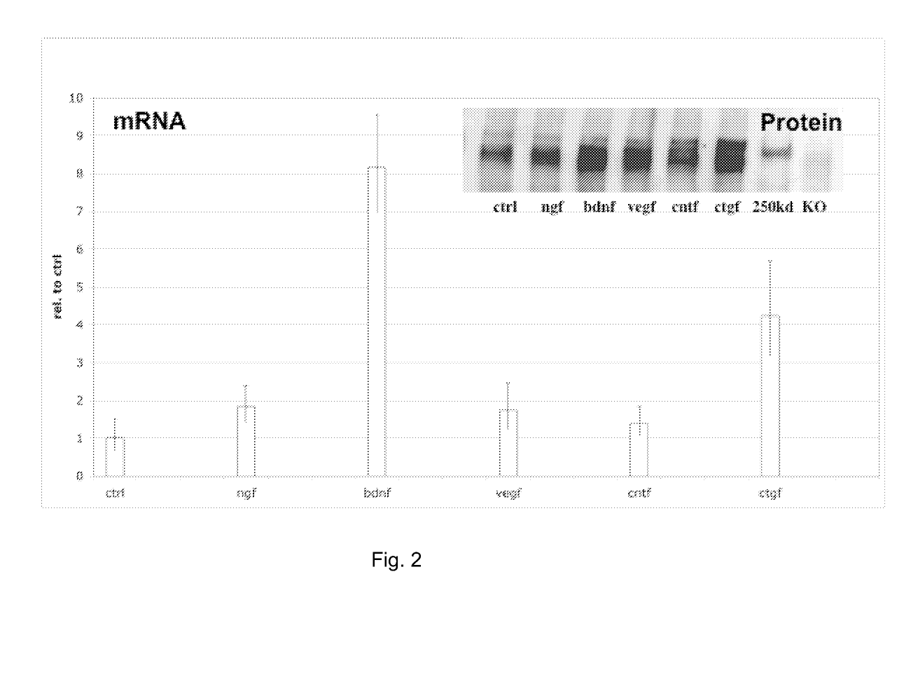

FIG. 2: Treatment with BDNF and CTGF induces SorLA expression in neurons: Primary neurons from newborn mouse brain were treated for 24 hours with 150 ng/ml of various neurotrophins. Subsequently, the amount of SorLA mRNA was determined by quantitative RT-PCR (bar graphs) and the amount of SorLA protein was determined by western blot analysis of cell lysates (inset). The amount of mRNA and protein were compared to untreated cells (ctrl). Both SorLA mRNA and protein levels are significantly increased in neurons treated with BDNF and CTGF. bdnf, brain neurotrophic factor; cntf, ciliary neurotrophic factor; ctrl, untreated control cells; ctgf, connecive tissue growth factor; ngf, nerve growth factor; vegf, vascular endothelial growth factor; KO, tissue extract from SorLA knockout mice as negative control for immunodetection of the receptor.

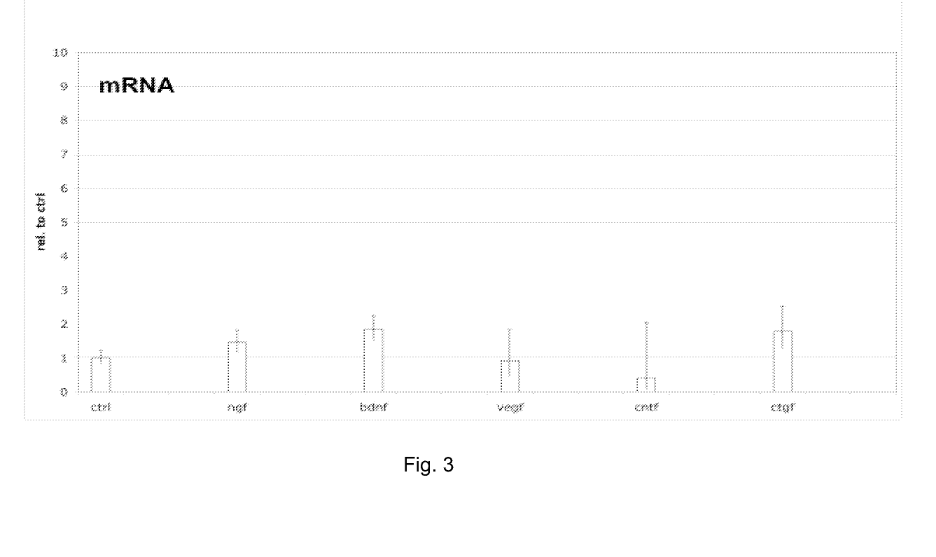

FIG. 3: Treatment with BDNF and CTGF does not induce Sortilin expression in neurons: Primary neurons from newborn mouse brain were treated for 24 hours with 150 ng/ml of various neurotrophins. Subsequently, the amount of Sortilin mRNA was determined by quantitative RT-PCR. The amount of mRNA was compared to untreated cells (ctrl). Sortilin mRNA levels are not significantly increased in neurons by treatment of any of the neurotrophic factors applied here. bdnf, brain neurotrophic factor; cntf, ciliary neurotrophic factor; ctrl, untreated control cells; ctgf, connecive tissue growth factor; ngf, nerve growth factor; vegf, vascular endothelial growth factor;

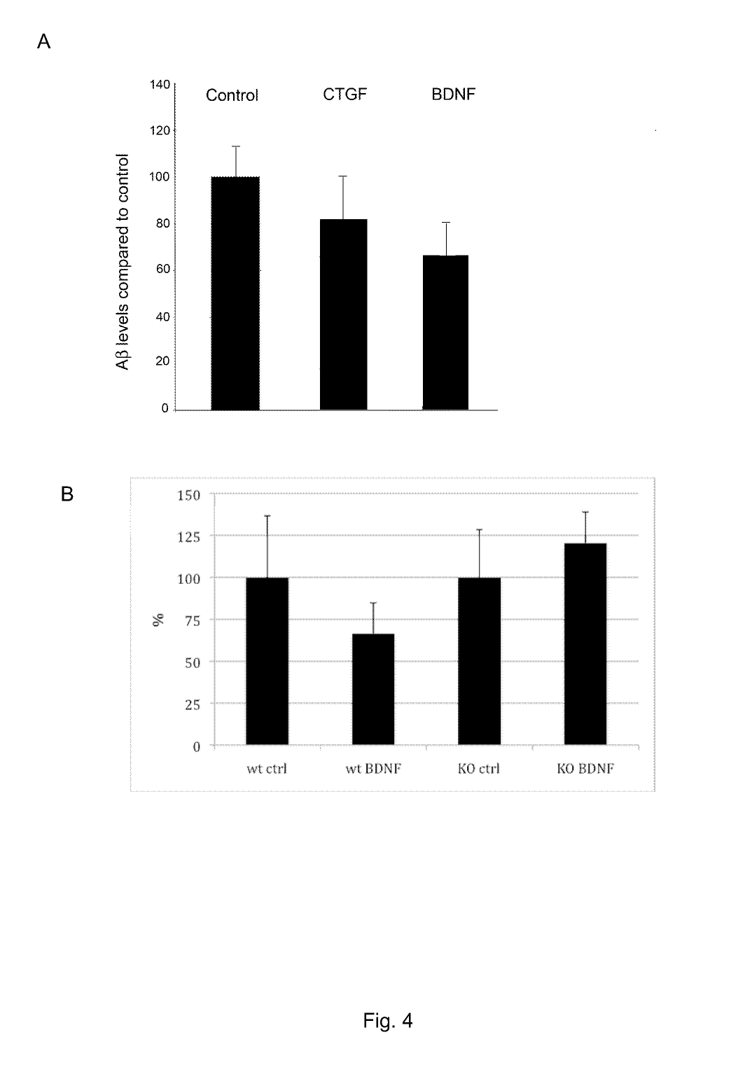

FIG. 4A: Treatment with BDNF and CTGF reduces AP production in primary neurons: Primary neurons from newborn wild type mice were treated with 200 ng/ml of CTGF or BDNF or control buffer (control). Thereafter, the amount of Aβ in the cell culture was determined by ELISA. Aβ production was significantly reduced in neurons treated with CTGF or BDNF compared to control cells.

FIG. 4B: Treatment with BDNF in vivo reduces Aβ production in a SORLA dependent manner. Wild type mice (wt) or mice genetically deficient for SORLA (KO) were treated with intracranial injections of either artificial cerebrospinal fluid (ctrl) or recombinant BDNF (mini pumps) for 7 days. Thereafter, the animals were sacrificed and Aβ levels determined in brain homogenates using ELISA. Application of BDNF reduced the levels of Aβ in wild type mice (wt BDNF) compared with wild types treated with CSF (wt ctrl) by approximately 40%. In contrast, the levels of Aβ in SORLA deficient mice treated with CSF (KO ctrl) or BDNF (KO BDNF) remain unchanged. This experiment provides experimental proof that application of BDNF to raise SORLA levels in the brain in vivo reduces amlyoidogenc processing.

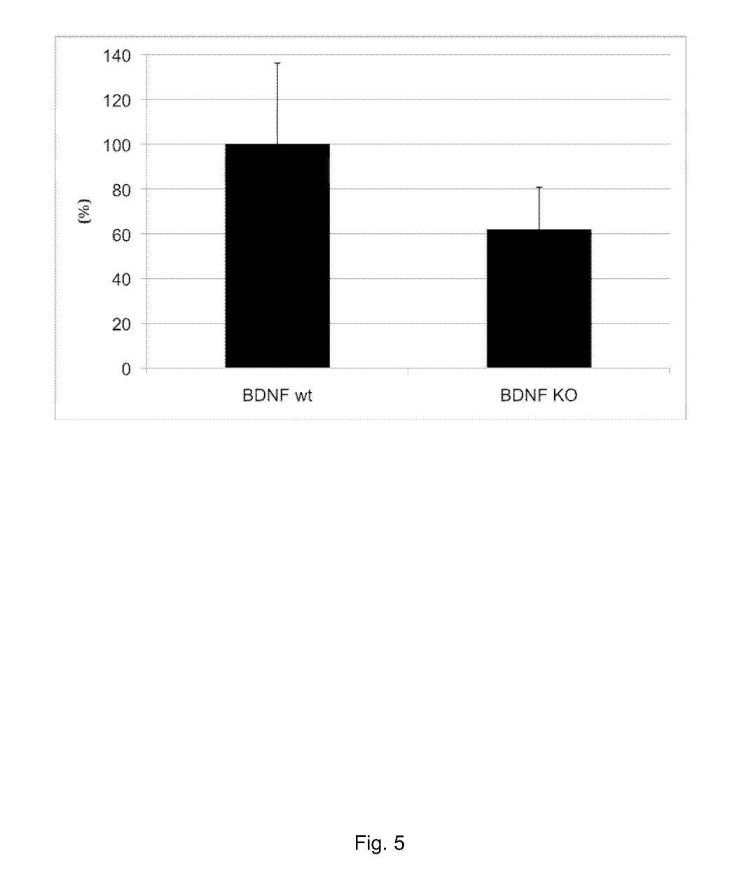

FIG. 5: Loss of BNDF expression in mice reduces SorLA levels. The brains of newborn mice genetically deficient for BDNF (BNDF−/−) and of wild type control littermates (BDNF+/+) were homogenized and subjected to Western blot analysis using antisera specific for SorLA. The intensities of the SorLA immunoreactive bands was determined by densitometric scanning and blotted as % of control levels (set at 100%). In mice lacking BNDF, the levels of SorLA in the brain are reduced to approximately 60% of the control level. This experiment proofs that BDNF acts in vivo as physiological inductor of SORLA gene expression.

FIG. 6: Sequence alignment CTGF

The alignment displays the following symbols denoting the degree of conservation observed:

“*” means that the residues in that column are identical in all sequences in the alignment.
“:” means that conserved substitutions have been observed according to common structure/charge of the residues (small+hydrophobic; acidic; basic; hydroxyl+amine+Basic−Q)
“.” means that semi-conserved substitutions are observed.

FIG. 7: Sequence alignment BDNF

The alignment displays the following symbols denoting the degree of conservation observed:

“*” means that the residues in that column are identical in all sequences in the alignment.
“:” means that conserved substitutions have been observed according to common structure/charge of the residues (small+hydrophobic; acidic; basic; hydroxyl+amine+Basic−Q)
“.” means that semi-conserved substitutions are observed.

### OVERVIEW OF Sequences

SEQ ID NO 1: Homo sapiens SorLA nucleotide
SEQ ID NO 2: Homo sapiens SorLA polypeptide (NM_{—}003105)
SEQ ID NO 3: pre-Homo sapiens CTGF nucleotide (NC_{—}000006)
SEQ ID NO 4: pre-Homo sapiens CTGF polypeptide (Swiss-Prot P29279)
SEQ ID NO 5: Mature Homo sapiens CTGF polypeptide (Swiss-Prot P29279)
SEQ ID NO 6: pre-Mus musculus CTGF nucleotide (NM_{—}010217)
SEQ ID NO 7: pre-Mus musculus CTGF polypeptide (P29268)
SEQ ID NO 8: Mature Mus musculus CTGF polypeptide (P29268)
SEQ ID NO 9: pre-Rattus Norvegicus CTGF nucleotide (AB023068)
SEQ ID NO 10: pre-Rattus Norvegicus CTGF polypeptide (Q9R1E9)
SEQ ID NO 11: Mature Rattus Norvegicus CTGF polypeptide (Q9R1E9)
SEQ ID NO 12: pre-Bos taurus CTGF nucleotide (NM_{—}174030.2)
SEQ ID NO 13: pre-Bos taurus CTGF polypeptide (O18739)
SEQ ID NO 14: Mature Bos taurus CTGF polypeptide
SEQ ID NO 15: pre-Sus scrofa CTGF nucleotide (NM_{—}213833)
SEQ ID NO 16: pre-Sus scrofa CTGF polypeptide (O97765)
SEQ ID NO 17: Mature Sus scrofa CTGF polypeptide (O97765)
SEQ ID NO 18: Pre-Xenopus CTGF nucleotide (BC094492)
SEQ ID NO 19: Pre-Xenopus CTGF polypeptide (BC094492)
SEQ ID NO 20: Mature Xenopus CTGF polypeptide (0505L5)
SEQ ID NO 21: pre-Danio rerio CTGF nucleotide (NM_{—}001015041)
SEQ ID NO 22: pre-Danio rerio CTGF polypeptide (NM_{—}001015041)
SEQ ID NO 23: Mature Danio rerio CTGF polypeptide (NM_{—}001015041)
SEQ ID NO 24: CTGFFRAG1-Sequences encoding exon 4 are nucleotide 748 to 959 of the human sequence (NM_{—}001901):
SEQ ID NO 25: CTGFFRAG2-Sequences encoding exon 5 are nucleotide 960 to 2344 of the human sequence (NM_{—}001901)
SEQ ID NO 26: Homo sapiens BDNF transcript variant 1 nucleotide (NM_{—}170735)
SEQ ID NO 27: Homo sapiens BDNF transcript variant 2 nucleotide (NM_{—}170732)
SEQ ID NO 28: Homo sapiens BDNF transcript variant 3 nucleotide (NM_{—}170731)
SEQ ID NO 29: Homo sapiens BDNF transcript variant 4 nucleotide (NM_{—}001709)
SEQ ID NO 30: Homo sapiens BDNF transcript variant 5 nucleotide (NM_{—}170733)
SEQ ID NO 31: Homo sapiens BDNF transcript variant 6 nucleotide (NM_{—}170734)
SEQ ID NO 32: Homo sapiens pre-pro-BDNF polypeptide (Swiss-Prot P23560)
SEQ ID NO 33: Homo sapiens pro-BDNF polypeptide (Swiss-Prot P23560)
SEQ ID NO 34: Homo sapiens mature BDNF polypeptide (Swiss-Prot P23560)
SEQ ID NO 35: Mus musculus BDNF transcript var 1 nucleotide (NM_{—}007540)
SEQ ID NO 36: Mus musculus BDNF transcript var 2 nucleotide (NM_{—}001048139)
SEQ ID NO 37: Mus musculus BDNF transcript var 3 nucleotide (NM_{—}001048141):
SEQ ID NO 38: Mus musculus BDNF transcript var 4 nucleotide (NM_{—}001048142):
SEQ ID NO 39: Mus musculus pre-pro BNDF polypeptide (Swiss-Prot P21237)
SEQ ID NO 40: Mus musculus pro-BNDF polypeptide (Swiss-Prot P21237)
SEQ ID NO 41: Mus musculus mature BNDF polypeptide (Swiss-Prot P21237)
SEQ ID NO 42: Rattus norvegicus BDNF nucleotide (NM_{—}012513)
SEQ ID NO 43: Rattus norvegicus pre-pro-BDNF polypeptide (NM_{—}012513)
SEQ ID NO 44: Rattus norvegicus pro-BDNF polypeptide (NM_{—}012513)
SEQ ID NO 45: Rattus norvegicus mature BDNF polypeptide (NM_{—}012513)
SEQ ID NO 46: Bos taurus BDNF nucleotide (NM_{—}001046607)
SEQ ID NO 47: Bos taurus pre-pro-BDNF polypeptide (Swiss-Prot Q95106)
SEQ ID NO 48: Bos taurus pro-BDNF polypeptide (Swiss-Prot Q95106)
SEQ ID NO 49: Bos taurus mature BDNF polypeptide (Swiss-Prot Q95106)
SEQ ID NO 50: Sus scrofa BDNF nucleotide (NP999424)
SEQ ID NO 51: Sus scrofa pre-pro-BDNF (Swiss-Prot P14082)
SEQ ID NO 52: Sus scrofa pro-BDNF polypeptide (Swiss-Prot P14082)
SEQ ID NO 53: Sus scrofa mature BDNF polypeptide (Swiss-Prot P14082)
SEQ ID NO 54: Xenopus laevis BDNF nucleotide (NM_{—}001085482)
SEQ ID NO 55: Xenopus laevis pre-pro-BDNF polypeptide (Swiss-Prot P25432)
SEQ ID NO 56: Xenopus laevis pro-BDNF polypeptide (Swiss-Prot P25432)
SEQ ID NO 57: Xenopus laevis mature BDNF polypeptide (Swiss-Prot P25432)

### SEQ ID NO 58: Chick BDNF

### SEQ ID NO 59: Dog BDNF

SEQ ID NO 60: Rhesus monkey BDNF

### SEQ ID NO 61: Cat BDNF

### SEQ ID NO 62: Fish BDNF

### SEQ ID NO 63: BDNFTRUNCMUT 1

### SEQ ID NO 64: BDNFTRUNCMUT 2

### SEQ ID NO 65: BDNFTRUNCMUT 3

### SEQ ID NO 66: BDNFTRUNCMUT 4

### SEQ ID NO 67: BDNFTRUNCMUT 5

### SEQ ID NO 68: BDNFTRUNCMUT 6

### Sequence Listing

| SEQ ID NO 1: Homo sapiens SorLA nucleotide | |
|---|---|
| 1 | ccggcccagc ggctctcctg gcctcgcgct gcacattctc tcctggcggc ggcgccacct |
| 61 | gcagtagcgt tcgcccgaac atggcgacac ggagcagcag gagggagtcg cgactcccgt |
| 121 | tcctattcac cctggtcgca ctgctgccgc ccggagctct ctgcgaagtc tggacgcaga |
| 181 | ggctgcacgg cggcagcgcg cccttgcccc aggaccgggg cttcctcgtg gtgcagggcg |
| 241 | acccgcgcga gctgcggctg tgggcgcgcg gggatgccag gggggcgagccgcgcggacg |
| 301 | agaagccgct ccggaggaaa cggagcgctg ccctgcagcc cgagcccatc aaggtgtacg |
| 361 | gacaggttag tctgaatgat tcccacaatc agatggtggt gcactgggct ggagagaaaa |
| 421 | gcaacgtgat cgtggccttg gcccgagata gcctggcatt ggcgaggccc aagagcagtg |
| 481 | atgtgtacgt gtcttacgac tatggaaaat cattcaagaa aatttcagac aagttaaact |
| 541 | ttggcttggg aaataggagt gaagctgtta tcgcccagtt ctaccacagc cctgcggaca |
| 601 | acaagcggta catctttgca gacgcttatg cccagtacct ctggatcacg tttgacttct |
| 661 | gcaacactct tcaaggcttt tccatcccat ttcgggcagc tgatctcctc ctacacagta |
| 721 | aggcctccaa ccttctcttg ggctttgaca ggtcccaccc caacaagcag ctgtggaagt |
| 781 | cagatgactt tggccagacc tggatcatga ttcaggaaca tgtcaagtcc ttttcttggg |
| 841 | gaattgatcc ctatgacaaa ccaaatacca tctacattga acgacatgaa ccctctggct |
| 901 | actccactgt cttccgaagt acagatttct tccagtcccg ggaaaaccag gaagtgatcc |
| 961 | ttgaggaagt gagagatttt cagcttcggg acaagtacat gtttgctaca aaggtggtgc |
| 1021 | atctcttggg cagtgaacag cagtcttctg tccagctctg ggtctccttt ggccggaagc |
| 1081 | ccatgagagc agcccagttt gtcacaagac atcctattaa tgaatattac atcgcagatg |
| 1141 | cctccgagga ccaggtgttt gtgtgtgtca gccacagtaa caaccgcacc aatttataca |
| 1201 | tctcagaggc agaggggctg aagttctccc tgtccttgga gaacgtgctc tattacagcc |
| 1261 | caggaggggc cggcagtgac accttggtga ggtattttgc aaatgaacca tttgctgact |
| 1321 | tccaccgagt ggaaggattg caaggagtct acattgctac tctgattaat ggttctatga |
| 1381 | atgaggagaa catgagatcg gtcatcacct ttgacaaagg gggaacctgg gagtttcttc |
| 1441 | aggctccagc cttcacggga tatggagaga aaatcaattg tgagctttcc cagggctgtt |
| 1501 | cccttcatct ggctcagcgc ctcagtcagc tcctcaacct ccagctccgg agaatgccca |
| 1561 | tcctgtccaa ggagtcggct ccaggcctca tcatcgccac tggctcagtg ggaaagaact |
| 1621 | tggctagcaa gacaaacgtg tacatctcta gcagtgctgg agccaggtgg cgagaggcac |
| 1681 | ttcctggacc tcactactac acatggggag accacggcgg aatcatcacg gccattgccc |
| 1741 | agggcatgga aaccaacgag ctaaaataca gtaccaatga aggggagacc tggaaaacat |
| 1801 | tcatcttctc tgagaagcca gtgtttgtgt atggcctcct cacagaacct ggggagaaga |
| 1861 | gcactgtctt caccatcttt ggctcgaaca aagagaatgt ccacagctgg ctgatcctcc |
| 1921 | aggtcaatgc cacggatgcc ttgggagttc cctgcacaga gaatgactac aagctgtggt |
| 1981 | caccatctga tgagcggggg aatgagtgtt tgctgggaca caagactgtt ttcaaacggc |
| 2041 | ggacccccca tgccacatgc ttcaatggag aggactttga caggccggtg gtcgtgtcca |
| 2101 | actgctcctg cacccgggag gactatgagt gtgacttcgg tttcaagatg agtgaagatt |
| 2161 | tgtcattaga ggtttgtgtt ccagatccgg aattttctgg aaagtcatac tcccctcctg |
| 2221 | tgccttgccc tgtgggttct acttacagga gaacgagagg ctaccggaag atttctgggg |
| 2281 | acacttgtag cggaggagat gttgaagcgc gactggaagg agagctggtc ccctgtcccc |
| 2341 | tggcagaaga gaacgagttc attctgtatg ctgtgaggaa atccatctac cgctatgacc |
| 2401 | tggcctcggg agccaccgag cagttgcctc tcaccgggct acgggcagca gtggccctgg |
| 2461 | actttgacta tgagcacaac tgtttgtatt ggtccgacct ggccttggac gtcatccagc |
| 2521 | gcctctgttt gaatggaagc acagggcaag aggtgatcat caattctggc ctggagacag |
| 2581 | tagaagcttt ggcttttgaa cccctcagcc agctgcttta ctgggtagat gcaggcttca |
| 2641 | aaaagattga ggtagctaat ccagatggcg acttccgact cacaatcgtc aattcctctg |
| 2701 | tgcttgatcg tcccagggct ctggtcctcg tgccccaaga gggggtgatg ttctggacag |
| 2761 | actggggaga cctgaagcct gggatttatc ggagcaatat ggatggttct gctgcctatc |
| 2821 | acctggtgtc tgaggatgtg aagtggccca atggcatctc tgtggacgac cagtggattt |
| 2881 | actggacgga tgcctacctg gagtgcatag agcggatcac gttcagtggc cagcagcgct |
| 2941 | ctgtcattct ggacaacctc ccgcacccct atgccattgc tgtctttaag aatgaaatct |
| 3001 | actgggatga ctggtcacag ctcagcatat tccgagcttc caaatacagt gggtcccaga |
| 3061 | tggagattct ggcaaaccag ctcacggggc tcatggacat gaagattttc tacaagggga |
| 3121 | agaacactgg aagcaatgcc tgtgtgccca ggccatgcag cctgctgtgc ctgcccaagg |
| 3181 | ccaacaacag tagaagctgc aggtgtccag aggatgtgtc cagcagtgtg cttccatcag |
| 3241 | gggacctgat gtgtgactgc cctcagggct atcagctcaa gaacaatacc tgtgtcaaag |
| 3301 | aagagaacac ctgtcttcgc aaccagtatc gctgcagcaa cgggaactgt atcaacagca |
| 3361 | tttggtggtg tgactttgac aacgactgtg gagacatgag cgatgagaga aactgcccta |
| 3421 | ccaccatctg tgacctggac acccagtttc gttgccagga gtctgggact tgtatcccac |
| 3481 | tgtcctataa atgtgacctt gaggatgact gtggagacaa cagtgatgaa agtcattgtg |
| 3541 | aaatgcacca gtgccggagt gacgagtaca actgcagttc cggcatgtgc atccgctcct |
| 3601 | cctgggtatg tgacggggac aacgactgca gggactggtc tgatgaagcc aactgtaccg |
| 3661 | ccatctatca cacctgtgag gcctccaact tccagtgccg aaacgggcac tgcatccccc |
| 3721 | agcggtgggc gtgtgacggg gatacggact gccaggatgg ttccgatgag gatccagtca |
| 3781 | actgtgagaa gaagtgcaat ggattccgct gcccaaacgg cacttgcatc ccatccagca |
| 3841 | aacattgtga tggtctgcgt gattgctctg atggctccga tgaacagcac tgcgagcccc |
| 3901 | tctgtacgca cttcatggac tttgtgtgta agaaccgcca gcagtgcctg ttccactcca |
| 3961 | tggtctgtga cggaatcatc cagtgccgcg acgggtccga tgaggatgcg gcgtttgcag |
| 4021 | gatgctccca agatcctgag ttccacaagg tatgtgatga gttcggtttc cagtgtcaga |
| 4081 | atggagtgtg catcagtttg atttggaagt gcgacgggat ggatgattgc ggcgattatt |
| 4141 | ctgatgaagc caactgcgaa aaccccacag aagccccaaa ctgctcccgc tacttccagt |
| 4201 | ttcggtgtga gaatggccac tgcatcccca acagatggaa atgtgacagg gagaacgact |
| 4261 | gtggggactg gtctgatgag aaggattgtg gagattcaca tattcttccc ttctcgactc |
| 4321 | ctgggccctc cacgtgtctg cccaattact accgctgcag cagtgggacc tgcgtgatgg |
| 4381 | acacctgggt gtgcgacggg taccgagatt gtgcagatgg ctctgacgag gaagcctgcc |
| 4441 | ccttgcttgc aaacgtcact gctgcctcca ctcccaccca acttgggcga tgtgaccgat |
| 4501 | ttgagttcga atgccaccaa ccgaagacgt gtattcccaa ctggaagcgc tgtgacggcc |
| 4561 | accaagattg ccaggatggc cgggacgagg ccaattgccc cacacacagc accttgactt |
| 4621 | gcatgagcag ggagttccag tgcgaggacg gggaggcctg cattgtgctc tcggagcgct |
| 4681 | gcgacggctt cctggactgc tcggacgaga gcgatgaaaa ggcctgcagt gatgagttga |
| 4741 | ctgtgtacaa agtacagaat cttcagtgga cagctgactt ctctggggat gtgactttga |
| 4801 | cctggatgag gcccaaaaaa atgccctctg cttcttgtgt atataatgtc tactacaggg |
| 4861 | tggttggaga gagcatatgg aagactctgg agacccacag caataagaca aacactgtat |
| 4921 | taaaagtctt gaaaccagat accacgtatc aggttaaagt acaggttcag tgtctcagca |
| 4981 | aggcacacaa caccaatgac tttgtgaccc tgaggacccc agagggattg ccagatgccc |
| 5041 | ctcgaaatct ccagctgtca ctccccaggg aagcagaagg tgtgattgta ggccactggg |
| 5101 | ctcctcccat ccacacccat ggcctcatcc gtgagtacat tgtagaatac agcaggagtg |
| 5161 | gttccaagat gtgggcctcc cagagggctg ctagtaactt tacagaaatc aagaacttat |
| 5221 | tggtcaacac tctatacacc gtcagagtgg ctgcggtgac tagtcgtgga ataggaaact |
| 5281 | ggagcgattc taaatccatt accaccataa aaggaaaagt gatcccacca ccagatatcc |
| 5341 | acattgacag ctatggtgaa aattatctaa gcttcaccct gaccatggag agtgatatca |
| 5401 | aggtgaatgg ctatgtggtg aaccttttct gggcatttga cacccacaag caagagagga |
| 5461 | gaactttgaa cttccgagga agcatattgt cacacaaagt tggcaatctg acagctcata |
| 5521 | catcctatga gatttctgcc tgggccaaga ctgacttggg ggatagccct ctggcatttg |
| 5581 | agcatgttat gaccagaggg gttcgcccac ctgcacctag cctcaaggcc aaagccatca |
| 5641 | accagactgc agtggaatgt acctggaccg gcccccggaa tgtggtttat ggtattttct |
| 5701 | atgccacgtc ctttcttgac ctctatcgca acccgaagag cttgactact tcactccaca |
| 5761 | acaagacggt cattgtcagt aaggatgagc agtatttgtt tctggtccgt gtagtggtac |
| 5821 | cctaccaggg gccatcctct gactacgttg tagtgaagat gatcccggac agcaggcttc |
| 5881 | caccccgtca cctgcatgtg gttcatacgg gcaaaacctc cgtggtcatc aagtgggaat |
| 5941 | caccgtatga ctctcctgac caggacttgt tgtatgcaat tgcagtcaaa gatctcataa |
| 6001 | gaaagactga caggagctac aaagtaaaat cccgtaacag cactgtggaa tacaccctta |
| 6061 | acaagttgga gcctggcggg aaataccaca tcattgtcca actggggaac atgagcaaag |
| 6121 | attccagcat aaaaattacc acagtttcat tatcagcacc tgatgcctta aaaatcataa |
| 6181 | cagaaaatga tcatgttctt ctgttttgga aaagcctggc tttaaaggaa aagcatttta |
| 6241 | atgaaagcag gggctatgag atacacatgt ttgatagtgc catgaatatc acagcttacc |
| 6301 | ttgggaatac tactgacaat ttctttaaaa tttccaacct gaagatgggt cataattaca |
| 6361 | cgttcaccgt ccaagcaaga tgcctttttg gcaaccagat ctgtggggag cctgccatcc |
| 6421 | tgctgtacga tgagctgggg tctggtgcag atgcatctgc aacgcaggct gccagatcta |
| 6481 | cggatgttgc tgctgtggtg gtgcccatct tattcctgat actgctgagc ctgggggtgg |
| 6541 | ggtttgccat cctgtacacg aagcaccgga ggctgcagag cagcttcacc gccttcgcca |
| 6601 | acagccacta cagctccagg ctggggtccg caatcttctc ctctggggat gacctggggg |
| 6661 | aagatgatga agatgcccct atgataactg gattttcaga tgacgtcccc atggtgatag |
| 6721 | cctgaaagag ctttcctcac tagaaaccaa atggtgtaaa tattttattt gataaagata |
| 6781 | gttgatggtt tattttaaaa gatgcacttt gagttgcaat atgttatttt tatatgggcc |
| 6841 | aaaaacaaaa aacaaaaaaa aaaaaaagga aagaaaggaa tgaataaact ttgtagtaat |
| 6901 | caactgtgaa cttcaaacca ggttgatttt agtaacccaa ttgctttgat ttgacattaa |
| 6961 | tgtagtctta cagggctgtg cttgctgggc atgcttttac gtctgtgaga taatttcggt |
| 7021 | tcagtaaatt ggccaatctt tttatttttc taagacacag aaatgtattt aataaaaacc |
| 7081 | tcgagagagt gatgggtgga accccttctc cttgaaagtg tgtacagata ttccattttg |
| 7141 | tttggatata gtttatagga aagtgtgtgg atgtattatg gcggaaggtt tctttatgtt |
| 7201 | attttgttaa tttattggga ctctgtgtaa ggccaggctt tagtggtcat tagacaccac |
| 7261 | atgtgttatg agccccttac ccatagggtt gggggtggga agagaagcat atttttttgc |
| 7321 | cattccggaa gcaatccatt tttattcact tgtgtgtcat gtaatggtct ttggcaggag |
| 7381 | agagcactga gtcattgctg gagttcagtt caacagagct gcagcttggg aagccctgta |
| 7441 | agcccacagc ttcctctctt atattaattg atggaatttt actgtatgtg cctctgtaca |
| 7501 | agatgtagct ttgagagcta caaaatgata acactgcttt attacacact ggtttcattg |
| 7561 | tcattgcaaa aacttaccct ggttgtgggg gagagttcta gatctgtgcc atgatccata |
| 7621 | cactggctaa tagagtacat aatttttcca ttttccattt tttgttttta cttactactg |
| 7681 | aaggatctca gatgtaaaat tatgtatttg gtttgagatg gccacttatt gtccttaaaa |
| 7741 | atccatactg atatatgcag tcattttgaa ttggacagtg ccttctcttt ttttttctcc |
| 7801 | tcttcttcca tctccctcac ccatgccccc acccaatcta aagagacagt gctgtacatt |
| 7861 | ctcatagaga tagagaagat ctaaaaagtt gagactactc aatccagtta acaacagcag |
| 7921 | gagcactaga gtttgttcat ttattctctc tgtaaaacaa gctgtgcttt ttttcttctg |
| 7981 | cctttaaaat gccacccgtg tattcaaacc atggccactt gatacttatg tagaatccat |
| 8041 | cgtgggctga tgcaagccct ttatttaggc ttagtgttgt gggcaccaat gtcgagcatc |
| 8101 | gttgtgactt gtgctgtatg attctcactg aagaatttcc tttcagccaa gaagcagtga |
| 8161 | ggtctgggaa tattccaaag tcatgtctct gaatatgtgt ccttgacgtg caagctttgt |
| 8221 | aaaaccccat ccccgcttag gtgcgaggca tcaccttctc acaagtgttt agtttctttt |
| 8281 | aaccacaagt atcattcttg ggtgataata tagtttcatt ctacttaggg attgtttaga |
| 8341 | aaacaaagaa agagccaatt aaatttttta gtttttgaaa tttttattta tatgtatact |
| 8401 | tagatgagta ttttaagctg tcgaccttta gtttgccata cgggtaggac tgtatttcat |
| 8461 | gttaacaact ggtggtaatg ataagccttc ttctagcgta ttttctcttc tttcctgtca |
| 8521 | ctttcctaag tttttttttt taaagactgg aatttttttt ggctttatct tgtcttaccg |
| 8581 | tagagatttg ttcaaaactc taagccctac cacctcccct ttaataagct ctttaaatag |
| 8641 | ttgaatcatt aacaacctgg tgggaggcaa gtcatttaat tgaaccacta ggaagtgtat |
| 8701 | tttcttttct ttttctgcca actttttggt ggcatttgta aaagctgata taaaaggctc |
| 8761 | tgagatgtta ttttcagtta ttccataggc aagccttttt acagagcata tgtctccagt |
| 8821 | tggcagcttg agatatttcc gagcatccgg ttctagctac cagtgcctcc caatgcttag |
| 8881 | tgcacagtac tgtagactgg ccatcacccc tctccttgga aaatgccact gtgctgtttg |
| 8941 | aaaaaaagca gccttttagg gctagagtat tttatataaa cagaagagct aagttcctga |
| 9001 | agactaagct agatagctgc agctatatgt aaattgtata tttttatgaa cttttgaagc |
| 9061 | acacactcct gtttccctct gtgtagcttt gtggggattt catgtatata tgctgtctga |
| 9121 | aagaatccag aggttggagt gccaatagaa aatgaaaaca aatgccttgt actacaggca |
| 9181 | gcctctgaag gtgaccacat aactgtcttc actgtgacca atcggagtcc ctgcttgctt |
| 9241 | gtgaagaagg ggcttttgta ccttgttgga gatgccacct cagaagttca cactgtgcag |
| 9301 | gaaaaaggtt ttattctctc ctggcataca ttagaatgtc agatgcttgc atccatgtgg |
| 9361 | accacgatgg gcctctaaaa attggtgggc agggggtttg cttatgagtt ttctctggaa |
| 9421 | accgatttta ctcctggatg tattgaatgc cccttgagct ttatgagata cgagtccaca |
| 9481 | tggataaaat gttagagagt ggagttctac agaggattcc aggaagaggc catgtctgtg |
| 9541 | cagtcctagt tccagacagg tgagaagctc caggaactac tggctacctt gacaagctgg |
| 9601 | gtaaatagtt atcattctgg gtaactggtt gaaactctga cttttggaca agtaattcct |
| 9661 | ggggttctgt ctttggtagc atcaccaggg atatttgggt gggacagaca gaagacacac |
| 9721 | agctgcctgt tctctcctgc ccatcatgtt tggcccacta gatgaagctg tactcagcaa |
| 9781 | tttagggaat gtaacccttc tcagaactgg ccattttcag gggaagcttg ggagagcaat |
| 9841 | agtatggtga gccccttaga gatgagcgcc tactccttct tggcgaatgc tgccttcaga |
| 9901 | tgcttaccaa gtggtcactg catctagtaa gattatattt ccagtacact tccttagggc |
| 9961 | agaaacacca tcctatcagg tttggtcagt cccttcttca tgaagggagt catggggaat |
| 10021 | tcctgaaaat tttcttcctt ctgcagacag ttggatgagt cccttagaga aggcatccag |
| 10081 | agacataact aaactgaata tcatcccata ttgattttag gaattgactc taaaactctg |
| 10141 | tgcagaatct tgtgttggga ttgtatcttg acattcctgt tgtgttattt ttcttaactg |
| 10201 | gagtgtgtgc tgcctttcag gtacaatttt tgtgtaataa aagccagtgc attaagttta |
| 10261 | tatagactac tttctatgca agactgagat atggaataga taggaagaga tatgtactgc |
| 10321 | tgggtacatg gacagtaagt gtgttttcag atggagtacc agcaccgaaa atgggttgag |
| 10381 | ggaggatggg ttgtatgtat gtttctgccc actaattttg agcagccata ttatgaatta |
| 10441 | aatcgtcaca gccaagtaat aacccaagaa tggtatgagt ttcatgtgta atagctcaaa |
| 10501 | tggaataagc atgaatgctg gagtggacca ttatcctcaa atattctatg tcacttctca |
| 10561 | tttaaagact cttgttatga actattagaa actttaggca aaatcaaaag tatttgcggc |
| 10621 | aaaataaagg cctattctac tcttatttaa agtgaaacac tgtatacttg tttctctcca |
| 10681 | aagcgaaatt aagtatttat aatttcaatt gcctcgataa gtttccaagt cactgaaatc |
| 10741 | tgctgaaggt tttactgtat tgttgcacaa ctttaagata atttttgtct caatgtcaac |
| 10801 | ttttttcact gaataaaaat ttaactgggt caagaaaaca cctctttgaa aatccactgt |
| 10861 | ctctgtgtgt ctcgagctgt tctttagagc gcaataaaga tggctgacgc agtctccaaa |
| 10921 | cccc |
| SEQ ID NO 2: Homo sapiens SorLA polypeptide (NM_003105) | |
| MATRSSRRESRLPFLFTLVALLPPGALCEVWTQRLHGGSAPLPQDRGFLVVQGDP | |
| RELRLWARGDARGASRADEKPLRRKRSAALQPEPIKVYGQVSLNDSHNQMVVHW | |
| AGEKSNVIVALARDSLALARPKSSDVYVSYDYGKSFKKISDKLNFGLGNRSEAVIAQ | |
| FYHSPADNKRYIFADAYAQYLWITFDFCNTLQGFSIPFRAADLLLHSKASNLLLGFD | |
| RSHPNKQLWKSDDFGQTWIMIQEHVKSFSWGIDPYDKPNTIYIERHEPSGYSTVFR | |
| STDFFQSRENQEVILEEVRDFQLRDKYMFATKVVHLLGSEQQSSVQLWVSFGRKP | |
| MRAAQFVTRHPINEYYIADASEDQVFVCVSHSNNRTNLYISEAEGLKFSLSLENVLY | |
| YSPGGAGSDTLVRYFANEPFADFHRVEGLQGVYIATLINGSMNEENMRSVITFDKG | |
| GTWEFLQAPAFTGYGEKINCELSQGCSLHLAQRLSQLLNLQLRRMPILSKESAPGL | |
| IIATGSVGKNLASKTNVYISSSAGARWREALPGPHYYTWGDHGGIITAIAQGMETNE | |
| LKYSTNEGETWKTFIFSEKPVFVYGLLTEPGEKSTVFTIFGSNKENVHSWLILQVNA | |
| TDALGVPCTENDYKLWSPSDERGNECLLGHKTVFKRRTPHATCFNGEDFDRPVV | |
| VSNCSCTREDYECDFGFKMSEDLSLEVCVPDPEFSGKSYSPPVPCPVGSTYRRT | |
| RGYRKISGDTCSGGDVEARLEGELVPCPLAEENEFILYAVRKSIYRYDLASGATEQ | |
| LPLTGLRAAVALDFDYEHNCLYWSDLALDVIQRLCLNGSTGQEVIINSGLETVEALA | |
| FEPLSQLLYWVDAGFKKIEVANPDGDFRLTIVNSSVLDRPRALVLVPQEGVMFWTD | |
| WGDLKPGIYRSNMDGSAAYHLVSEDVKWPNGISVDDQWIYWTDAYLECIERITFS | |
| GQQRSVILDNLPHPYAIAVFKNEIYWDDWSQLSIFRASKYSGSQMEILANQLTGLM | |
| DMKIFYKGKNTGSNACVPRPCSLLCLPKANNSRSCRCPEDVSSSVLPSGDLMCDC | |
| PQGYQLKNNTCVKEENTCLRNQYRCSNGNCINSIWWCDFDNDCGDMSDERNCP | |
| TTICDLDTQFRCQESGTCIPLSYKCDLEDDCGDNSDESHCEMHQCRSDEYNCSSG | |
| MCIRSSWVCDGDNDCRDWSDEANCTAIYHTCEASNFQCRNGHCIPQRWACDGD | |
| TDCQDGSDEDPVNCEKKCNGFRCPNGTCIPSSKHCDGLRDCSDGSDEQHCEPLC | |
| THFMDFVCKNRQQCLFHSMVCDGIIQCRDGSDEDAAFAGCSQDPEFHKVCDEFG | |
| FQCQNGVCISLIWKCDGMDDCGDYSDEANCENPTEAPNCSRYFQFRCENGHCIP | |
| NRWKCDRENDCGDWSDEKDCGDSHILPFSTPGPSTCLPNYYRCSSGTCVMDTW | |
| VCDGYRDCADGSDEEACPLLANVTAASTPTQLGRCDRFEFECHQPKTCIPNWKR | |
| CDGHQDCQDGRDEANCPTHSTLTCMFQCEDGEACIVLSERCDGFLDCSDESDEK | |
| ACSDELTVYKVQNLQWTADFSGDVTLTWMRPKKMPSASCVYNVYYRVVGESIWK | |
| TLETHSNKTNTVLKVLKPDTTYQVKVQVQCLSKAHNTNDFVTLRTPEGLPDAPRNL | |
| QLSLPREAEGVIVGHWAPPIHTHGLIREYIVEYSRSGSKMWASQRAASNFTEIKNLL | |
| VNTLYTVRVAAVTSRGIGNWSDSKSITTIKGVIPPPDIHIDSYGENYLSFTLTMESDIK | |
| VNGYVVNLFWAFDTHKQERRTLNFRGSILSHKVGNLTAHTSYEISAWAKTDLGDS | |
| PLAFEHVMTRGVRPPAPSLKAKAIQTAVECTWTGPRNVVYGIFYATSFLDLYRNPK | |
| SLTTSLHNKTVIVSKDEQYLFLVRVVVPYQGPSSDYVVVKMIPDSRLPPRHLHVVH | |
| TGKTSVVIKWESPYDSPDQDLLYAIAVKDLIRKTDRSYKVKSRNSTVEYTLNKLEPG | |
| GKYHIIVQLGNMSKDSSIKITTVSLSAPDALKIITENDHVLLFWKSLALKEKHFNESR | |
| GYEIHMFDSAMNITAYLGNTTDNFFKISNLKMGHNYTFTVQARCLFGNQICGEPAIL | |
| LYDELGSGADASATQAARSTVAAVVVPILFLILLSLGVGFAILYTKHRRLQSSFTAFA | |
| NSHYSSRLGSAIFSSGDDLGEDDEDAPMITGFSDDVPMVIA | |
| SEQ ID NO 3: pre-Homo sapiens CTGF nucleotide (NC_000006) | |
| 1 | aaactcacac aacaactctt ccccgctgag aggagacagc cagtgcgact ccaccctcca |
| 61 | gctcgacggc agccgccccg gccgacagcc ccgagacgac agcccggcgc gtcccggtcc |
| 121 | ccacctccga ccaccgccag cgctccaggc cccgccgctc cccgctcgcc gccaccgcgc |
| 181 | cctccgctcc gcccgcagtg ccaaccatga ccgccgccag tatgggcccc gtccgcgtcg |
| 241 | ccttcgtggt cctcctcgcc ctctgcagcc gggtaagcgc cgggagcccc cgctgcggcc |
| 301 | ggcggctgcc agggagggac tcggggccgg ccggggaggg cgtgcgcgcc gaccgagcgc |
| 361 | cgctgaccgc cctgtcctcc ctgcagccgg ccgtcggcca gaactgcagc gggccgtgcc |
| 421 | ggtgcccgga cgagccggcg ccgcgctgcc cggcgggcgt gagcctcgtg ctggacggct |
| 481 | gcggctgctg ccgcgtctgc gccaagcagc tgggcgagct gtgcaccgag cgcgacccat |
| 541 | gcgacccgca caagggccta ttctgtcact tcggctcccc ggccaaccgc aagatcggcg |
| 601 | tgtgcaccgg taagacccgc agcccccacc gctaggtgtc cggccgcctc ctccctcacg |
| 661 | cccacccgcc cgctggaaaa agaaaccgct cggactgagt ttctttctcc agctgctgcc |
| 721 | agcccgcccc ctgcagccca gatcccaact cgcatccctg acgctctgga tgtgagagtg |
| 781 | ccccaatgcc tgacctctgc atcccccacc cctctcttcc cttcctcttc tccagccaaa |
| 841 | gatggtgctc cctgcatctt cggtggtacg gtgtaccgca gcggagagtc cttccagagc |
| 901 | agctgcaagt accagtgcac gtgcctggac ggggcggtgg gctgcatgcc cctgtgcagc |
| 961 | atggacgttc gtctgcccag ccctgactgc cccttcccga ggagggtcaa gctgcccggg |
| 1021 | aaatgctgcg aggagtgggt gtgtgacgag cccaaggacc aaaccgtggt tgggcctgcc |
| 1081 | ctcgcgggtg agtcgagtct tcctctaagt cagggtcgtg attctctccc agggagggag |
| 1141 | tcctaactgt gccgaccgaa cgggggaaat accttatcca ggcgttttac atggtgtttg |
| 1201 | tgtgctctgc tctcgcagct taccgactgg aagacacgtt tggcccagac ccaactatga |
| 1261 | ttagagccaa ctgcctggtc cagaccacag agtggagcgc ctgttccaag acctgtggga |
| 1321 | tgggcatctc cacccgggtt accaatgaca acgcctcctg caggctagag aagcagagcc |
| 1381 | gcctgtgcat ggtcaggcct tgcgaagctg acctggaaga gaacattaag gtacatgttc |
| 1441 | tgctcctatt aactattttt cacaggaaaa acagtggata ggacccaact tagggctctt |
| 1501 | gccacgcttg ttagtataag cccgttatct ccaaaactat ctaaccattg agctgttttg |
| 1561 | ctggaatgag agcttgtgta atagcaacca ccagttttcc actacgaaat cttccacagg |
| 1621 | gttagttaat tcaagacatt ccaagagagg ctctggctat ttttggacat agcaaatgag |
| 1681 | actcaaactt cctcccctca aaatataaac agaagtcaga caacagaaga ctaaaacaca |
| 1741 | gagggttgaa gaaagccact cctcttgtag agtcgctgat tttttttttt cctctctctt |
| 1801 | ttcccttgtc ttccttagaa gggcaaaaag tgcatccgta ctcccaaaat ctccaagcct |
| 1861 | atcaagtttg agctttctgg ctgcaccagc atgaagacat accgagctaa attctgtgga |
| 1921 | gtatgtaccg acggccgatg ctgcaccccc cacagaacca ccaccctgcc ggtggagttc |
| 1981 | aagtgccctg acggcgaggt catgaagaag aacatgatgt tcatcaagac ctgtgcctgc |
| 2041 | cattacaact gtcccggaga caatgacatc tttgaatcgc tgtactacag gaagatgtac |
| 2101 | ggagacatgg catgaagcca gagagtgaga gacattaact cattagactg gaacttgaac |
| 2161 | tgattcacat ctcatttttc cgtaaaaatg atttcagtag cacaagttat ttaaatctgt |
| 2221 | ttttctaact gggggaaaag attcccaccc aattcaaaac attgtgccat gtcaaacaaa |
| 2281 | tagtctatca accccagaca ctggtttgaa gaatgttaag acttgacagt ggaactacat |
| 2341 | tagtacacag caccagaatg tatattaagg tgtggcttta ggagcagtgg gagggtacca |
| 2401 | gcagaaaggt tagtatcatc agatagcatc ttatacgagt aatatgcctg ctatttgaag |
| 2461 | tgtaattgag aaggaaaatt ttagcgtgct cactgacctg cctgtagccc cagtgacagc |
| 2521 | taggatgtgc attctccagc catcaagaga ctgagtcaag ttgttcctta agtcagaaca |
| 2581 | gcagactcag ctctgacatt ctgattcgaa tgacactgtt caggaatcgg aatcctgtcg |
| 2641 | attagactgg acagcttgtg gcaagtgaat ttgcctgtaa caagccagat tttttaaaat |
| 2701 | ttatattgta aatattgtgt gtgtgtgtgt gtgtgtatat atatatatat gtacagttat |
| 2761 | ctaagttaat ttaaagttgt ttgtgccttt ttatttttgt ttttaatgct ttgatatttc |
| 2821 | aatgttagcc tcaatttctg aacaccatag gtagaatgta aagcttgtct gatcgttcaa |
| 2881 | agcatgaaat ggatacttat atggaaattc tgctcagata gaatgacagt ccgtcaaaac |
| 2941 | agattgtttg caaaggggag gcatcagtgt ccttggcagg ctgatttcta ggtaggaaat |
| 3001 | gtggtagcct cacttttaat gaacaaatgg cctttattaa aaactgagtg actctatata |
| 3061 | gctgatcagt tttttcacct ggaagcattt gtttctactt tgatatgact gtttttcgga |
| 3121 | cagtttattt gttgagagtg tgaccaaaag ttacatgttt gcacctttct agttgaaaat |
| 3181 | aaagtgtata ttttttctat aaa |
| SEQ ID NO 4: pre-Homo sapiens CTGF polypeptide (Swiss-Prot P29279) | |
| MTAASMGPVR VAFVVLLALC SRPAVGQNCS GPCRCPDEPA PRCPAGVSLV | |
| LDGCGCCRVCAKQLGELCTE RDPCDPHKGL FCHFGSPANR KIGVCTAKDG AP- | |
| CIFGGTVY RSGESFQSSCKYQCTCLDGA VGCMPLCSMD VRLPSPDCPF | |
| PRRVKLPGKC CEEWVCDEPK DQTVVGPALA AYRLEDTFGP DPTMIRANCL | |
| VQTTEWSACS KTCGMGISTR VTNDNASCRL EKQSRLCMVR PCEADLEENI | |
| KKGKKCIRTP KISKPIKFEL SGCTSMKTYR AKFCGVCTDG RCCTPHRTTT | |
| LPVEFKCPDG EVMKKNMMFI KTCACHYNCP GDNDIFESLY YRKMYGDMA | |
| SEQ ID NO 5: Mature Homo sapiens CTGF polypeptide (Swiss-Prot P29279) | |
| QNCS GPCRCPDEPA PRCPAGVSLV LDGCGCCRVCAKQLGELCTE RDPCDPHKGL | |
| FCHFGSPANR KIGVCTAKDG APCIFGGTVY RSGES- | |
| FQSSCKYQCTCLDGAVGCMPLCSMD VRLPSPDCPF PRRVKLPGKC CEEWVCDEPK | |
| DQTVVGPALAAYRLEDTFGP DPTMIRANCL VQTTEWSACS KTCGMGISTR | |
| VTNDNASCRL EKQSRLCMVRPCEADLEENI KKGKKCIRTP KISKPIKFEL SGCTSMKTYR | |
| AKFCGVCTDG RCCTPHRTTT LPVEFKCPDG EVMKKNMMFI KTCACHYNCP | |
| GDNDIFESLY YRKMYGDMA | |
| SEQ ID NO 6: pre-Mus musculus CTGF nucleotide (NM_010217) | |
| 1 | agttctttgg cgagccggct cccgggagcg tataaaagcc agcgccgccc gcctagtctc |
| 61 | acacagctct tctctccaag aagactcagc cagatccact ccagctccga ccccaggaga |
| 121 | ccgacctcct ccagacggca gcagccccag cccagccgac aaccccagac gccaccgcct |
| 181 | ggagcgtcca gacaccaacc tccgcccctg tccgaatcca ggctccggcc gcgcctctcg |
| 241 | tcgcctctgc accctgctgt gcatcctcct accgcgtccc gatcatgctc gcctccgtcg |
| 301 | caggtcccat cagcctcgcc ttggtgctcc tcgctctctg cacccggcct gctatgggcc |
| 361 | aggactgcag cgcgcaatgt cagtgcgcag ccgaagcagc gccgcactgc cccgccggcg |
| 421 | tgagcctggt gctggacggc tgcggctgct gccgcgtctg cgccaagcag ctgggagaac |
| 481 | tgtgtacgga gcgtgacccc tgcgacccac acaagggcct cttctgcgat ttcggctccc |
| 541 | ccgccaaccg caagatcgga gtgtgcactg ccaaagatgg tgcaccctgt gtcttcggtg |
| 601 | ggtcggtgta ccgcagcggt gagtccttcc aaagcagctg caaataccaa tgcacttgcc |
| 661 | tggatggggc cgtgggctgc gtgcccctgt gcagcatgga cgtgcgcctg cccagccctg |
| 721 | actgcccctt cccgagaagg gtcaagctgc ctgggaaatg ctgcgaggag tgggtgtgtg |
| 781 | acgagcccaa ggaccgcaca gcagttggcc ctgccctagc tgcctaccga ctggaagaca |
| 841 | catttggccc agacccaact atgatgcgag ccaactgcct ggtccagacc acagagtgga |
| 901 | gcgcctgttc taagacctgt gggatgggca tctccacccg agttaccaat gacaatacct |
| 961 | tctgcagact ggagaagcag agccgcctct gcatggtcag gccctgcgaa gctgacctgg |
| 1021 | aggaaaacat taagaagggc aaaaagtgca tccggacacc taaaatcgcc aagcctgtca |
| 1081 | agtttgagct ttctggctgc accagtgtga agacatacag ggctaagttc tgcggggtgt |
| 1141 | gcacagacgg ccgctgctgc acaccgcaca gaaccaccac tctgccagtg gagttcaaat |
| 1201 | gccccgatgg cgagatcatg aaaaagaata tgatgttcat caagacctgt gcctgccatt |
| 1261 | acaactgtcc tggggacaat gacatctttg agtccctgta ctacaggaag atgtacggag |
| 1321 | acatggcgta aagccaggaa gtaagggaca cgaactcatt agactataac ttgaactgag |
| 1381 | ttgcatctca ttttcttctg taaaaacaat tacagtagca cattaattta aatctgtgtt |
| 1441 | tttaactacc gtgggaggaa ctatcccacc aaagtgagaa cgttatgtca tggccataca |
| 1501 | agtagtctgt caacctcaga cactggtttc gagacagttt acacttgaca gttgttcatt |
| 1561 | agcgcacagt gccagaatgc acactgaggt gagtctcctg gaacagtgga gatgccagga |
| 1621 | gaaagaaaga caggtactag ctgaggttat tttaaaagca gcagtgtgcc tactttttgg |
| 1681 | agtgtaaccg gggagggaaa ttatagcatg cttgcagaca gacctgctct agcgagagct |
| 1741 | gagcatgtgt cctccactag atgaggctga gtccagctgt tctttaagaa cagcagtttc |
| 1801 | agctctgacc attctgattc cagtgacact tgtcaggagt cagagccttg tctgttagac |
| 1861 | tggacagctt gtggcaagta agtttgcctg taacaagcca gatttttatt gatattgtaa |
| 1921 | atattgtgga tatatatata tatatatata tatatatttg tacagttatc taagttaatt |
| 1981 | taaagtcatt tgtttttgtt ttaagtgctt ttgggatttt aaactgatag cctcaaactc |
| 2041 | caaacaccat aggtaggaca cgaagcttat ctgtgattca aaacaaagga gatactgcag |
| 2101 | tgggaattgt gacctgagtg actctctgtc agaacaaatg ctgtgcaggt gataaagcta |
| 2161 | tgtattggaa gtcagatttc tagtaggaaa tgtggtcaaa tccctgttgg tgaacaaatg |
| 2221 | gcctttatta agaaatggct ggctcagggt aaggtccgat tcctaccagg aagtgcttgc |
| 2281 | tgcttctttg attatgactg gtttggggtg gggggcagtt tatttgttga gagtgtgacc |
| 2341 | aaaagttaca tgtttgcacc tttctagttg aaaataaagt atatatatat tttttatatg |
| 2401 | aaaaaaaaaa aaaaaaaa |
| SEQ ID NO 7: pre-Mus musculus CTGF polypeptide (P29268) | |
| MLASVAGPIS LALVLLALCT RPATGQDCSA QCQCAAEAAP HCPAGVSLVL | |
| DGCGCCRVCA KQLGELCTER DPCDPHKGLF CDFGSPANRK IGVCTAKDGA | |
| PCVFGGSVYR SGESFQSSCK YQCTCLDGAV GCVPLCSMDV RLPSPDCPFP | |
| RRVKLPGKCC EEWVCDEPKD RTAVGPALAA YRLEDTFGPD PTMMRANCLV | |
| QTTEWSACSK TCGMGISTRV TNDNTFCRLE KQSRLCMVRP CEADLEENIK | |
| KGKKCIRTPK IAKPVKFELS GCTSVKTYRA KFCGVCTDGR CCTPHRTTTL | |
| PVEFKCPDGE IMKKNMMFIK TCACHYNCPG DNDIFESLYY RKMYGDMA | |
| SEQ ID NO 8: Mature Mus musculus CTGF polypeptide (P29268) | |
| QDCSAQCQCAAEAAPHCPAGVSLVLDGCGCCRVCAKQLGELCTERDPCDPHKGLFCDFG | |
| SPANRKIGVCTAKDGAPCVFGGSVYRSGESFQSSCKYQCTCLDGAVGCVPLCSMDVRLPS | |
| PDCPFPRRVKLPGKCCEEWVCDEPKDRTAVGPALAAYRLEDTFGPDPTMMRANCLVQTT | |
| EWSACSKTCGMGISTRVTNDNTFCRLEKQSRLCMVRPCEADLEENIKKGKKCIRTPKIAKPV | |
| KFELSGCTSVKTYRAKFCGVCTDGRCCTPHRTTTLPVEFKCPDGEIMKKNMMFIKTCACHY | |
| NCPGDNDIFESLYYRKMYGDMA | |
| SEQ ID NO 9: pre-Rattus Norvegicus CTGF nucleotide (AB023068) | |
| 1 | ctccaagaag actcagccag acccactcca gctccgaccc taggagaccg acctcctcca |
| 61 | gacggcagca gccccagccc agtggacaac cccaggagcc accacctgga gcgtccggac |
| 121 | accaacctcc gccccgagac cgagtccagg ctccggccgc gcccctcgtc gcctctgcac |
| 181 | cccgctgtgc gtcctcctgc cgcgccccga ccatgctcgc ctccgtcgcg ggtcccgtta |
| 241 | gcctcgcctt ggtgctcctc ctctgcaccc ggcctgccac cggccaggac tgcagcgcgc |
| 301 | agtgtcagtg cgcacgtgaa gcggcgccgc gctgccccgc cggcgtgagc ctggtgctgg |
| 361 | acggctgcgg ctgctgccgc gtctgcgcca agcagctggg agaactgtgc acggagcgtg |
| 421 | atccctgcga cccacacaag ggtctcttct gcgacttcgg ctcccccgcc aaccgcaaga |
| 481 | ttggcgtgtg ccctgccaaa gatggtgcac cctgtgtctt cggtgggtcc gtgtaccgca |
| 541 | gcggcgagtc cttccaaagc agttgcaaat accagtgcac ttgcctggat ggggccgtgg |
| 601 | gctgtgtgcc cctgtgcagc atggacgtgc gcctgcccag ccctgactgc cccttcccga |
| 661 | gaagggtcaa gctgcccggg aaatgctgtg aggagtgggt gtgtgatgag cccaaggacc |
| 721 | gcacagtggt tggccctgcc ctagctgcct accgactgga agacacattt ggccctgacc |
| 781 | caactatgat gcgagccaac tgcctggtcc agaccacaga gtggagcgcc tgttctaaga |
| 841 | cctgtgggat gggcatctcc acccgggtta ccaatgacaa taccttctgc aggctggaga |
| 901 | agcagagtcg tctctgcatg gtcaggccct gtgaagctga cctagaggaa aacattaaga |
| 961 | agggcaaaaa gtgcatccgg acgcctaaaa ttgccaagcc tgtcaagttt gagctttctg |
| 1021 | gctgcaccag tgtgaagacc taccgggcta agttctgtgg ggtgtgcacg gacggccgct |
| 1081 | gctgcacacc gcacagaacc accacactgc cggtggagtt caagtgcccc gatggcgaga |
| 1141 | tcatgaaaaa gaacatgatg ttcatcaaga cctgtgcctg ccattacaac tgtcccgggg |
| 1201 | acaatgacat ctttgagtcc ttgtactaca ggaagatgta tggagacatg gcgtaaagcc |
| 1261 | agggagtaag ggacacgaac tcatttagac tataacttga actgagttac atctcatttt |
| 1321 | cttctgtaaa aaaaacaaaa agggttacag tagcacatta atttaaatct gggttcctaa |
| 1381 | ctgctgtggg agaaaacacc ccaccgaagt gagaaccgtg tgtcattgtc atgcaaatag |
| 1441 | cctgtcaatc tcagacactg gtttcgagac agtttagact tgacagttgt tcactagcgc |
| 1501 | acagtgacag aacgcacact aaggtgagcc tcctggaaga gtggagatgc caggagaaag |
| 1561 | acaggtacta gctgaggtca ttttaaaagc agcgatatgc ctactttttg gagtgtgaca |
| 1621 | ggggagggac attatagctt gcttgcagac agacctgctc tagcaagagc tgggtgtgtg |
| 1681 | tcctccactc ggtgaggctg aagccagcta ttctttcagt aagaacagca gtttcagcgc |
| 1741 | tgacattctg attccagtga cactggtcgg gagtcagaac cttgtctatt agactggaca |
| 1801 | gcttgtggca agtgaatttg ccggtaacaa gccagatttt tatggatctt gtaaatattg |
| 1861 | tggataaata tatatatttg tacagttatc taagttaatt taaagacgtt tgtgcctatt |
| 1921 | gttcttgttt taagtgcttt tggaattttt aaactgatag cctcaaactc caaacaccat |
| 1981 | cgataggaca taaagcttgt ctgtgattca aaacaaagga gatactgcag tggaaactgt |
| 2041 | aacctgagtg actgtctgtc agaacatatg gtacgtagac ggtaaagcaa tggatcagaa |
| 2101 | gtcagatttc tagtaggaaa tgtaaaatca ctgttggcga acaaatggcc tttattaaga |
| 2161 | aatggcttgc tcagggtaac tggtcagatt tccacgagga agtgtttgct gcttctttga |
| 2221 | ctatgactgg tttgggaggc agtttatttg ttgagagtgt gaccaaaagt tacatgtttg |
| 2281 | cacctttcta gttgaaaata aagtatatat attttttata tgaaaaaaaa aaaaaaaa |
| SEQ ID NO 10: pre-Rattus Norvegicus CTGF polypeptide (Q9R1E9) | |
| MLASVAGPVS LALVLLLCTR PATGQDCSAQ CQCAAEAAPR CPAGVSLVLD | |
| GCGCCRVCAK QLGELCTERD PCDPHKGLFC DFGSPANRKI GVCTAKDGAP | |
| CVFGGSVYRS GESFQSSCKYQCTCLDGAVG CVPLCSMDVR LPSPDCPFPR | |
| RVKLPGKCCE EWVCDEPKDR TVVGPALAAYRLEDTFGPDP TMMRANCLVQ | |
| TTEWSACSKT CGMGISTRVT NDNTFCRLEK QSRLCMVRPC EADLEENIKK | |
| GKKCIRTPKI AKPVKFELSG CTSVKTYRAK FCGVCTDGRC CTPHRTTTLP | |
| VEFKCPDGEI MKKNMMFIKT CACHYNCPGD NDIFESLYYR KMYGDMA | |
| SEQ ID NO 11: Mature Rattus Norvegicus CTGF polypeptide (Q9R1E9) | |
| QDCSAQCQCAAEAAPRCPAGVSLVLDGCGCCRVCAKQLGELCTERDPCDPHKGLFCDFG | |
| SPANRKIGVCTAKDGAPCVFGGSVYRSGESFQSSCKYQCTCLDGAVGCVPLCSMDVRLPS | |
| PDCPFPRRVKLPGKCCEEWVCDEPKDRTVVGPALAAYRLEDTFGPDPTMMRANCLVQTT | |
| EWSACSKTCGMGISTRVTNDNTFCRLEKQSRLCMVRPCEADLEENIKKGKKCIRTPKIAKPV | |
| KFELSGCTSVKTYRAKFCGVCTDGRCCTPHRTTTLPVEFKCPDGEIMKKNMMFIKTCACHY | |
| NCPGDNDIFESLYYRKMYGDMA | |
| SEQ ID NO 12: pre-Bos taurus CTGF nucleotide (NM_174030.2) | |
| 1 | gaggcagcca gtgcgagtag gcagccagtg cgactcccac cgccggcgac ccacctcctc |
| 61 | cagtccgacg gcagccgccc cggccgacag ctcccgagac agcagcccgg cgctccccgg |
| 121 | cccacgcctc cggcccaaac ctgcgccgcc cgcccggcca gccgcctctc gccgcccgcc |
| 181 | gccctccgga cacagcgccc cgaccatgtc agccaccggc ctgggcccgg tccgctgcgc |
| 241 | cttcgtgctc ctgctcgccc tctgcagccg gcccgcctcc agccaggact gctgcagcgc |
| 301 | cccgtgccag tgccctgccg gcccggcgcc gcgctgcccc gccggcgtca gcttggtgct |
| 361 | ggacggctgc ggctgctgcg tgtgcgccaa gcagctgagc gagttgtgta ccgagcgcga |
| 421 | cccctgcgat ccgcacaagg gcctcttctg cgacttcggc tccccaacca accgcaagat |
| 481 | cggcgtgtgc accgctaaag atggtgcccc ctacattttc ggaggaactg tgtaccagag |
| 541 | cggagagtct ttccagagca gctgcaaata ccagtgcacg tgtctggacg ggtcggtggg |
| 601 | ctgcgtgccc ctatgcagcg tggacgtccg cctgcccagc cccgactgcc ccttcccacg |
| 661 | gagggtcaaa ctgcccggga aatgctgcga ggaatgggtg tcccgtgatg agaaggagca |
| 721 | caccgtggtc ggccctgcgc tggcagctta ccggctggaa gacacgtttg gcccagaccc |
| 781 | aaccatgatc cgagccaact gccaggtcca gaccacagag tggagtgcct attccaagac |
| 841 | ctgcggaatg ggcatctcca cccgggttac caatgacaac gcattctgca ggctggagaa |
| 901 | gcagagccgc ctctgcatgg tcaggccttg cgaagctgac ctggaggaga acattaagaa |
| 961 | aggcaaaaag tgcatccgga ctcccaaaat ctccaagcct atcaagtttc agctttctgg |
| 1021 | ctgcaccagc atgaagacat accgagctaa attcttcgga gtgtgcacag acgggcggtg |
| 1081 | ctgcaccccc cacagaacca ccacccttcc cgtggagttc aagtgtcctg atggggaggt |
| 1141 | catgaagaag agcatgatgt tcatcaagac ctgtgcctgc cattacaact gccccggaga |
| 1201 | caatgacatc ttcgagtcac tgtactacag gaagatgtat ggagacatgg cctaaagcca |
| 1261 | gagacagtga gacacgtgaa cattttaggc tgtcacttga atcgattcac atctcatttt |
| 1321 | tgtgtacacg tgatttcagt ggcacaagtt atttaaatct gtgcttctaa ctggggaaaa |
| 1381 | gaaaaattcc caccaaattc aaaatactgt gccatgtgat attcaaacga atagtccgtc |
| 1441 | aaccccagac actggtttga agaaattgag acttgatcat aggactgtat tagtgcacag |
| 1501 | caccagcatg tatgctagga gcagtgggag gaggccagta gaaagccttg tcatctttag |
| 1561 | gggtagtgat gtgactgcta tttggagtgt cactgaaaag gaaaacttta gcatgctcac |
| 1621 | tgatctgcct atagctccag caacagctcg gatgtgcgtt ctccagccat catgaggctg |
| 1681 | agtcaagttc gtctctaagt cagaacagca gattcagcta tgacattctg attcaaggac |
| 1741 | attgttcagg aatcagaatt ctgtctatta gactggacag cttgtggcaa gctaatttgc |
| 1801 | ctgtaacaag ccagattttt ttttattgat actgtaaata ttgtgtgtat atatatatat |
| 1861 | ttgtacagtt atctaagtta atttaaagtt gtttgtgcct ttttgtttat gtttttaatg |
| 1921 | ctttgatatt tcaagcgtta gcctcaattc tgaacaccat aggtaggacg gaaagcttgt |
| 1981 | ctgataattc aaagcatgaa atggatattc aaatagaatt tctgtgcagt tggagcaaca |
| 2041 | gtccatccaa acaggttgtt tgctgaaggt gaggcagtga tgtccttcaa agtctgatct |
| 2101 | gtaggttgga aacgtggtag cctcctttta acgaacaaat gacctttatt aaaaatgagt |
| 2161 | agctctgtat agctgattgg tttttccacc tggaagcatt tgtctctact ttgactatga |
| 2221 | ctgttttttg gacagattta tttgttgaga gtgtgaccaa aagttacatg tttgcacctt |
| 2281 | tctagttgaa aataaagtat atattttttc tataaaaaaa aaaaaaaaaa |
| SEQ ID NO 13: pre-Bos taurus CTGF polypeptide (O18739) | |
| MSATGLGPVR CAFVLLLALC SRPASSQDCS APCQCPAGPA PRCPAGVSLV | |
| LDGCGCCRVC AKQLSELCTE RDPCDPHKGL FCDFGSPANR KIGVCTAKDG | |
| APCVFGGTVY QSGESFQSSC KYQCTCLDGS VGCVPLCSVD VRLPSPDCPF | |
| PRRVKLPGKC CEEWVCDEPK EHTVVGPALA AYRPEDTFGP DPTMIRANCL | |
| VQTTEWSACS KTCGMGISTR VTNDNAFCRL EKQSRLCMVR PCEADLEENI | |
| KKGKKCIRTP KISKPIKFEL SGCTSMKTYR AKFCGVCTDG RCCTPHRTTT | |
| LPVEFKCPDG EVMKKSMMFI KTCACHYNCP GDNDIFESLY YRKMYGDMA | |
| SEQ ID NO 14: Mature Bos taurus CTGF polypeptide | |
| QDCSAPCQCPAGPAPRCPAGVSLVLDGCGCCRVCAKQLSELCTERDPCDPHKGLFCDFG | |
| SPANRKIGVCTAKDGAPCVFGGTVYQSGESFQSSCKYQCTCLDGSVGCVPLCSVDVRLPS | |
| PDCPFPRRVKLPGKCCEEWVDEPKEHTVVGPALAAYRPEDTFGPDPTMIRANCLVQTTE | |
| WSACSKTCGMGISTRVTNDNAFCRLEKQSRLCMVRPCEADLEENIKKGKKCIRTPKISKPIK | |
| FELSGCTSMKTYRAKFCGVCTDGRCCTPHRTTTLPVEFKCPDGEVMKKSMMFIKTCACHY | |
| NCPGDNDIFESLYYRKMYGDMA | |
| SEQ ID NO 15: pre-Sus scrofa CTGF nucleotide (NM_213833) | |
| 1 | gcacgagctg agaggaggca gtcagcccga ctcccaccgc cgacgaccca ccgccttcag |
| 61 | tccgacggca gccgccccgg ccgacagctc ccgagacagc cgcccggcgc gttccaggct |
| 121 | cccgcctccg gcccgaaccc gcgccgcccg ctcggcccgc cacgccaccc cagccccgcc |
| 181 | gccagccgcc cgcagcgccc cgaccatgtc cgccaccggc ctgagcccgg tccgctgcgc |
| 241 | cttcgtgctc ctgctcgccc tctgcagccg gcccgcctct ggccaggact gcagcggcca |
| 301 | atgccagtgc gcggccggga agcgccgcgc ttgccccgcc ggcgtcagct cgttgctgga |
| 361 | aggctgcggc tgctgccgat tgtgcgccaa gcacttgggt gacttgtgca cggagcgcgc |
| 421 | accctgcgac ccgcacaagg gcctcttctg tgacttcggc tccccggcca accgcaagat |
| 481 | cggagtgtgc acagccaaag attgtgcccc ctgcgtcttt ggaggaacgg tgtaccggag |
| 541 | cggagagtcc ttccagagca gctgcaaata ccagtgcact tgcctggacg gggccgtggg |
| 601 | ctgcgtgccc ctgtgcagca tggacgtccg cctgcccagc cccgactgcc ccttcccacg |
| 661 | gagggtcaag ctgcccggga aatgctgcga ggagtgggtg tgtgacgagc ccaaggacca |
| 721 | caccgtggtc gggcctgccc tggcggctta ccgactggaa gacacgtttg gcccagaccc |
| 781 | aactatgatg cgagccaact gcctggtcca gaccacagag tggagtgcct gttccaagac |
| 841 | ctgtgggatg ggcatctcca cccgggttac caatgacaac gcttcttgca gactggagaa |
| 901 | gcagagccgc ctctgcatgg tcaggccttg tgaagctgac ctggaagaga acattaagaa |
| 961 | gggcaaaaag tgcatccgta cccccaaaat ctccaagccc gtcaagtttg agctttccgg |
| 1021 | ctgcaccagt gtgaagacat accgggctaa gttctgcggg gtatgcacag acggccgctg |
| 1081 | ctgcacccct cacagaacca ccacccttcc tgtggagttc aagtgtcccg acggtgaggt |
| 1141 | catgaagaag agcatgatgt tcatcaagac ttgtgcctgc cattacaact gccccgggga |
| 1201 | caatgacatc ttcgagtccc tgtactacag gaagatgtat ggagacatgg cctaaagcca |
| 1261 | gagagagtga gaccatgaac acttgagact gtcactttga acttgattca catctcattt |
| 1321 | ttgcgtaaac atgattttca gtagcacagg ttatttaaag ccagagagag tgagacccat |
| 1381 | gaacacttga gactgtcact tgaactgatt cacatctcat ttttgcgtaa acatgatttc |
| 1441 | agtagcacag gttatttaaa tctgtttttc taatggggga caaaggaaaa ttcccaccaa |
| 1501 | cattcaaaat cgtgtgccat gtgacattca aaaaaagatc tatcaaaccc agacactggt |
| 1561 | ttgaagaaag ttcagacttg accgtgggga cggtatta |
| SEQ ID NO 16: pre-Sus scrofa CTGF polypeptide (O97765) | |
| MSATGLSPVR CAFVLLLALC SRPASGQDCS GQCQCAAGKR RACPAGVSSL | |
| LEGCGCCRLC AKHLGDLCTE RAPCDPHKGL FCDFGSPANR KIGVCTAKDC | |
| APCVFGGTVY RSGESFQSSC KYQCTCLDGA VGCVPLCSMD VRLPSPDCPF | |
| PRRVKLPGKC CEEWVCDEPK DHTVVGPALA AYRLEDTFGP DPTMMRANCL | |
| VQTTEWSACS KTCGMGISTR VTNDNASCRL EKQSRLCMVR PCEADLEENI | |
| KKGKKCIRTP KISKPVKFEL SGCTSVKTYR AKFCGVCTDG RCCTPHRTTT | |
| LPVEFKCPDG EVMKKSMMFI KTCACHYNCP GDNDIFESLY YRKMYGDMA | |
| SEQ ID NO 17: Mature Sus scrofa CTGF polypeptide (O97765) | |
| QDCSGQCQCAAGKRRACPAGVSLVLDGCGCCRLCAKQLGELCTERDPCDPHKGLFCDFG | |
| SPANRKIGVCTAKDGAPCVFGGTVYRSGESFQSSCKYQCTCLDGAVGCVPLCSMDVRLPS | |
| PDCPFPRRVKLPGKCCEEWVCDEPKDHTVVGPALAAYRLEDTFGPDPTMMRANCLVQTT | |
| EWSACSKTCGMGISTRVTNDNAFCRLEKQSRLCMVRPCEADLEENIKKGKKCIRTPKISKP | |
| VKFELSGCTSVKTYRAKFCGVCTDGRCCTPHRTTTLPVEFKCPDGEVMKKSMMFIKTCAC | |
| HYNCPGDNDIFESLYYRKMYGDMA | |
| SEQ ID NO 18: Pre-Xenopus CTGF nucleotide (BC094492) | |
| 1 | tcccaagaca aggaaccaaa tacagcgaac gagggaaaag gaaagaggct tgagagaaaa |
| 61 | gaaactaagg gaactacaca atcgccaacg agatcacttt gagatcaaga cttgagagaa |
| 121 | cgacttttct ttggagtaga agagaactaa tctgctgctg tacaatgtct gcaggaaaag |
| 181 | tgacagctgt gctcctcttt gctctcttct gctgggtgtc tgatgcccag gagtgtaatg |
| 241 | gggaatgcca gtgcccaaat aaagtgcctg tgtgtgatcc tggagtcagc ctggtgcagg |
| 301 | atggctgtgg ctgctgcaag gtgtgctcca agcagctagg ggagctgtgc accgaaagag |
| 361 | atgtgtgcga cccacacaaa gggctcttct gtgacttcgg atccagagtg aacaggaaaa |
| 421 | ttggagtttg cactgccagg gaaggtgccc cttgtgtgtt cggaggcact gtgtatagaa |
| 481 | gtggggagtc tttccagagc agctgcaagt accaatgtac ttgtatcgat gggggtgtgg |
| 541 | gatgtgttcc actttgcagc atggacatcc gcctgcccag ccccgagtgc cccttcccac |
| 601 | gaagagtcaa actgcctggc aagtgctgtg aagaatgggt ctgtgatcaa cctcaagaga |
| 661 | gaaccttagt cggacctgct ttgcctgctt tcagaatgga agaaacctat ggtcctgatc |
| 721 | catccctaat ccgcgccaac tgcctagtac agactactga atggagtgct tgctcaaaga |
| 781 | cctgtggcat gggaatctcc accagggtca ctaatgacaa tgagcactgc agactggaga |
| 841 | aacagagcag actctgcatg gtcaggccct gtgaagctga cctagaagaa aacatcaaga |
| 901 | aagggaaaaa gtgcatccgt acaccaaaaa tttcaaaacc agtgaagttt gaattttccg |
| 961 | gctgcaccag cgtaaaaacc tacagagcca agttctgcgg tgtgtgcact gatggtcgtt |
| 1021 | gttgcactcc tcacagaacc gctaccctcc cagttgagtt caagtgccct gatggtgagg |
| 1081 | tcatgaagaa gaacatgatg ttcatcaaga catgcgcatg ccatttcaac tgtccaggag |
| 1141 | acaacgacat ctttgaggcc atgtactatc ggaaaatgta tggagacatg gcatagaggc |
| 1201 | agagaaacac acttcaaaga ctttttcact tgaaataact ttgcatctca ttgctaaaca |
| 1261 | taaatgtata gcacaagagc atatttaaat gtaattaatg aaaagtcttt attttgaaga |
| 1321 | cttaaaacgt taaatgaccc cagtcgttgg tttaaagaaa ttggcacaaa acaagccaca |
| 1381 | gccactgact tgagtgtaat gtgctgtcac tgtactccca aaattccggt acagtatagg |
| 1441 | gatgtccatc atgtgacctt tacctgaaac tgtcatgtca tgagagcatc acatattggc |
| 1501 | cacaggttgg acatatctgg taatctgcat tgcacccagt caagctactg gaaaaaaatt |
| 1561 | tctatttaca gtatatgatg aactggcgct atgtcagtga ccgctgaagt tttatagccc |
| 1621 | aactacctta ttgttctgaa gaaagccgtc cttgtgggac agctgtttta gaaaccccta |
| 1681 | agtacaaatt gggaggaact ttaactacgt ctgttacaag actggaaaga ttgtagcata |
| 1741 | tcaatgtaca tgctgtaatc ttgttttatt attattgtat ataatgtata tatatttttg |
| 1801 | tacagttatc taacttaact taaatttgtt tcgttcttca ttttttacaa tgctttaata |
| 1861 | tgtgtatgtt aaccgttttt atttttgttt gtttgcgata aatctatgct tatctgaaaa |
| 1921 | agaaaccatt caaagcacga aatagattct caaatagaaa ttgttctcag ttaaagagag |
| 1981 | ggacagtcca gtgactgctt acagttctgt ttattacgag ccatccggca gtgttgattg |
| 2041 | tactggaaac attgtattaa cttgacttcc tttaggtgcc agagaggtat ccagtgcatt |
| 2101 | tcacaagaaa gcaagggcaa cttctctggt acatatgggg tgaatgaaca aataaccagt |
| 2161 | tgagtgtata tcctgaaacg gatccatgta ttttgaacca gatcatctgt ttcctccttt |
| 2221 | gaccatgact gttctatttt caacagtttg ttacgtgttg agaagtgtga ccaaatggta |
| 2281 | catgtttgca cttttctaga tgaaaaataa aattacttta tttttatata aaaaaaaaaa |
| 2341 | aaaaaaaaaa aaaaaaaaaa a |
| SEQ ID NO 19: Pre-Xenopus CTGF polypeptide (BC094492) | |
| MSAGKVTAVLLFALFCWVSDAQECNGECQCPNKVPVCDPGVSLVQDGCGCCKV | |
| CSKQLGELCTERDVCDPHKGLFCDFGSRVNRKIGVCTAREGAPCVFGGTVYRSG | |
| ESFQSSCKYQCTCIDGGVGCVPLCSMDIRLPSPECPFPRVKLPGKCCEEVVVCDQ | |
| PQERTLVGPALPAFRMEETYGPDPSLIRANCLVQTTEWSACSKTCGMGISTRVTN | |
| DNEHCRLEKQSRLCMVRPCEADLEENIKKGKKCIRTPKISKVKFEFSGCTSVKTRA | |
| KFCGVCTDGRCCTPHRTATLPVEFKCPDGEVMKKNMMFIKTCACHFNCPGDNDIF | |
| EAMYYRKMYGDMA | |
| SEQ ID NO 20: Mature Xenopus CTGF polypeptide (Q505L5) | |
| QECNGECQCPNKVPVCDPGVSLVQDGCGCCKVCSKQLGELCTERDVCDPHKGLFCDFGS | |
| RVNRKIGVCTAREGAPCVFGGTVYRSGESFQSSCKYQCTCIDGGVGCVPLCSMDIRLPSP | |
| ECPFPRRVKLPGKCCEEWVCDQPQERTLVGPALPAFRMEETYGPDPSLIRANCLVQTTEW | |
| SACSKTCGMGISTRVTNDNEHCRLEKQSRLCMVRPCEADLEENIKKGKKCIRTPKISKPVKF | |
| EFSGCTSVKTYRAKFCGVCTDGRCCTPHRTATLPVEFKCPDGEVMKKNMMFIKTCACHFN | |
| CPGDNDIFEAMYYRKMYGDMA | |
| SEQ ID NO 21: pre-Danio rerio CTGF nucleotide (NM_001015041) | |
| 1 | gtcactcgac tcggctcagc ctaaacttct taccaaagct aaactctagc tttcactcaa |
| 61 | cacgatcagg aggaactgga ttcaaagact acacaacgag atcgacttga tcaaatctga |
| 121 | agagaagact ttgtaggatc tcaaaagaag atttttcttt ttgctacaaa ctttaaagaa |
| 181 | gtcatcatgt tttctggaat gactcaaagt gtgattgctc tgctgttcct gactttctta |
| 241 | agatgggctg tggctcaaga gtgcagtgga caatgccact gccctgaagt gccgccccag |
| 301 | tgttcacctg gtgtaagcct agttctggac acctgtgggt gctgccgggt gtgtgccaag |
| 361 | cagctgggcg agctgtgcac agaacgagat gtttgcgacc cccacaaagg tctttactgt |
| 421 | gactacggct ccccaagtaa ccgtcgtatt ggtgtctgca cagccagaga tggtgccact |
| 481 | tgtgtgtttg gtggaatggt gtaccgcagt ggagagtctt tccagagcag ttgtaaatac |
| 541 | cagtgcacgt gtctggacgg tgctgtaggt tgcgtgcccc tctgtggaat ggacatcagg |
| 601 | ctccccagcc cagactgccc aatgccccgc agggtgaaag tgcctgggaa gtgctgcgag |
| 661 | gagtgggtgt gtgactcccc tcgtcaaaac acctttgtgg gatcagcttt agcagcttac |
| 721 | agagaggagg agacatacgg tcccgatccc tccatgatga gagagaactg cctggttcag |
| 781 | actacagaat ggagcgcatg ctcaaaaacc tgtggcttgg gaatctctac tcgtgtgacc |
| 841 | aatgacaacc gtgagtgccg ccttgagaag cagtctcgcc tttgcatggt ccgcccttgc |
| 901 | gagtcacacc tggaggagaa aataaggaaa gggaaaaagt gcatccgcac accacgagtc |
| 961 | tccaaaccca tgaagtttga gatttccggc tgtaccacta ccaagtctta cagacccaag |
| 1021 | ttctgcggcg tttgcacaga tggtcgctgt tgcactcctc acagaaccgc caccttgccc |
| 1081 | atggagttca agtgccccga tggccaagtc atgaagaagc agatgatgtt catcaagacc |
| 1141 | tgcgcatgcc actacaactg ccctggggag aacgacatct ttgaatccat gtactacaag |
| 1201 | aagatggttg gcgacatggc gtgaaaagtc atgacctgac aaggagttcc atgcagtgac |
| 1261 | tgtccacttg aattgaacag atatccatct catatctcag cacaaattac ttgtttgtct |
| 1321 | ttttatggtt tgttgctttc attttacgtc tttttgcgtg tatgtgtttg catcttgttg |
| 1381 | gtggactctg gatttctatg ctgtatgtgt attcatttgt catggtggat gtctaagcct |
| 1441 | agggggtggg gatcgtaaaa tgaccgggcc ctgttcggac gcccctaaaa aacaatactg |
| 1501 | cactatagtc cgatgctgtg acgcccaatg tcactgattg gcagatgacg ccaagcccct |
| 1561 | cctctaaact gtggtgagca gattcagatt ctgagagcaa aaatagtaag aaatggatgg |
| 1621 | cagctttaac cccagaagct aatcgcctca ccaagctaag aaggcggaaa aaagagacag |
| 1681 | agagaaattg atgtgctctt atgtttagtt gatattgtca gctcagagtt tgattgagct |
| 1741 | gatccaagac atgaagtttg gccaagaact atgaaggtta atatagcact gagtgtttga |
| 1801 | ctgtctggcc cctcctgttg ggaaagagcc agtatccctg tcaaaaagac tggatgaatt |
| 1861 | gtagcttcat tcttttttat gacaatatct atttttgaaa gctgtaaata atgtacatat |
| 1921 | tttgtacagc ttaagttaat ttaaattgaa agcattttgt cttcttattt ccttgcttcg |
| 1981 | tcatgtttaa tgatagcttc atgtgctgac ttgactattc ttggtacagc ttgtccaata |
| 2041 | ttttattgag aagtgtgacc aaaatgttac atgttttcac ttttgtagtt tacaataaaa |
| 2101 | tattatattt ttatacaaa |
| SEQ ID NO 22: pre-Danio rerio CTGF polypeptide (NM_001015041) | |
| MFSGMTQSVIALLFLTFLRWAVAQECSGQCHCPEVPPQCSPGVSLVLDTCGCCR | |
| VCAKQLGELCTERDVCDPHKGLYCDYGSPSNRRIGVCTARDGATCVFGGMVYRS | |
| GESFQSSCKYQCTCLDGAVGCVPLCGMDIRLPSPDCPMPRRVKVPGKCCEEWV | |
| CDSPRQNTFVGSALAAYREEETYGPDPSMMRENCLVQTTEWSACSKTCGLGIST | |
| RVTNDNRECRLEKQSRLCMVRPCESHLEEKIRKGKKCIRTPRVSKPMKFEISGCT- | |
| TTKSYR | |
| PKFCGVCTDGRCCTPHRTATLPMEFKCPDGQVMKKQMMFIKTCACHYNCPGEND | |
| IFESMYYKKMVGDMA | |
| SEQ ID NO 23: Mature Danio rerio CTGF polypeptide (NM_001015041) | |
| QECSGQCHCPEVPPQCSPGVSLVLDTCGCCRVCAKQLGELCTERDVCDPHKGLYCDYGS | |
| PSNRRIGVCTARDGATCVFGGMVYRSGESFQSSCKYQCTCLDGAVGCVPLCGMDIRLPSP | |
| DCPMPRRVKVPGKCCEEWVCDSPRQNTFVGSALAAYREEETYGPDPSMMRENCLVQTTE | |
| WSACSKTCGLGISTRVTNDNRECRLEKQSRLCMVRPCESHLEEKIRKGKKCIRTPRVSKPM | |
| KFEISGCTTTKSYRPKFCGVCTDGRCCTPHRTATLPMEFKCPDGQVMKKQMMFIKTCACH | |
| YNCPGENDIFESMYYKKMVGDMA | |
| SEQ ID NO 24: CTGFFRAG1 - Sequences encoding exon 4 are nucleotide 748 | |
| to 959 of the human sequence (NM_001901): | |
| ctt accgactgga agacacgttt ggcccagacc caactatgat tagagccaac tgcctggtcc agacca- | |
| caga gtggagcgcc tgttccaaga cctgtgggat gggcatctcc acccgggtta ccaatgacaa | |
| cgcctcctgc aggctagaga agcagagccg cctgtgcatg gtcaggcctt gcgaagctga cctggaagag | |
| aacattaag | |
| SEQ ID NO 25: CTGFFRAG2 - Sequences encoding exon 5 are nucleotide 960 | |
| to 2344 of the human sequence (NM_001901) | |
| aagggcaaaaa gtgcatccgt actcccaaaa tctccaagcc tatcaagttt gagctttctg gctgcaccag | |
| catgaagaca taccgagcta aattctgtgg agtatgtacc gacggccgat gctgcacccc ccacagaacc | |
| accaccctgc cggtggagtt caagtgccct gacggcgagg tcatgaagaa gaacatgatg ttcatcaaga | |
| cctgtgcctg ccattacaac tgtcccggag acaatgacat ctttgaatcg ctgtactaca ggaagatgta | |
| cggagacatg gcatgaagcc agagagtgag agacattaac tcattagact ggaacttgaa ctgattcaca | |
| tctcattttt ccgtaaaaat gatttcagta gcacaagtta tttaaatctg tttttctaac tgggggaaaa | |
| gattcccacc caattcaaaa cattgtgcca tgtcaaacaa atagtctatc aaccccagac actggtttga | |
| agaatgttaa gacttgacag tggaactaca ttagtacaca gcaccagaat gtatattaag gtgtggcttt ag- | |
| gagcagtg ggagggtacc agcagaaagg ttagtatcat cagatagcat cttatacgag taatatgcct | |
| gctatttgaa gtgtaattga gaaggaaaat tttagcgtgc tcactgacct gcctgtagcc ccagtgacag ctag- | |
| gatgtg cattctccag ccatcaagag actgagtcaa gttgttcctt aagtcagaac agcagactca | |
| gctctgacat tctgattcga atgacactgt tcaggaatcg gaatcctgtc gattagactg gacagcttgt | |
| ggcaagtgaa tttgcctgta acaagccaga ttttttaaaa tttatattgt aaatattgtg tgtgtgtgtg tgtgtgtata | |
| tatatatata tgtacagtta tctaagttaa tttaaagttg tttgtgcctt tttatttttg tttttaatgc tttgatattt | |
| caatgttagc ctcaatttct gaacaccata ggtagaatgt aaagcttgtc tgatcgttca aagcatgaaa tgga- | |
| tactta tatggaaatt ctgctcagat agaatgacag tccgtcaaaa cagattgttt gcaaagggga ggcat- | |
| cagtg tccttggcag gctgatttct aggtaggaaa tgtggtagcc tcacttttaa tgaacaaatg gcctttatta | |
| aaaactgagt gactctatat agctgatcag ttttttcacc tggaagcatt tgtttctact ttgatatgac tgtttttcgg | |
| acagtttatt tgttgagagt gtgaccaaaa gttacatgtt tgcacctttc tagttgaaaa taaagtgtat attttttcta | |
| taaa | |
| SEQ ID NO 26: Homo sapiens BDNF transcript variant 1 nucleotide | |
| (NM_170735) | |
| 1 | gttccccaac tgctgtttta ttgtgctatt catgcctaga catcacatag ctagaaaggc |
| 61 | ccatcagacc cctcaggcca ctgctgttcc tgtcacacat tcctgcaaag gaccatgttg |
| 121 | ctaacttgaa aaaaattact attaattaca cttgcagttg ttgcttagta acatttatga |
| 181 | ttttgtgttt ctcgtgacag catgagcaga gatcattaaa aattaaactt acaaagctgc |
| 241 | taaagtggga agaaggagaa cttgaagcca caatttttgc acttgcttag aagccatcta |
| 301 | atctcaggtt tatatgctag atcttggggg aaacactgca tgtctctggt ttatattaaa |
| 361 | ccacatacag cacactactg acactgattt gtgtctggtg cagctggagt ttatcaccaa |
| 421 | gacataaaaa aaccttgacc ctgcagaatg gcctggaatt acaatcagat gggccacatg |
| 481 | gcatcccggt gaaagaaagc cctaaccagt tttctgtctt gtttctgctt tctccctaca |
| 541 | gttccaccag gtgagaagag tgatgaccat ccttttcctt actatggtta tttcatactt |
| 601 | tggttgcatg aaggctgccc ccatgaaaga agcaaacatc cgaggacaag gtggcttggc |
| 661 | ctacccaggt gtgcggaccc atgggactct ggagagcgtg aatgggccca aggcaggttc |
| 721 | aagaggcttg acatcattgg ctgacacttt cgaacacgtg atagaagagc tgttggatga |
| 781 | ggaccagaaa gttcggccca atgaagaaaa caataaggac gcagacttgt acacgtccag |
| 841 | ggtgatgctc agtagtcaag tgcctttgga gcctcctctt ctctttctgc tggaggaata |
| 901 | caaaaattac ctagatgctg caaacatgtc catgagggtc cggcgccact ctgaccctgc |
| 961 | ccgccgaggg gagctgagcg tgtgtgacag tattagtgag tgggtaacgg cggcagacaa |
| 1021 | aaagactgca gtggacatgt cgggcgggac ggtcacagtc cttgaaaagg tccctgtatc |
| 1081 | aaaaggccaa ctgaagcaat acttctacga gaccaagtgc aatcccatgg gttacacaaa |
| 1141 | agaaggctgc aggggcatag acaaaaggca ttggaactcc cagtgccgaa ctacccagtc |
| 1201 | gtacgtgcgg gcccttacca tggatagcaa aaagagaatt ggctggcgat tcataaggat |
| 1261 | agacacttct tgtgtatgta cattgaccat taaaagggga agatagtgga tttatgttgt |
| 1321 | atagattaga ttatattgag acaaaaatta tctatttgta tatatacata acagggtaaa |
| 1381 | ttattcagtt aagaaaaaaa taattttatg aactgcatgt ataaatgaag tttatacagt |
| 1441 | acagtggttc tacaatctat ttattggaca tgtccatgac cagaagggaa acagtcattt |
| 1501 | gcgcacaact taaaaagtct gcattacatt ccttgataat gttgtggttt gttgccgttg |
| 1561 | ccaagaactg aaaacataaa aagttaaaaa aaataataaa ttgcatgctg ctttaattgt |
| 1621 | gaattgataa taaactgtcc tctttcagaa aacagaaaaa aaacacacac acacacaaca |
| 1681 | aaaatttgaa ccaaaacatt ccgtttacat tttagacagt aagtatcttc gttcttgtta |
| 1741 | gtactatatc tgttttactg cttttaactt ctgatagcgt tggaattaaa acaatgtcaa |
| 1801 | ggtgctgttg tcattgcttt actggcttag gggatggggg atggggggta tatttttgtt |
| 1861 | tgttttgtgt ttttttttcg tttgtttgtt ttgtttttta gttcccacag ggagtagaga |
| 1921 | tggggaaaga attcctacaa tatatattct ggctgataaa agatacattt gtatgttgtg |
| 1981 | aagatgtttg caatatcgat cagatgacta gaaagtgaat aaaaattaag gcaactgaac |
| 2041 | aaaaaaatgc tcacactcca catcccgtga tgcacctccc aggccccgct cattctttgg |
| 2101 | gcgttggtca gagtaagctg cttttgacgg aaggacctat gtttgctcag aacacattct |
| 2161 | ttccccccct ccccctctgg tctcctcttt gttttgtttt aaggaagaaa aatcagttgc |
| 2221 | gcgttctgaa atattttacc actgctgtga acaagtgaac acattgtgtc acatcatgac |
| 2281 | actcgtataa gcatggagaa cagtgatttt tttttagaac agaaaacaac aaaaaataac |
| 2341 | cccaaaatga agattatttt ttatgaggag tgaacatttg ggtaaatcat ggctaagctt |
| 2401 | aaaaaaaact catggtgagg cttaacaatg tcttgtaagc aaaaggtaga gccctgtatc |
| 2461 | aacccagaaa cacctagatc agaacaggaa tccacattgc cagtgacatg agactgaaca |
| 2521 | gccaaatgga ggctatgtgg agttggcatt gcatttaccg gcagtgcggg aggaatttct |
| 2581 | gagtggccat cccaaggtct aggtggaggt ggggcatggt atttgagaca ttccaaaacg |
| 2641 | aaggcctctg aaggaccctt cagaggtggc tctggaatga catgtgtcaa gctgcttgga |
| 2701 | cctcgtgctt taagtgccta cattatctaa ctgtgctcaa gaggttctcg actggaggac |
| 2761 | cacactcaag ccgacttatg cccaccatcc cacctctgga taattttgca taaaattgga |
| 2821 | ttagcctgga gcaggttggg agccaaatgt ggcatttgtg atcatgagat tgatgcaatg |
| 2881 | agatagaaga tgtttgctac ctgaacactt attgctttga aactagactt gaggaaacca |
| 2941 | gggtttatct tttgagaact tttggtaagg gaaaagggaa caggaaaaga aaccccaaac |
| 3001 | tcaggccgaa tgatcaaggg gacccatagg aaatcttgtc cagagacaag acttcgggaa |
| 3061 | ggtgtctgga cattcagaac accaagactt gaaggtgcct tgctcaatgg aagaggccag |
| 3121 | gacagagctg acaaaatttt gctccccagt gaaggccaca gcaaccttct gcccatcctg |
| 3181 | tctgttcatg gagagggtcc ctgcctcacc tctgccattt tgggttagga gaagtcaagt |
| 3241 | tgggagcctg aaatagtggt tcttggaaaa atggatcccc agtgaaaact agagctctaa |
| 3301 | gcccattcag cccatttcac acctgaaaat gttagtgatc accacttgga ccagcatcct |
| 3361 | taagtatcag aaagccccaa gcaattgctg catcttagta gggtgaggga taagcaaaag |
| 3421 | aggatgttca ccataaccca ggaatgaaga taccatcagc aaagaatttc aatttgttca |
| 3481 | gtctttcatt tagagctagt ctttcacagt accatctgaa tacctctttg aaagaaggaa |
| 3541 | gactttacgt agtgtagatt tgttttgtgt tgtttgaaaa tattatcttt gtaattattt |
| 3601 | ttaatatgta aggaatgctt ggaatatctg ctatatgtca actttatgca gcttcctttt |
| 3661 | gagggacaaa tttaaaacaa acaacccccc atcacaaact taaaggattg caagggccag |
| 3721 | atctgttaag tggtttcata ggagacacat ccagcaattg tgtggtcagt ggctctttta |
| 3781 | cccaataaga tacatcacag tcacatgctt gatggtttat gttgacctaa gatttatttt |
| 3841 | gttaaaatct ctctctgttg tgttcgttct tgttctgttt tgttttgttt tttaaagtct |
| 3901 | tgctgtggtc tctttgtggc agaagtgttt catgcatggc agcaggcctg ttgctttttt |
| 3961 | atggcgattc ccattgaaaa tgtaagtaaa tgtctgtggc cttgttctct ctatggtaaa |
| 4021 | gatattattc accatgtaaa acaaaaaaca atatttattg tattttagta tatttatata |
| 4081 | attatgttat tgaaaaaaat tggcattaaa acttaaccgc atcagaacct attgtaaata |
| 4141 | caagttctat ttaagtgtac taattaacat ataatatatg ttttaaatat agaattttta |
| 4201 | atgtttttaa atatattttc aaagtacata aaaaaaaaaa aaaaaaa |
| SEQ ID NO 27: Homo sapiens BDNF transcript variant 2 nucleotide | |
| (NM_170732) | |
| 1 | gtgtgtaatc cgggcgatag gagtccattc agcaccttgg acagagccaa cggatttgtc |
| 61 | cgaggtggcg gtacccccag gtagtcttct tggccccgct gtaaagccaa ccctgtgtcg |
| 121 | cccttaaaaa gcgtcttttc tgaggttcgg ctcacactga gatcggggct ggagagagag |
| 181 | tcagattttg gagcggagcg tttggaaagc gagccccagt ttggtcccct cattgagctc |
| 241 | gctgaagttg gcttcctagc ggtgtaggct ggaatagact cttggcaagc tccgggttgg |
| 301 | tatactgggt taactttggg aaatgcaagt gtttatctcc aggatctagc caccggggtg |
| 361 | gtgtaagccg caaagaagtt ccaccaggtg agaagagtga tgaccatcct tttccttact |
| 421 | atggttattt catactttgg ttgcatgaag gctgccccca tgaaagaagc aaacatccga |
| 481 | ggacaaggtg gcttggccta cccaggtgtg cggacccatg ggactctgga gagcgtgaat |
| 541 | gggcccaagg caggttcaag aggcttgaca tcattggctg acactttcga acacgtgata |
| 601 | gaagagctgt tggatgagga ccagaaagtt cggcccaatg aagaaaacaa taaggacgca |
| 661 | gacttgtaca cgtccagggt gatgctcagt agtcaagtgc ctttggagcc tcctcttctc |
| 721 | tttctgctgg aggaatacaa aaattaccta gatgctgcaa acatgtccat gagggtccgg |
| 781 | cgccactctg accctgcccg ccgaggggag ctgagcgtgt gtgacagtat tagtgagtgg |
| 841 | gtaacggcgg cagacaaaaa gactgcagtg gacatgtcgg gcgggacggt cacagtcctt |
| 901 | gaaaaggtcc ctgtatcaaa aggccaactg aagcaatact tctacgagac caagtgcaat |
| 961 | cccatgggtt acacaaaaga aggctgcagg ggcatagaca aaaggcattg gaactcccag |
| 1021 | tgccgaacta cccagtcgta cgtgcgggcc cttaccatgg atagcaaaaa gagaattggc |
| 1081 | tggcgattca taaggataga cacttcttgt gtatgtacat tgaccattaa aaggggaaga |
| 1141 | tagtggattt atgttgtata gattagatta tattgagaca aaaattatct atttgtatat |
| 1201 | atacataaca gggtaaatta ttcagttaag aaaaaaataa ttttatgaac tgcatgtata |
| 1261 | aatgaagttt atacagtaca gtggttctac aatctattta ttggacatgt ccatgaccag |
| 1321 | aagggaaaca gtcatttgcg cacaacttaa aaagtctgca ttacattcct tgataatgtt |
| 1381 | gtggtttgtt gccgttgcca agaactgaaa acataaaaag ttaaaaaaaa taataaattg |
| 1441 | catgctgctt taattgtgaa ttgataataa actgtcctct ttcagaaaac agaaaaaaaa |
| 1501 | cacacacaca cacaacaaaa atttgaacca aaacattccg tttacatttt agacagtaag |
| 1561 | tatcttcgtt cttgttagta ctatatctgt tttactgctt ttaacttctg atagcgttgg |
| 1621 | aattaaaaca atgtcaaggt gctgttgtca ttgctttact ggcttagggg atgggggatg |
| 1681 | gggggtatat ttttgtttgt tttgtgtttt tttttcgttt gtttgttttg ttttttagtt |
| 1741 | cccacaggga gtagagatgg ggaaagaatt cctacaatat atattctggc tgataaaaga |
| 1801 | tacatttgta tgttgtgaag atgtttgcaa tatcgatcag atgactagaa agtgaataaa |
| 1861 | aattaaggca actgaacaaa aaaatgctca cactccacat cccgtgatgc acctcccagg |
| 1921 | ccccgctcat tctttgggcg ttggtcagag taagctgctt ttgacggaag gacctatgtt |
| 1981 | tgctcagaac acattctttc cccccctccc cctctggtct cctctttgtt ttgttttaag |
| 2041 | gaagaaaaat cagttgcgcg ttctgaaata ttttaccact gctgtgaaca agtgaacaca |
| 2101 | ttgtgtcaca tcatgacact cgtataagca tggagaacag tgattttttt ttagaacaga |
| 2161 | aaacaacaaa aaataacccc aaaatgaaga ttatttttta tgaggagtga acatttgggt |
| 2221 | aaatcatggc taagcttaaa aaaaactcat ggtgaggctt aacaatgtct tgtaagcaaa |
| 2281 | aggtagagcc ctgtatcaac ccagaaacac ctagatcaga acaggaatcc acattgccag |
| 2341 | tgacatgaga ctgaacagcc aaatggaggc tatgtggagt tggcattgca tttaccggca |
| 2401 | gtgcgggagg aatttctgag tggccatccc aaggtctagg tggaggtggg gcatggtatt |
| 2461 | tgagacattc caaaacgaag gcctctgaag gacccttcag aggtggctct ggaatgacat |
| 2521 | gtgtcaagct gcttggacct cgtgctttaa gtgcctacat tatctaactg tgctcaagag |
| 2581 | gttctcgact ggaggaccac actcaagccg acttatgccc accatcccac ctctggataa |
| 2641 | ttttgcataa aattggatta gcctggagca ggttgggagc caaatgtggc atttgtgatc |
| 2701 | atgagattga tgcaatgaga tagaagatgt ttgctacctg aacacttatt gctttgaaac |
| 2761 | tagacttgag gaaaccaggg tttatctttt gagaactttt ggtaagggaa aagggaacag |
| 2821 | gaaaagaaac cccaaactca ggccgaatga tcaaggggac ccataggaaa tcttgtccag |
| 2881 | agacaagact tcgggaaggt gtctggacat tcagaacacc aagacttgaa ggtgccttgc |
| 2941 | tcaatggaag aggccaggac agagctgaca aaattttgct ccccagtgaa ggccacagca |
| 3001 | accttctgcc catcctgtct gttcatggag agggtccctg cctcacctct gccattttgg |
| 3061 | gttaggagaa gtcaagttgg gagcctgaaa tagtggttct tggaaaaatg gatccccagt |
| 3121 | gaaaactaga gctctaagcc cattcagccc atttcacacc tgaaaatgtt agtgatcacc |
| 3181 | acttggacca gcatccttaa gtatcagaaa gccccaagca attgctgcat cttagtaggg |
| 3241 | tgagggataa gcaaaagagg atgttcacca taacccagga atgaagatac catcagcaaa |
| 3301 | gaatttcaat ttgttcagtc tttcatttag agctagtctt tcacagtacc atctgaatac |
| 3361 | ctctttgaaa gaaggaagac tttacgtagt gtagatttgt tttgtgttgt ttgaaaatat |
| 3421 | tatctttgta attattttta atatgtaagg aatgcttgga atatctgcta tatgtcaact |
| 3481 | ttatgcagct tccttttgag ggacaaattt aaaacaaaca accccccatc acaaacttaa |
| 3541 | aggattgcaa gggccagatc tgttaagtgg tttcatagga gacacatcca gcaattgtgt |
| 3601 | ggtcagtggc tcttttaccc aataagatac atcacagtca catgcttgat ggtttatgtt |
| 3661 | gacctaagat ttattttgtt aaaatctctc tctgttgtgt tcgttcttgt tctgttttgt |
| 3721 | tttgtttttt aaagtcttgc tgtggtctct ttgtggcaga agtgtttcat gcatggcagc |
| 3781 | aggcctgttg cttttttatg gcgattccca ttgaaaatgt aagtaaatgt ctgtggcctt |
| 3841 | gttctctcta tggtaaagat attattcacc atgtaaaaca aaaaacaata tttattgtat |
| 3901 | tttagtatat ttatataatt atgttattga aaaaaattgg cattaaaact taaccgcatc |
| 3961 | agaacctatt gtaaatacaa gttctattta agtgtactaa ttaacatata atatatgttt |
| 4021 | taaatataga atttttaatg tttttaaata tattttcaaa gtacataaaa aaaaaaaaaa |
| 4081 | aaaa |
| SEQ ID NO 28: Homo sapiens BDNF transcript variant 3 nucleotide | |
| (NM_170731) | |
| 1 | gtgtgtaatc cgggcgatag gagtccattc agcaccttgg acagagccaa cggatttgtc |
| 61 | cgaggtggcg gtacccccag gtagtcttct tggccccgct gtaaagccaa ccctgtgtcg |
| 121 | cccttaaaaa gcgtcttttc tgaggttcgg ctcacactga gatcggggct ggagagagag |
| 181 | tcagattttg gagcggagcg tttggaaagc gagccccagt ttggtcccct cattgagctc |
| 241 | gctgaagttg gcttcctagc ggtgtaggct ggaatagact cttggcaagc tccgggttgg |
| 301 | tatactgggt taactttggg aaatgcaagt gtttatctcc aggatctagc caccggggtg |
| 361 | gtgtaagccg caaagaagtt ccaccaggtg agaagagtga tgaccatcct tttccttact |
| 421 | atggttattt catactttgg ttgcatgaag gctgccccca tgaaagaagc aaacatccga |
| 481 | ggacaaggtg gcttggccta cccaggtgtg cggacccatg ggactctgga gagcgtgaat |
| 541 | gggcccaagg caggttcaag aggcttgaca tcattggctg acactttcga acacgtgata |
| 601 | gaagagctgt tggatgagga ccagaaagtt cggcccaatg aagaaaacaa taaggacgca |
| 661 | gacttgtaca cgtccagggt gatgctcagt agtcaagtgc ctttggagcc tcctcttctc |
| 721 | tttctgctgg aggaatacaa aaattaccta gatgctgcaa acatgtccat gagggtccgg |
| 781 | cgccactctg accctgcccg ccgaggggag ctgagcgtgt gtgacagtat tagtgagtgg |
| 841 | gtaacggcgg cagacaaaaa gactgcagtg gacatgtcgg gcgggacggt cacagtcctt |
| 901 | gaaaaggtcc ctgtatcaaa aggccaactg aagcaatact tctacgagac caagtgcaat |
| 961 | cccatgggtt acacaaaaga aggctgcagg ggcatagaca aaaggcattg gaactcccag |
| 1021 | tgccgaacta cccagtcgta cgtgcgggcc cttaccatgg atagcaaaaa gagaattggc |
| 1081 | tggcgattca taaggataga cacttcttgt gtatgtacat tgaccattaa aaggggaaga |
| 1141 | tagtggattt atgttgtata gattagatta tattgagaca aaaattatct atttgtatat |
| 1201 | atacataaca gggtaaatta ttcagttaag aaaaaaataa ttttatgaac tgcatgtata |
| 1261 | aatgaagttt atacagtaca gtggttctac aatctattta ttggacatgt ccatgaccag |
| 1321 | aagggaaaca gtcatttgcg cacaacttaa aaagtctgca ttacattcct tgataatgtt |
| 1381 | gtggtttgtt gccgttgcca agaactgaaa acataaaaag ttaaaaaaaa taataaattg |
| 1441 | catgctgctt taattgtgaa ttgataataa actgtcctct ttcagaaaac agaaaaaaaa |
| 1501 | cacacacaca cacaacaaaa atttgaacca aaacattccg tttacatttt agacagtaag |
| 1561 | tatcttcgtt cttgttagta ctatatctgt tttactgctt ttaacttctg atagcgttgg |
| 1621 | aattaaaaca atgtcaaggt gctgttgtca ttgctttact ggcttagggg atgggggatg |
| 1681 | gggggtatat ttttgtttgt tttgtgtttt tttttcgttt gtttgttttg ttttttagtt |
| 1741 | cccacaggga gtagagatgg ggaaagaatt cctacaatat atattctggc tgataaaaga |
| 1801 | tacatttgta tgttgtgaag atgtttgcaa tatcgatcag atgactagaa agtgaataaa |
| 1861 | aattaaggca actgaacaaa aaaatgctca cactccacat cccgtgatgc acctcccagg |
| 1921 | ccccgctcat tctttgggcg ttggtcagag taagctgctt ttgacggaag gacctatgtt |
| 1981 | tgctcagaac acattctttc cccccctccc cctctggtct cctctttgtt ttgttttaag |
| 2041 | gaagaaaaat cagttgcgcg ttctgaaata ttttaccact gctgtgaaca agtgaacaca |
| 2101 | ttgtgtcaca tcatgacact cgtataagca tggagaacag tgattttttt ttagaacaga |
| 2161 | aaacaacaaa aaataacccc aaaatgaaga ttatttttta tgaggagtga acatttgggt |
| 2221 | aaatcatggc taagcttaaa aaaaactcat ggtgaggctt aacaatgtct tgtaagcaaa |
| 2281 | aggtagagcc ctgtatcaac ccagaaacac ctagatcaga acaggaatcc acattgccag |
| 2341 | tgacatgaga ctgaacagcc aaatggaggc tatgtggagt tggcattgca tttaccggca |
| 2401 | gtgcgggagg aatttctgag tggccatccc aaggtctagg tggaggtggg gcatggtatt |
| 2461 | tgagacattc caaaacgaag gcctctgaag gacccttcag aggtggctct ggaatgacat |
| 2521 | gtgtcaagct gcttggacct cgtgctttaa gtgcctacat tatctaactg tgctcaagag |
| 2581 | gttctcgact ggaggaccac actcaagccg acttatgccc accatcccac ctctggataa |
| 2641 | ttttgcataa aattggatta gcctggagca ggttgggagc caaatgtggc atttgtgatc |
| 2701 | atgagattga tgcaatgaga tagaagatgt ttgctacctg aacacttatt gctttgaaac |
| 2761 | tagacttgag gaaaccaggg tttatctttt gagaactttt ggtaagggaa aagggaacag |
| 2821 | gaaaagaaac cccaaactca ggccgaatga tcaaggggac ccataggaaa tcttgtccag |
| 2881 | agacaagact tcgggaaggt gtctggacat tcagaacacc aagacttgaa ggtgccttgc |
| 2941 | tcaatggaag aggccaggac agagctgaca aaattttgct ccccagtgaa ggccacagca |
| 3001 | accttctgcc catcctgtct gttcatggag agggtccctg cctcacctct gccattttgg |
| 3061 | gttaggagaa gtcaagttgg gagcctgaaa tagtggttct tggaaaaatg gatccccagt |
| 3121 | gaaaactaga gctctaagcc cattcagccc atttcacacc tgaaaatgtt agtgatcacc |
| 3181 | acttggacca gcatccttaa gtatcagaaa gccccaagca attgctgcat cttagtaggg |
| 3241 | tgagggataa gcaaaagagg atgttcacca taacccagga atgaagatac catcagcaaa |
| 3301 | gaatttcaat ttgttcagtc tttcatttag agctagtctt tcacagtacc atctgaatac |
| 3361 | ctctttgaaa gaaggaagac tttacgtagt gtagatttgt tttgtgttgt ttgaaaatat |
| 3421 | tatctttgta attattttta atatgtaagg aatgcttgga atatctgcta tatgtcaact |
| 3481 | ttatgcagct tccttttgag ggacaaattt aaaacaaaca accccccatc acaaacttaa |
| 3541 | aggattgcaa gggccagatc tgttaagtgg tttcatagga gacacatcca gcaattgtgt |
| 3601 | ggtcagtggc tcttttaccc aataagatac atcacagtca catgcttgat ggtttatgtt |
| 3661 | gacctaagat ttattttgtt aaaatctctc tctgttgtgt tcgttcttgt tctgttttgt |
| 3721 | tttgtttttt aaagtcttgc tgtggtctct ttgtggcaga agtgtttcat gcatggcagc |
| 3781 | aggcctgttg cttttttatg gcgattccca ttgaaaatgt aagtaaatgt ctgtggcctt |
| 3841 | gttctctcta tggtaaagat attattcacc atgtaaaaca aaaaacaata tttattgtat |
| 3901 | tttagtatat ttatataatt atgttattga aaaaaattgg cattaaaact taaccgcatc |
| 3961 | agaacctatt gtaaatacaa gttctattta agtgtactaa ttaacatata atatatgttt |
| 4021 | taaatataga atttttaatg tttttaaata tattttcaaa gtacataaaa aaaaaaaaaa |
| 4081 | aaaa |
| SEQ ID NO 29: Homo sapiens BDNF transcript variant 4 nucleotide | |
| (NM_001709) | |
| 1 | gctgccgccg ccgcgcccgg gcgcacccgc ccgctcgctg tcccgcgcac cccgtagcgc |
| 61 | ctcgggctcc cgggccggac agaggagcca gcccggtgcg cccctccacc tcctgctcgg |
| 121 | ggggctttaa tgagacaccc accgctgctg tggggccggc ggggagcagc accgcgacgg |
| 181 | ggaccggggc tgggcgctgg agccagaatc ggaaccacga tgtgactccg ccgccgggga |
| 241 | cccgtgaggt ttgtgtggac cccgagttcc accaggtgag aagagtgatg accatccttt |
| 301 | tccttactat ggttatttca tactttggtt gcatgaaggc tgcccccatg aaagaagcaa |
| 361 | acatccgagg acaaggtggc ttggcctacc caggtgtgcg gacccatggg actctggaga |
| 421 | gcgtgaatgg gcccaaggca ggttcaagag gcttgacatc attggctgac actttcgaac |
| 481 | acgtgataga agagctgttg gatgaggacc agaaagttcg gcccaatgaa gaaaacaata |
| 541 | aggacgcaga cttgtacacg tccagggtga tgctcagtag tcaagtgcct ttggagcctc |
| 601 | ctcttctctt tctgctggag gaatacaaaa attacctaga tgctgcaaac atgtccatga |
| 661 | gggtccggcg ccactctgac cctgcccgcc gaggggagct gagcgtgtgt gacagtatta |
| 721 | gtgagtgggt aacggcggca gacaaaaaga ctgcagtgga catgtcgggc gggacggtca |
| 781 | cagtccttga aaaggtccct gtatcaaaag gccaactgaa gcaatacttc tacgagacca |
| 841 | agtgcaatcc catgggttac acaaaagaag gctgcagggg catagacaaa aggcattgga |
| 901 | actcccagtg ccgaactacc cagtcgtacg tgcgggccct taccatggat agcaaaaaga |
| 961 | gaattggctg gcgattcata aggatagaca cttcttgtgt atgtacattg accattaaaa |
| 1021 | ggggaagata gtggatttat gttgtataga ttagattata ttgagacaaa aattatctat |
| 1081 | ttgtatatat acataacagg gtaaattatt cagttaagaa aaaaataatt ttatgaactg |
| 1141 | catgtataaa tgaagtttat acagtacagt ggttctacaa tctatttatt ggacatgtcc |
| 1201 | atgaccagaa gggaaacagt catttgcgca caacttaaaa agtctgcatt acattccttg |
| 1261 | ataatgttgt ggtttgttgc cgttgccaag aactgaaaac ataaaaagtt aaaaaaaata |
| 1321 | ataaattgca tgctgcttta attgtgaatt gataataaac tgtcctcttt cagaaaacag |
| 1381 | aaaaaaaaca cacacacaca caacaaaaat ttgaaccaaa acattccgtt tacattttag |
| 1441 | acagtaagta tcttcgttct tgttagtact atatctgttt tactgctttt aacttctgat |
| 1501 | agcgttggaa ttaaaacaat gtcaaggtgc tgttgtcatt gctttactgg cttaggggat |
| 1561 | gggggatggg gggtatattt ttgtttgttt tgtgtttttt tttcgtttgt ttgttttgtt |
| 1621 | ttttagttcc cacagggagt agagatgggg aaagaattcc tacaatatat attctggctg |
| 1681 | ataaaagata catttgtatg ttgtgaagat gtttgcaata tcgatcagat gactagaaag |
| 1741 | tgaataaaaa ttaaggcaac tgaacaaaaa aatgctcaca ctccacatcc cgtgatgcac |
| 1801 | ctcccaggcc ccgctcattc tttgggcgtt ggtcagagta agctgctttt gacggaagga |
| 1861 | cctatgtttg ctcagaacac attctttccc cccctccccc tctggtctcc tctttgtttt |
| 1921 | gttttaagga agaaaaatca gttgcgcgtt ctgaaatatt ttaccactgc tgtgaacaag |
| 1981 | tgaacacatt gtgtcacatc atgacactcg tataagcatg gagaacagtg attttttttt |
| 2041 | agaacagaaa acaacaaaaa ataaccccaa aatgaagatt attttttatg aggagtgaac |
| 2101 | atttgggtaa atcatggcta agcttaaaaa aaactcatgg tgaggcttaa caatgtcttg |
| 2161 | taagcaaaag gtagagccct gtatcaaccc agaaacacct agatcagaac aggaatccac |
| 2221 | attgccagtg acatgagact gaacagccaa atggaggcta tgtggagttg gcattgcatt |
| 2281 | taccggcagt gcgggaggaa tttctgagtg gccatcccaa ggtctaggtg gaggtggggc |
| 2341 | atggtatttg agacattcca aaacgaaggc ctctgaagga cccttcagag gtggctctgg |
| 2401 | aatgacatgt gtcaagctgc ttggacctcg tgctttaagt gcctacatta tctaactgtg |
| 2461 | ctcaagaggt tctcgactgg aggaccacac tcaagccgac ttatgcccac catcccacct |
| 2521 | ctggataatt ttgcataaaa ttggattagc ctggagcagg ttgggagcca aatgtggcat |
| 2581 | ttgtgatcat gagattgatg caatgagata gaagatgttt gctacctgaa cacttattgc |
| 2641 | tttgaaacta gacttgagga aaccagggtt tatcttttga gaacttttgg taagggaaaa |
| 2701 | gggaacagga aaagaaaccc caaactcagg ccgaatgatc aaggggaccc ataggaaatc |
| 2761 | ttgtccagag acaagacttc gggaaggtgt ctggacattc agaacaccaa gacttgaagg |
| 2821 | tgccttgctc aatggaagag gccaggacag agctgacaaa attttgctcc ccagtgaagg |
| 2881 | ccacagcaac cttctgccca tcctgtctgt tcatggagag ggtccctgcc tcacctctgc |
| 2941 | cattttgggt taggagaagt caagttggga gcctgaaata gtggttcttg gaaaaatgga |
| 3001 | tccccagtga aaactagagc tctaagccca ttcagcccat ttcacacctg aaaatgttag |
| 3061 | tgatcaccac ttggaccagc atccttaagt atcagaaagc cccaagcaat tgctgcatct |
| 3121 | tagtagggtg agggataagc aaaagaggat gttcaccata acccaggaat gaagatacca |
| 3181 | tcagcaaaga atttcaattt gttcagtctt tcatttagag ctagtctttc acagtaccat |
| 3241 | ctgaatacct ctttgaaaga aggaagactt tacgtagtgt agatttgttt tgtgttgttt |
| 3301 | gaaaatatta tctttgtaat tatttttaat atgtaaggaa tgcttggaat atctgctata |
| 3361 | tgtcaacttt atgcagcttc cttttgaggg acaaatttaa aacaaacaac cccccatcac |
| 3421 | aaacttaaag gattgcaagg gccagatctg ttaagtggtt tcataggaga cacatccagc |
| 3481 | aattgtgtgg tcagtggctc ttttacccaa taagatacat cacagtcaca tgcttgatgg |
| 3541 | tttatgttga cctaagattt attttgttaa aatctctctc tgttgtgttc gttcttgttc |
| 3601 | tgttttgttt tgttttttaa agtcttgctg tggtctcttt gtggcagaag tgtttcatgc |
| 3661 | atggcagcag gcctgttgct tttttatggc gattcccatt gaaaatgtaa gtaaatgtct |
| 3721 | gtggccttgt tctctctatg gtaaagatat tattcaccat gtaaaacaaa aaacaatatt |
| 3781 | tattgtattt tagtatattt atataattat gttattgaaa aaaattggca ttaaaactta |
| 3841 | accgcatcag aacctattgt aaatacaagt tctatttaag tgtactaatt aacatataat |
| 3901 | atatgtttta aatatagaat ttttaatgtt tttaaatata ttttcaaagt acataaaaaa |
| 3961 | aaaaaaaaaa aa |
| SEQ ID NO 30: Homo sapiens BDNF transcript variant 5 nucleotide | |
| (NM_170733) | |
| 1 | ggctgctctc gctgccgctc cccccggcga actagcatga aatctccctg cctctgccga |
| 61 | gatcaaatgg agcttctcgc tgatggggtg cgagtattac ctccgccatg caatttccac |
| 121 | tatcaataat ttaacttctt tgctgcagaa cagaaggagt acataccggg caccaaagac |
| 181 | tcgcgccccc tccccccttt aattaagcga agggaacgtg aaaaaataat agagtgtggg |
| 241 | agttttgggg ccgaagtctt tcccggagca gctgccttga tggttacttt gacaagtagt |
| 301 | gactgaaaag ttccaccagg tgagaagagt gatgaccatc cttttcctta ctatggttat |
| 361 | ttcatacttt ggttgcatga aggctgcccc catgaaagaa gcaaacatcc gaggacaagg |
| 421 | tggcttggcc tacccaggtg tgcggaccca tgggactctg gagagcgtga atgggcccaa |
| 481 | ggcaggttca agaggcttga catcattggc tgacactttc gaacacgtga tagaagagct |
| 541 | gttggatgag gaccagaaag ttcggcccaa tgaagaaaac aataaggacg cagacttgta |
| 601 | cacgtccagg gtgatgctca gtagtcaagt gcctttggag cctcctcttc tctttctgct |
| 661 | ggaggaatac aaaaattacc tagatgctgc aaacatgtcc atgagggtcc ggcgccactc |
| 721 | tgaccctgcc cgccgagggg agctgagcgt gtgtgacagt attagtgagt gggtaacggc |
| 781 | ggcagacaaa aagactgcag tggacatgtc gggcgggacg gtcacagtcc ttgaaaaggt |
| 841 | ccctgtatca aaaggccaac tgaagcaata cttctacgag accaagtgca atcccatggg |
| 901 | ttacacaaaa gaaggctgca ggggcataga caaaaggcat tggaactccc agtgccgaac |
| 961 | tacccagtcg tacgtgcggg cccttaccat ggatagcaaa aagagaattg gctggcgatt |
| 1021 | cataaggata gacacttctt gtgtatgtac attgaccatt aaaaggggaa gatagtggat |
| 1081 | ttatgttgta tagattagat tatattgaga caaaaattat ctatttgtat atatacataa |
| 1141 | cagggtaaat tattcagtta agaaaaaaat aattttatga actgcatgta taaatgaagt |
| 1201 | ttatacagta cagtggttct acaatctatt tattggacat gtccatgacc agaagggaaa |
| 1261 | cagtcatttg cgcacaactt aaaaagtctg cattacattc cttgataatg ttgtggtttg |
| 1321 | ttgccgttgc caagaactga aaacataaaa agttaaaaaa aataataaat tgcatgctgc |
| 1381 | tttaattgtg aattgataat aaactgtcct ctttcagaaa acagaaaaaa aacacacaca |
| 1441 | cacacaacaa aaatttgaac caaaacattc cgtttacatt ttagacagta agtatcttcg |
| 1501 | ttcttgttag tactatatct gttttactgc ttttaacttc tgatagcgtt ggaattaaaa |
| 1561 | caatgtcaag gtgctgttgt cattgcttta ctggcttagg ggatggggga tggggggtat |
| 1621 | atttttgttt gttttgtgtt tttttttcgt ttgtttgttt tgttttttag ttcccacagg |
| 1681 | gagtagagat ggggaaagaa ttcctacaat atatattctg gctgataaaa gatacatttg |
| 1741 | tatgttgtga agatgtttgc aatatcgatc agatgactag aaagtgaata aaaattaagg |
| 1801 | caactgaaca aaaaaatgct cacactccac atcccgtgat gcacctccca ggccccgctc |
| 1861 | attctttggg cgttggtcag agtaagctgc ttttgacgga aggacctatg tttgctcaga |
| 1921 | acacattctt tccccccctc cccctctggt ctcctctttg ttttgtttta aggaagaaaa |
| 1981 | atcagttgcg cgttctgaaa tattttacca ctgctgtgaa caagtgaaca cattgtgtca |
| 2041 | catcatgaca ctcgtataag catggagaac agtgattttt ttttagaaca gaaaacaaca |
| 2101 | aaaaataacc ccaaaatgaa gattattttt tatgaggagt gaacatttgg gtaaatcatg |
| 2161 | gctaagctta aaaaaaactc atggtgaggc ttaacaatgt cttgtaagca aaaggtagag |
| 2221 | ccctgtatca acccagaaac acctagatca gaacaggaat ccacattgcc agtgacatga |
| 2281 | gactgaacag ccaaatggag gctatgtgga gttggcattg catttaccgg cagtgcggga |
| 2341 | ggaatttctg agtggccatc ccaaggtcta ggtggaggtg gggcatggta tttgagacat |
| 2401 | tccaaaacga aggcctctga aggacccttc agaggtggct ctggaatgac atgtgtcaag |
| 2461 | ctgcttggac ctcgtgcttt aagtgcctac attatctaac tgtgctcaag aggttctcga |
| 2521 | ctggaggacc acactcaagc cgacttatgc ccaccatccc acctctggat aattttgcat |
| 2581 | aaaattggat tagcctggag caggttggga gccaaatgtg gcatttgtga tcatgagatt |
| 2641 | gatgcaatga gatagaagat gtttgctacc tgaacactta ttgctttgaa actagacttg |
| 2701 | aggaaaccag ggtttatctt ttgagaactt ttggtaaggg aaaagggaac aggaaaagaa |
| 2761 | accccaaact caggccgaat gatcaagggg acccatagga aatcttgtcc agagacaaga |
| 2821 | cttcgggaag gtgtctggac attcagaaca ccaagacttg aaggtgcctt gctcaatgga |
| 2881 | agaggccagg acagagctga caaaattttg ctccccagtg aaggccacag caaccttctg |
| 2941 | cccatcctgt ctgttcatgg agagggtccc tgcctcacct ctgccatttt gggttaggag |
| 3001 | aagtcaagtt gggagcctga aatagtggtt cttggaaaaa tggatcccca gtgaaaacta |
| 3061 | gagctctaag cccattcagc ccatttcaca cctgaaaatg ttagtgatca ccacttggac |
| 3121 | cagcatcctt aagtatcaga aagccccaag caattgctgc atcttagtag ggtgagggat |
| 3181 | aagcaaaaga ggatgttcac cataacccag gaatgaagat accatcagca aagaatttca |
| 3241 | atttgttcag tctttcattt agagctagtc tttcacagta ccatctgaat acctctttga |
| 3301 | aagaaggaag actttacgta gtgtagattt gttttgtgtt gtttgaaaat attatctttg |
| 3361 | taattatttt taatatgtaa ggaatgcttg gaatatctgc tatatgtcaa ctttatgcag |
| 3421 | cttccttttg agggacaaat ttaaaacaaa caacccccca tcacaaactt aaaggattgc |
| 3481 | aagggccaga tctgttaagt ggtttcatag gagacacatc cagcaattgt gtggtcagtg |
| 3541 | gctcttttac ccaataagat acatcacagt cacatgcttg atggtttatg ttgacctaag |
| 3601 | atttattttg ttaaaatctc tctctgttgt gttcgttctt gttctgtttt gttttgtttt |
| 3661 | ttaaagtctt gctgtggtct ctttgtggca gaagtgtttc atgcatggca gcaggcctgt |
| 3721 | tgctttttta tggcgattcc cattgaaaat gtaagtaaat gtctgtggcc ttgttctctc |
| 3781 | tatggtaaag atattattca ccatgtaaaa caaaaaacaa tatttattgt attttagtat |
| 3841 | atttatataa ttatgttatt gaaaaaaatt ggcattaaaa cttaaccgca tcagaaccta |
| 3901 | ttgtaaatac aagttctatt taagtgtact aattaacata taatatatgt tttaaatata |
| 3961 | gaatttttaa tgtttttaaa tatattttca aagtacataa aaaaaaaaaa aaaaaa |
| SEQ ID NO 31: Homo sapiens BDNF transcript variant 6 nucleotide | |
| (NM_170734) | |
| 1 | agtggactta caagtccgaa gccaatgtag cttggaaaac ttgggaggcg gaattcctac |
| 61 | cgctgggaac tgaaagggtc tgcgacactc tcgggcaggc cgaacccaca tctctaccca |
| 121 | tcctgcgccc ctcttctgaa gcgccctcca gggaagttaa gagttttgac tttcggggag |
| 181 | tggttgggat gtacgtgggg gattcttgac tcgggttagt ctctggggat gcagagccgg |
| 241 | gaagaggaat ggttccacca ggtgagaaga gtgatgacca tccttttcct tactatggtt |
| 301 | atttcatact ttggttgcat gaaggctgcc cccatgaaag aagcaaacat ccgaggacaa |
| 361 | ggtggcttgg cctacccagg tgtgcggacc catgggactc tggagagcgt gaatgggccc |
| 421 | aaggcaggtt caagaggctt gacatcattg gctgacactt tcgaacacgt gatagaagag |
| 481 | ctgttggatg aggaccagaa agttcggccc aatgaagaaa acaataagga cgcagacttg |
| 541 | tacacgtcca gggtgatgct cagtagtcaa gtgcctttgg agcctcctct tctctttctg |
| 601 | ctggaggaat acaaaaatta cctagatgct gcaaacatgt ccatgagggt ccggcgccac |
| 661 | tctgaccctg cccgccgagg ggagctgagc gtgtgtgaca gtattagtga gtgggtaacg |
| 721 | gcggcagaca aaaagactgc agtggacatg tcgggcggga cggtcacagt ccttgaaaag |
| 781 | gtccctgtat caaaaggcca actgaagcaa tacttctacg agaccaagtg caatcccatg |
| 841 | ggttacacaa aagaaggctg caggggcata gacaaaaggc attggaactc ccagtgccga |
| 901 | actacccagt cgtacgtgcg ggcccttacc atggatagca aaaagagaat tggctggcga |
| 961 | ttcataagga tagacacttc ttgtgtatgt acattgacca ttaaaagggg aagatagtgg |
| 1021 | atttatgttg tatagattag attatattga gacaaaaatt atctatttgt atatatacat |
| 1081 | aacagggtaa attattcagt taagaaaaaa ataattttat gaactgcatg tataaatgaa |
| 1141 | gtttatacag tacagtggtt ctacaatcta tttattggac atgtccatga ccagaaggga |
| 1201 | aacagtcatt tgcgcacaac ttaaaaagtc tgcattacat tccttgataa tgttgtggtt |
| 1261 | tgttgccgtt gccaagaact gaaaacataa aaagttaaaa aaaataataa attgcatgct |
| 1321 | gctttaattg tgaattgata ataaactgtc ctctttcaga aaacagaaaa aaaacacaca |
| 1381 | cacacacaac aaaaatttga accaaaacat tccgtttaca ttttagacag taagtatctt |
| 1441 | cgttcttgtt agtactatat ctgttttact gcttttaact tctgatagcg ttggaattaa |
| 1501 | aacaatgtca aggtgctgtt gtcattgctt tactggctta ggggatgggg gatggggggt |
| 1561 | atatttttgt ttgttttgtg tttttttttc gtttgtttgt tttgtttttt agttcccaca |
| 1621 | gggagtagag atggggaaag aattcctaca atatatattc tggctgataa aagatacatt |
| 1681 | tgtatgttgt gaagatgttt gcaatatcga tcagatgact agaaagtgaa taaaaattaa |
| 1741 | ggcaactgaa caaaaaaatg ctcacactcc acatcccgtg atgcacctcc caggccccgc |
| 1801 | tcattctttg ggcgttggtc agagtaagct gcttttgacg gaaggaccta tgtttgctca |
| 1861 | gaacacattc tttccccccc tccccctctg gtctcctctt tgttttgttt taaggaagaa |
| 1921 | aaatcagttg cgcgttctga aatattttac cactgctgtg aacaagtgaa cacattgtgt |
| 1981 | cacatcatga cactcgtata agcatggaga acagtgattt ttttttagaa cagaaaacaa |
| 2041 | caaaaaataa ccccaaaatg aagattattt tttatgagga gtgaacattt gggtaaatca |
| 2101 | tggctaagct taaaaaaaac tcatggtgag gcttaacaat gtcttgtaag caaaaggtag |
| 2161 | agccctgtat caacccagaa acacctagat cagaacagga atccacattg ccagtgacat |
| 2221 | gagactgaac agccaaatgg aggctatgtg gagttggcat tgcatttacc ggcagtgcgg |
| 2281 | gaggaatttc tgagtggcca tcccaaggtc taggtggagg tggggcatgg tatttgagac |
| 2341 | attccaaaac gaaggcctct gaaggaccct tcagaggtgg ctctggaatg acatgtgtca |
| 2401 | agctgcttgg acctcgtgct ttaagtgcct acattatcta actgtgctca agaggttctc |
| 2461 | gactggagga ccacactcaa gccgacttat gcccaccatc ccacctctgg ataattttgc |
| 2521 | ataaaattgg attagcctgg agcaggttgg gagccaaatg tggcatttgt gatcatgaga |
| 2581 | ttgatgcaat gagatagaag atgtttgcta cctgaacact tattgctttg aaactagact |
| 2641 | tgaggaaacc agggtttatc ttttgagaac ttttggtaag ggaaaaggga acaggaaaag |
| 2701 | aaaccccaaa ctcaggccga atgatcaagg ggacccatag gaaatcttgt ccagagacaa |
| 2761 | gacttcggga aggtgtctgg acattcagaa caccaagact tgaaggtgcc ttgctcaatg |
| 2821 | gaagaggcca ggacagagct gacaaaattt tgctccccag tgaaggccac agcaaccttc |
| 2881 | tgcccatcct gtctgttcat ggagagggtc cctgcctcac ctctgccatt ttgggttagg |
| 2941 | agaagtcaag ttgggagcct gaaatagtgg ttcttggaaa aatggatccc cagtgaaaac |
| 3001 | tagagctcta agcccattca gcccatttca cacctgaaaa tgttagtgat caccacttgg |
| 3061 | accagcatcc ttaagtatca gaaagcccca agcaattgct gcatcttagt agggtgaggg |
| 3121 | ataagcaaaa gaggatgttc accataaccc aggaatgaag ataccatcag caaagaattt |
| 3181 | caatttgttc agtctttcat ttagagctag tctttcacag taccatctga atacctcttt |
| 3241 | gaaagaagga agactttacg tagtgtagat ttgttttgtg ttgtttgaaa atattatctt |
| 3301 | tgtaattatt tttaatatgt aaggaatgct tggaatatct gctatatgtc aactttatgc |
| 3361 | agcttccttt tgagggacaa atttaaaaca aacaaccccc catcacaaac ttaaaggatt |
| 3421 | gcaagggcca gatctgttaa gtggtttcat aggagacaca tccagcaatt gtgtggtcag |
| 3481 | tggctctttt acccaataag atacatcaca gtcacatgct tgatggttta tgttgaccta |
| 3541 | agatttattt tgttaaaatc tctctctgtt gtgttcgttc ttgttctgtt ttgttttgtt |
| 3601 | ttttaaagtc ttgctgtggt ctctttgtgg cagaagtgtt tcatgcatgg cagcaggcct |
| 3661 | gttgcttttt tatggcgatt cccattgaaa atgtaagtaa atgtctgtgg ccttgttctc |
| 3721 | tctatggtaa agatattatt caccatgtaa aacaaaaaac aatatttatt gtattttagt |
| 3781 | atatttatat aattatgtta ttgaaaaaaa ttggcattaa aacttaaccg catcagaacc |
| 3841 | tattgtaaat acaagttcta tttaagtgta ctaattaaca tataatatat gttttaaata |
| 3901 | tagaattttt aatgttttta aatatatttt caaagtacat aaaaaaaaaa aaaaaaaa |
| SEQ ID NO 32: Homo sapiens pre-pro-BDNF polypeptide (UniProtKB/Swiss- | |
| Prot P23560) | |
| MTILFLTMVI SYFGCMKAAP MKEANIRGQG GLAYPGVRTH GTLESVNGPK | |
| AGSRGLTSLA DTFEHVIEEL LDEDQKVRPN EENNKDADLY TSRVMLSSQV | |
| PLEPPLLFLL EEYKNYLDAA NMSMRVRRHS DPARRGELSV CDSISEWVTA | |
| ADKKTAVDMS GGTVTVLEKV PVSKGQLKQY FYETKCNPMG YTKEGCRGID | |
| KRHWNSQCRT TQSYVRALTM DSKKRIGWRF IRIDTSCVCTLTIKRGR | |
| SEQ ID NO 33: Homo sapiens pro-BDNF polypeptide (UniProtKB/Swiss-Prot | |
| P23560) | |
| AP MKEANIRGQG GLAYPGVRTH GTLESVNGPK AGSRGLTSLADTFEHVIEEL | |
| LDEDQKVRPN EENNKDADLY TSRVMLSSQV PLEPPLLFLL | |
| EEYKNYLDAANMSMRVRRHS DPARRGELSV CDSISEWVTA ADKKTAVDMS | |
| GGTVTVLEKV PVSKGQLKQY FYETKCNPMG YTKEGCRGID KRHWNSQCRT | |
| TQSYVRALTM DSKKRIGWRF IRIDTSCVCT LTIKRGR | |
| SEQ ID NO 34: Homo sapiens mature BDNF polypeptide (UniProtKB/Swiss- | |
| Prot P23560) | |
| HS DPARRGELSV CDSISEWVTA ADKKTAVDMS GGTVTVLEKV PVSKGQLKQY | |
| FYETKCNPMG YTKEGCRGID KRHWNSQCRT TQSYVRALTM DSKKRIGWRF | |
| IRIDTSCVCTLTIKRGR | |
| SEQ ID NO 35: Mus musculus BDNF transcript var 1 nucleotide (NM_007540) | |
| 1 | taaagcagta gccggctggt gcagaaaagc aacaagttcc ccagcggtct tcccgcccta |
| 61 | gcttgacaag gcgaagggtt tcttacctgg cgacagggaa atctcctgag ccgagctcat |
| 121 | ctttgccaga gccccaggtg tgacctgagc agtgggcaaa ggatcggcgt gcaaattgga |
| 181 | ttatttttat gggggtactc tgaaactccc tcactttctc tgggaacttt ttgtgctagg |
| 241 | gctcagtgac aggcgttgag aaagctgctt caggaaacgc ccgctatata gcagggcaat |
| 301 | tggacagtca ttggtaacct cgctcattca ttagaatcac gtaagaactc aaagggaaac |
| 361 | gtgtctctca gaatgagggc gtttgcgtaa atctataggt ttttcaacat cgatgccagt |
| 421 | tgctttgtct tctgtagtcg ccaaggtgga tgagagttga agctttgcgg atattgcgaa |
| 481 | gggttattag attcataagt cacaccaagt ggtgggcgat ccactgagca aagccgaact |
| 541 | tctcacatga tgacttcaaa caagacacat taccttcctg catctgttgg ggagacaaga |
| 601 | ttttaagaca ctgagtctcc aggacagcaa agccacaatg ttccaccagg tgagaagagt |
| 661 | gatgaccatc cttttcctta ctatggttat ttcatacttc ggttgcatga aggcggcgcc |
| 721 | catgaaagaa gtaaacgtcc acggacaagg caacttggcc tacccaggtg tgcggaccca |
| 781 | tgggactctg gagagcgtga atgggcccag ggcaggttcg agaggtctga cgacgacatc |
| 841 | actggctgac acttttgagc acgtcatcga agagctgctg gatgaggacc agaaggttcg |
| 901 | gcccaacgaa gaaaaccata aggacgcgga cttgtacact tcccgggtga tgctcagcag |
| 961 | tcaagtgcct ttggagcctc ctctactctt tctgctggag gaatacaaaa attacctgga |
| 1021 | tgccgcaaac atgtctatga gggttcggcg ccactccgac cctgcccgcc gtggggagct |
| 1081 | gagcgtgtgt gacagtatta gcgagtgggt cacagcggca gataaaaaga ctgcagtgga |
| 1141 | catgtctggc gggacggtca cagtcctaga gaaagtcccg gtatccaaag gccaactgaa |
| 1201 | gcagtatttc tacgagacca agtgtaatcc catgggttac accaaggaag gctgcagggg |
| 1261 | catagacaaa aggcactgga actcgcaatg ccgaactacc caatcgtatg ttcgggccct |
| 1321 | tactatggat agcaaaaaga gaattggctg gcgattcata aggatagaca cttcctgtgt |
| 1381 | atgtacactg accattaaaa ggggaagata gtggatttat gttgtataga ttatattgag |
| 1441 | acaaaattat ctatttgtat atatacataa cagggtaaat tattcagtta agaaaaaata |
| 1501 | attttatgaa ctgcatgtat aaatgaagtt tatacagtac agtggttcta caatctattt |
| 1561 | attggacata tccatgacct gaaaggaaac agtcatttgc gcacaacttt aaaagtctgc |
| 1621 | attacattcc tcgataatgt tgtggtttgt tgccgttgcc aagaattgaa aacaaaaagt |
| 1681 | ttaaaaaaaa taataataaa ttgcatgctg ctttaattgt gaattgataa taaactgtcc |
| 1741 | ctctttcaga aaacagatta aaaaaacaaa aaacaaaaaa aaaaaaacaa aaaacaaaaa |
| 1801 | caaaaattgg aaccaaaaca ttccgtttac attttagaca ctaagtatct tcgttcttgt |
| 1861 | tagtactctg ttttactgct ttcgacttct gatagcgttg gaattaaaac aatgtcaagg |
| 1921 | tgctgttgtc attgctttac tggcgtaagg gacggggaat gggaggggta gatttctgtt |
| 1981 | tgttttgtgt tttattttgt ttgtttgttt gttttgtttt ttagttccac ccggagtagg |
| 2041 | gatggagaaa atttcttcac tatccattct ggttgataaa gcgttacatt tgtatgttgt |
| 2101 | aaagatgttt gcaaaatcca atcagatgac tggaaaacaa ataaaaatta aggcaactga |
| 2161 | ataaaatgct cacactccac tgcccatgat gtatctccct ggtccccctc agctcactct |
| 2221 | tctggcatgg gtcagggaaa attgctttta ttggaaagac cagcatttgt tcaaagcata |
| 2281 | ctctttccct ccctcctccc attttggtcc cttctttttg ttttgtttta agaaagaaaa |
| 2341 | ttaagttgcg cgctttaaaa tattttacta ctgctacaaa cagatgaaca atgtgtgtca |
| 2401 | ttttatgaca ctcatggaaa acagtgattt ttttttaccc taaagaaaaa caaataaaaa |
| 2461 | taacccaaaa tattcttttt ttaaaaggca taaatattgg gtaaattgta atatggccta |
| 2521 | acagtgtttg cagataaaag ttattgtata cacccagata cttagataag agcagggatc |
| 2581 | cacactgcca ttgaaatagg actgaatggc cctgcggagg ctaagtggag ctgacatact |
| 2641 | atttcctggc agtgcaggag gaatttctga gtggccatcc taaggtctag gatggaggtg |
| 2701 | gggaatggta cttgagacat tcctaaagga aggctcggaa gcacccttca gagcaggctc |
| 2761 | tggaatgatg tgtcaagttg cttaggcctt ctgctttaag tgcctacatt acctaacagt |
| 2821 | gctcaagagg ttctcgattg gagaaccaca ctcaaatcca tttatagcct ccatcccatt |
| 2881 | tctaaataat tgtgtataaa gttggattaa cctggagcaa ctttggatcc aaatatggca |
| 2941 | cagcaataat gatattaatg cagcatgatg ggaaatgttt gctgtgaaga gaattgattt |
| 3001 | gctttgagct tagacttcag gaagcctagg ttttttattt ttttattttt gagacatttt |
| 3061 | ggtaaaagga aaaaaagaaa acaaacaaac aaacaaacaa aaccagaaaa agcat- |
| | caaaa |
| 3121 | ctcaggcaga atgagcaatg tctgaaaggg ctagaaaaac aagacatagc aaggtgcttt |
| 3181 | cactgtgaaa gagacaagaa cacaggagga aatattgctt cagtgaagag cacagacggc |
| 3241 | tcctgccaat ttattacaag agtcccgtct gtactttacc ctttggggtt agaagtcaag |
| 3301 | ttggaagcct gaatgaatgg acccaatgag aactagtgtt aagcccattt ccctagtcag |
| 3361 | gtttttttca agcgtgaatg tgttagtggt tactctcctg ggttcctgag catcagaaaa |
| 3421 | aaaaaaaaaa agaggcaaac aatcgcttca tcttaggagt ggaaaggaaa cagaagtgga |
| 3481 | cgtccgctgt gactcaggga gtgaagatac catcagcaaa tagtttcttt tttgttcatt |
| 3541 | cgttcctttc gagttagcct gtcttttgga ataccactga atatgctgtt tttgaaagac |
| 3601 | ttcatgtagc atagattgtt ttgtgccgtt taccaaatta acctttgtca tcgtttttta |
| 3661 | acctattcag gaatgcttgg aatatctgct ctatgttaac tttttgcagc ttcattctga |
| 3721 | gagacattag tcaaacaaac aaaaggatcc ccatcacaat cttacagtac tgcaagggcc |
| 3781 | aggtctgtta atcggcttca caggagacat cagcaattgt gtggtcagtg gctggctctc |
| 3841 | ttacccacta agatacatca tagctacatg ttggtggttt atgttgacct gagatttatt |
| 3901 | tgttaaaatc tcttcttcgt ttctgttcgt tctggttctg ttctgttctg ttctgttctg |
| 3961 | ttttggtttt aaagtcttgc tgtggtctct tgttggcaga aatgttttat gcatggcagc |
| 4021 | aggcctgttg cttttttata gtgattccca ttgaaactgt aagtaaatgt ctgtggcctt |
| 4081 | gttctctcta tggtaaagat attattcacc atgtaaaaca agaaaaaata tttattgtat |
| 4141 | tttagtatat ttatataatt atgttattga aaaaattggc attaaaactt aaccacatca |
| 4201 | gaagcctatt gtaaatacag gttctattta agtgtaccaa ttaacatata atatatgttt |
| 4261 | taaatataga atttttaatg tttttaaata tattttcaaa gt |
| SEQ ID NO 36: Mus musculus BDNF transcript var 2 nucleotide | |
| (NM_001048139) | |
| 1 | gctttggcaa agccatccac acgtgacaaa acgtaaggaa gtggaagaaa ccgtctagag |
| 61 | caatatcaag taccacttaa ttagagaata tttttttaac cttttcctcc tcctgcgccg |
| 121 | ggtgtgtgat cccggagagc agagtccatt cagcaccttg gacagagcca gcggatttgt |
| 181 | ccgaggtggt agtacttcat ccaggtattc ttttcctcgc tgtcaagcca acccggtgtc |
| 241 | gcccttaaaa agcgtctttt ccgaggttcg gctcacaccg agatcggggc tggagagaga |
| 301 | gtcagatttt ggagcggagc gtttggagag cgagccccag tttggtcccc tcattgagct |
| 361 | cgctgaagtt ggcttcctag cggtgtaggc tggaatagac tcttggcaag ctccgggttg |
| 421 | gtatactggg ttaactttgg gaaatgcaag tgtttatcac caggatctag ccaccggggt |
| 481 | ggtgtaagcc gcaaagaagt tccaccaggt gagaagagtg atgaccatcc ttttccttac |
| 541 | tatggttatt tcatacttcg gttgcatgaa ggcggcgccc atgaaagaag taaacgtcca |
| 601 | cggacaaggc aacttggcct acccaggtgt gcggacccat gggactctgg agagcgtgaa |
| 661 | tgggcccagg gcaggttcga gaggtctgac gacgacatca ctggctgaca cttttgagca |
| 721 | cgtcatcgaa gagctgctgg atgaggacca gaaggttcgg cccaacgaag aaaaccataa |
| 781 | ggacgcggac ttgtacactt cccgggtgat gctcagcagt caagtgcctt tggagcctcc |
| 841 | tctactcttt ctgctggagg aatacaaaaa ttacctggat gccgcaaaca tgtctatgag |
| 901 | ggttcggcgc cactccgacc ctgcccgccg tggggagctg agcgtgtgtg acagtattag |
| 961 | cgagtgggtc acagcggcag ataaaaagac tgcagtggac atgtctggcg ggacggtcac |
| 1021 | agtcctagag aaagtcccgg tatccaaagg ccaactgaag cagtatttct acgagaccaa |
| 1081 | gtgtaatccc atgggttaca ccaaggaagg ctgcaggggc atagacaaaa ggcactggaa |
| 1141 | ctcgcaatgc cgaactaccc aatcgtatgt tcgggccctt actatggata gcaaaaagag |
| 1201 | aattggctgg cgattcataa ggatagacac ttcctgtgta tgtacactga ccattaaaag |
| 1261 | gggaagatag tggatttatg ttgtatagat tatattgaga caaaattatc tatttgtata |
| 1321 | tatacataac agggtaaatt attcagttaa gaaaaaataa ttttatgaac tgcatgtata |
| 1381 | aatgaagttt atacagtaca gtggttctac aatctattta ttggacatat ccatgacctg |
| 1441 | aaaggaaaca gtcatttgcg cacaacttta aaagtctgca ttacattcct cgataatgtt |
| 1501 | gtggtttgtt gccgttgcca agaattgaaa acaaaaagtt taaaaaaaat aataataaat |
| 1561 | tgcatgctgc tttaattgtg aattgataat aaactgtccc tctttcagaa aacagattaa |
| 1621 | aaaaacaaaa aacaaaaaaa aaaaaacaaa aaacaaaaac aaaaattgga accaaaacat |
| 1681 | tccgtttaca ttttagacac taagtatctt cgttcttgtt agtactctgt tttactgctt |
| 1741 | tcgacttctg atagcgttgg aattaaaaca atgtcaaggt gctgttgtca ttgctttact |
| 1801 | ggcgtaaggg acggggaatg ggaggggtag atttctgttt gttttgtgtt ttattttgtt |
| 1861 | tgtttgtttg ttttgttttt tagttccacc cggagtaggg atggagaaaa tttcttcact |
| 1921 | atccattctg gttgataaag cgttacattt gtatgttgta aagatgtttg caaaatccaa |
| 1981 | tcagatgact ggaaaacaaa taaaaattaa ggcaactgaa taaaatgctc acactccact |
| 2041 | gcccatgatg tatctccctg gtccccctca gctcactctt ctggcatggg tcagggaaaa |
| 2101 | ttgcttttat tggaaagacc agcatttgtt caaagcatac tctttccctc cctcctccca |
| 2161 | ttttggtccc ttctttttgt tttgttttaa gaaagaaaat taagttgcgc gctttaaaat |
| 2221 | attttactac tgctacaaac agatgaacaa tgtgtgtcat tttatgacac tcatggaaaa |
| 2281 | cagtgatttt tttttaccct aaagaaaaac aaataaaaat aacccaaaat attctttttt |
| 2341 | taaaaggcat aaatattggg taaattgtaa tatggcctaa cagtgtttgc agataaaagt |
| 2401 | tattgtatac acccagatac ttagataaga gcagggatcc acactgccat tgaaatagga |
| 2461 | ctgaatggcc ctgcggaggc taagtggagc tgacatacta tttcctggca gtgcaggagg |
| 2521 | aatttctgag tggccatcct aaggtctagg atggaggtgg ggaatggtac ttgagacatt |
| 2581 | cctaaaggaa ggctcggaag cacccttcag agcaggctct ggaatgatgt gtcaagttgc |
| 2641 | ttaggccttc tgctttaagt gcctacatta cctaacagtg ctcaagaggt tctcgattgg |
| 2701 | agaaccacac tcaaatccat ttatagcctc catcccattt ctaaataatt gtgtataaag |
| 2761 | ttggattaac ctggagcaac tttggatcca aatatggcac agcaataatg atattaatgc |
| 2821 | agcatgatgg gaaatgtttg ctgtgaagag aattgatttg ctttgagctt agacttcagg |
| 2881 | aagcctaggt tttttatttt tttatttttg agacattttg gtaaaaggaa aaaaagaaaa |
| 2941 | caaacaaaca aacaaacaaa accagaaaaa gcatcaaaac tcaggcagaa tgagcaatgt |
| 3001 | ctgaaagggc tagaaaaaca agacatagca aggtgctttc actgtgaaag agacaagaac |
| 3061 | acaggaggaa atattgcttc agtgaagagc acagacggct cctgccaatt tattacaaga |
| 3121 | gtcccgtctg tactttaccc tttggggtta gaagtcaagt tggaagcctg aatgaatgga |
| 3181 | cccaatgaga actagtgtta agcccatttc cctagtcagg tttttttcaa gcgtgaatgt |
| 3241 | gttagtggtt actctcctgg gttcctgagc atcagaaaaa aaaaaaaaaa gaggcaaaca |
| 3301 | atcgcttcat cttaggagtg gaaaggaaac agaagtggac gtccgctgtg actcagggag |
| 3361 | tgaagatacc atcagcaaat agtttctttt ttgttcattc gttcctttcg agttagcctg |
| 3421 | tcttttggaa taccactgaa tatgctgttt ttgaaagact tcatgtagca tagattgttt |
| 3481 | tgtgccgttt accaaattaa cctttgtcat cgttttttaa cctattcagg aatgcttgga |
| 3541 | atatctgctc tatgttaact ttttgcagct tcattctgag agacattagt caaacaaaca |
| 3601 | aaaggatccc catcacaatc ttacagtact gcaagggcca ggtctgttaa tcggcttcac |
| 3661 | aggagacatc agcaattgtg tggtcagtgg ctggctctct tacccactaa gatacatcat |
| 3721 | agctacatgt tggtggttta tgttgacctg agatttattt gttaaaatct cttcttcgtt |
| 3781 | tctgttcgtt ctggttctgt tctgttctgt tctgttctgt tttggtttta aagtcttgct |
| 3841 | gtggtctctt gttggcagaa atgttttatg catggcagca ggcctgttgc ttttttatag |
| 3901 | tgattcccat tgaaactgta agtaaatgtc tgtggccttg ttctctctat ggtaaagata |
| 3961 | ttattcacca tgtaaaacaa gaaaaaatat ttattgtatt ttagtatatt tatataatta |
| 4021 | tgttattgaa aaaattggca ttaaaactta accacatcag aagcctattg taaatacagg |
| 4081 | ttctatttaa gtgtaccaat taacatataa tatatgtttt aaatatagaa tttttaatgt |
| 4141 | ttttaaatat attttcaaag t |
| SEQ ID NO 37: Mus musculus BDNF transcript var 3 nucleotide | |
| (NM_001048141): | |
| 1 | acccactttc ccattcaccg aggagaggac tgctctcgct gccgctcccc ccacccaccc |
| 61 | ccggcgagct agcatgaaat ctcccagcct ctgcctagat caaatggagc ttctcgctga |
| 121 | aggcgtgcga gtattacctc cgccatgcaa tttccactat caataattta acttctttgc |
| 181 | tgcagaacag gagtacatat cggccaccaa agactcgccc cctccccctt ttaactgaag |
| 241 | agaaggggaa atatatagta agagtctaga accttgggga ccggtcttcc ccagagcagc |
| 301 | tgccttgatg tttactttga caagtagtga ctgaaaaagt tccaccaggt gagaagagtg |
| 361 | atgaccatcc ttttccttac tatggttatt tcatacttcg gttgcatgaa ggcggcgccc |
| 421 | atgaaagaag taaacgtcca cggacaaggc aacttggcct acccaggtgt gcggacccat |
| 481 | gggactctgg agagcgtgaa tgggcccagg gcaggttcga gaggtctgac gacgacatca |
| 541 | ctggctgaca cttttgagca cgtcatcgaa gagctgctgg atgaggacca gaaggttcgg |
| 601 | cccaacgaag aaaaccataa ggacgcggac ttgtacactt cccgggtgat gctcagcagt |
| 661 | caagtgcctt tggagcctcc tctactcttt ctgctggagg aatacaaaaa ttacctggat |
| 721 | gccgcaaaca tgtctatgag ggttcggcgc cactccgacc ctgcccgccg tggggagctg |
| 781 | agcgtgtgtg acagtattag cgagtgggtc acagcggcag ataaaaagac tgcagtggac |
| 841 | atgtctggcg ggacggtcac agtcctagag aaagtcccgg tatccaaagg ccaactgaag |
| 901 | cagtatttct acgagaccaa gtgtaatccc atgggttaca ccaaggaagg ctgcaggggc |
| 961 | atagacaaaa ggcactggaa ctcgcaatgc cgaactaccc aatcgtatgt tcgggccctt |
| 1021 | actatggata gcaaaaagag aattggctgg cgattcataa ggatagacac ttcctgtgta |
| 1081 | tgtacactga ccattaaaag gggaagatag tggatttatg ttgtatagat tatattgaga |
| 1141 | caaaattatc tatttgtata tatacataac agggtaaatt attcagttaa gaaaaaataa |
| 1201 | ttttatgaac tgcatgtata aatgaagttt atacagtaca gtggttctac aatctattta |
| 1261 | ttggacatat ccatgacctg aaaggaaaca gtcatttgcg cacaacttta aaagtctgca |
| 1321 | ttacattcct cgataatgtt gtggtttgtt gccgttgcca agaattgaaa acaaaaagtt |
| 1381 | taaaaaaaat aataataaat tgcatgctgc tttaattgtg aattgataat aaactgtccc |
| 1441 | tctttcagaa aacagattaa aaaaacaaaa aacaaaaaaa aaaaaacaaa aaacaaaaac |
| 1501 | aaaaattgga accaaaacat tccgtttaca ttttagacac taagtatctt cgttcttgtt |
| 1561 | agtactctgt tttactgctt tcgacttctg atagcgttgg aattaaaaca atgtcaaggt |
| 1621 | gctgttgtca ttgctttact ggcgtaaggg acggggaatg ggaggggtag atttctgttt |
| 1681 | gttttgtgtt ttattttgtt tgtttgtttg ttttgttttt tagttccacc cggagtaggg |
| 1741 | atggagaaaa tttcttcact atccattctg gttgataaag cgttacattt gtatgttgta |
| 1801 | aagatgtttg caaaatccaa tcagatgact ggaaaacaaa taaaaattaa ggcaactgaa |
| 1861 | taaaatgctc acactccact gcccatgatg tatctccctg gtccccctca gctcactctt |
| 1921 | ctggcatggg tcagggaaaa ttgcttttat tggaaagacc agcatttgtt caaagcatac |
| 1981 | tctttccctc cctcctccca ttttggtccc ttctttttgt tttgttttaa gaaagaaaat |
| 2041 | taagttgcgc gctttaaaat attttactac tgctacaaac agatgaacaa tgtgtgtcat |
| 2101 | tttatgacac tcatggaaaa cagtgatttt tttttaccct aaagaaaaac aaataaaaat |
| 2161 | aacccaaaat attctttttt taaaaggcat aaatattggg taaattgtaa tatggcctaa |
| 2221 | cagtgtttgc agataaaagt tattgtatac acccagatac ttagataaga gcagggatcc |
| 2281 | acactgccat tgaaatagga ctgaatggcc ctgcggaggc taagtggagc tgacatacta |
| 2341 | tttcctggca gtgcaggagg aatttctgag tggccatcct aaggtctagg atggaggtgg |
| 2401 | ggaatggtac ttgagacatt cctaaaggaa ggctcggaag cacccttcag agcaggctct |
| 2461 | ggaatgatgt gtcaagttgc ttaggccttc tgctttaagt gcctacatta cctaacagtg |
| 2521 | ctcaagaggt tctcgattgg agaaccacac tcaaatccat ttatagcctc catcccattt |
| 2581 | ctaaataatt gtgtataaag ttggattaac ctggagcaac tttggatcca aatatggcac |
| 2641 | agcaataatg atattaatgc agcatgatgg gaaatgtttg ctgtgaagag aattgatttg |
| 2701 | ctttgagctt agacttcagg aagcctaggt tttttatttt tttatttttg agacattttg |
| 2761 | gtaaaaggaa aaaaagaaaa caaacaaaca aacaaacaaa accagaaaaa gcat- |
| | caaaac |
| 2821 | tcaggcagaa tgagcaatgt ctgaaagggc tagaaaaaca agacatagca aggtgctttc |
| 2881 | actgtgaaag agacaagaac acaggaggaa atattgcttc agtgaagagc acagacggct |
| 2941 | cctgccaatt tattacaaga gtcccgtctg tactttaccc tttggggtta gaagtcaagt |
| 3001 | tggaagcctg aatgaatgga cccaatgaga actagtgtta agcccatttc cctagtcagg |
| 3061 | tttttttcaa gcgtgaatgt gttagtggtt actctcctgg gttcctgagc atcagaaaaa |
| 3121 | aaaaaaaaaa gaggcaaaca atcgcttcat cttaggagtg gaaaggaaac agaagtggac |
| 3181 | gtccgctgtg actcagggag tgaagatacc atcagcaaat agtttctttt ttgttcattc |
| 3241 | gttcctttcg agttagcctg tcttttggaa taccactgaa tatgctgttt ttgaaagact |
| 3301 | tcatgtagca tagattgttt tgtgccgttt accaaattaa cctttgtcat cgttttttaa |
| 3361 | cctattcagg aatgcttgga atatctgctc tatgttaact ttttgcagct tcattctgag |
| 3421 | agacattagt caaacaaaca aaaggatccc catcacaatc ttacagtact gcaagggcca |
| 3481 | ggtctgttaa tcggcttcac aggagacatc agcaattgtg tggtcagtgg ctggctctct |
| 3541 | tacccactaa gatacatcat agctacatgt tggtggttta tgttgacctg agatttattt |
| 3601 | gttaaaatct cttcttcgtt tctgttcgtt ctggttctgt tctgttctgt tctgttctgt |
| 3661 | tttggtttta aagtcttgct gtggtctctt gttggcagaa atgttttatg catggcagca |
| 3721 | ggcctgttgc ttttttatag tgattcccat tgaaactgta agtaaatgtc tgtggccttg |
| 3781 | ttctctctat ggtaaagata ttattcacca tgtaaaacaa gaaaaaatat ttattgtatt |
| 3841 | ttagtatatt tatataatta tgttattgaa aaaattggca ttaaaactta accacatcag |
| 3901 | aagcctattg taaatacagg ttctatttaa gtgtaccaat taacatataa tatatgtttt |
| 3961 | aaatatagaa tttttaatgt ttttaaatat attttcaaag t |
| SEQ ID NO 38: Mus musculus BDNF transcript var 4 nucleotide | |
| (NM_001048142): | |
| 1 | ccaatcgaag ctcaaccgaa gagctaaata atgtctgacc ccagtgcctg gctctggctg |
| 61 | agctctgggt gcccgtcgct gctgccgtgc cggggcgcac ccgctggctg gctgtcgcac |
| 121 | ggttcccagt gcgcccggga ctcccgggct tggagaagga aaccgcctgg ggcggcgcgc |
| 181 | cacctccgcc tgggaggctt tgatgagacc cggttccttc aactgccacc actgccttgg |
| 241 | ggcagacgag aaagcgcacg gggcccaggg cagggcgcag ggaccagaag cgtgacaaca |
| 301 | atgtgactcc actgccgggg atccgagagc tttgtgtgga ccctgagttc caccaggtga |
| 361 | gaagagtgat gaccatcctt ttccttacta tggttatttc atacttcggt tgcatgaagg |
| 421 | cggcgcccat gaaagaagta aacgtccacg gacaaggcaa cttggcctac ccaggtgtgc |
| 481 | ggacccatgg gactctggag agcgtgaatg ggcccagggc aggttcgaga ggtctgacga |
| 541 | cgacatcact ggctgacact tttgagcacg tcatcgaaga gctgctggat gaggaccaga |
| 601 | aggttcggcc caacgaagaa aaccataagg acgcggactt gtacacttcc cgggtgatgc |
| 661 | tcagcagtca agtgcctttg gagcctcctc tactctttct gctggaggaa tacaaaaatt |
| 721 | acctggatgc cgcaaacatg tctatgaggg ttcggcgcca ctccgaccct gcccgccgtg |
| 781 | gggagctgag cgtgtgtgac agtattagcg agtgggtcac agcggcagat aaaaagactg |
| 841 | cagtggacat gtctggcggg acggtcacag tcctagagaa agtcccggta tccaaaggcc |
| 901 | aactgaagca gtatttctac gagaccaagt gtaatcccat gggttacacc aaggaaggct |
| 961 | gcaggggcat agacaaaagg cactggaact cgcaatgccg aactacccaa tcgtatgttc |
| 1021 | gggcccttac tatggatagc aaaaagagaa ttggctggcg attcataagg atagacactt |
| 1081 | cctgtgtatg tacactgacc attaaaaggg gaagatagtg gatttatgtt gtatagatta |
| 1141 | tattgagaca aaattatcta tttgtatata tacataacag ggtaaattat tcagttaaga |
| 1201 | aaaaataatt ttatgaactg catgtataaa tgaagtttat acagtacagt ggttctacaa |
| 1261 | tctatttatt ggacatatcc atgacctgaa aggaaacagt catttgcgca caactttaaa |
| 1321 | agtctgcatt acattcctcg ataatgttgt ggtttgttgc cgttgccaag aattgaaaac |
| 1381 | aaaaagttta aaaaaaataa taataaattg catgctgctt taattgtgaa ttgataataa |
| 1441 | actgtccctc tttcagaaaa cagattaaaa aaacaaaaaa caaaaaaaaa aaaacaaaaa |
| 1501 | acaaaaacaa aaattggaac caaaacattc cgtttacatt ttagacacta agtatcttcg |
| 1561 | ttcttgttag tactctgttt tactgctttc gacttctgat agcgttggaa ttaaaacaat |
| 1621 | gtcaaggtgc tgttgtcatt gctttactgg cgtaagggac ggggaatggg aggggtagat |
| 1681 | ttctgtttgt tttgtgtttt attttgtttg tttgtttgtt ttgtttttta gttccacccg |
| 1741 | gagtagggat ggagaaaatt tcttcactat ccattctggt tgataaagcg ttacatttgt |
| 1801 | atgttgtaaa gatgtttgca aaatccaatc agatgactgg aaaacaaata aaaattaagg |
| 1861 | caactgaata aaatgctcac actccactgc ccatgatgta tctccctggt ccccctcagc |
| 1921 | tcactcttct ggcatgggtc agggaaaatt gcttttattg gaaagaccag catttgttca |
| 1981 | aagcatactc tttccctccc tcctcccatt ttggtccctt ctttttgttt tgttttaaga |
| 2041 | aagaaaatta agttgcgcgc tttaaaatat tttactactg ctacaaacag atgaacaatg |
| 2101 | tgtgtcattt tatgacactc atggaaaaca gtgatttttt tttaccctaa agaaaaacaa |
| 2161 | ataaaaataa cccaaaatat tcttttttta aaaggcataa atattgggta aattgtaata |
| 2221 | tggcctaaca gtgtttgcag ataaaagtta ttgtatacac ccagatactt agataagagc |
| 2281 | agggatccac actgccattg aaataggact gaatggccct gcggaggcta agtggagctg |
| 2341 | acatactatt tcctggcagt gcaggaggaa tttctgagtg gccatcctaa ggtctaggat |
| 2401 | ggaggtgggg aatggtactt gagacattcc taaaggaagg ctcggaagca cccttcagag |
| 2461 | caggctctgg aatgatgtgt caagttgctt aggccttctg ctttaagtgc ctacattacc |
| 2521 | taacagtgct caagaggttc tcgattggag aaccacactc aaatccattt atagcctcca |
| 2581 | tcccatttct aaataattgt gtataaagtt ggattaacct ggagcaactt tggatccaaa |
| 2641 | tatggcacag caataatgat attaatgcag catgatggga aatgtttgct gtgaagagaa |
| 2701 | ttgatttgct ttgagcttag acttcaggaa gcctaggttt tttatttttt tatttttgag |
| 2761 | acattttggt aaaaggaaaa aaagaaaaca aacaaacaaa caaacaaaac cagaaaaagc |
| 2821 | atcaaaactc aggcagaatg agcaatgtct gaaagggcta gaaaaacaag acatagcaag |
| 2881 | gtgctttcac tgtgaaagag acaagaacac aggaggaaat attgcttcag tgaagagcac |
| 2941 | agacggctcc tgccaattta ttacaagagt cccgtctgta ctttaccctt tggggttaga |
| 3001 | agtcaagttg gaagcctgaa tgaatggacc caatgagaac tagtgttaag cccatttccc |
| 3061 | tagtcaggtt tttttcaagc gtgaatgtgt tagtggttac tctcctgggt tcctgagcat |
| 3121 | cagaaaaaaa aaaaaaaaga ggcaaacaat cgcttcatct taggagtgga aaggaaacag |
| 3181 | aagtggacgt ccgctgtgac tcagggagtg aagataccat cagcaaatag tttctttttt |
| 3241 | gttcattcgt tcctttcgag ttagcctgtc ttttggaata ccactgaata tgctgttttt |
| 3301 | gaaagacttc atgtagcata gattgttttg tgccgtttac caaattaacc tttgtcatcg |
| 3361 | ttttttaacc tattcaggaa tgcttggaat atctgctcta tgttaacttt ttgcagcttc |
| 3421 | attctgagag acattagtca aacaaacaaa aggatcccca tcacaatctt acagtactgc |
| 3481 | aagggccagg tctgttaatc ggcttcacag gagacatcag caattgtgtg gtcagtggct |
| 3541 | ggctctctta cccactaaga tacatcatag ctacatgttg gtggtttatg ttgacctgag |
| 3601 | atttatttgt taaaatctct tcttcgtttc tgttcgttct ggttctgttc tgttctgttc |
| 3661 | tgttctgttt tggttttaaa gtcttgctgt ggtctcttgt tggcagaaat gttttatgca |
| 3721 | tggcagcagg cctgttgctt ttttatagtg attcccattg aaactgtaag taaatgtctg |
| 3781 | tggccttgtt ctctctatgg taaagatatt attcaccatg taaaacaaga aaaaatattt |
| 3841 | attgtatttt agtatattta tataattatg ttattgaaaa aattggcatt aaaacttaac |
| 3901 | cacatcagaa gcctattgta aatacaggtt ctatttaagt gtaccaatta acatataata |
| 3961 | tatgttttaa atatagaatt tttaatgttt ttaaatatat tttcaaagt |
| SEQ ID NO 39: Mus musculus pre-pro BNDF polypeptide (Swiss-Prot P21237) | |
| MTILFLTMVI SYFGCMKAAP MKEVNVHGQG NLAYPGVRTH GTLESVNGPR | |
| AGSRGLTTTS LADTFEHVIE ELLDEDQKVR PNEENHKDAD LYTSRVMLSS | |
| QVPLEPPLLF LLEEYKNYLD AANMSMRVRR HSDPARRGEL SVCDSISEWV | |
| TAADKKTAVD MSGGTVTVLE KVPVSKGQLK QYFYETKCNP MGYTKEGCRG | |
| IDKRHWNSQC RTTQSYVRAL TMDSKKRIGW RFIRIDTSCV CTLTIKRGR | |
| SEQ ID NO 40: Mus musculus pro-BNDF polypeptide (Swiss-Prot P21237) | |
| AP MKEVNVHGQG NLAYPGVRTH GTLESVNGPR AGSRGLTTTS LADTFEHVIE | |
| ELLDEDQKVR PNEENHKDAD LYTSRVMLSS QVPLEPPLLF LLEEYKNYLD | |
| AANMSMRVRR HSDPARRGEL SVCDSISEWV TAADKKTAVD MSGGTVTVLE | |
| KVPVSKGQLK QYFYETKCNP MGYTKEGCRG IDKRHWNSQC RTTQSYVRAL | |
| TMDSKKRIGW CTLTIKRGR | |
| SEQ ID NO 41: Mus musculus mature BNDF polypeptide (Swiss-Prot P21237) | |
| HSDPARRGELSVCDSISEWVTAADKKTAVDMSGGTVTVLEKVPVSKGQLKQYFYE | |
| TKCNPMGYTKEGCRGIDKRHWNSQCRTTQSYVRALTMDSKKRIGWRFIRIDTSCV | |
| CTLTIKRGR | |
| SEQ ID NO 42: Rattus Norvegicus BDNF nucleotide (NM_012513) | |
| 1 | taaagcggta gccggctggt gcaggaaagc aacaagttcc ccagcggtct tcccgcccta |
| 61 | gcctgacaag gcgaaggttt tcttacctgg cgacagggaa atctcctgag ccgagctcat |
| 121 | ctttgccaca gccccaggtg tgacctgagc agtgggcaaa ggagcggcgt gcaaattgga |
| 181 | ttatttgtat gggggtactc tgaaactccc tcactttttc tgggaacttt ttgtgctagg |
| 241 | gcgcagtgac aggcgttgag aaagctgctt caggaaacgc ccgctatata gcagggcagt |
| 301 | tggacagtca ttggtaacct cgctcattca ttagaatcac gtaagaactc aaagggaaac |
| 361 | gtgtctctca gaatgagggc gtttgcgtaa atctataggt ttttcaacat cgatgccagt |
| 421 | tgctttgtct tctgtaatcg ccaaggtgga tgagagttga agcttgcgga tattgcaaag |
| 481 | ggttattaga ttcataagtc acaccaagtg gtgggcgatc cactgagcaa agccgaactt |
| 541 | ctcacatgat gacttcaaac aagacacatt accttccagc atctgttggg gagacgagat |
| 601 | tttaagacac tgagtctcca ggacagcaaa gccacaatgt tccaccaggt gagaagagtg |
| 661 | atgaccatcc ttttccttac tatggttatt tcatacttcg gttgcatgaa ggctgcgccc |
| 721 | atgaaagaag caaacgtcca cggacaaggc aacttggcct acccagctgt gcggacccat |
| 781 | gggactctgg agagcgtgaa tgggcccagg gcaggttcga gaggtctgac gacgacgtcc |
| 841 | ctggctgaca cttttgagca cgtgatcgaa gagctgctgg atgaggacca gaaggttcgg |
| 901 | cccaacgaag aaaaccataa ggacgcggac ttgtacactt cccgggtgat gctcagcagt |
| 961 | caagtgcctt tggagcctcc tctgctcttt ctgctggagg aatacaaaaa ttacctggat |
| 1021 | gccgcaaaca tgtctatgag ggttcggcgc cactccgacc ccgcccgccg tggggagctg |
| 1081 | agcgtgtgtg acagtattag cgagtgggtc acagcggcag ataaaaagac tgcagtggac |
| 1141 | atgtccggtg ggacggtcac agtcctggag aaagtcccgg tatcaaaagg ccaactgaag |
| 1201 | caatatttct acgagaccaa gtgtaatccc atgggttaca cgaaggaagg ctgcaggggc |
| 1261 | atagacaaaa ggcactggaa ctcgcaatgc cgaactaccc aatcgtatgt tcgggccctt |
| 1321 | actatggata gcaaaaagag aattggctgg cggttcataa ggatagacac ttcctgtgta |
| 1381 | tgtacactga ccattaaaag gggaagatag tggatttatg ttgtatagat tatattgaga |
| 1441 | caaaaattat ctatttgtat atatacataa cagggtaaat tattcagtta agaaaaagat |
| 1501 | aattttatga actgcatgta taaatgaagt ttatacagta cagtggttct acaatctatt |
| 1561 | tattggacat atccatgacc agaaagaaac agtcatttgc gcacaacttt aaaagtctgc |
| 1621 | attacattcc tcgataatgt tgtggtttgt tgccgttgcc aagaattgaa aacaaaaagt |
| 1681 | taaaaaaaat aataaattgc atgctgcttt aattgtgaat tgataataaa ctgtccctct |
| 1741 | ttcagaaaac agacaaaaaa acaaaaaaca aaaaaaagca aaaacaaaaa tttgaaccaa |
| 1801 | aacattccgt ttacatttta gacactaagt atcttcgttc ttgttagtac tctgttctac |
| 1861 | tgctttcaac ttctcatagc gttggaatta aaactatgtc aaggtgctgt tgtcattgct |
| 1921 | ttactggctt aggggatggg gaacgggagg ggtagatttc tgtttgtttt gtgttttatt |
| 1981 | tcgtttgttt gtttgttttg tttttttagt tccacccgga gtagggatgg agaaaatttc |
| 2041 | ttcactctcc attctggttg ataaagcgtt acatttgtat gttgtaaaaa atgtttgcaa |
| 2101 | aatccaatca gatgactgga aaacgaataa aaattaaggc aactgaataa aatgctcaca |
| 2161 | caacactgcc catgatgtat ctccctggtc ccccaggtca ctcttctggc atgggtcagg |
| 2221 | gaaagctgct tttattggaa agaccagcat ttgtttaaag cacattcttt ccctccctcc |
| 2281 | tcccattttg gtccctttct tttttgtttt gttttaagaa agaaaattaa gttgcgcgct |
| 2341 | ttgaaatatt ttatcactgc tgtgaacaga tgaacaatgt gtgtcatttc atgacactcg |
| 2401 | tggaaaacag tgattttttt tttatttttt gccctaagga gaaacaagta agaataaccg |
| 2461 | aaaatgttct ttttttttaa aggcataaac agtggataag ttataatatg gcctaacaat |
| 2521 | gtttgcagat aaaagatatt gcatacagcc agatactaga gcagggatcc acactgccac |
| 2581 | tgaaatgcga ctgaatggcc ctgtggaggc taagtggagc tgacatacta tttcctggca |
| 2641 | gagcaggagg aatttctgag tggccatcct gaggtctaga tggaggtggg gaatggtact |
| 2701 | tgagacattc ctaaaggaag gctcggaagc acccttcaga gcaggctctg gaatgatgtg |
| 2761 | tcaagtttct taggccttct gctttaagtg cctacgttac ctaacagtgc tcaagaggtt |
| 2821 | ctcaattgga gaaccacact caaatccatt tatggcctcc atcccatttt aaataattat |
| 2881 | ggataaagtt ggattaacct ggagcagctt tggatccaaa tatggcatag cagtgatgct |
| 2941 | atcagtgcag catgatggga aatgtttgct gtgaagagac ttaactttct ttgcgcttag |
| 3001 | acttcaggaa gcctaggttt tatttattta tttttttgag acattttggt gaaaggaaaa |
| 3061 | aagaaagaag aaaacaaaca aacaaaacca gaaaaagcac caaaacttag gcagaatgag |
| 3121 | caatgtctgt ctgtaagggc tagaatgaca aggcatagga aggtgctttc actgtgaaag |
| 3181 | agacaagaac acaggaggaa atactgctta agtgaagagc acagaaagct cctgatagtt |
| 3241 | ctgtccattc agcacaaggg tcccttctac actttacctc ttggggttag gagaagtcaa |
| 3301 | gctggaagcc tgaatgaatg gccccaatga gaactagtgt taagcccatt tccctagtga |
| 3361 | ggttttccgc cagcgcgaat gtgttagtgg ttacctgact gggctcctgg gcatcagaaa |
| 3421 | aagaggcaaa caattgcttc atcttaggag tggaaagggt gaaacaaagt ggctgtccac |
| 3481 | tgtgactcag ggagtgaaga taccatcagc aaatagtttc tttttgttca actgttcctt |
| 3541 | tagaactagt ctgtcttctg gagtcccact gaatcccctg tttttggaag acttcacgta |
| 3601 | gcctagattg ttttgtgccg tttgacaaca ttaatctctg tcatcatttt taacctatta |
| 3661 | aggaatgctt tgaatatctg ctatatgcta actttttgca gcttcattct gagagacgtt |
| 3721 | agtcaaacaa ataaaaggag ccccatcaca atctcacggt attcgaaggg ccaggtcgat |
| 3781 | taggtggctt cataggagac cctccgcaac tgtgtggtca gtggctggct ctcataccca |
| 3841 | ctaagataca tcatagctcc atgtcggtgg tttatgttga cctgagattg atttgttaaa |
| 3901 | atctctcctc tgtttctgtt cgttctgttt ccgtcctgtt ctgttctgtt ctgttctgaa |
| 3961 | agtcttgctg tggtctcttt ttggcagaag tgtttcatgc atggcagcag gcctgatgct |
| 4021 | ttttatagtg attcccattg aaactgtaag taaatgtctg tggccttgtt ctctctatgg |
| 4081 | taaagatatt attcaccatg taaaacaaga aaaatattta ttgtatttta gtatatttat |
| 4141 | ataattatgt tattgaaaaa aattggcatt aaaacttaac cacatcagaa gcctattgta |
| 4201 | aatacaggtt ctatttaagt gtaccaatta acatataata tatgttttaa at |
| SEQ ID NO 43: Rattus Norvegicus pre-pro-BDNF polypeptide (NM_012513) | |
| MTILFLTMVI SYFGCMKAAP MKEANVHGQG NLAYPAVRTH GTLESVNGPR | |
| AGSRGLTTTSLADTFEHVIE ELLDEDQKVR PNEENHKDAD LYTSRVMLSS | |
| QVPLEPPLLF LLEEYKNYLDAANMSMRVRR HSDPARRGEL SVCDSISEWV | |
| TAADKKTAVD MSGGTVTVLE KVPVSKGQLK QYFYETKCNP MGYTKEGCRG | |
| IDKRHWNSQC RTTQSYVRAL TMDSKKRIGW RFIRIDTSCV CTLTIKRGR | |
| SEQ ID NO 44: Rattus Norvegicus pro-BDNF polypeptide (NM_012513) | |
| AP MKEANVHGQG NLAYPAVRTH GTLESVNGPR | |
| AGSRGLTTTSLADTFEHVIEELLDEDQKVR PNEENHKDAD LYTSRVMLSS | |
| QVPLEPPLLF LLEEYKNYLDAANMSMRVRR HSDPARRGEL SVCDSISEWV | |
| TAADKKTAVD MSGGTVTVLE KVPVSKGQLKQYFYETKCNP MGYTKEGCRG | |
| IDKRHWNSQC RTTQSYVRAL TMDSKKRIGW RFIRIDTSCVCTLTIKRGR | |
| SEQ ID NO 45: Rattus Norvegicus mature BDNF polypeptide (NM_012513) | |
| HSDPARRGEL SVCDSISEWV TAADKKTAVD MSGGTVTVLE KVPVSKGQLKQY- | |
| FYETKCNP MGYTKEGCRG IDKRHWNSQC RTTQSYVRAL TMDSKKRIGW | |
| RFIRIDTSCVCTLTIKRGR | |
| SEQ ID NO 46: Bos taurus BDNF nucleotide (NM_001046607) | |
| 1 | ggacgcttga actgcagctg ccaccgctgc tgtggggccg gcggcgagcg gctccgcgac |
| 61 | ggggacctgg gctgggcgca ggggccagga gcgggacgac gatgtgactc cgccgccggg |
| 121 | gacccgtgag ctttgtgtgg accccgagtt ccaccaggtg agaagagtga tgaccatcct |
| 181 | tttccttact atggttattt catacttcgg ttgcatgaag gctgccccca tgaaagaagc |
| 241 | caacctccga gcccaaggca gcttggccta cccaggtgtg cggacccatg ggactctgga |
| 301 | gagcatgaat gggcccaagg tgggttcaag aggcctgacg tcctcgtcgt cgttggctga |
| 361 | cacttttgaa cacgtgatcg aagagctgtt ggacgaggac cagaaagttc ggcccagcga |
| 421 | ggaaaacaat aaagacgcgg acatgtacac gtcccgggtg atgctcagca gtcaagtgcc |
| 481 | tttggagccc cctctcctct tcctgctcga ggaatacaaa aattacctgg atgccgcaaa |
| 541 | catgtccatg agggtccggc gccactcgga ccccgcccgc cgcggggagc tgagcgtgtg |
| 601 | tgacagcatc agcgagtggg tgaccgcagc ggataaaaag actgcagtgg acatgtcggg |
| 661 | cgggacggtc acggtccttg aaaaagtccc cgtctcaaaa ggccagctga agcagtactt |
| 721 | ctacgagacc aagtgcaatc ccatgggtta cacgaaggag ggctgcaggg gcatagacaa |
| 781 | gaggcattgg aactcccagt gccgaactac ccagtcgtac gtgcgggccc tcaccatgga |
| 841 | tagcaaaaag cgtattggct ggcggttcat acggatagac acttcttgtg tatgtacact |
| 901 | gaccattaag aggggaagat agtggcttta tgttgtatag attatattga gacaaaaatg |
| 961 | atctatttgt atatatacat aacagggtaa attattcagt taagaaaaaa aataatttta |
| 1021 | tgaactgcat gtataaatga agtttataca gtacagtggt tctacaatct atttattgga |
| 1081 | catttccatg accagaaggg aaacagtcat tttttgcgca caactttaaa aagtctgctt |
| 1141 | tacattcctc aatgttgtgg tttgttgccg ttgccaagaa ttgaaaaacg taaaaaaaaa |
| 1201 | aaaaaaa |
| SEQ ID NO 47: Bos taurus pre-pro-BDNF polypeptide (UniProtKB/Swiss-Prot | |
| Q95106) | |
| MTILFLTMVI SYFGCMKAAP MKEANLRAQG SLAYPGVRTH GTLESMNGPK | |
| VGSRGLTSSS SLADTFEHVI EELLDEDQKV RPSEENNKDA DMYTSRVMLS | |
| SQVPLEPPLL FLLEEYKNYL DAANMSMRVR RHSDPARRGE LSVCDSISEW | |
| VTAADKKTAV DMSGGTVTVL EKVPVSKGQL KQYFYETKCN PMGYTKEGCR | |
| GIDKRHWNSQ CRTTQSYVRA LTMDSKKRIG WRFIRIDTSC VCTLTIKRGR | |
| SEQ ID NO 48: Bos taurus pro-BDNF polypeptide (Swiss-Prot Q95106) | |
| AP MKEANLRAQG SLAYPGVRTH GTLESMNGPK VGSRGLTSSS SLADTFEHVI | |
| EELLDEDQKV RPSEENNKDA DMYTSRVMLS SQVPLEPPLL FLLEEYKNYL | |
| DAANMSMRVR RHSDPARRGE LSVCDSISEW VTAADKKTAV DMSGGTVTVL | |
| EKVPVSKGQL KQYFYETKCN PMGYTKEGCR GIDKRHWNSQ CRTTQSYVRA | |
| LTMDSKKRIG WRFIRIDTSC VCTLTIKRGR | |
| SEQ ID NO 49: Bos taurus mature BDNF polypeptide (Swiss-Prot Q95106) | |
| HSDPARRGELSVCDSISEWVTAADKKTAVDMSGGTVTVLEKVPVSKGQLKQYFYE | |
| TKCNPMGYTKEGCRGIDKRHWNSQCRTTQSYVRALTMDSKKRIGWRFIRIDTSCV | |
| CTLTIKRGR | |
| SEQ ID NO 50: Sus scrofa BDNF nucleotide (NP_999424) | |
| 1 | aaaccgggca ccaaagattc ccccctaccc cttctttttg accaaaggga acgtgaaaaa |
| 61 | ataatagagt ctggggattt cggggccgaa gtcttcccca gagcagctgc cttgatgttt |
| 121 | actttgacaa gtagtgactg aaaagttcca ccaggtgaga agagtgatga ccatcctttt |
| 181 | ccttactatg gttatttcat acttcggttg catgaaggct gcccccatga aagaagccaa |
| 241 | cgtccgagga caaggcagct tggcctaccc aggtgtgcgg acccatggga ctctggagag |
| 301 | cgtgaatggg cccaaggcag gttcaagagg cctgacatcg tcgtcatcgt cgtcgttggc |
| 361 | ggacactttt gaacacgtga tcgaggagct gttggacgag gaccagaaag ttcggcccaa |
| 421 | tgaggaaaac aataaggacg cggacatgta tacgtcccga gtcatgctca gcagtcaagt |
| 481 | gcctttggag cctcctcttc tctttctgct ggaggaatac aaaaattacc tggatgctgc |
| 541 | aaacatgtcc atgagggtcc ggcgccactc ggaccccgcc cgccgcgggg agctgagcgt |
| 601 | gtgcgacagc attagcgagt gggtgacggc ggcggataaa aagacggcag tggacatgtc |
| 661 | gggtggcacg gtcacggtcc tcgaaaaagt ccccgtctcg aaaggccaac tgaagcagta |
| 721 | cttctacgag accaagtgca atcctatggg gtacacaaag gagggctgca ggggcataga |
| 781 | caagaggcac tggaactccc agtgccgaac tacccagtcg tatgtgcggg ccctcaccat |
| 841 | ggatagcaaa aaacgaattg gctggcggtt cataaggata gacacttcct gtgtatgtac |
| 901 | tttgaccatt aagaggggaa gatagtggct ttatgttgta tagattatat tgagacaaaa |
| 961 | attatctatt tgtatatata cataacaggg taaattattc agttaagaaa aaaaataatt |
| 1021 | ttatgaactg catgtataaa tgaagtttat acagtacagt ggttctacaa tctatttatt |
| 1081 | ggacatttcc atgaccagag ggaaacagtc attttttgcg cacaacttta aaaaaaaagt |
| 1141 | ctgcattaca ttcctcgata atgttgtggt ttgttgccgt tgct |
| SEQ ID NO 51: Sus scrofa pre-pro-BDNF (Swiss-Prot P14082) | |
| MTILFLTMVI SYFGCMKAAP MKEANVRGQG SLAYPGVRTH GTLESVNGPK | |
| AGSRGLTSSS SSSLADTFEH VIEELLDEDQ KVRPNEENNK DADMYTSRVM | |
| LSSQVPLEPP LLFLLEEYKN YLDAANMSMR VRRHSDPARR GELSVCDSIS | |
| EWVTAADKKT AVDMSGGTVT VLEKVPVSKG QLKQYFYETK CNPMGYTKEG | |
| CRGIDKRHWN SQCRTTQSYV RALTMDSKKR IGWRFIRIDT SCVCTLTIKR GR | |
| SEQ ID NO 52: Sus scrofa pro-BDNF polypeptide (Swiss-Prot P14082) | |
| AP MKEANVRGQG SLAYPGVRTH GTLESVNGPK AGSRGLTSSS SSSLADTFEH | |
| VIEELLDEDQ KVRPNEENNK DADMYTSRVM LSSQVPLEPP LLFLLEEYKN | |
| YLDAANMSMR VRRHSDPARR GELSVCDSIS EWVTAADKKT AVDMSGGTVT | |
| VLEKVPVSKG QLKQYFYETK CNPMGYTKEG CRGIDKRHWN SQCRTTQSYV | |
| RALTMDSKKR IGWRFIRIDT SCVCTLTIKR GR | |
| SEQ ID NO 53: Sus scrofa mature BDNF polypeptide (Swiss-Prot P14082) | |
| HSDPARRGELSVCDSISEWVTAADKKTAVDMSGGTVTVLEKVPVSKGQLKQYFYE | |
| TKCNPMGYTKEGCRGIDKRHWNSQCRTTQSYVRALTMDSKKRIGWRFIRIDTSCV | |
| CTLTIKRGR | |
| SEQ ID NO 54: Xenopus laevis BDNF nucleotide (NM_001085482) | |
| 1 | caaatggagc catttaatga agagatgcca ttacgtccac catgcagctt ccactatcaa |
| 61 | taatttaaat atcactcgct gagagcccac gaagagcctg aattattttt tttttcctcc |
| 121 | cgttgtttat tgtttgtagc cttttaattt tttcccaagg aaagctggaa agaaaacttc |
| 181 | accccaacaa acaaaaaaac ccaaaaccta tattatccag agtttcagct gagatcccca |
| 241 | atacaagtgt gttgatgtgt tctttgacaa gtactagcta aaataagttc catcaggtga |
| 301 | gaagagtgat gaccatcctt ttccttacta tggttatttc atacttcagt tgcatgaaag |
| 361 | ctgcccccat gaaagaagcc agtgtcagag gacaaaatgg cctggcctat ccgggtcttc |
| 421 | ggacccatgg tactcttgag agcataggtg gtcccagtgg ctcaagagga ggtggacttc |
| 481 | cttcactgac agatactttt gagcaagtca tagaagaact cctggaagag gaacaaagca |
| 541 | taaggcaaag tgaggaaagc aaggactctg acttgtattc atctagagta atgctaagca |
| 601 | gtcaagtacc tttggagcca ccattgcttt tcctacttga ggagtacaaa aactacttgg |
| 661 | atgcggcaaa catgtccatg agggtccggc gccactctga cccagccagg cgtggagagc |
| 721 | tgagtgtgtg tgacagtatt agtgaatggg ttacagcagc aaacaagaaa actgcagtgg |
| 781 | acatgtcggg gcagacagtt actgtcctag aaaaagtccc agtatccaaa ggccaactga |
| 841 | agcaatattt ctacgagacc aaatgcaacc ctatgggtta catgaaagaa ggctgcagag |
| 901 | gcatagacaa aaggtactgg aactctcagt gccgaactac tcagtcttac gtgcgggctt |
| 961 | tcaccatgga tagcaaaaaa aaagttggtt ggcgctttat aagaatagac acttcttgtg |
| 1021 | tatgtacact gaccattaaa aggggaagat agtgaattta tcttgtatag attatattga |
| 1081 | gaagaaaata tctatttgta tatatacata acagggtaaa ttattccgta agaaaaagaa |
| 1141 | aattaatttt atggactgca tgtagaaaaa aaaaaagaag tttatacagt aaagtggttc |
| 1201 | tacaatctat ttattgaaca tatccatacc attaaaaaaa cagagtcttc tgcgcacaat |
| 1261 | gtaacctgtt tcccgctgaa gtatcttgga acccattgcc gtgcaatgtt ttcctggttc |
| 1321 | ctccagccgg ttatgggtta aagggaaaat attcccccag tttctcttcc atttcttttc |
| 1381 | tttcacatga acagtaaact atgataatgc atattgttgg gtgtagagaa gaccctttct |
| 1441 | ctagcatgta tgagcacagc cgccatattg ctgatcttga ctgaggcatc tccatagatt |
| 1501 | tcagcagacg gtgttgtttc tgccggtaaa caccttggct cagatggttc taccgaagat |
| 1561 | tggcaaacat ttaagtgttg gacttcttct agatggtgat gctctctgaa agtctatgga |
| 1621 | ggactcccag ttcccagttg tagggtttaa atgcaaatat gcagtaggct atattatcat |
| 1681 | ccttcactgt ctgtttgggg aacaagaaaa tggaagaggg tatattctcc cttaaagaat |
| 1741 | taagcctgcg ttatgttcct caataacatt gtggtttgtt gctgttgcca agaattgaaa |
| 1801 | agcataaaag gtaaaaacca agaattgcat gctgcttcag ttgtgaattg acaatatgtc |
| 1861 | ttctattcag acaaaatttg aaccaaaaca ttccgtttac atttcacaca gtatactttc |
| 1921 | ccgtcggtat tatatctgtt tactgctttt aaacttctgg tcgcgttgga attaaacaat |
| 1981 | gtcaaggtgc tgttacagct ttcactgttt taatttaatt tttattttta ttgggggttt |
| 2041 | tttttttttt gtttttttaa ctcctgaaaa aagttgcaac gagcttgtca tgaatacatg |
| 2101 | tgttctggtt aaaaaaaaaa aaaaaatttt gtatgttgta aaggtgtttg caaactcgaa |
| 2161 | tcaaactact gatcaaaaaa ataaaataaa aaaaaaaaac agcaaggcaa acaagaaaat |
| 2221 | gatgctcgtg gtacactttt gggactcgca ccggcaacca aagtgtaagc ttcatgtgta |
| 2281 | acagaggtaa ttgctttata aatctgctct ttcaacaggc ttctgtgtgc tgctttaaat |
| 2341 | gtgtatggcc atttttaggg ggggaaatgg aggtaaatgg tgtcctcaac ctaatgaaag |
| 2401 | gctgttgcac caatctatat ataaaataaa gtctgacagc tcatatataa aggaaattgt |
| 2461 | aagtcagcac cagaataatg ctggtattag tcagtatttc caatgcaaat gctgcctgaa |
| 2521 | tattgtttac ctacgtccca ttggccccgt aacagtgtcc tcaaaatgct aattggaaaa |
| 2581 | caaatcagat ctccagtgat cacagcattt ccaaggaaaa tggaaattgg caatgaaatt |
| 2641 | tgttttggaa tgaagaagcg taggagaaca tgggccggtt gtgtgtccta agtcaataaa |
| 2701 | ggagccatta acacgtgaat ggcacgtagg ctggcatctt gattaaatag cacatctgac |
| 2761 | cattaggaga tcttattctg ttgatagtga aagagagatg gaagtgtgct gtagctgaac |
| 2821 | ataaaagcca catatacctc agaaccactt ggaaggaaaa gctgtgaatg gctttaaagg |
| 2881 | gatactgtcg tctcaaattc ctccctttta tgtatataaa ttgatataat caaaagctac |
| 2941 | attacagttg tgttattcag aaggtacacc tttatcagtg cttctgttca ttctacctct |
| 3001 | gccaagcttt tgtatatctt tttagtctca ttaataatca gccacataca tatccaataa |
| 3061 | gcagtgcagt aaagattggg ccatcaaaat caagagtaat tcattctact ttgccttgtg |
| 3121 | ggtcaggaaa ctgataattt aaaaaaaaaa aattacatta catgatacaa taactactac |
| 3181 | agtgtccctt taaaggagaa ctaaacccgt gcaatgttaa gtccccactg gcccccctcc |
| 3241 | gttgaccccc cttcctgctt cccccttacc ccagaattct tttccctctt gaaaaagtga |
| 3301 | ccgcacatgc agagtgagcg cagcggagct cacgggcgcc attttattct cttcggtaat |
| 3361 | cttcttgtct tcttcctcca cttcggcaat ttccgtctct ttcagcgcat gcgcagttgt |
| 3421 | cgcataccgg aagattgctc caacagctca tgcgccatta agccgctcac ttcacgaata |
| 3481 | ttaacaaaga gaagaagatg gcgtctgtga gcttctctgc tctcacgctg ggagggggcc |
| 3541 | aacggagtgg gccagtggga acttaacatc agctttagtt ctcctttaag gccacacttt |
| 3601 | tatacttcat caagcagcct agtaccccca tgaggttctg cagtagctca ggcctaatgt |
| 3661 | ggtagaacaa atattttgtg ctgttcccaa atttgctaaa tcttaacttg cctttctatt |
| 3721 | gttcataatt ggcttcaggc tccattttag ggttgggcat gaatttgtga aattagaaga |
| 3781 | atcagacggg aaaccaaaaa aaacagctaa tctgaaaacc aagctattta aaggtcagct |
| 3841 | aggaaaatga tgcttaggac cttgccatca cttcttgaca gtggaaaatt caattacatc |
| 3901 | tgataagtag gataatgttt ggagtggcat ttaccccagc attgttcatt aaagattccc |
| 3961 | tctaacaatc tgtcagcgga aagtgaaata agacatggta gtcccttact gcaccactgt |
| 4021 | aagccttaga aggaggctca gatttgggaa tactggtcct gaaccttact ttacttgcct |
| 4081 | tagcaggagg gttggataaa gtggccatgt tttcaggtca tggtggtcat gttttaggtg |
| 4141 | ccaggtgcaa ccctgttgct gaaatgcaac tccttagtct tagtttagca acagctagag |
| 4201 | agttagtatg tgagctatca aaagttctgg gcatcttaag tcatcatcta tagtttacac |
| 4261 | caattttact aggtaaagaa gacctcatca agactaaatg agctatactg gacatagagc |
| 4321 | acaactggcc ccaaaatcca cagtataatg ccattcactg tatcaaaaat taaatgatgt |
| 4381 | aatgaagcta aggaacaaga atgtctcgcc cagcggatct agccaaagat gtgctaggag |
| 4441 | aaagctgaag gcaatgtcaa ttgaagatca atatgagtta atcacatgtc tagggaatgg |
| 4501 | acaattaaag tggaaacaac agcagctaca caatgagcgt ggatttcata ttactatcta |
| 4561 | cagtagagct gaaattctct agagtgtacc ataatatctt tgtttacttt atttaatcta |
| 4621 | gtgatgtgta tctttatcta ctgcccaaag ccagatataa tattaataac gtccaaagcc |
| 4681 | taaattgttg gttccttctc aacaagttgg ggaagccttc taatcattga acaaagcttg |
| 4741 | agttttgaga aggcatgacc ttgattagta ttggtttcac tgtttgcttt gtcatatatg |
| 4801 | ggtgtgcgca tgtgtatata tagacatata ggctcatcgg tctgtgaccg cgctcttatt |
| 4861 | tctccatgta cacgtggaca caaatatatg tataaacccc tacttgattt cttttatatg |
| 4921 | gagtatgaga aggctccctc actcgcagga gaaatataaa tatatttttt tttcaactaa |
| 4981 | cactattcag tctatgcatt tacgctagga tgatcaacga gtcatctgga tgccctaact |
| 5041 | tttgccttga acctttgcct agaaaaaaaa ggcgtgtatg tgtgtttgtc tgtaaaaaga |
| 5101 | agttaaaaaa aaattccatc agtcaatgag aagatgttct tgaagcttaa agctccctta |
| 5161 | aatatgggtt gcagctcata gacttttttg tttcctacac ccagatctac aaatctatat |
| 5221 | gataaagcag ggggagaact catgagagcc ggagaagtca agaagtggcc ttccttagct |
| 5281 | gtcagatata tagcggccat gaccctctat catgtccccc aatactgtgg tctaaaaaaa |
| 5341 | gaaatgtcct cctatatgtt tgtgtttgtc tgtatataca agtacgcaat cagtgtatac |
| 5401 | atgactttaa aaaatatgca aaactattaa gtggaaacaa tatgaggcag ttagaataga |
| 5461 | ttcagcagtg cgaggatata ttgtctgttc tgttggacgc aaagcgacgt aacgtacaaa |
| 5521 | aaaagctact acatgccact gaatatacct gtcggatact aggcttggca gttttagctg |
| 5581 | aactgagctg caacacagac ttttttttat tttatttatt ttcgttattt ttttctttgt |
| 5641 | ctttttcgat aattttgttt tgccgtggtc tgtttgtggc aaacgtatat tctgcatgtc |
| 5701 | acctgcctac cgccttgtta aaaaaagtga gacactttga aaatgtaagt tttatgtatg |
| 5761 | tgaccttgtt ctttctggaa aaaaaaaggt atatattttc acgatgtgta agaaatattt |
| 5821 | attgtatgtt aagtatattt atataattat gttattgaaa catggagatt ggcatttaaa |
| 5881 | aatggaagca tttgaaacat attgtaaata ggattacctc tatttaagtg tactacataa |
| 5941 | catataatat atgttgaaaa atagaaattt ttaatgtttt taaatatatt ttcaaaatat |
| 6001 | ataaactaaa aaaaaaaaaa aaaaaaaaaa aaaaaaaaaa aa |

SEQ ID NO 55: Xenopus laevis pre-pro-BDNF polypeptide (Swiss Prot
Q63ZM5)
MTILFLTMVI SYFSCMKAAP MKEASVRGQN GLAYPGLRTH GTLESIGGPS
GSRGGGLPSLTDTFEQVIEE LLEEEQSIRQ SEESKDSDLY SSRVMLSSQV
PLEPPLLFLL EEYKNYLDAANMSMRVRRHS DPARRGELSV CDSISEWVTA
ANKKTAVDMS GQTVTVLEKV PVSKGQLKQYFYETKCNPMG YMKEGCRGID
KRYWNSQCRT TQSYVRAFTM DSKKKVGWRF IRIDTSCVCTLTIKRGR
SEQ ID NO 56: Xenopus laevis pro-BDNF polypeptide (Swiss-Prot P25432)
SEQ ID NO 57: Xenopus laevis mature BDNF polypeptide (Swiss-Prot P25432)
RHSDPARRGELSVCDSISEWVTAANKKTAVDMSGATVTVLEKVPVSKGQLKQYFY
ETKCNPMGYMKEGCRGIEKRYWNSQCRTTQSYVRAFTMDSKKKVGWRFIRID-
TSCVCTL
SEQ ID NO 58: Chick BDNF mature polypeptide
HSDPARRGELSVCDSTSEWVTAAEKKTAVDMSGATVTVLEKVPVPKGQLKQYFYETKCNP
KGYTKEGCRGIDKRHWNSQCRTTQSYVRALTMDNKKRVGWRFIRIDTSCVCTLTIKRGR
SEQ ID NO 59: Dog BDNF mature polypeptide
HSDPARRGELSVCDSISEWVTAADKKTAVDMSGGTVTVLEKVPVSKGQLKQYFYETKCNP
MGYTKEGCRGIDKRHWNSQCRTTQSYVRALTMDSKKRIGWRFIRIDTSCVCTLTIKRGR
SEQ ID NO 60: Rhesus monkey BDNF mature polypeptide mature polypeptide
HSDPARRGELSVCDSISEWVTAADKKTAVDMSGGTVTVLEKVPVSKGQLKQYFYETKCNP
MGYTKEGCRGIDKRHWNSQCRTTQSYVRALTMDSKKRIGWRFIRIDTSCVCTL
SEQ ID NO 61: Cat BDNF mature polypeptide
HSDPARRGELSVCDGISEWVTAADKKTAVDMSGGTVTVLEKVPVSKGQLKQYFYETKCNP
MGYTKEGCRGIDKRHWNSQCRTTQSYVRALTMDSKKRIGWRFIRIDTSCVCTLTIKRGR
SEQ ID NO 62: BDNFTRUNCMUT 1
CVPVSKGQLC
SEQ ID NO 63: BDNFTRUNCMUT 2
CVCVSKGQLC
SEQ ID NO 64: BDNFTRUNCMUT 3
CVPCSKGQLC
SEQ ID NO 65: BDNFTRUNCMUT 4
CVPVCKGQLC
SEQ ID NO 66: BDNFTRUNCMUT 5
CVPVSKGQLCE
SEQ ID NO 67: BDNFTRUNCMUT 6
CVPVCKGQLCE

### EXAMPLES

### Example 1

### Identification of Novel Substances that Upregulate SorLA Protein Expression in Neurons

Primary cortical neurons were prepared from newborn mice (day 1-2 postnatal). Animals were sacrificed by decapitation. The brain cortex was dissected in HBSS (4° C.), incubated in 1 ml enzyme solution for 1 h at 37° C. while shaking at 900 rpm (solution: 2 mg cysteine, 1 mM CaCl₂, 0.5 mM EDTA in 10 ml DMEM with 25 units/ml papain; solution carbonized for 15 min and preincubated for 1 h after adding papain). Thereafter, the enzymatic reaction was stopped by incubating the tissue for 5 min at 37° C. (shaking with 900 rpm) in 1 ml stop solution (containing 25 mg albumin, 25 mg trypsin-inhibitor in 10 ml DMEM, 5% FCS, 100 U penicillin/ml, 0.1 mg streptomycin/ml). Then, cells were dissociated in 250 μl dissociation medium by pipetting the solution up and down with a 200 μl pipette several times avoiding air bubbles. The cells were collected by centrifugation for 10 min at 80×g. The pellet was resuspended in neuronal medium and plated on PDL/collagen coated plates. The cells were kept at 37° C. with 5% CO₂ in atmosphere in neuronal medium (100 ml NeurobasalA, 2 ml B27, 1 ml Glutamax 100×, 100 U penicillin/ml, 0.1 mg streptomycin/ml). The neurons were kept for seven days for differentiation before starting the experiments. The medium was changed every 3-4 days by replacing half of the old medium.

For screening of substances, primary neuronal cultures were incubated with test substances (such as neurotrophic factors, or fragments thereof) added to the cell medium. After 24 to 48 hours of incubation, the cells were harvested on ice in PBS including protease inhibitors (Complete Protease Inhibitor Cocktail, Roche) and phosphatase inhibitors (Halt Phosphatase Inhibitor Cocktail, Pierce). Cells were collected by centrifugation at 2700 g for 10 min at 4° C. The cell pellet was resuspended in RIPA buffer (50 mM Tris, 150 mM NaCl, 0,5% deoxycholate, 1% NP40, 0,1% SDS, including protease inhibitors and phosphatase inhibitors), and incubated on ice for 1 h for complete lysis. Subsequently, 50 μg cell lysate per lane was loaded onto denaturating polyacrylamide gels and subjected to standard SDS-PAGE and Western blot analysis using antibodies specific for SorLA (commercially available). As displayed in the inset in FIG. 2, the identity of substances that upregulate SorLA protein expression (such as CTGF and BDNF) were easily revealed by demonstrating a stronger immunoreactive signal for SorLA as compared to control cells which had not been treated with such substances.

### Example 2

### Identification of Novel Substances that Upregulate SorLA Gene Expression in Neurons

Primary cortical neurons were prepared from newborn mice (day 1-2 postnatal) as described in example 1 above. For screening of substances, the primary neuronal cultures were incubated with test substances (such as neurotrophic factors, or fragments thereof) added to the cell medium. After 24 to 48 hours, RNA was isolated with TRIZOL reagent (Invitrogen, USA). Cells were scraped off in 0.5 ml of TRIZOL reagent per well of a 6-well plate and incubated for 5 min at room temperature. 0.2 ml of chloroform was added to the sample per 1 ml of TRIZOL reagent. Samples were shaken by hand for 5 min. The phases were separated by centrifugation (12000×g; 10 min; 4° C.). Following centrifugation, the upper aqueous phase was transferred to a fresh tube and precipitated by adding 0.5 ml of isopropyl alcohol per 1 ml of TRIZOL. The RNA was collected by centrifugation (12000×g, 10 min, 4° C.) and washed once with 1 ml of 85% ethanol per 1 ml TRIZOL. The pellet was air-dried for 5 min, dissolved in RNase-free water, and used for generation of first strand cDNA. Generation of cDNA from RNA was done using the Superscript II Reverse Transcriptase (Invitrogen). Subsequently, quantitative reverse-transcriptase (RT) polymerase chain reaction (PCR) was used to quantify the amount of mRNA specific for SorLA in the various samples. Several quantitative RT-PCR methods are commercially available (such as TaqMan real-time PCR). The amount of SorLA mRNA was standardized to the amount of GAPDH mRNA in each sample. As shown in FIG. 2, the identity of substances that upregulate SorLA gene expression (such as CTGF and BDNF) were easily revealed by demonstrating an increased ratio of SorLA signal relative to GAPDH control signal (such as in CFTG and BDNF treated cells).

The primers to be used for quantitative RT-PCR of GAPDH and SorLA mRNA may be deduced from freely available sequence information of the respective genes and may include the following examples:

| | |
|---|---|
| GAPDHmouse 154f: | GGC-AAA-TTC-AAC-GGC-ACA-GT |
| GAPDHmouse 223r: | AGA-TGG-TGA-TGG-GCT-TCC-C |
| SORLA mouse 2391F: | TGA-ACG-CAA-CTG-CTT-GTA-TTG-G |
| SORLA mouse 2492R: | CCA-GGC-CGG-AAT-TGA-TGA-T |

### Example 3

### Testing the Effect of SorLA Upregulation on Aβ Production in Primary Neurons

Primary cortical neurons were prepared from newborn mice (day 1-2 postnatal) and treated with test substances as described in example 1 and 2 above. Test substances included those shown to upregulate SorLA mRNA and protein levels in neurons. After 24 to 48 hours incubation with test substances, such as CTGF and BDNF, the medium was harvested from the cells and used to determine Aβ levels using commercially available kits. For detection of human Aβ₄₀ the human β amyloid 1-40 Kit (BioSource, #KHB3481) may be used, for the detection of human Aβ₄₂ the Human β Amyloid 1-42 Kit (BioSource, #KHB3442). For detection of murine or rat Aβ variants, kits are commercially available as well (IBL, Hamburg, Germany). The ELISA reactions were performed according to manufacturers instructions. As illustrated in FIG. 4A, the inhibitory effect of substances upregulating SorLA expression on Aβ production could easily be scored (such as for BDNF and CTFG).

### Example 4

### Testing the Effect of SorLA Upregulation on Aβ Production in Mouse Brain in Vivo

Substances shown to upregulate SorLA gene expression in cultured neurons are applied to the brain of mice using intravenous injection, intracranial injection, or biocompatible capsule implants according to standard procedures. Daily doses are in the range of 5-15 μg (rat) and 1-2.5 μg (mouse) per animal. After 5 to 10 days of daily repeated injections, the animals are sacrificed and the brains removed. To generate protein lysates, tissues are homogenized in RIPA buffer containing protease and phosphatase inhibitors using an Ultra Turrax, incubated on ice for 1 hour, and cleared by centrifugation 15000×g for 10 min at 4° C. thereafter. Tissue supernatants are used for determination of Aβ concentrations by commercial ELISA as described under example 3. As shown in FIG. 4B, substances (such as BDNF) that upregulate SorLA gene expression in the brain in vivo will result in a decrease in Aβ production compared to buffer injected control animals.

### Example 5

### Testing the Effect of SorLA Upregulation on Neurodegenerative Disease Processes in the Mouse Brain in Vivo

Substances shown to upregulate SorLA gene expression in cultured neurons are applied to the brain of mice using intravenous injection, intracranial injection, or biocompatible capsule implants according to standard procedures. After one to four weeks of repeated injections, the animals are sacrificed and the brains removed for immunohistological analysis. Brains are fixed in 4% formalin by transcardiac perfusion, post-fixed at 4° C. for 24 hours, and infiltrated in 30% sucrose. Then, the brains are embedded in paraffin and cut into 5 μm sections. Several well-established parameters of neurodegenerative disease processes may be scored. These parameters include detection of amyloid (senile) plaques using thioflavin S staining or anti-APP immunodetection. Furthermore, quantification of neuronal cell death may be applied using DNA fragmentation testing in apoptotic nuclei with the in situ cell death detection kit (TUNEL assay; Roche Diagnostics GmbH, Germany). Also, markers of cholinergic neuronal cell loss (anti-acetylcholine transferase antiserum, Chemicon, Schwalbach, Germany), markers of reactive microglia (macrophage/microglial marker F4/80; Serotec, Dusseldorf, Germany) or of activated astrocytes (glial fibrillary acidic protein; Advanced Immunochemical Inc., Long Beach, Calif., USA) may be detected using standard immunohistology protocols. Substances that protect the brain from the neurotoxic effects of Aβ production (through activation of SorLA) will result in a reduced extent of senile plaque formation, neuronal cell death and astroglia activation.

### Example 6

### Testing the Effect of SorLA Upregulation on Cognitive Functions in Mice

A number of standard tests for evaluating cognitive function in mice are available to those skilled in the art. Mouse models of Alzheimer'disease score poorly in these tests, indicating impairment of cognitive competence as a result of neurodegeneration. Numerous mouse models of Alzheimer'disease are freely available (e.g., JAX.org) and typically include mouse line expressing a human APP transgene (either in wild type form or mutant variant found in patients with familial Alzheimer's disease).

Substances shown to upregulate SorLA gene expression in cultured neurons are applied to the brain of mice using biocompatible capsule implants according to standard procedures. After one to four months of treatment, the animals are subjected to cognitive testing according to standard procedures. Several tests are applicable of which the Morris water maze is the most commonly used. Spatial learning and memory is examined in the water maze task using a hidden platform, whereby the platform is placed 1 cm below the water surface and remained at fixed position. The water is colored opaque by white paint to preclude animals from visually recognizing the platform. Animals are trained to find the platform for four consecutive days with six trials per day and a 10 s interval between trials. The four starting points are varied daily. Swim path to the platform and latency are recorded by an automated video tracking system (San Diego Instruments). After a maximum of 40 s the animal is placed on the platform, if it did not succeed in finding it. On the fifth day, a probe trial is carried out with the platform removed. For the testing period, a 60 s the swim path is recorded and analyzed. Mouse models of Alzheimer'disease related cognitive impairment score poorly in this test. Substances that protect the brain from the neurotoxic effects of Aβ production (through activation of the protective factor SorLA) will result in improvement of learning and memory competence compared to untreated control mice.

### Example 7

### Preparation of the Pharmaceutical Composition

The pharmaceutical composition of the present invention (including but not limited to BDNF and/or CTGF and fragments and variants thereof) are either freeze-dried to be dissolved before use or as a ready to use solution so that it can be given for parenteral administration route (e.g. intravenously (I.V.), intramuscularly (I.M.) or subcutaneously (S.C.).

If the resulting developed active agent is of chemical nature a formulation for oral administration as well as a potential route is prepared e.g. for S.C. or I.M. use. The pharmaceutical composition of the present invention is either used for prophylactic purpose or given chronically for long life treatment.

Active agents (including but not limited to BDNF and/or CTGF and fragments and variants thereof) are applied in native form or may be modified as to enhance passage across the blood-brain-barrier (BBB). A number of protocols to improve BBB passage are well known to those skilled in the art. For example, coupling the active agent to peptidomimetic monoclonal antibodies against the transferring receptor (Zhang et al., Brain Research 1111 (2006) 227-229) or the insulin receptor (Boado et al., Biotechnology and Bioengineering 100 (2007) 387-396) may be applied. Both receptors are expressed at the BBB and will transfer complexes of the active agent and the antibody into the brain. Alternatively, active agent can be covalently modified with an 11 amino acid motif from HIV Tat protein that confers upon said agent the capability to actively shuttle across the BBB. The relevant sequences and the methodology to do so are well known (Dietz et al., Brain Research 1082 (2006) 61-66). The feasibility of such concepts has been well documented for several neurotrophins (such as BDNF and GDNF).

### Example 8

### Treatment of a Patient Suffering from Alzheimer's Disease with Substances Upregulating SorLA Expression in the Brain

A 64-year-old woman is diagnosed as suffering from Alzheimer's Disease (AD). The diagnosis methods used biomarker analysis and cognitive testing, both methods well known to those skilled in the art.

The neurologist decides that the patient should receive chronical treatment of the pharmaceutical composition of the present invention by repeated injections into the spinal canal (liquor). Alternatively, pharmaceutical composition may be applied by repeated intravenous injection.

### Example 9

### Treatment of a Patient Suffering from Alzheimer's Disease with Substances Upregulating SorLA Expression in the Brain

A 50-year-old man is diagnosed as suffering from Alzheimer's Disease (AD). The diagnosis methods used biomarker analysis and cognitive testing, both methods well known to those skilled in the art. As the patient is in an early stage of AD and at a relatively young age, the neurologist in charge recommends that the patient should receive a brain implant securely providing local administration of the pharmaceutical composition of the present invention.

### Example 10

### Treatment of a Patient Suffering from Alzheimer's Disease with Substances Upregulating SorLA Expression in the Brain

A 70-year-old woman is diagnosed as suffering from Alzheimer's Disease (AD). The diagnosis methods used biomarker analysis and cognitive testing, both methods well known to those skilled in the art.

The neurologist in charge decides that the patient should receive a combination of intraveneous and intramuscular treatment of the pharmaceutical composition. The treatment will be performed daily at home by the patient using a dose-adapted device comprising the pharmaceutical composition of the present Invention. Good effect is obtained and the patient is set on lifelong treatment.

1-173. (canceled)

174. A method of treating a disease resulting from formation of amyloid plaque, comprising administering to a subject in need thereof a therapeutically effective amount of at least one isolated agent capable of upregulating the Vps10p-domain receptor SorLA, wherein the agent inhibits formation of amyloid beta peptides.

175. The method of claim 174, wherein the at least one isolated agent is a polypeptide selected from the group consisting of Brain Derived Neurotrophic Factor (BDNF) and/or Connective Tissue Growth Factor (CTGF), or a biologically active fragment or variant thereof.

176. The method of claim 175, wherein the polypeptide comprises:
a) an amino acid sequence selected from the group consisting of SEQ ID NO. 5, 8, 11, 14, 17, 20, 23, 34, 41, 45, 49, 53, 57, 58, 59, 60, 61 and 62; or
b) an amino acid sequence variant of the amino acid sequence selected from the group consisting of SEQ ID NO. 5, 8, 11, 14, 17, 20, 23, 34, 41, 45, 49, 53, 57, 58, 59, 60, 61 and 62, wherein the sequence variant has at least 70% sequence identity to said SEQ ID NO. 5, 8, 11, 14, 17, 20, 23, 34, 41, 45, 49, 53, 57, 58, 59, 60, 61 or 62; or
a biologically active fragment thereof, said fragment comprising at least 50 contiguous amino acids, wherein any amino acid specified in the selected sequence is altered to a different amino acid, provided that no more than 15 of the amino acid residues in the sequence are so altered.

177. The method of claim 175, wherein the polypeptide is a naturally occurring allelic variant of the sequence selected from the group consisting of SEQ ID NO. 5, 8, 11, 14, 17, 20, 23, 34, 41, 45, 49, 53, 57, 58, 59, 60, 61 and 62.

178. The method of claim 177, wherein the allelic variant comprises an amino acid sequence that is the translation of a nucleic acid sequence differing by a single nucleotide from a nucleic acid sequence selected from the group consisting of SEQ ID NO 3, 6, 9, 12, 15, 18, 21, 26, 27, 28, 29, 30, 31, 35, 36, 37, 38, 42, 46, 50 and 54.

179. The method of claim 175, wherein the polypeptide is a variant polypeptide, wherein any amino acid is altered to provide a conservative substitution.

180. A method of treating a disease resulting from formation of amyloid plaque, comprising administering to a subject in need thereof a therapeutically effective amount of at least one isolated nucleic acid molecule having a nucleic acid sequence encoding upon expression, a polypeptide, wherein the polypeptide inhibits formation of amyloid beta peptides.

181. The method of claim 180, wherein the nucleic acid sequence encodes a polypeptide selected from the group consisting of Brain Derived Neurotrophic Factor (BDNF) and Connective Tissue Growth Factor (CTGF).

182. The method of claim 181, wherein the polypeptide comprises:
a) an amino acid sequence selected from the group consisting of SEQ ID NO. 5, 8, 11, 14, 17, 20, 23, 34, 41, 45, 49, 53, 57, 58, 59, 60, 61 and 62 or a naturally occurring precursor protein thereof; or
b) an amino acid sequence variant having at least 70% sequence identity to SEQ ID NO. 5, 8, 11, 14, 17, 20, 23, 34, 41, 45, 49, 53, 57, 58, 59, 60, 61 or 62;
or a biologically active fragment thereof, said fragment comprising at least 50 contiguous amino acids, wherein any amino acid specified in the selected sequence is altered to a different amino acid, provided that no more than 15 of the amino acid residues in the sequence are so altered.

183. The method of claim 181, wherein the nucleic acid sequence comprises a naturally occurring allelic nucleic acid variant.

184. The method of claim 182, wherein the variant polypeptide has the amino acid sequence of a naturally occurring polypeptide variant.

185. The method of claim 180, wherein the nucleic acid molecule is selected from the group consisting of SEQ ID NO. 3, 6, 9, 12, 15, 18, 21, 26, 27, 28, 29, 30, 31, 35, 36, 37, 38, 42, 46, 50 and 54.

186. The method of claim 180, wherein the nucleic acid molecule differs by a single nucleotide from a nucleic acid sequence selected from the group consisting of SEQ ID NO. 3, 6, 9, 12, 15, 18, 21, 26, 27, 28, 29, 30, 31, 35, 36, 37, 38, 42, 46, 50 and 54.

187. The method of claim 180, wherein the nucleic acid molecule comprises:
a) a nucleic acid sequence selected from the group consisting of SEQ ID NO. 3, 6, 9, 12, 15, 18, 21, 26, 27, 28, 29, 30, 31, 35, 36, 37, 38, 42, 46, 50 and 54;
b) a nucleic acid sequence having at least 70% sequence identity to a nucleic acid sequence selected from the group consisting of SEQ ID NO. 3, 6, 9, 12, 15, 18, 21, 26, 27, 28, 29, 30, 31, 35, 36, 37, 38, 42, 46, 50 and 54;
c) a nucleic acid sequence of at least 150 contiguous nucleotides of a sequence selected from the group consisting of SEQ ID NO. 3, 6, 9, 12, 15, 18, 21, 26, 27, 28, 29, 30, 31, 35, 36, 37, 38, 42, 46, 50 and 54;
c) the complement of a nucleic acid molecule capable of hybridizing with nucleic acid molecule having the nucleic acid sequence selected from the group consisting of SEQ ID NO.: 3, 6, 9, 12, 15, 18, 21, 26, 27, 28, 29, 30, 31, 35, 36, 37, 38, 42, 46, 50 and 54, under conditions of high stringency; and
d) the nucleic acid sequence of the complement of any of the above.

188. The method of claim 180, wherein the nucleic acid molecule comprises:
a. a nucleic acid sequence having at least 70%, 75%, 80%, 85%, 95%, or 98% sequence identity to a nucleic acid sequence selected from the group consisting of SEQ ID NO. 3, 6 and 9; or
b. a nucleic acid sequence selected from the group consisting of SEQ ID NO. 3, 6 and 9.

189. The method of claim 180, wherein the nucleic acid molecule comprises:
c. a nucleic acid sequence having at least 70%, 75%, 80%, 85%, 95%, or 98% sequence identity to a nucleic acid sequence selected from the group consisting of SEQ ID NO. 21, 26 and 27; or
d. a nucleic acid sequence selected from the group consisting of SEQ ID NO. 21, 26 and 27.

190. The method of claim 180, wherein the nucleic acid molecule comprises:
e. a nucleic acid sequence having at least 70%, 75%, 80%, 85%, 95%, or 98% sequence identity to a nucleic acid sequence selected from the group consisting of SEQ ID NO. 27, 28 and 29; or
f. a nucleic acid sequence selected from the group consisting of SEQ ID NO. 27, 28 and 29.

191. The method of claim 180, wherein the nucleic acid molecule comprises:
g. a nucleic acid sequence having at least 70%, 75%, 80%, 85%, 95%, or 98% sequence identity to a nucleic acid sequence selected from the group consisting of SEQ ID NO. 29, 30 and 31; or
h. a nucleic acid molecule having the sequence selected from the group consisting of SEQ ID NO. 29, 30 and 31.

192. A method of treating a disease resulting from formation of amyloid plaque, comprising administering to a subject in need thereof a therapeutically effective amount of an antibody capable of binding specifically to a receptor selected from the group consisting of TrkA, TrkB, p75^{NTR}, Low-density lipoprotein receptor-related protein/alpha2-macroglobulin receptor (LRP), αvβ3 integrin, αIIbβ3 integrin and α6β1 integrin, wherein the antibody inhibits formation of amyloid beta peptides.

193. The method of claim 19, wherein the antibody is selected from the group consisting of: polyclonal antibodies, monoclonal antibodies, humanized antibodies, single chain antibodies, and recombinant antibodies.
